SEQUENCE LISTING (417 sequences)

SEQ ID NO: 1 - DNA, 379 bp, Artificial Sequence
  Synthetic sequence nCoV-3 mAb VH
gaagtgcagttgttggagtctgggggaggcttggttcagccgggggggtccctgagactctcctgtacagcctctggattcacctttgccagctatgccatgacctgggtccgccaggctccaggcaaggggctggagtgggtctcaactattagtggtggtggtggtgacacatactccgcagactccgtgaagggccggttcaccatctccagagacaattccaagagcacgctgtatttgcaaatgaacagcctgagagccgcggacacggccctatattactgtgcgagattggaaagcgcgacgcagcccctcggctactatttctacggtatggacgtctggggccaagggactacggtcaccgtctcctcag

SEQ ID NO: 2 - DNA, 24 bp, Artificial Sequence
  Synthetic sequence nCoV-3 mAb CDRH1
ggattcacctttgccagctatgcc

SEQ ID NO: 3 - DNA, 24 bp, Artificial Sequence
  Synthetic sequence nCoV-3 mAb CDRH2
attagtggtggtggtggtgacaca

SEQ ID NO: 4 - DNA, 57 bp, Artificial Sequence
  Synthetic sequence nCoV-3 mAb CDRH3
gcgagattggaaagcgcgacgcagcccctcggctactatttctacggtatggacgtc

SEQ ID NO: 5 - protein, 126 aa, Artificial Sequence
  Synthetic sequence nCoV-3 mAb VH
EVQLLESGGGLVQPGGSLRLSCTASGFTFASYAMTWVRQAPGKGLEWVSTISGGGGDTYSADSVKGRFTISRDNSKSTLYLQMNSLRAADTALYYCARLESATQPLGYYFYGMDVWGQGTTVTVSS

SEQ ID NO: 6 - protein, 8 aa, Artificial Sequence
  Synthetic sequence nCoV-3 mAb CDRH1
GFTFASYA

SEQ ID NO: 7 - protein, 8 aa, Artificial Sequence
  Synthetic sequence nCoV-3 mAb CDRH2
ISGGGGDT

SEQ ID NO: 8 - protein, 19 aa, Artificial Sequence
  Synthetic sequence nCoV-3 mAb CDRH3
ARLESATQPLGYYFYGMDV

SEQ ID NO: 9 - DNA, 324 bp, Artificial Sequence
  Synthetic sequence nCoV-3 mAb VL
tcctatgagctgacacagccaccctcggtgtcagtgtccccaggacagacggccaggatcacctgctctggagatgcattgccaaagcaatatgctttttggtaccagcagaggccaggccaggcccctgtgttggtgatatctaaagacagtgagaggccctcagggatccctgagcgattctctggctccagctcagggacaacagtcacgttgaccatcagtggagtccaggcagaagacgaggctgactattactgtcattcagcagacatcagtgctacttcttgggttttcggcggagggaccaagctgaccgtcgtt

SEQ ID NO: 10 - DNA, 18 bp, Artificial Sequence
  Synthetic sequence nCoV-3 mAb CDRL1
gcattgccaaagcaatat

SEQ ID NO: 11 - DNA, 9 bp, Artificial Sequence
  Synthetic sequence nCoV-3 mAb CDRL2
aaagacagt

SEQ ID NO: 12 - DNA, 33 bp, Artificial Sequence
  Synthetic sequence nCoV-3 mAb CDRL3
cattcagcagacatcagtgctacttcttgggtt

SEQ ID NO: 13 - protein, 108 aa, Artificial Sequence
  Synthetic sequence nCoV-3 mAb VL
SYELTQPPSVSVSPGQTARITCSGDALPKQYAFWYQQRPGQAPVLVISKDSERPSGIPERFSGSSSGTTVTLTISGVQAEDEADYYCHSADISATSWVFGGGTKLTVV

SEQ ID NO: 14 - protein, 6 aa, Artificial Sequence
  Synthetic sequence nCoV-3 mAb CDRL1
ALPKQY

SEQ ID NO: 15 - protein, 3 aa, Artificial Sequence
  Synthetic sequence nCoV-3 mAb CDRL2
KDS

SEQ ID NO: 16 - protein, 11 aa, Artificial Sequence
  Synthetic sequence nCoV-3 mAb CDRL3
HSADISATSWV

SEQ ID NO: 17 - DNA, 376 bp, Artificial Sequence
  Synthetic sequence nCoV-10 mAb VH
caggtgcagctggtggagtctgggggaggcgtggtccagcctgggaggtccctgagactctcctgtgcagcctctggattcaccttcagtaactatggcatgcactgggtccgccaggctccaggcaaggggctggagtggctggcagttatatcatctgatggaagaattaagttctatgcagactccgtgaagggccgattcaccatgtccagagacagttccaagaacacgctgtatctgcaaatgaacagcctgagagctgaggacacggctgtgtattactgtgcgaaagatcggttccagtttgccagaagctggtacggtgactactttgactactggggccagggaacccaggtcaccgtctcctcag

SEQ ID NO: 18 - DNA, 24 bp, Artificial Sequence
  Synthetic sequence nCoV-10 mAb CDRH1
ggattcaccttcagtaactatggc

SEQ ID NO: 19 - DNA, 24 bp, Artificial Sequence
  Synthetic sequence nCoV-10 mAb CDRH2
atatcatctgatggaagaattaag

SEQ ID NO: 20 - DNA, 54 bp, Artificial Sequence
  Synthetic sequence nCoV-10 mAb CDRH3
gcgaaagatcggttccagtttgccagaagctggtacggtgactactttgactac

SEQ ID NO: 21 - protein, 125 aa, Artificial Sequence
  Synthetic sequence nCoV-10 mAb VH
QVQLVESGGGVVQPGRSLRLSCAASGFTFSNYGMHWVRQAPGKGLEWLAVISSDGRIKFYADSVKGRFTMSRDSSKNTLYLQMNSLRAEDTAVYYCAKDRFQFARSWYGDYFDYWGQGTQVTVSS

SEQ ID NO: 22 - protein, 8 aa, Artificial Sequence
  Synthetic sequence nCoV-10 mAb CDRH1
GFTFSNYG

SEQ ID NO: 23 - protein, 8 aa, Artificial Sequence
  Synthetic sequence nCoV-10 mAb CDRH2
ISSDGRIK

SEQ ID NO: 24 - protein, 18 aa, Artificial Sequence
  Synthetic sequence nCoV-10 mAb CDRH3
AKDRFQFARSWYGDYFDY

SEQ ID NO: 25 - DNA, 337 bp, Artificial Sequence
  Synthetic sequence nCoV-10 mAb VL (Vk)
gctgttgcgatgactcagtctccactctccctgcccgtcacccttggacagccggcctccatctcctgcaggtctaatcaaagcctcgtatacagtgatggaaacatctacttgaattggtttcaacagaggccaggccaatctccaatgcgcctaatttatagggtttctaaccgggactctggggtcccagacagattcagcggcagtgggtcaggcactgatttcacactgaaaatcagcagggtggaggctgaagatgttgggatttattactgcatgcaagggacacactggcctccgactttcggcggagggaccaaggtggagatcaaac

SEQ ID NO: 26 - DNA, 33 bp, Artificial Sequence
  Synthetic sequence nCoV-10 mAb CDRL1
caaagcctcgtatacagtgatggaaacatctac

SEQ ID NO: 27 - DNA, 9 bp, Artificial Sequence
  Synthetic sequence nCoV-10 mAb CDRL2
agggtttct

SEQ ID NO: 28 - DNA, 27 bp, Artificial Sequence
  Synthetic sequence nCoV-10 mAb CDRL3
atgcaagggacacactggcctccgact

SEQ ID NO: 29 - protein, 112 aa, Artificial Sequence
  Synthetic sequence nCoV-10 mAb VL (Vk)
AVAMTQSPLSLPVTLGQPASISCRSNQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 30 - protein, 11 aa, Artificial Sequence
  Synthetic sequence nCoV-10 mAb CDRL1
QSLVYSDGNIY

SEQ ID NO: 31 - protein, 3 aa, Artificial Sequence
  Synthetic sequence nCoV-10 mAb CDRL2
RVS

SEQ ID NO: 32 - protein, 9 aa, Artificial Sequence
  Synthetic sequence nCoV-10 mAb CDRL3
MQGTHWPPT

SEQ ID NO: 33 - DNA, 376 bp, Artificial Sequence; identical to SEQ ID NO: 17
  Synthetic sequence nCoV-10-v2 mAb VH

SEQ ID NO: 34 - DNA, 24 bp, Artificial Sequence
  Synthetic sequence nCoV-10-v2 mAb CDRH1
ggattcaccttcagtaactatggc

SEQ ID NO: 35 - DNA, 24 bp, Artificial Sequence
  Synthetic sequence nCoV-10-v2 mAb CDRH2
atatcatctgatggaagaattaag

SEQ ID NO: 36 - DNA, 54 bp, Artificial Sequence; identical to SEQ ID NO: 20
  Synthetic sequence nCoV-10-v2 mAb CDRH3

SEQ ID NO: 37 - protein, 125 aa, Artificial Sequence; identical to SEQ ID NO: 21
  Synthetic sequence nCoV-10-v2 mAb VH

SEQ ID NO: 38 - protein, 8 aa, Artificial Sequence
  Synthetic sequence nCoV-10-v2 mAb CDRH1
GFTFSNYG

SEQ ID NO: 39 - protein, 8 aa, Artificial Sequence
  Synthetic sequence nCoV-10-v2 mAb CDRH2
ISSDGRIK

SEQ ID NO: 40 - protein, 18 aa, Artificial Sequence
  Synthetic sequence nCoV-10-v2 mAb CDRH3
AKDRFQFARSWYGDYFDY

SEQ ID NO: 41 - DNA, 337 bp, Artificial Sequence
  Synthetic sequence nCoV-10-v2 (N to S) mAb VL (Vk)
gctgttgcgatgactcagtctccactctccctgcccgtcacccttggacagccggcctccatctcctgcaggtctagtcaaagcctcgtatacagtgatggaaacatctacttgaattggtttcaacagaggccaggccaatctccaatgcgcctaatttatagggtttctaaccgggactctggggtcccagacagattcagcggcagtgggtcaggcactgatttcacactgaaaatcagcagggtggaggctgaagatgttgggatttattactgcatgcaagggacacactggcctccgactttcggcggagggaccaaggtggagatcaaac

SEQ ID NO: 42 - DNA, 33 bp, Artificial Sequence; identical to SEQ ID NO: 26
  Synthetic sequence nCoV-10-v2 mAb CDRL1

SEQ ID NO: 43 - DNA, 9 bp, Artificial Sequence
  Synthetic sequence nCoV-10-v2 mAb CDRL2
agggtttct

SEQ ID NO: 44 - DNA, 27 bp, Artificial Sequence
  Synthetic sequence nCoV-10-v2 mAb CDRL3
atgcaagggacacactggcctccgact

SEQ ID NO: 45 - protein, 112 aa, Artificial Sequence
  Synthetic sequence nCoV-10-v2 (N to S) mAb VL (Vk)
AVAMTQSPLSLPVTLGQPASISCRSSQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 46 - protein, 11 aa, Artificial Sequence
  Synthetic sequence nCoV-10-v2 mAb CDRL1
QSLVYSDGNIY

SEQ ID NO: 47 - protein, 3 aa, Artificial Sequence
  Synthetic sequence nCoV-10-v2 mAb CDRL2
RVS

SEQ ID NO: 48 - protein, 9 aa, Artificial Sequence
  Synthetic sequence nCoV-10-v2 mAb CDRL3
MQGTHWPPT

SEQ ID NO: 49 - DNA, 370 bp, Artificial Sequence
  Synthetic sequence nCoV-17 mAb VH
caggtgcagctggtggagtcggggggaggcgtggtccagcctgggaggtccctgagactctcctgtgcagcctctggattcactttcagttcctatgctatacactgggtccgccaggctccaggcaagggtctggagtggctggcagttatatcatttgatggaagacataaatactacgcagactccgtgaagggccgattcaccatctccagagacaattccaagaacacggtgtatctgcaaatgaacagcctgagagttgaggacacggctgtctattcctgtgcgagagatattcgtcatttgatagtagtggtttccgatatggacgtctggggccaagggaccacggtcaccgtctcctcag

SEQ ID NO: 50 - DNA, 24 bp, Artificial Sequence
  Synthetic sequence nCoV-17 mAb CDRH1
ggattcactttcagttcctatgct

SEQ ID NO: 51 - DNA, 24 bp, Artificial Sequence
  Synthetic sequence nCoV-17 mAb CDRH2
atatcatttgatggaagacataaa

SEQ ID NO: 52 - DNA, 48 bp, Artificial Sequence
  Synthetic sequence nCoV-17 mAb CDRH3
gcgagagatattcgtcatttgatagtagtggtttccgatatggacgtc

SEQ ID NO: 53 - protein, 123 aa, Artificial Sequence
  Synthetic sequence nCoV-17 mAb VH
QVQLVESGGGVVQPGRSLRLSCAASGFTFSSYAIHWVRQAPGKGLEWLAVISFDGRHKYYADSVKGRFTISRDNSKNTVYLQMNSLRVEDTAVYSCARDIRHLIVVVSDMDVWGQGTTVTVSS

SEQ ID NO: 54 - protein, 8 aa, Artificial Sequence
  Synthetic sequence nCoV-17 mAb CDRH1
GFTFSSYA

SEQ ID NO: 55 - protein, 8 aa, Artificial Sequence
  Synthetic sequence nCoV-17 mAb CDRH2
ISFDGRHK

SEQ ID NO: 56 - protein, 16 aa, Artificial Sequence
  Synthetic sequence nCoV-17 mAb CDRH3
ARDIRHLIVVVSDMDV

SEQ ID NO: 57 - DNA, 337 bp, Artificial Sequence
  Synthetic sequence nCoV-17 mAb VL (Vk)
gatgttgtgatgactcagtctccactctccctgcccgtcacccttggacagccggcctccatctcctgcaggtctagtcaaagcctcgtaaacagtgatggagacacctccttgaattggtttcagcagaggccaggccaatctccaaggcgcctgatttataaggtttctaaccgggactctggggtcccagacagaatcagcggcagtgggtcaggcactgatttcacactgaaaatcagcagggtggaggctgaggatgttggggtttattactgcatgcaaggtacacactggcctccgacttttggccaggggaccaagctggagatcaaac

SEQ ID NO: 58 - DNA, 33 bp, Artificial Sequence
  Synthetic sequence nCoV-17 mAb CDRL1
caaagcctcgtaaacagtgatggagacacctcc

SEQ ID NO: 59 - DNA, 33 bp, Artificial Sequence; identical to SEQ ID NO: 58
  Synthetic sequence nCoV-17 mAb CDRL2

SEQ ID NO: 60 - DNA, 27 bp, Artificial Sequence
  Synthetic sequence nCoV-17 mAb CDRL3
atgcaaggtacacactggcctccgact

SEQ ID NO: 61 - protein, 112 aa, Artificial Sequence
  Synthetic sequence nCoV-17 mAb VL (Vk)
DVVMTQSPLSLPVTLGQPASISCRSSQSLVNSDGDTSLNWFQQRPGQSPRRLIYKVSNRDSGVPDRISGSGSGTDFTLKISRVEAEDVGVYYCMQGTHWPPTFGQGTKLEIK

SEQ ID NO: 62 - protein, 11 aa, Artificial Sequence
  Synthetic sequence nCoV-17 mAb CDRL1
QSLVNSDGDTS

SEQ ID NO: 63 - protein, 3 aa, Artificial Sequence
  Synthetic sequence nCoV-17 mAb CDRL2
KVS

SEQ ID NO: 64 - protein, 9 aa, Artificial Sequence
  Synthetic sequence nCoV-17 mAb CDRL3
MQGTHWPPT

SEQ ID NO: 65 - DNA, 376 bp, Artificial Sequence
  Synthetic sequence nCoV-6 mAb VH
caggtcaccttgaaggagtctggtcctgtgctggtgaaacccacagagaccctcacgctgacctgcaccgtctctgtgttctcactcagcaatgctagaatgggtgtgagctggatccgtcagcccccagggaaggccctggagtggcttgcacacattttttcgagtgaccaaaaatcctacagcacatctctgaagagcaggctcaccatctccaaggacacctccaaaagccaggtggtccttaccatgaccaacatggaccctgaggacacaggcacatattactgtgcacgaataaacacggcggcgtatgattatgatagtaccacctttgatatctggggccaagggacaatggtcaccgtctcttcag

SEQ ID NO: 66 - DNA, 30 bp, Artificial Sequence
  Synthetic sequence nCoV-6 mAb CDRH1
gtgttctcactcagcaatgctagaatgggt

SEQ ID NO: 67 - DNA, 21 bp, Artificial Sequence
  Synthetic sequence nCoV-6 mAb CDRH2
attttttcgagtgaccaaaaa

SEQ ID NO: 68 - DNA, 51 bp, Artificial Sequence
  Synthetic sequence nCoV-6 mAb CDRH3
gcacgaataaacacggcggcgtatgattatgatagtaccacctttgatatc

SEQ ID NO: 69 - protein, 125 aa, Artificial Sequence
  Synthetic sequence nCoV-6 mAb VH
QVTLKESGPVLVKPTETLTLTCTVSVFSLSNARMGVSWIRQPPGKALEWLAHIFSSDQKSYSTSLKSRLTISKDTSKSQVVLTMTNMDPEDTGTYYCARINTAAYDYDSTTFDIWGQGTMVTVSS

SEQ ID NO: 70 - protein, 10 aa, Artificial Sequence
  Synthetic sequence nCoV-6 mAb CDRH1
VFSLSNARMG

SEQ ID NO: 71 - protein, 7 aa, Artificial Sequence
  Synthetic sequence nCoV-6 mAb CDRH2
IFSSDQK

SEQ ID NO: 72 - protein, 17 aa, Artificial Sequence
  Synthetic sequence nCoV-6 mAb CDRH3
ARINTAAYDYDSTTFDI

SEQ ID NO: 73 - DNA, 322 bp, Artificial Sequence
  Synthetic sequence nCoV-6 mAb VL (Vk)
gacatccagatgacccagtctccatcctccctgtctgcatctgtaggagacagagtcaccatcacttgccgggcaagtcagaccattagcaactatttaaattggtatcagcagaaaccagggaaagcccctaagctcctgctctatgctgcatccagtttgcaaagtggggtcccatcaaggttcagtggcagtggatctgggacagatttcactctcaccatcagcagtctgcaacttgaagattttgcaacttactactgtcaacagagttacagtacccctcccactttcggcggagggaccaaggtggagatcaaac

SEQ ID NO: 74 - DNA, 18 bp, Artificial Sequence
  Synthetic sequence nCoV-6 mAb CDRL1
cagaccattagcaactat

SEQ ID NO: 75 - DNA, 9 bp, Artificial Sequence
  Synthetic sequence nCoV-6 mAb CDRL2
gctgcatcc

SEQ ID NO: 76 - DNA, 27 bp, Artificial Sequence
  Synthetic sequence nCoV-6 mAb CDRL3
caacagagttacagtacccctcccact

SEQ ID NO: 77 - protein, 107 aa, Artificial Sequence
  Synthetic sequence nCoV-6 mAb VL (Vk)
DIQMTQSPSSLSASVGDRVTITCRASQTISNYLNWYQQKPGKAPKLLLYAASSLQSGVPSRFSGSGSGTDFTLTISSLQLEDFATYYCQQSYSTPPTFGGGTKVEIK

SEQ ID NO: 78 - protein, 6 aa, Artificial Sequence
  Synthetic sequence nCoV-6 mAb CDRL1
QTISNY

SEQ ID NO: 79 - protein, 3 aa, Artificial Sequence
  Synthetic sequence nCoV-6 mAb CDRL2
AAS

SEQ ID NO: 80 - protein, 9 aa, Artificial Sequence
  Synthetic sequence nCoV-6 mAb CDRL3
QQSYSTPPT

SEQ ID NO: 81 - DNA, 376 bp, Artificial Sequence
  Synthetic sequence nCoV-16 mAb VH
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcatttatatcatatgatggaagtaatatacagtatgcagactccgtgcagggccgattcaccatctccagagacaattccaagaccacgttgtatctgcaaatgaacagcctgagagttgaggacacggctatgtactactgtgcgaaagatcttttcggatattgtaggagtaccagctgcgagtctcttgacgactggggccagggagccctggtcaccgtctcctcag

SEQ ID NO: 82 - DNA, 24 bp, Artificial Sequence
  Synthetic sequence nCoV-16 mAb CDRH1
ggattcaccttcagtagctatggc

SEQ ID NO: 83 - DNA, 24 bp, Artificial Sequence
  Synthetic sequence nCoV-16 mAb CDRH2
atatcatatgatggaagtaatata

SEQ ID NO: 84 - DNA, 54 bp, Artificial Sequence
  Synthetic sequence nCoV-16 mAb CDRH3
gcgaaagatcttttcggatattgtaggagtaccagctgcgagtctcttgacgac

SEQ ID NO: 85 - protein, 125 aa, Artificial Sequence
  Synthetic sequence nCoV-16 mAb VH
QVQLVESGGGVVQPGRSLRLSCAASGFTFSSYGMHWVRQAPGKGLEWVAFISYDGSNIQYADSVQGRFTISRDNSKTTLYLQMNSLRVEDTAMYYCAKDLFGYCRSTSCESLDDWGQGALVTVSS

SEQ ID NO: 86 - protein, 8 aa, Artificial Sequence
  Synthetic sequence nCoV-16 mAb CDRH1
GFTFSSYG

SEQ ID NO: 87 - protein, 8 aa, Artificial Sequence
  Synthetic sequence nCoV-16 mAb CDRH2
ISYDGSNI

SEQ ID NO: 88 - protein, 18 aa, Artificial Sequence
  Synthetic sequence nCoV-16 mAb CDRH3
AKDLFGYCRSTSCESLDD

SEQ ID NO: 89 - DNA, 322 bp, Artificial Sequence
  Synthetic sequence nCoV-16 mAb VL (Vk)
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcactggatatgggacagaattcactctcacaatcagcagcctgcagcctgaagattttgcaacttattactgtcaacagcttaataattaccctctcactttcggcggagggaccaaggtggagatcaaac

SEQ ID NO: 90 - DNA, 18 bp, Artificial Sequence
  Synthetic sequence nCoV-16 mAb CDRL1
cagggcattagcagttat

SEQ ID NO: 91 - DNA, 9 bp, Artificial Sequence
  Synthetic sequence nCoV-16 mAb CDRL2
gctgcatcc

SEQ ID NO: 92 - DNA, 27 bp, Artificial Sequence
  Synthetic sequence nCoV-16 mAb CDRL3
caacagcttaataattaccctctcact

SEQ ID NO: 93 - protein, 107 aa, Artificial Sequence
  Synthetic sequence nCoV-16 mAb VL (Vk)
DIQLTQSPSFLSASVGDRVTITCRASQGISSYLAWYQQKPGKAPKLLIYAASTLQSGVPSRFSGTGYGTEFTLTISSLQPEDFATYYCQQLNNYPLTFGGGTKVEIK

SEQ ID NO: 94 - protein, 6 aa, Artificial Sequence
  Synthetic sequence nCoV-16 mAb CDRL1
QGISSY

SEQ ID NO: 95 - protein, 3 aa, Artificial Sequence
  Synthetic sequence nCoV-16 mAb CDRL2
AAS

SEQ ID NO: 96 - protein, 9 aa, Artificial Sequence
  Synthetic sequence nCoV-16 mAb CDRL3
QQLNNYPLT

SEQ ID NO: 97 - DNA, 373 bp, Artificial Sequence
  Synthetic sequence nCoV-20 mAb VH
caggtcaccttgcgggagtctggtcctgcgctggtgaaacccacacagaccctcacactgacctgcaccttctctgggttctcactcaccactactgaaatgtgtgtgggctggatccgtcagcccccagggaaggccctggagtggcttgcactcattgattgggatgatgataaatactacagcacatctctgaggaccaggctcaccatctccaaggacacctccaaaaaccaggtggtccttaccatgaccaacatggaccctgtggacacagccacatattactgtgcacggatgttcgggcgaaagcgtctggcaccaactgggttcgacccctggggccagggaaccctggtcaccgtctcctcag

SEQ ID NO: 98 - DNA, 30 bp, Artificial Sequence
  Synthetic sequence nCoV-20mAb CDRH1
gggttctcactcaccactactgaaatgtgt

SEQ ID NO: 99 - DNA, 21 bp, Artificial Sequence
  Synthetic sequence nCoV-20 mAb CDRH2
attgattgggatgatgataaa

SEQ ID NO: 100 - DNA, 48 bp, Artificial Sequence
  Synthetic sequence nCoV-20 mAb CDRH3
gcacggatgttcgggcgaaagcgtctggcaccaactgggttcgacccc

SEQ ID NO: 101 - protein, 124 aa, Artificial Sequence
  Synthetic sequence nCoV-20 mAb VH
QVTLRESGPALVKPTQTLTLTCTFSGFSLTTTEMCVGWIRQPPGKALEWLALIDWDDDKYYSTSLRTRLTISKDTSKNQVVLTMTNMDPVDTATYYCARMFGRKRLAPTGFDPWGQGTLVTVSS

SEQ ID NO: 102 - protein, 10 aa, Artificial Sequence
  Synthetic sequence nCoV-20 mAb CDRH1
GFSLTTTEMC

SEQ ID NO: 103 - protein, 7 aa, Artificial Sequence
  Synthetic sequence nCoV-20mAb CDRH2
IDWDDDK

SEQ ID NO: 104 - protein, 16 aa, Artificial Sequence
  Synthetic sequence nCoV-20 mAb CDRH3
ARMFGRKRLAPTGFDP

SEQ ID NO: 105 - DNA, 337 bp, Artificial Sequence
  Synthetic sequence nCoV-20 mAb VL (Vk)
gatattgtgatgactcagtctccactctccctgcccgtcacccctggagagccggcctccatctcctgcaggtctagtcagagcctcctgcatagtaatggaaagaactatttggattggtacctgcagaagccagggcagtctccacagctcctgatctgtttgggttcttatcgggcctccggggtccctgacaggttcagtggcagtggatcaggcacagattttacactgaaaatcagcagagtggaggctgaagatgttggggtttattactgcatgcaagctctacaaactccctggacgttcggccaagggaccaaggtggaaatcaaac

SEQ ID NO: 106 - DNA, 33 bp, Artificial Sequence
  Synthetic sequence nCoV-20 mAb CDRL1
cagagcctcctgcatagtaatggaaagaactat

SEQ ID NO: 107 - DNA, 9 bp, Artificial Sequence
  Synthetic sequence nCoV-20 mAb CDRL2
ttgggttct

SEQ ID NO: 108 - DNA, 27 bp, Artificial Sequence
  Synthetic sequence nCoV-20 mAb CDRL3
atgcaagctctacaaactccctggacg

SEQ ID NO: 109 - protein, 112 aa, Artificial Sequence
  Synthetic sequence nCoV-20 mAb VL (Vk)
DIVMTQSPLSLPVTPGEPASISCRSSQSLLHSNGKNYLDWYLQKPGQSPQLLICLGSYRASGVPDRFSGSGSGTDFTLKISRVEAEDVGVYYCMQALQTPWTFGQGTKVEIK

SEQ ID NO: 110 - protein, 11 aa, Artificial Sequence
  Synthetic sequence nCoV-20 mAb CDRL1
QSLLHSNGKNY

SEQ ID NO: 111 - protein, 3 aa, Artificial Sequence
  Synthetic sequence nCoV-20 mAb CDRL2
LGS

SEQ ID NO: 112 - protein, 9 aa, Artificial Sequence
  Synthetic sequence nCoV-20 mAb CDRL3
MQALQTPWT

SEQ ID NO: 113 - DNA, 370 bp, Artificial Sequence
  Synthetic sequence nCoV-4 mAb VH
caggtgcaactggtggagtctgggggaggcgtggtccagcctgggaggtccctgagactctcctgtgaagcctctggattcaccctcagaaccagtagtctccactgggtccgccaggctccaggcaaggggctggagtgggtggcagttatatcaaatgatggagccactaaattctacgcagacgccgtgaagggccgattcaccatctccagagacaactccaacaacaaaatatatctgcaactgaacggcctgaaacctgaggacacggctgtctattactgtgcgagagaaacacgtcattacagccatggtttgaactggttcgacccctggggccagggaaccctggtcaacgtctcctcag

SEQ ID NO: 114 - DNA, 24 bp, Artificial Sequence
  Synthetic sequence nCoV-4 mAb CDRH1
ggattcaccctcagaaccagtagt

SEQ ID NO: 115 - DNA, 24 bp, Artificial Sequence
  Synthetic sequence nCoV-4 mAb CDRH2
atatcaaatgatggagccactaaa

SEQ ID NO: 116 - DNA, 48 bp, Artificial Sequence
  Synthetic sequence nCoV-4 mAb CDRH3
gcgagagaaacacgtcattacagccatggtttgaactggttcgacccc

SEQ ID NO: 117 - protein, 98 aa, Artificial Sequence
  Synthetic sequence nCoV-4 mAb VH
GFTLRTSSLHWVRQAPGKGLEWVAVISNDGATKFYADAVKGRFTISRDNSNNKIYLQLNGLKPEDTAVYYCARETRHYSHGLNWFDPWGQGTLVNVSS

SEQ ID NO: 118 - protein, 8 aa, Artificial Sequence
  Synthetic sequence nCoV-4 mAb CDRH1
GFTLRTSS

SEQ ID NO: 119 - protein, 8 aa, Artificial Sequence
  Synthetic sequence nCoV-4 mAb CDRH2
ISNDGATK

SEQ ID NO: 120 - protein, 16 aa, Artificial Sequence
  Synthetic sequence nCoV-4 mAb CDRH3
ARETRHYSHGLNWFDP

SEQ ID NO: 121 - DNA, 322 bp, Artificial Sequence
  Synthetic sequence nCoV-4 mAb VL (Vk)
gaaagagtgatgacgcagtctccagtcaccctgtctgtgtctccaggggaaagagccaccctctcctgcagggccagtcagagtgttagcagcgacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgaccaccttcggccaagggacacgactggacattaaac

SEQ ID NO: 122 - DNA, 18 bp, Artificial Sequence
  Synthetic sequence nCoV-4 mAb CDRL1
cagagtgttagcagcgac

SEQ ID NO: 123 - DNA, 9 bp, Artificial Sequence
  Synthetic sequence nCoV-4 mAb CDRL2
ggtgcatcc

SEQ ID NO: 124 - DNA, 27 bp, Artificial Sequence
  Synthetic sequence nCoV-4 mAb CDRL3
cagcagtataataactggccgaccacc

SEQ ID NO: 125 - protein, 107 aa, Artificial Sequence
  Synthetic sequence nCoV-4 mAb VL (Vk)
ERVMTQSPVTLSVSPGERATLSCRASQSVSSDLAWYQQKPGQAPRLLIYGASTRATGIPARFSGSGSGTEFTLTISSLQSEDFAVYYCQQYNNWPTTFGQGTRLDIK

SEQ ID NO: 126 - protein, 6 aa, Artificial Sequence
  Synthetic sequence nCoV-4 mAb CDRL1
QSVSSD

SEQ ID NO: 127 - protein, 3 aa, Artificial Sequence
  Synthetic sequence nCoV-4 mAb CDRL2
GAS

SEQ ID NO: 128 - protein, 9 aa, Artificial Sequence
  Synthetic sequence nCoV-4 mAb CDRL3
QQYNNWPTT

SEQ ID NO: 129 - DNA, 370 bp, Artificial Sequence
  Synthetic sequence nCoV-4-v2(N to T) mAb VH
caggtgcaactggtggagtctgggggaggcgtggtccagcctgggaggtccctgagactctcctgtgaagcctctggattcaccctcagaaccagtagtctccactgggtccgccaggctccaggcaaggggctggagtgggtggcagttatatcaaatgatggagccactaaattctacgcagacgccgtgaagggccgattcaccatctccagagacaactccaacaacaaaatatatctgcaactgaacggcctgaaacctgaggacacggctgtctattactgtgcgagagaaacacgtcattacagccatggtttgaactggttcgacccctggggccagggaaccctggtcaccgtctcctcag

SEQ ID NO: 130 - DNA, 24 bp, Artificial Sequence
  Synthetic sequence nCoV-4-v2 mAb CDRH1
ggattcaccctcagaaccagtagt

SEQ ID NO: 131 - DNA, 24 bp, Artificial Sequence
  Synthetic sequence nCoV-4-v2 mAb CDRH2
atatcaaatgatggagccactaaa

SEQ ID NO: 132 - DNA, 48 bp, Artificial Sequence; identical to SEQ ID NO: 116
  Synthetic sequence nCoV-4-v2 mAb CDRH3

SEQ ID NO: 133 - protein, 123 aa, Artificial Sequence
  Synthetic sequence nCoV-4-v2 (NVS to TVS) mAb VH
QVQLVESGGGVVQPGRSLRLSCEASGFTLRTSSLHWVRQAPGKGLEWVAVISNDGATKFYADAVKGRFTISRDNSNNKIYLQLNGLKPEDTAVYYCARETRHYSHGLNWFDPWGQGTLVTVSS

SEQ ID NO: 134 - protein, 8 aa, Artificial Sequence
  Synthetic sequence nCoV-4-v2 mAb CDRH1
GFTLRTSS

SEQ ID NO: 135 - protein, 8 aa, Artificial Sequence
  Synthetic sequence nCoV-4-v2 mAb CDRH2
ISNDGATK

SEQ ID NO: 136 - protein, 16 aa, Artificial Sequence
  Synthetic sequence nCoV-4-v2 mAb CDRH3
ARETRHYSHGLNWFDP

SEQ ID NO: 137 - DNA, 322 bp, Artificial Sequence; identical to SEQ ID NO: 121
  Synthetic sequence nCoV-4-v2 mAb VL (Vk)

SEQ ID NO: 138 - DNA, 18 bp, Artificial Sequence
  Synthetic sequence nCoV-4-v2 mAb CDRL1
cagagtgttagcagcgac

SEQ ID NO: 139 - DNA, 9 bp, Artificial Sequence
  Synthetic sequence nCoV4-v2 mAb CDRL2
ggtgcatcc

SEQ ID NO: 140 - DNA, 27 bp, Artificial Sequence
  Synthetic sequence nCoV-4-v2 mAb CDRL3
cagcagtataataactggccgaccacc

SEQ ID NO: 141 - protein, 107 aa, Artificial Sequence; identical to SEQ ID NO: 125
  Synthetic sequence nCoV-4-v2 mAb VL (Vk)

SEQ ID NO: 142 - protein, 6 aa, Artificial Sequence
  Synthetic sequence nCoV-4-v2 mAb CDRL1
QSVSSD

SEQ ID NO: 143 - protein, 3 aa, Artificial Sequence
  Synthetic sequence nCoV-4-v2 mAb CDRL2
GAS

SEQ ID NO: 144 - protein, 9 aa, Artificial Sequence
  Synthetic sequence nCoV-4-v2 mAb CDRL3
QQYNNWPTT

SEQ ID NO: 145 - DNA, 370 bp, Artificial Sequence; identical to SEQ ID NO: 129
  Synthetic sequence nCoV-4-v3(N to T) mAb VH

SEQ ID NO: 146 - DNA, 24 bp, Artificial Sequence
  Synthetic sequence nCoV-4-v3 mAb CDRH1
ggattcaccctcagaaccagtagt

SEQ ID NO: 147 - DNA, 24 bp, Artificial Sequence
  Synthetic sequence nCoV-4-v3 mAb CDRH2
atatcaaatgatggagccactaaa

SEQ ID NO: 148 - DNA, 48 bp, Artificial Sequence; identical to SEQ ID NO: 116
  Synthetic sequence nCoV-4-v3 mAb CDRH3

SEQ ID NO: 149 - protein, 123 aa, Artificial Sequence; identical to SEQ ID NO: 133
  Synthetic sequence nCoV-4-v3(N to T) mAb VH

SEQ ID NO: 150 - protein, 8 aa, Artificial Sequence
  Synthetic sequence nCoV-4-v3 mAb CDRH1
GFTLRTSS

SEQ ID NO: 151 - protein, 8 aa, Artificial Sequence
  Synthetic sequence nCoV-4-v3 mAb CDRH2
ISNDGATK

SEQ ID NO: 152 - protein, 16 aa, Artificial Sequence
  Synthetic sequence nCoV-4-v3 mAb CDRH3
ARETRHYSHGLNWFDP

SEQ ID NO: 153 - DNA, 322 bp, Artificial Sequence
  Synthetic sequence nCoV-4-v3 (W to F) mAb VL (Vk)
gaaagagtgatgacgcagtctccagtcaccctgtctgtgtctccaggggaaagagccaccctctcctgcagggccagtcagagtgttagcagcgacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataacttcccgaccaccttcggccaagggacacgactggacattaaac

SEQ ID NO: 154 - DNA, 18 bp, Artificial Sequence
  Synthetic sequence nCoV-4-v3 mAb CDRL1
cagagtgttagcagcgac

SEQ ID NO: 155 - DNA, 9 bp, Artificial Sequence
  Synthetic sequence nCoV-4-v3 mAb CDRL2
ggtgcatcc

SEQ ID NO: 156 - DNA, 27 bp, Artificial Sequence
  Synthetic sequence nCoV-4-v3 mAb CDRL3
cagcagtataataacttcccgaccacc

SEQ ID NO: 157 - protein, 107 aa, Artificial Sequence
  Synthetic sequence nCoV-4-v3 (W to F) mAb VL (Vk)
ERVMTQSPVTLSVSPGERATLSCRASQSVSSDLAWYQQKPGQAPRLLIYGASTRATGIPARFSGSGSGTEFTLTISSLQSEDFAVYYCQQYNNFPTTFGQGTRLDIK

SEQ ID NO: 158 - protein, 6 aa, Artificial Sequence
  Synthetic sequence nCoV-4-v3 mAb CDRL1
QSVSSD

SEQ ID NO: 159 - protein, 3 aa, Artificial Sequence
  Synthetic sequence nCoV-4-v3 mAb CDRL2
GAS

SEQ ID NO: 160 - protein, 9 aa, Artificial Sequence
  Synthetic sequence nCoV-4-v3 mAb CDRL3
QQYNNFPTT

SEQ ID NO: 161 - DNA, 352 bp, Artificial Sequence
  Synthetic sequence nCoV-15 mAb VH
gaggtgcagctggtggagtctgggggaggcttggtaacgccagggcggtccctgagactctcctgtacagcttctggattcacgtttggtgattatgctctgagctggttccgccaggctccagggaaggggctggagtgggtagctttcattagaagcaaagcttatggtgggacaacagaatacgccgcgtctgtgaaaggcagattcaccatctcaagagatgattcgcaaatcattgtctatctgcaaatggacagcctgaaaaccgaagacacagccgtatattattgtagttggatacaccggatagtctcctggggccagggaaccctggtcaccgtctcctcag

SEQ ID NO: 162 - DNA, 24 bp, Artificial Sequence
  Synthetic sequence nCoV-15 mAb CDRH1
ggattcacgtttggtgattatgct

SEQ ID NO: 163 - DNA, 30 bp, Artificial Sequence
  Synthetic sequence nCoV-15 mAb CDRH2
attagaagcaaagcttatggtgggacaaca

SEQ ID NO: 164 - DNA, 24 bp, Artificial Sequence
  Synthetic sequence nCoV-15 mAb CDRH3
agttggatacaccggatagtctcc

SEQ ID NO: 165 - protein, 117 aa, Artificial Sequence
  Synthetic sequence nCoV-15 mAb VH
EVQLVESGGGLVTPGRSLRLSCTASGFTFGDYALSWFRQAPGKGLEWVAFIRSKAYGGTTEYAASVKGRFTISRDDSQIIVYLQMDSLKTEDTAVYYCSWIHRIVSWGQGTLVTVSS

SEQ ID NO: 166 - protein, 8 aa, Artificial Sequence
  Synthetic sequence nCoV-15 mAb CDRH1
GFTFGDYA

SEQ ID NO: 167 - protein, 10 aa, Artificial Sequence
  Synthetic sequence nCoV-15 mAb CDRH2
IRSKAYGGTT

SEQ ID NO: 168 - protein, 8 aa, Artificial Sequence
  Synthetic sequence nCoV-15 mAb CDRH3
SWIHRIVS

SEQ ID NO: 169 - DNA, 322 bp, Artificial Sequence
  Synthetic sequence nCoV-15 mAb VL (Vk)
gacatccagatgacccagtctccatcctccctgtctgcatctgaaggagacagagtcaccatcacttgccaggcgagtcaggacattagcatctatttaaattggtttcagcagaaaccagggaaagcccctaagctcctgatctacgaggcatccaatttgaaaacaggggtcccatcaaggttcagtggaagtggatctgggacagattttactttcaccatcagcagcctgcagcctgaagatattgcaacatattactgtcaacagtatgataatctcccctacacttttggccaggggaccaagctggagatcaaac

SEQ ID NO: 170 - DNA, 18 bp, Artificial Sequence
  Synthetic sequence nCoV-15 mAb CDRL1
caggacattagcatctat

SEQ ID NO: 171 - DNA, 9 bp, Artificial Sequence
  Synthetic sequence nCoV-15 mAb CDRL2
gaggcatcc

SEQ ID NO: 172 - DNA, 27 bp, Artificial Sequence
  Synthetic sequence nCoV-15 mAb CDRL3
caacagtatgataatctcccctacact

SEQ ID NO: 173 - protein, 107 aa, Artificial Sequence
  Synthetic sequence nCoV-15 mAb VL (Vk)
DIQMTQSPSSLSASEGDRVTITCQASQDISIYLNWFQQKPGKAPKLLIYEASNLKTGVPSRFSGSGSGTDFTFTISSLQPEDIATYYCQQYDNLPYTFGQGTKLEIK

SEQ ID NO: 174 - protein, 6 aa, Artificial Sequence
  Synthetic sequence nCoV-15 mAb CDRL1
QDISIY

SEQ ID NO: 175 - protein, 3 aa, Artificial Sequence
  Synthetic sequence nCoV-15 mAb CDRL2
EAS

SEQ ID NO: 176 - protein, 9 aa, Artificial Sequence
  Synthetic sequence nCoV-15 mAb CDRL3
QQYDNLPYT

SEQ ID NO: 177 - DNA, 370 bp, Artificial Sequence
  Synthetic sequence nCoV-12 mAb VH
caggtgcagctggtggagtctgggggaggcgtggtccagcctgggaggtccctgagactctcctgtgcaggctctggattcaccttcagtagctttggtttgcactgggtccgccaggcgccaggcaagggactggagtggttggcacttatttcagatgagggacgcattaaatactacgcaaactccgtgaagggccgattctttatctccagagacaattccaagaacacgctgtatctgcaaatgaacagcctgagaggtgaggacacggctgtatattactgtgcgagagatgtcaaaggacatattgtggtgatgacttctcttgactactggggccagggagccctggtcaccgtctcctcag

SEQ ID NO: 178 - DNA, 24 bp, Artificial Sequence
  Synthetic sequence nCoV-12 mAb CDRH1
ggattcaccttcagtagctttggt

SEQ ID NO: 179 - DNA, 24 bp, Artificial Sequence
  Synthetic sequence nCoV-12 mAb CDRH2
atttcagatgagggacgcattaaa

SEQ ID NO: 180 - DNA, 48 bp, Artificial Sequence
  Synthetic sequence nCoV-12 mAb CDRH3
gcgagagatgtcaaaggacatattgtggtgatgacttctcttgactac

SEQ ID NO: 181 - protein, 123 aa, Artificial Sequence
  Synthetic sequence nCoV-12 mAb VH
QVQLVESGGGVVQPGRSLRLSCAGSGFTFSSFGLHWVRQAPGKGLEWLALISDEGRIKYYANSVKGRFFISRDNSKNTLYLQMNSLRGEDTAVYYCARDVKGHIVVMTSLDYWGQGALVTVSS

SEQ ID NO: 182 - protein, 8 aa, Artificial Sequence
  Synthetic sequence nCoV-12 mAb CDRH1
GFTFSSFG

SEQ ID NO: 183 - protein, 8 aa, Artificial Sequence
  Synthetic sequence nCoV-12 mAb CDRH2
ISDEGRIK

SEQ ID NO: 184 - protein, 16 aa, Artificial Sequence
  Synthetic sequence nCoV-12 mAb CDRH3
ARDVKGHIVVMTSLDY

SEQ ID NO: 185 - DNA, 337 bp, Artificial Sequence
  Synthetic sequence nCoV-12 mAb VL (Vk)
gatgttgtgatgactcagtctccactctccctgcccgtcacccttggacagccggcctccatctcctgtaggtctagtcaaagcctcgtctccagtgatggagacacctccttgagttggtttcagcagaggccaggccaatctccaaggcgcctaatttatgaggtttctaaccgggactctggggtcccagacagattcagcggcagtgggtcaggcactgatttcacactgaaaatcagcagggtggaggctgaggatgttggggtttattactgcatgcaaggtacacactggcctccgacgttcggccaagggaccaaggtggaaatcaaac

SEQ ID NO: 186 - DNA, 33 bp, Artificial Sequence
  Synthetic sequence nCoV-12 mAb CDRL1
caaagcctcgtctccagtgatggagacacctcc

SEQ ID NO: 187 - DNA, 9 bp, Artificial Sequence
  Synthetic sequence nCoV-12 mAb CDRL2
gaggtttct

SEQ ID NO: 188 - DNA, 27 bp, Artificial Sequence
  Synthetic sequence nCoV-12 mAb CDRL3
atgcaaggtacacactggcctccgacg

SEQ ID NO: 189 - protein, 112 aa, Artificial Sequence
  Synthetic sequence nCoV-12 mAb VL (Vk)
DVVMTQSPLSLPVTLGQPASISCRSSQSLVSSDGDTSLSWFQQRPGQSPRRLIYEVSNRDSGVPDRFSGSGSGTDFTLKISRVEAEDVGVYYCMQGTHWPPTFGQGTKVEIK

SEQ ID NO: 190 - protein, 11 aa, Artificial Sequence
  Synthetic sequence nCoV-12 mAb CDRL1
QSLVSSDGDTS

SEQ ID NO: 191 - protein, 3 aa, Artificial Sequence
  Synthetic sequence nCoV-12 mAb CDRL2
EVS

SEQ ID NO: 192 - protein, 9 aa, Artificial Sequence
  Synthetic sequence nCoV-12 mAb CDRL3
MQGTHWPPT

SEQ ID NO: 193 - DNA, 376 bp, Artificial Sequence
  Synthetic sequence nCoV-5 mAb VH
caaatgcagctggtgcagtctgggcctgagatgaagaagcctgggacctcagtgaaggtctcctgcaaggcctctggattcacttttagtaaaactaccttgcagtgggtacgtcaggctcgtggacaacgccctgagtggataggatggatcgtccttggcagcggtaacacaaactacgcacattacttccaggcaagagtcaccattaccagggacatgtccacgagcacagcctacatggaactgagcagcctgagttccgaggacacggccgtctattactgtgcggcagagatggctacaattcagaattactattactactatggtatggacgtctggggccaagggaccacggtcaccgtctcctcag

SEQ ID NO: 194 - DNA, 24 bp, Artificial Sequence
  Synthetic sequence nCoV-5 mAb CDRH1
ggattcacttttagtaaaactacc

SEQ ID NO: 195 - DNA, 24 bp, Artificial Sequence
  Synthetic sequence nCoV-5 mAb CDRH2
atcgtccttggcagcggtaacaca

SEQ ID NO: 196 - DNA, 54 bp, Artificial Sequence
  Synthetic sequence nCoV-5 mAb CDRH3
gcggcagagatggctacaattcagaattactattactactatggtatggacgtc

SEQ ID NO: 197 - protein, 125 aa, Artificial Sequence
  Synthetic sequence nCoV-5 mAb VH
QMQLVQSGPEMKKPGTSVKVSCKASGFTFSKTTLQWVRQARGQRPEWIGWIVLGSGNTNYAHYFQARVTITRDMSTSTAYMELSSLSSEDTAVYYCAAEMATIQNYYYYYGMDVWGQGTTVTVSS

SEQ ID NO: 198 - protein, 8 aa, Artificial Sequence
  Synthetic sequence nCoV-5 mAb CDRH1
GFTFSKTT

SEQ ID NO: 199 - protein, 8 aa, Artificial Sequence
  Synthetic sequence nCoV-5 mAb CDRH2
IVLGSGNT

SEQ ID NO: 200 - protein, 18 aa, Artificial Sequence
  Synthetic sequence nCoV-5 mAb CDRH3
AAEMATIQNYYYYYGMDV

SEQ ID NO: 201 - DNA, 322 bp, Artificial Sequence
  Synthetic sequence nCoV-5 mAb VL (Vk)
gacatccagatgacccagtctccatcctccctgtctgcatctgtcggggacagagtcaccatcacttgccgggcaagtcagagcattaactactatttaaattggtatcagcagaaacctgggaaagcccctaacctcctgatctatgctgcatccagtttgcctggtggggtcccatcaaggttcagtggcagtggatctgggacagatttcactctcaccatcaccagtctgcaacctgaggattttgcaacttacttctgtcaacagagttacagtacccctccgacttttggccaggggaccaagctggagatcaaac

SEQ ID NO: 202 - DNA, 18 bp, Artificial Sequence
  Synthetic sequence nCoV-5 mAb CDRL1
cagagcattaactactat

SEQ ID NO: 203 - DNA, 9 bp, Artificial Sequence
  Synthetic sequence nCoV-5 mAb CDRL2
gctgcatcc

SEQ ID NO: 204 - DNA, 27 bp, Artificial Sequence
  Synthetic sequence nCoV-5 mAb CDRL3
caacagagttacagtacccctccgact

SEQ ID NO: 205 - protein, 107 aa, Artificial Sequence
  Synthetic sequence nCoV-5 mAb VL (Vk)
DIQMTQSPSSLSASVGDRVTITCRASQSINYYLNWYQQKPGKAPNLLIYAASSLPGGVPSRFSGSGSGTDFTLTITSLQPEDFATYFCQQSYSTPPTFGQGTKLEIK

SEQ ID NO: 206 - protein, 6 aa, Artificial Sequence
  Synthetic sequence nCoV-5 mAb CDRL1
QSINYY

SEQ ID NO: 207 - protein, 3 aa, Artificial Sequence
  Synthetic sequence nCoV-5 mAb CDRL2
AAS

SEQ ID NO: 208 - protein, 9 aa, Artificial Sequence
  Synthetic sequence nCoV-5 mAb CDRL3
QQSYSTPPT

SEQ ID NO: 209 - DNA, 367 bp, Artificial Sequence
  Synthetic sequence nCoV-14 mAb VH
caggtgcagctggtgcactctggggctgaggtgaagaagcctggggcctcagtgagggtctcctgcaaggcctctggatacacctacaccgcctactatatacactggctgcggcaggcccctggacaagggcttgagtggatgggatggatcaacgctgacaatggtgacacacactctgtgcagaagtttcagggcagggtcaccatgaccagggacacgtccatcaatacagcctacctggaggtgagcaggctgaagtctgacgacacggccgtctattattgtgcgagaggagacgttccagttggtactggctgggtctttgacttctggggccagggaaccctggtcaccgtctcctcag

SEQ ID NO: 210 - DNA, 24 bp, Artificial Sequence
  Synthetic sequence nCoV-14 mAb CDRH1
ggatacacctacaccgcctactat

SEQ ID NO: 211 - DNA, 24 bp, Artificial Sequence
  Synthetic sequence nCoV-14 mAb CDRH2
atcaacgctgacaatggtgacaca

SEQ ID NO: 212 - DNA, 45 bp, Artificial Sequence
  Synthetic sequence nCoV-14 mAb CDRH3
gcgagaggagacgttccagttggtactggctgggtctttgacttc

SEQ ID NO: 213 - protein, 122 aa, Artificial Sequence
  Synthetic sequence nCoV-14 mAb VH
QVQLVHSGAEVKKPGASVRVSCKASGYTYTAYYIHWLRQAPGQGLEWMGWINADNGDTHSVQKFQGRVTMTRDTSINTAYLEVSRLKSDDTAVYYCARGDVPVGTGWVFDFWGQGTLVTVSS

SEQ ID NO: 214 - protein, 8 aa, Artificial Sequence
  Synthetic sequence nCoV-14 mAb CDRH1
GYTYTAYY

SEQ ID NO: 215 - protein, 8 aa, Artificial Sequence
  Synthetic sequence nCoV-14 mAb CDRH2
INADNGDT

SEQ ID NO: 216 - protein, 15 aa, Artificial Sequence
  Synthetic sequence nCoV-14 mAb CDRH3
ARGDVPVGTGWVFDF

SEQ ID NO: 217 - DNA, 319 bp, Artificial Sequence
  Synthetic sequence nCoV-14 mAb VL (Vk)
gacatccagatgacccagtctccatcctccctgtctgcatctgtgggagacagcgtcaccatcagttgccgggcaagtcagaccattaacaactttttaaattggtaccagcagaagccagggaaagcccctaacctcctgatctatggtgcatacaatttgcaaagtggggtcccatcaaggttcagtggcagtggatccgggacagatttcactctcaccatcagcagcctgcaacctgaggatgttgcaatttactattgtcaacagagtctcagtatggtcaccttcggccaagggacacgactggacattaaac

SEQ ID NO: 218 - DNA, 18 bp, Artificial Sequence
  Synthetic sequence nCoV-14 mAb CDRL1
cagaccattaacaacttt

SEQ ID NO: 219 - DNA, 9 bp, Artificial Sequence
  Synthetic sequence nCoV-14 mAb CDRL2
ggtgcatac

SEQ ID NO: 220 - DNA, 24 bp, Artificial Sequence
  Synthetic sequence nCoV-14 mAb CDRL3
caacagagtctcagtatggtcacc

SEQ ID NO: 221 - protein, 106 aa, Artificial Sequence
  Synthetic sequence nCoV-14 mAb VL (Vk)
DIQMTQSPSSLSASVGDSVTISCRASQTINNFLNWYQQKPGKAPNLLIYGAYNLQSGVPSRFSGSGSGTDFTLTISSLQPEDVAIYYCQQSLSMVTFGQGTRLDIK

SEQ ID NO: 222 - protein, 6 aa, Artificial Sequence
  Synthetic sequence nCoV-14 mAb CDRL1
QTINNF

SEQ ID NO: 223 - protein, 3 aa, Artificial Sequence
  Synthetic sequence nCoV-14 mAb CDRL2
GAY

SEQ ID NO: 224 - protein, 8 aa, Artificial Sequence
  Synthetic sequence nCoV-14 mAb CDRL3
QQSLSMVT

SEQ ID NO: 225 - DNA, 379 bp, Artificial Sequence
  Synthetic sequence nCoV-9 mAb VH
caggtgcttctggtggagtctgggggaggcgtggtccagcctgggatgtccctgagactctcctgtgcagcctctggattcaccttcagttcctatgctatgcactgggtccgccaggctccaggcaaggggctggagtgggtggcagttatctcatatgatggaagcactaaattctacgcagactccgtgaggggccgattccccatctccagagacaattccaagaacacggtgtatctgcaaatgaacagcctgagacctgaggacacggcagtctattactgtgcgacagttagtgtcgaggggtataccagtggctggtatttgggaacccttgacttctggggccagggaaccccggtcaccgtctcctcag

SEQ ID NO: 226 - DNA, 24 bp, Artificial Sequence
  Synthetic sequence nCoV-9 mAb CDRH1
ggattcaccttcagttcctatgct

SEQ ID NO: 227 - DNA, 24 bp, Artificial Sequence
  Synthetic sequence nCoV-9 mAb CDRH2
atctcatatgatggaagcactaaa

SEQ ID NO: 228 - DNA, 57 bp, Artificial Sequence
  Synthetic sequence nCoV-9 mAb CDRH3
gcgacagttagtgtcgaggggtataccagtggctggtatttgggaacccttgacttc

SEQ ID NO: 229 - protein, 126 aa, Artificial Sequence
  Synthetic sequence nCoV-9 mAb VH
QVLLVESGGGVVQPGMSLRLSCAASGFTFSSYAMHWVRQAPGKGLEWVAVISYDGSTKFYADSVRGRFPISRDNSKNTVYLQMNSLRPEDTAVYYCATVSVEGYTSGWYLGTLDFWGQGTPVTVSS

SEQ ID NO: 230 - protein, 8 aa, Artificial Sequence
  Synthetic sequence nCoV-9 mAb CDRH1
GFTFSSYA

SEQ ID NO: 231 - protein, 8 aa, Artificial Sequence
  Synthetic sequence nCoV-9 mAb CDRH2
ISYDGSTK

SEQ ID NO: 232 - protein, 20 aa, Artificial Sequence
  Synthetic sequence nCoV-9 mAb CDRH3
CATVSVEGYTSGWYLGTLDF

SEQ ID NO: 233 - DNA, 322 bp, Artificial Sequence
  Synthetic sequence nCoV-9 mAb VL (Vk)
gaaagagtgatgacgcagtttccagccaccctgtctgtgtctccaggggaaagagccaccctctcctgcagggccagtcagagtgttagtagcaacttagcctggtaccagcagaaacctggccaggctcccaggctcctcatctatggtgcatccaccagggccattggtgtcccagccaggttcagtggcagtgggtctgggacagagttcactctcaccatcagcagcctgcagtctgaagattttgcagtttattactgtcagcagtataataactggccgggaacttttggccaggggaccaagctggagatcaaac

SEQ ID NO: 234 - DNA, 18 bp, Artificial Sequence
  Synthetic sequence nCoV-9 mAb CDRL1
cagagtgttagtagcaac

SEQ ID NO: 235 - DNA, 9 bp, Artificial Sequence
  Synthetic sequence nCoV-9 mAb CDRL2
ggtgcatcc

SEQ ID NO: 236 - DNA, 27 bp, Artificial Sequence
  Synthetic sequence nCoV-9 mAb CDRL3
cagcagtataataactggccgggaact

SEQ ID NO: 237 - protein, 107 aa, Artificial Sequence
  Synthetic sequence nCoV-9 mAb VL (Vk)
ERVMTQFPATLSVSPGERATLSCRASQSVSSNLAWYQQKPGQAPRLLIYGASTRAIGVPARFSGSGSGTEFTLTISSLQSEDFAVYYCQQYNNWPGTFGQGTKLEIK

SEQ ID NO: 238 - protein, 6 aa, Artificial Sequence
  Synthetic sequence nCoV-9 mAb CDRL1
QSVSSN

SEQ ID NO: 239 - protein, 3 aa, Artificial Sequence
  Synthetic sequence nCoV-9 mAb CDRL2
GAS

SEQ ID NO: 240 - protein, 9 aa, Artificial Sequence
  Synthetic sequence nCoV-9 mAb CDRL3
QQYNNWPGT

SEQ ID NO: 241 - DNA, 367 bp, Artificial Sequence
  Synthetic sequence nCoV-18 mAb VH
caggttcagctggtgcagtctggagctgaggtgaggcagcctgggacctcagtgaaggtctcctgcaaggcttctggttacacctttatgaataatgatatcacctgggtgcgacaggcccctggacaggtgcttcagtggatggggtggatcaggccttccaatggaaacacaaactacgcacagaagttccagggcagagtcaccctgaccacagacacatccacgaacacagcccacatggaactgaggagcctgaaatctgacgacacggccgtgtattattgtgcgagacaatctcattcgacgcggggtggctggcacttcagcccctggggccagggaaccctggtcaccgtctcctcag

SEQ ID NO: 242 - DNA, 24 bp, Artificial Sequence
  Synthetic sequence nCoV-18 mAb CDRH1
ggttacacctttatgaataatgat

SEQ ID NO: 243 - DNA, 24 bp, Artificial Sequence
  Synthetic sequence nCoV-18 mAb CDRH2
atcaggccttccaatggaaacaca

SEQ ID NO: 244 - DNA, 45 bp, Artificial Sequence
  Synthetic sequence nCoV-18 mAb CDRH3
gcgagacaatctcattcgacgcggggtggctggcacttcagcccc

SEQ ID NO: 245 - protein, 122 aa, Artificial Sequence
  Synthetic sequence nCoV-18 mAb VH
QVQLVQSGAEVRQPGTSVKVSCKASGYTFMNNDITWVRQAPGQVLQWMGWIRPSNGNTNYAQKFQGRVTLTTDTSTNTAHMELRSLKSDDTAVYYCARQSHSTRGGWHFSPWGQGTLVTVSS

SEQ ID NO: 246 - protein, 8 aa, Artificial Sequence
  Synthetic sequence nCoV-18 mAb CDRH1
GYTFMNND

SEQ ID NO: 247 - protein, 8 aa, Artificial Sequence
  Synthetic sequence nCoV-18 mAb CDRH2
IRPSNGNT

SEQ ID NO: 248 - protein, 15 aa, Artificial Sequence
  Synthetic sequence nCoV-18 mAb CDRH3
ARQSHSTRGGWHFSP

SEQ ID NO: 249 - DNA, 322 bp, Artificial Sequence
  Synthetic sequence nCoV-18 mAb VL (Vk)
gacatccagatgacccagtctccatcctccctgtctgcatctgtaggagacagagtcaccatcacttgccggccaagtcaacccattagctactttttaaattggtatcagcagacaccagggaaagcccctaagctcctcatctatgctgcatccagtttgcaaagtggggtcccatcaaggttcagtggcagtggatctgggacagatttcactctcaccatcagcagtctgcaacctgaagattctgcaacttactactgtcaacagagttatagtgtcccgtacacttttggccaggggaccaagctggagatcaaac

SEQ ID NO: 250 - DNA, 18 bp, Artificial Sequence
  Synthetic sequence nCoV-18 mAb CDRL1
caacccattagctacttt

SEQ ID NO: 251 - DNA, 9 bp, Artificial Sequence
  Synthetic sequence nCoV-18 mAb CDRL2
gctgcatcc

SEQ ID NO: 252 - DNA, 27 bp, Artificial Sequence
  Synthetic sequence nCoV-18 mAb CDRL3
caacagagttatagtgtcccgtacact

SEQ ID NO: 253 - protein, 107 aa, Artificial Sequence
  Synthetic sequence nCoV-18 mAb VL (Vk)
DIQMTQSPSSLSASVGDRVTITCRPSQPISYFLNWYQQTPGKAPKLLIYAASSLQSGVPSRFSGSGSGTDFTLTISSLQPEDSATYYCQQSYSVPYTFGQGTKLEIK

SEQ ID NO: 254 - protein, 6 aa, Artificial Sequence
  Synthetic sequence nCoV-18 mAb CDRL1
QPISYF

SEQ ID NO: 255 - protein, 3 aa, Artificial Sequence
  Synthetic sequence nCoV-18 mAb CDRL2
AAS

SEQ ID NO: 256 - protein, 9 aa, Artificial Sequence
  Synthetic sequence nCoV-18 mAb CDRL3
QQSYSVPYT

SEQ ID NO: 257 - DNA, 382 bp, Artificial Sequence
  Synthetic sequence nCoV-8 mAb VH
gaagtgctcctggtggagtctgggggaggcttggtacagcctggcaggtccctgagactctcctgtgtagtctctggattcacctttgattattatgccatgcactgggtccggcaagctccagggaagggcctggagtgggtctcaggtattagttggaatagtgataacacagactatgcggactctgtgaagggccgattcaccatctccagagacaacgccaagaactccctgtatctgcaaatgaacagtctgaaaactgaggacacggccttgtattactgtgcaaaagatattagtctagttttttggagtgttaaccctccccgtaacggaatggacgtctggggccaagggaccacggtcaccgtctcctcag

SEQ ID NO: 258 - DNA, 24 bp, Artificial Sequence
  Synthetic sequence nCoV-8 mAb CDRH1
ggattcacctttgattattatgcc

SEQ ID NO: 259 - DNA, 24 bp, Artificial Sequence
  Synthetic sequence nCoV-8 mAb CDRH2
attagttggaatagtgataacaca

SEQ ID NO: 260 - DNA, 60 bp, Artificial Sequence
  Synthetic sequence nCoV-8 mAb CDRH3
gcaaaagatattagtctagttttttggagtgttaaccctccccgtaacggaatggacgtc

SEQ ID NO: 261 - protein, 127 aa, Artificial Sequence
  Synthetic sequence nCoV-8 mAb VH
EVLLVESGGGLVQPGRSLRLSCVVSGFTFDYYAMHWVRQAPGKGLEWVSGISWNSDNTDYADSVKGRFTISRDNAKNSLYLQMNSLKTEDTALYYCAKDISLVFWSVNPPRNGMDVWGQGTTVTVSS

SEQ ID NO: 262 - protein, 9 aa, Artificial Sequence
  Synthetic sequence nCoV-8 mAb CDRH1
GFTFDYYAM

SEQ ID NO: 263 - protein, 8 aa, Artificial Sequence
  Synthetic sequence nCoV-8 mAb CDRH2
ISWNSDNT

SEQ ID NO: 264 - protein, 20 aa, Artificial Sequence
  Synthetic sequence nCoV-8 mAb CDRH3
AKDISLVFWSVNPPRNGMDV

SEQ ID NO: 265 - DNA, 322 bp, Artificial Sequence
  Synthetic sequence nCoV-8 mAb VL (Vk)
gacatccagatgacccagtctccatcctccctgtctgcatctgtaggagacagagtcaccatcacttgccgggcaagtcagagcattcgcagctatttaaattggtatcagcagaaaccagggaaagcccctaacctcctgatctatactgcatccagtttgcaaagtggggtcccatcaaggttcagtggcagtggatctgggacagatttcactctcaccatcagcagtctgcaacctgaagattttgcgacttactactgtcaacagagttacagttcccctctcactttcggcggagggaccaaggtggagatcaaac

SEQ ID NO: 266 - DNA, 18 bp, Artificial Sequence
  Synthetic sequence nCoV-8 mAb CDRL1
cagagcattcgcagctat

SEQ ID NO: 267 - DNA, 9 bp, Artificial Sequence
  Synthetic sequence nCoV-8 mAb CDRL2
actgcatcc

SEQ ID NO: 268 - DNA, 27 bp, Artificial Sequence
  Synthetic sequence nCoV-8 mAb CDRL3
caacagagttacagttcccctctcact

SEQ ID NO: 269 - protein, 107 aa, Artificial Sequence
  Synthetic sequence nCoV-8 mAb VL (Vk)
DIQMTQSPSSLSASVGDRVTITCRASQSIRSYLNWYQQKPGKAPNLLIYTASSLQSGVPSRFSGSGSGTDFTLTISSLQPEDFATYYCQQSYSSPLTFGGGTKVEIK

SEQ ID NO: 270 - protein, 6 aa, Artificial Sequence
  Synthetic sequence nCoV-8 mAb CDRL1
QSIRSY

SEQ ID NO: 271 - protein, 3 aa, Artificial Sequence
  Synthetic sequence nCoV-8 mAb CDRL2
TAS

SEQ ID NO: 272 - protein, 9 aa, Artificial Sequence
  Synthetic sequence nCoV-8 mAb CDRL3
QQSYSSPLT

SEQ ID NO: 273 - DNA, 367 bp, Artificial Sequence
  Synthetic sequence nCoV-7 mAb VH
caggagcaggtggtggagtctgggggaggcgtggtccagcctgggaagtccctgagactctcctgtgcagcctctggattcagcttgaatgactttgctatccactgggtccgccaggctccaggcaagaggctggagtgggtggcaatcatatcatacgatggaagggttaaattttacgcagactcagtgaagggccgattcaccatctcccgagactcttccgtgcatctgcaaatggacagcctgagacctgaggacacgggtctgtattactgtgcgagagactcttcgtggcagagcactgggtggcctataaactggttcgaccgctggggccggggaaccctggtcaccgtctcctcag

SEQ ID NO: 274 - DNA, 24 bp, Artificial Sequence
  Synthetic sequence nCoV-7 mAb CDRH1
ggattcagcttgaatgactttgct

SEQ ID NO: 275 - DNA, 24 bp, Artificial Sequence
  Synthetic sequence nCoV-7 mAb CDRH2
atatcatacgatggaagggttaaa

SEQ ID NO: 276 - DNA, 54 bp, Artificial Sequence
  Synthetic sequence nCoV-7 mAb CDRH3
gcgagagactcttcgtggcagagcactgggtggcctataaactggttcgaccgc

SEQ ID NO: 277 - protein, 122 aa, Artificial Sequence
  Synthetic sequence nCoV-7 mAb VH
QEQVVESGGGVVQPGKSLRLSCAASGFSLNDFAIHWVRQAPGKRLEWVAIISYDGRVKFYADSVKGRFTISRDSSVHLQMDSLRPEDTGLYYCARDSSWQSTGWPINWFDRWGRGTLVTVSS

SEQ ID NO: 278 - protein, 8 aa, Artificial Sequence
  Synthetic sequence nCoV-7 mAb CDRH1
GFSLNDFA

SEQ ID NO: 279 - protein, 8 aa, Artificial Sequence
  Synthetic sequence nCoV-7 mAb CDRH2
ISYDGRVK

SEQ ID NO: 280 - protein, 18 aa, Artificial Sequence
  Synthetic sequence nCoV-7 mAb CDRH3
ARDSSWQSTGWPINWFDR

SEQ ID NO: 281 - DNA, 322 bp, Artificial Sequence
  Synthetic sequence nCoV-7 mAb VL (Vk)
gaaattgtgttgacacagtctccagccaccctgtctttgtctccagggcaaagagccaccctctcctgcagggccagtcagagtgttctcagctccttagcctggtaccagcacaaacctggccaggctcccaggctcctcatctatgatgcatccagcagggccactggcgtcccagccaggttcagtggcagtgggtctgagacagacttcactctcaccatcagcagcctagagcctgaagattttgcagtttattactgtcagcagcgtagcaactggcctccgacgttcggccaagggaccaaggtggaaatcaaac

SEQ ID NO: 282 - DNA, 18 bp, Artificial Sequence
  Synthetic sequence nCoV-7 mAb CDRL1
cagagtgttctcagctcc

SEQ ID NO: 283 - DNA, 9 bp, Artificial Sequence
  Synthetic sequence nCoV-7 mAb CDRL2
gatgcatcc

SEQ ID NO: 284 - DNA, 27 bp, Artificial Sequence
  Synthetic sequence nCoV-7 mAb CDRL3
cagcagcgtagcaactggcctccgacg

SEQ ID NO: 285 - protein, 107 aa, Artificial Sequence
  Synthetic sequence nCoV-7 mAb VL (Vk)
EIVLTQSPATLSLSPGQRATLSCRASQSVLSSLAWYQHKPGQAPRLLIYDASSRATGVPARFSGSGSETDFTLTISSLEPEDFAVYYCQQRSNWPPTFGQGTKVEIK

SEQ ID NO: 286 - protein, 6 aa, Artificial Sequence
  Synthetic sequence nCoV-7 mAb CDRL1
QSVLSS

SEQ ID NO: 287 - protein, 3 aa, Artificial Sequence
  Synthetic sequence nCoV-7 mAb CDRL2
DAS

SEQ ID NO: 288 - protein, 9 aa, Artificial Sequence
  Synthetic sequence nCoV-7 mAb CDRL3
QQRSNWPPT

SEQ ID NO: 289 - DNA, 358 bp, Artificial Sequence
  Synthetic sequence nCoV-2 mAb VH
gacgtgcagctgttggagtctgggggaggcttggtacagcctggggggtccctgagactctcctgtgcagcctctggattcagctttagcagctatgccatgacctgggtccgccaggctccagggaaggggctggagtgggtcgcaactatgagtgctagtggggatagcacaaacgacgcagactccgtgaagggccggttcaccatctccagagacaattccaagaacacgctgtttctgcaaatgaacagcctcagacccgaggacacggccgtatattactgtgcgtccccccttcggaattatggtgacttgctctactggggccagggaaccctggtcaccgtctcctccg

SEQ ID NO: 290 - DNA, 24 bp, Artificial Sequence
  Synthetic sequence nCoV-2 mAb CDRH1
ggattcagctttagcagctatgcc

SEQ ID NO: 291 - DNA, 24 bp, Artificial Sequence
  Synthetic sequence nCoV-2 mAb CDRH2
atgagtgctagtggggatagcaca

SEQ ID NO: 292 - DNA, 36 bp, Artificial Sequence
  Synthetic sequence nCoV-2 mAb CDRH3
gcgtccccccttcggaattatggtgacttgctctac

SEQ ID NO: 293 - protein, 119 aa, Artificial Sequence
  Synthetic sequence nCoV-2 mAb VH
DVQLLESGGGLVQPGGSLRLSCAASGFSFSSYAMTWVRQAPGKGLEWVATMSASGDSTNDADSVKGRFTISRDNSKNTLFLQMNSLRPEDTAVYYCASPLRNYGDLLYWGQGTLVTVSS

SEQ ID NO: 294 - protein, 8 aa, Artificial Sequence
  Synthetic sequencenCoV-2 mAb CDRH1
GFSFSSYA

SEQ ID NO: 295 - protein, 8 aa, Artificial Sequence
  Synthetic sequence nCoV-2 mAb CDRH2
MSASGDST

SEQ ID NO: 296 - protein, 12 aa, Artificial Sequence
  Synthetic sequence nCoV-2 mAb CDRH3
ASPLRNYGDLLY

SEQ ID NO: 297 - DNA, 322 bp, Artificial Sequence
  Synthetic sequence nCoV-2 mAb VL (Vk)
gacatccagatgacccagtctccttccaccctgtctgcatctgtaggagacagagtcaccatcacttgccgggccagtcagaatattcatcgttttttggcctggtatcagcagaaaccagggaaagcccctaaactcctgatctatacggcgtctagtttagaaagtggggtcccatcaaggttcagcggcagtggatttgggacagaattcactctcaccatcagcagcctgcagcctgatgattttgcaacttattactgccaacaatataatagttactcgtggacgttcggccaagggaccaaggtggaaatcaaac

SEQ ID NO: 298 - DNA, 18 bp, Artificial Sequence
  Synthetic sequence nCoV-2 mAb CDRL1
cagaatattcatcgtttt

SEQ ID NO: 299 - DNA, 9 bp, Artificial Sequence
  Synthetic sequence nCoV-2 mAb CDRL2
acggcgtct

SEQ ID NO: 300 - DNA, 27 bp, Artificial Sequence
  Synthetic sequence nCoV-2 mAb CDRL3
caacaatataatagttactcgtggacg

SEQ ID NO: 301 - protein, 107 aa, Artificial Sequence
  Synthetic sequence nCoV-2 mAb VL (Vk)
DIQMTQSPSTLSASVGDRVTITCRASQNIHRFLAWYQQKPGKAPKLLIYTASSLESGVPSRFSGSGFGTEFTLTISSLQPDDFATYYCQQYNSYSWTFGQGTKVEIK

SEQ ID NO: 302 - protein, 6 aa, Artificial Sequence
  Synthetic sequence nCoV-2 mAb CDRL1
QNIHRF

SEQ ID NO: 303 - protein, 3 aa, Artificial Sequence
  Synthetic sequence nCoV-2 mAb CDRL2
TAS

SEQ ID NO: 304 - protein, 9 aa, Artificial Sequence
  Synthetic sequence nCoV-2 mAb CDRL3
QQYNSYSWT

SEQ ID NO: 305 - DNA, 370 bp, Artificial Sequence
  Synthetic sequence nCoV-19 mAb VH
caggtgaagctggtggagtctgggggaggcgtggtccagcctgggaggtccctgagactctcctgtgcagcctctggattcaccttcagtagcaatgctatgcactgggtccgccagactccaggcaaggggctggagtgggtggcacttatatcatatgatgaaaggaataaatactacgcagagtccgtgaagggccgattcaccatctccagagacaattccaagaacacgctgtatctgcaaatgaacagcctgagacatgaggacacggctgtgtattactgtgcgagagatctgcaaatgagagtagtggttgtttcaaactttgactactggggccagggaaccctggtcaccgtctcctcag

SEQ ID NO: 306 - DNA, 24 bp, Artificial Sequence
  Synthetic sequence nCoV-19 mAb CDRH1
ggattcaccttcagtagcaatgct

SEQ ID NO: 307 - DNA, 24 bp, Artificial Sequence
  Synthetic sequence nCoV-19 mAb CDRH2
atatcatatgatgaaaggaataaa

SEQ ID NO: 308 - DNA, 49 bp, Artificial Sequence
  Synthetic sequence nCoV-19 mAb CDRH3
gcgagagatctgcaaatgagagtagtggttgtttcaaactttgactact

SEQ ID NO: 309 - protein, 123 aa, Artificial Sequence
  Synthetic sequence nCoV-19 mAb VH
QVKLVESGGGVVQPGRSLRLSCAASGFTFSSNAMHWVRQTPGKGLEWVALISYDERNKYYAESVKGRFTISRDNSKNTLYLQMNSLRHEDTAVYYCARDLQMRVVVVSNFDYWGQGTLVTVSS

SEQ ID NO: 310 - protein, 8 aa, Artificial Sequence
  Synthetic sequence nCoV-19 mAb CDRH1
GFTFSSNA

SEQ ID NO: 311 - protein, 8 aa, Artificial Sequence
  Synthetic sequence nCoV-19 mAb CDRH2
ISYDERNK

SEQ ID NO: 312 - protein, 16 aa, Artificial Sequence
  Synthetic sequence nCoV-19 mAb CDRH3
ARDLQMRVVVVSNFDY

SEQ ID NO: 313 - DNA, 337 bp, Artificial Sequence
  Synthetic sequence nCoV-19 mAb VL (Vk)
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttatgaggtttctaactgggactctggggtcccagacagattcagcggcagtgggtcaggcactgatttcacactgaaaatcagcagggtggaggctgaggatgttggggtttattactgcatgcaagctacacactggcctcccacttttggccaggggaccaagctggagatcaaac

SEQ ID NO: 314 - DNA, 33 bp, Artificial Sequence
  Synthetic sequence nCoV-19 mAb CDRL1
caaagcctcgtatacagtgatggaaacacctac

SEQ ID NO: 315 - DNA, 9 bp, Artificial Sequence
  Synthetic sequence nCoV-19 mAb CDRL2
gaggtttct

SEQ ID NO: 316 - DNA, 27 bp, Artificial Sequence
  Synthetic sequence nCoV-19 mAb CDRL3
atgcaagctacacactggcctcccact

SEQ ID NO: 317 - protein, 112 aa, Artificial Sequence
  Synthetic sequence nCoV-19 mAb VL (Vk)
DVVMTQSPLSLPVTLGQPASISCRSSQSLVYSDGNTYLNWFQQRPGQSPRRLIYEVSNWDSGVPDRFSGSGSGTDFTLKISRVEAEDVGVYYCMQATHWPPTFGQGTKLEIK

SEQ ID NO: 318 - protein, 11 aa, Artificial Sequence
  Synthetic sequence nCoV-19 mAb CDRL1
QSLVYSDGNTY

SEQ ID NO: 319 - protein, 3 aa, Artificial Sequence
  Synthetic sequence nCoV-19 mAb CDRL2
EVS

SEQ ID NO: 320 - protein, 9 aa, Artificial Sequence
  Synthetic sequence nCoV-19 mAb CDRL3
MQATHWPPT

SEQ ID NO: 321 - DNA, 382 bp, Artificial Sequence
  Synthetic sequence nCoV-1 mAb VH
caggcgcaactggtggagtctgggggagccttggtccagcctgggaggtccctgagactctcctgtgcagcctctggattcaccttcaggaattatgctatgcactgggtccgccaggctccagccacggggctgcagtggctggcagtcataacatctgatggaaggaataaattctatgcagactccgtgaagggccgattcaccatctccagagaggattccaagaacacgctgtatctgcaaatggatagcctgagaggagaggacacggctgtctactactgcgtgacacagcgtgataatagtcgcgattacttcccccactacttccacgacatggacgtctggggccaagggaccacggtcgccgtctcctcag

SEQ ID NO: 322 - DNA, 24 bp, Artificial Sequence
  Synthetic sequence nCoV-1 mAb CDRH1
ggattcaccttcaggaattatgct

SEQ ID NO: 323 - DNA, 24 bp, Artificial Sequence
  Synthetic sequence nCoV-1 mAb CDRH2
ataacatctgatggaaggaataaa

SEQ ID NO: 324 - DNA, 60 bp, Artificial Sequence
  Synthetic sequence nCoV-1 mAb CDRH3
gtgacacagcgtgataatagtcgcgattacttcccccactacttccacgacatggacgtc

SEQ ID NO: 325 - protein, 127 aa, Artificial Sequence
  Synthetic sequence nCoV-1 mAb VH
QAQLVESGGALVQPGRSLRLSCAASGFTFRNYAMHWVRQAPATGLQWLAVITSDGRNKFYADSVKGRFTISREDSKNTLYLQMDSLRGEDTAVYYCVTQRDNSRDYFPHYFHDMDVWGQGTTVAVSS

SEQ ID NO: 326 - protein, 8 aa, Artificial Sequence
  Synthetic sequence nCoV-1 mAb CDRH1
GFTFRNYA

SEQ ID NO: 327 - protein, 8 aa, Artificial Sequence
  Synthetic sequence nCoV-1 mAb CDRH2
ITSDGRNK

SEQ ID NO: 328 - protein, 20 aa, Artificial Sequence
  Synthetic sequence nCoV-1 mAb CDRH3
VTQRDNSRDYFPHYFHDMDV

SEQ ID NO: 329 - DNA, 337 bp, Artificial Sequence
  Synthetic sequence nCoV-1 mAb VL (Vk)
gatgttgtgctgactcagtctccactctccctgcccgtcacccttggacagccggcctccatctcctgcaggtctagtcaaagcctcgtttacagtgatggagacacctacttgaattggtttcagcagaggccaggccaatctccaaggcgcctaatttatcaggtttctaatcgggactctggggtcccagacagatttagcggcagtgggtcaggcactgatttcacactgaaaatcagcagggtggaggctgaggatgttggggtttattactgcatgcaaggttcacactggcctccgacgttcggccaagggaccaaggtggaaatcaaac

SEQ ID NO: 330 - DNA, 33 bp, Artificial Sequence
  Synthetic sequence nCoV-1 mAb CDRL1
caaagcctcgtttacagtgatggagacacctac

SEQ ID NO: 331 - DNA, 9 bp, Artificial Sequence
  Synthetic sequence nCoV-1 mAb CDRL2
caggtttct

SEQ ID NO: 332 - DNA, 27 bp, Artificial Sequence
  Synthetic sequence nCoV-1 mAb CDRL3
atgcaaggttcacactggcctccgacg

SEQ ID NO: 333 - protein, 112 aa, Artificial Sequence
  Synthetic sequence nCoV-1 mAb VL (Vk)
DVVLTQSPLSLPVTLGQPASISCRSSQSLVYSDGDTYLNWFQQRPGQSPRRLIYQVSNRDSGVPDRFSGSGSGTDFTLKISRVEAEDVGVYYCMQGSHWPPTFGQGTKVEIK

SEQ ID NO: 334 - protein, 11 aa, Artificial Sequence
  Synthetic sequence nCoV-1 mAb CDRL1
QSLVYSDGDTY

SEQ ID NO: 335 - protein, 3 aa, Artificial Sequence
  Synthetic sequence nCoV-1 mAb CDRL2
QVS

SEQ ID NO: 336 - protein, 9 aa, Artificial Sequence
  Synthetic sequence nCoV-1 mAb CDRL3
MQGSHWPPT

SEQ ID NO: 337 - DNA, 373 bp, Artificial Sequence
  Synthetic sequence nCoV-11 mAb VH
ctggtgcaactggtagagtctgggggaggcgtggtccagcctgggaggtccctgagactctcctgtgcaggctctggattcacctttagcagctatggcatgcactgggtccgccagactccaggcaaggggctggagtgggtggcagttatatcgtttgatggaaggaacaaattctacgcagaccccgtgaagggtcgattcaccatctccagagacaattccaagaacacggtgttcttggaattggatagcctgacaactgaggacacggctttttattactgtgcgagagacgacaacttggacagacactggccccttcgactcgggggttactggggccagggaaccctggtcaccgtctcctcag

SEQ ID NO: 338 - DNA, 24 bp, Artificial Sequence
  Synthetic sequence nCoV-11 mAb CDRH1
ggattcacctttagcagctatggc

SEQ ID NO: 339 - DNA, 24 bp, Artificial Sequence
  Synthetic sequence nCoV-11 mAb CDRH2
atatcgtttgatggaaggaacaaa

SEQ ID NO: 340 - DNA, 51 bp, Artificial Sequence
  Synthetic sequence nCoV-11 mAb CDRH3
gcgagagacgacaacttggacagacactggccccttcgactcgggggttac

SEQ ID NO: 341 - protein, 124 aa, Artificial Sequence
  Synthetic sequence nCoV-11 mAb VH
LVQLVESGGGVVQPGRSLRLSCAGSGFTFSSYGMHWVRQTPGKGLEWVAVISFDGRNKFYADPVKGRFTISRDNSKNTVFLELDSLTTEDTAFYYCARDDNLDRHWPLRLGGYWGQGTLVTVSS

SEQ ID NO: 342 - protein, 8 aa, Artificial Sequence
  Synthetic sequence nCoV-11 mAb CDRH1
GFTFSSYG

SEQ ID NO: 343 - protein, 8 aa, Artificial Sequence
  Synthetic sequence nCoV-11 mAb CDRH2
ISFDGRNK

SEQ ID NO: 344 - protein, 17 aa, Artificial Sequence
  Synthetic sequence nCoV-11 mAb CDRH3
ARDDNLDRHWPLRLGGY

SEQ ID NO: 345 - DNA, 337 bp, Artificial Sequence
  Synthetic sequence nCoV-11 mAb VL (Vk)
gaaattgtgatgactcagtctccactctccctgcccgtcacccttggacagccggcctccatctcctgcaggtctagtcaagacctcctatacaatgatggaggcaccgacttgaactggtttcagcagaggccaggccaatctccaaggcgcctaatttacagggtttctaaccgggactctggggtcccagacagattcagcggcagtgggtcaggcagtgatttcacactgaaaatcagcagggtggaggctgaggatgttggaatttattactgcatgcaaggtgcacactggcctccgactttcggccctgggaccaaagtggagatcaaac

SEQ ID NO: 346 - DNA, 33 bp, Artificial Sequence
  Synthetic sequence nCoV-11 mAb CDRL1
caagacctcctatacaatgatggaggcaccgac

SEQ ID NO: 347 - DNA, 9 bp, Artificial Sequence
  Synthetic sequence nCoV-11 mAb CDRL2
agggtttct

SEQ ID NO: 348 - DNA, 27 bp, Artificial Sequence
  Synthetic sequence nCoV-11 mAb CDRL3
atgcaaggtgcacactggcctccgact

SEQ ID NO: 349 - protein, 112 aa, Artificial Sequence
  Synthetic sequence nCoV-11 mAb VL (Vk)
EIVMTQSPLSLPVTLGQPASISCRSSQDLLYNDGGTDLNWFQQRPGQSPRRLIYRVSNRDSGVPDRFSGSGSGSDFTLKISRVEAEDVGIYYCMQGAHWPPTFGPGTKVEIK

SEQ ID NO: 350 - protein, 11 aa, Artificial Sequence
  Synthetic sequence nCoV-11 mAb CDRL1
QDLLYNDGGTD

SEQ ID NO: 351 - protein, 3 aa, Artificial Sequence
  Synthetic sequence nCoV-11 mAb CDRL2
RVS

SEQ ID NO: 352 - protein, 9 aa, Artificial Sequence
  Synthetic sequence nCoV-11 mAb CDRL3
MQGAHWPPT

SEQ ID NO: 353 - DNA, 355 bp, Artificial Sequence
  Synthetic sequence nCoV-13 mAb VH
gaggtgcagctggtggagtctgggggaggcctggtcaagcctggggggtccctgagactctcctgtgcagcctctggattcaccttcagtaactataacatgaactgggtccgccaggctccagggaaggggctggagtgggtctcatccattggtagtagtagcacttacatatactccgcagactcagtgaagggccgattcaccatctccagagacaacgccatgaactcgctgtttctgcaaatgaagagcctgagagccgaggacacggctgtgtattactgtgcgagaggtttcgagaggtactactttgactcctggggccagggaaccctggtcaccgtctcctcag

SEQ ID NO: 354 - DNA, 24 bp, Artificial Sequence
  Synthetic sequence nCoV-13 mAb CDRH1
ggattcaccttcagtaactataac

SEQ ID NO: 355 - DNA, 24 bp, Artificial Sequence
  Synthetic sequence nCoV-13 mAb CDRH2
attggtagtagtagcacttacata

SEQ ID NO: 356 - DNA, 33 bp, Artificial Sequence
  Synthetic sequence nCoV-13 mAb CDRH3
gcgagaggtttcgagaggtactactttgactcc

SEQ ID NO: 357 - protein, 118 aa, Artificial Sequence
  Synthetic sequence nCoV-13 mAb VH
EVQLVESGGGLVKPGGSLRLSCAASGFTFSNYNMNWVRQAPGKGLEWVSSIGSSSTYIYSADSVKGRFTISRDNAMNSLFLQMKSLRAEDTAVYYCARGFERYYFDSWGQGTLVTVSS

SEQ ID NO: 358 - protein, 8 aa, Artificial Sequence
  Synthetic sequence nCoV-13 mAb CDRH1
GFTFSNYN

SEQ ID NO: 359 - protein, 8 aa, Artificial Sequence
  Synthetic sequence nCoV-13 mAb CDRH2
IGSSSTYI

SEQ ID NO: 360 - protein, 11 aa, Artificial Sequence
  Synthetic sequence nCoV-13 mAb CDRH3
ARGFERYYFDS

SEQ ID NO: 361 - DNA, 331 bp, Artificial Sequence
  Synthetic sequence nCoV-13 mAb VL
cagtctgtgctgactcagccaccctcagcgtctgggacccccgggcagagggtcaccatctcttgttctggaagcacctccaacatcggaactaatgttgtaaactggtaccagcagctcccaggaacggcccccaaactcctcatctatagtaataatcagcggccctcttgggtccctgaccgattctctggctccaagtctggcacctcagcctccctggccatcagtgggctccagtctgaggatgaggctgattattactgtgtggcatgggatgacatcctgaatgctgtggtcttcgccggagggaccaagctgaccgtcctag

SEQ ID NO: 362 - DNA, 24 bp, Artificial Sequence
  Synthetic sequence nCoV-13 mAb CDRL1
acctccaacatcggaactaatgtt

SEQ ID NO: 363 - DNA, 9 bp, Artificial Sequence
  Synthetic sequence nCoV-13 mAb CDRL2
agtaataat

SEQ ID NO: 364 - DNA, 33 bp, Artificial Sequence
  Synthetic sequence nCoV-13 mAb CDRL3
gtggcatgggatgacatcctgaatgctgtggtc

SEQ ID NO: 365 - protein, 110 aa, Artificial Sequence
  Synthetic sequence nCoV-13 mAb VL
QSVLTQPPSASGTPGQRVTISCSGSTSNIGTNVVNWYQQLPGTAPKLLIYSNNQRPSWVPDRFSGSKSGTSASLAISGLQSEDEADYYCVAWDDILNAVVFAGGTKLTVL

SEQ ID NO: 366 - protein, 8 aa, Artificial Sequence
  Synthetic sequence nCoV-13 mAb CDRL1
TSNIGTNV

SEQ ID NO: 367 - protein, 3 aa, Artificial Sequence
  Synthetic sequence nCoV-13 mAb CDRL2
SNN

SEQ ID NO: 368 - protein, 11 aa, Artificial Sequence
  Synthetic sequence nCoV-13 mAb CDRL3
VAWDDILNAVV

SEQ ID NO: 369 - DNA, 29902 bp, betacoronavirus SARS coronavirus 2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggccgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 370 - protein, 7096 aa, betacoronavirus SARS coronavirus 2 [sequence omitted]

SEQ ID NO: 371 - protein, 1273 aa, betacoronavirus SARS coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 372 - DNA, 375 bp, Artificial Sequence
  Synthetic sequence nCoV-10 mAb VH
caagtgcaactcgtggagtccggcggcggcgtcgtccagccaggtcggtctctaaggctgtcgtgcgctgcaagtgggtttacattctctaattatggtatgcattgggtgaggcaggctcccggcaaggggctggaatggctcgctgtgatttcaagcgatggtcgaattaaattttatgctgactctgttaaaggcagatttactatgagtagagactcatcaaaaaacactctgtacctgcaaatgaatagcctgcgcgccgaggatacggctgtctactactgcgccaaagaccggttccaattcgcaagatcatggtatggagactacttcgattattgggggcaaggtactcaggtgactgtctcctca

SEQ ID NO: 373 - DNA, 375 bp, Artificial Sequence
  Synthetic sequence nCoV-10 mAb VH-v3
caggtgcaacttgtggaaagtggcggaggcgtggtgcagccggggcgatccttgcgcctctcctgtgccgccagcgggtttacattttccaattatggcatgcactgggtgcgtcaggcccccgggaagggcctggaatggcttgccgtgatcagcagtgacggacgcattaaattctacgccgattctgtcaagggtcggttcaccatgagtagagactcctcaaagaacactctgtacctgcagatgaatagcctgcgggccgaagacaccgctgtgtactactgtgctaaggaccggtttcaatttgctagatcattctacggggactacttcgactattggggccaaggcacgcaggtgacagtcagctca

SEQ ID NO: 374 - protein, 125 aa, Artificial Sequence
  Synthetic sequence nCoV-10 mAb VH-v3
QVQLVESGGGVVQPGRSLRLSCAASGFTFSNYGMHWVRQAPGKGLEWLAVISSDGRIKFYADSVKGRFTMSRDSSKNTLYLQMNSLRAEDTAVYYCAKDRFQFARSFYGDYFDYWGQGTQVTVSS

SEQ ID NO: 375 - protein, 18 aa, Artificial Sequence
  Synthetic sequence nCoV-10 mAb VH-v3 CDRH3
AKDRFQFARSFYGDYFDY

SEQ ID NO: 376 - DNA, 375 bp, Artificial Sequence
  Synthetic sequence nCoV-10 mAb VH-v4
caagtccagcttgtcgagtctggagggggcgtagtgcagcccgggcgcagtttgagattaagctgcgccgcctctggtttcacgttctccaattacggtatgcactgggtgcgacaggcacccggcaaggggctggaatggctggccgtcattagttcagacgggcggatcaaattttacgctgacagtgtgaagggtaggtttaccatgtcaagagactccagcaaaaacacattatacctgcagatgaacagcctgcgcgccgaggacaccgccgtatattattgcgctaaggaccgctttcagttcgctaggtctgcctacggtgactattttgattattggggacagggaactcaggtaacggtcagctca

SEQ ID NO: 377 - protein, 125 aa, Artificial Sequence
  Synthetic sequence nCoV-10 mAb VH-v4
QVQLVESGGGVVQPGRSLRLSCAASGFTFSNYGMHWVRQAPGKGLEWLAVISSDGRIKFYADSVKGRFTMSRDSSKNTLYLQMNSLRAEDTAVYYCAKDRFQFARSAYGDYFDYWGQGTQVTVSS

SEQ ID NO: 378 - protein, 18 aa, Artificial Sequence
  Synthetic sequence nCoV-10 mAb VH-v4
AKDRFQFARSAYGDYFDY

SEQ ID NO: 379 - DNA, 336 bp, Artificial Sequence
  Synthetic sequence nCoV-10 mAb VL (Vk)
gcggttgcaatgactcagtccccactctccttgcccgtgacactcgggcaaccggcctccatctcttgccggagcaatcagtcccttgtttatagcgacgggaatatctatctgaattggttccagcagaggcccgggcagtcaccaatgcgcctcatctatcgggtgtccaaccgggattcaggtgtgcccgaccggttttctggcagcggatcaggaacggactttacactcaaaatttcccgcgtggaggctgaggatgtcggaatctattattgcatgcagggcacgcactggcctccaaccttcggcggaggaaccaaggtggagatcaag

SEQ ID NO: 380 - protein, 112 aa, Artificial Sequence
  Synthetic sequence nCoV-10 mAb VL-v3 (Vk)
DVVMTQSPLSLPVTLGQPASISCRSQQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 381 - DNA, 336 bp, Artificial Sequence
  Synthetic sequence nCoV-10 mAb VL-v3 (Vk)
gatgtggtaatgacccaaagccccctgtcattgccagtgacactgggccagccggcctcaattagttgcaggtctcagcagtcactggtgtactccgatggcaatatctacctaaactggttccagcagcggcctgggcaatctcctatgcgacttatatatagagtgagtaaccgggacagtggggtccctgatagattttcaggtagcggcagtggcacagatttcaccctgaagatatctcgggtagaagcagaggacgtggggatatattattgcatgcagggaacgcactggccccctaccttcggcggcggaacaaaagtggaaataaaa

SEQ ID NO: 382 - protein, 220 aa, betacoronavirus SARS coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 383 - protein, 112 aa, Artificial Sequence
  Synthetic sequence nCoV-10 mAb VL-v3 (Vk)
DVVMTQSPLSLPVTLGQPASISCRSSQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 384 - DNA, 336 bp, Artificial Sequence
  Synthetic sequence nCoV-10 mAb VL-v4 (Vk)
gacgtggtgatgacacagagcccactatcccttccggtcaccttgggccagccagcaagcatttcctgtcggtcgagccagtctctggtctatagtgacggaaacatttatttgaattggtttcaacagaggcccggacagtcaccaatgcgcctcatctaccgcgtctctaacagagactctggagtccccgatcgcttttccggtagcgggtctgggaccgattttacattgaagatctccagggtggaagccgaggacgttggtatctactactgcatgcagggtacccattggccaccgaccttcggcgggggcaccaaagtggagatcaaa

SEQ ID NO: 385 - protein, 112 aa, Artificial Sequence
  Synthetic sequence nCoV-10 mAb VL-v5 (Vk)
DVVMTQSPLSLPVTLGQPASISCRSSQSLVYSDGNIYLNWFQQRPGQSPMRLIYRVSNRDSGVPDRFSGSGSGTDFTLKISRVEAEDVGIYYCMQGTHFPPTFGGGTKVEIK

SEQ ID NO: 386 - protein, 9 aa, Artificial Sequence
  Synthetic sequence nCoV-10 mAb VL-v5 (Vk) CDRL3
MQGTHFPPT

SEQ ID NO: 387 - DNA, 336 bp, Artificial Sequence
  Synthetic sequence nCoV-10 mAb VL-v5 (Vk)
gatgtcgtgatgacacagtcgccactgagtctaccggtgacactggggcagccggcctccatttcatgtaggagcagtcagtcgttagtgtactctgacggtaacatctatcttaattggttccagcaaagacctggccagtcacctatgcggctgatatatagagtttccaacagagactccggggtgccagacaggttctctggaagtggctctggaactgactttacactgaagatcagcagggtggaggcagaggacgtgggtatctattactgtatgcaagggactcatttccctccaacattcggaggagggacaaaagtggagataaaa

SEQ ID NO: 388 - protein, 112 aa, Artificial Sequence
  Synthetic sequence nCoV-10 mAb VL-v6 (Vk)
DVVMTQSPLSLPVTLGQPASISCRSQQSLVYSDGNIYLNWFQQRPGQSPMRLIYRVSNRDSGVPDRFSGSGSGTDFTLKISRVEAEDVGIYYCMQGTHFPPTFGGGTKVEIK

SEQ ID NO: 389 - DNA, 336 bp, Artificial Sequence
  Synthetic sequence nCoV-10 mAb VL-v6 (Vk)
gacgtggtgatgactcaatcacccctctccctaccggtcacactgggccagccagcaagcatttcatgccgttcgcagcagtccctcgtctattcggacggcaatatttacctcaactggtttcagcagcgacccgggcagtcacccatgcgacttatctacagggtgagcaacagagattccggggtccccgatcgcttctcaggcagtggcagcggcaccgatttcaccctaaaaattagccgcgtggaagcggaagatgtgggtatctactactgcatgcagggaactcactttccacccacttttggaggggggaccaaagttgaaatcaag

SEQ ID NO: 390 - protein, 72 aa, betacoronavirus SARS coronavirus 2
NSNNLDSKVGGNYNYLYRLFRKSNLKPFERDISTEIYQAGSTPCNGVEGFNCYFPLQSYGFQPTNGVGYQPY

SEQ ID NO: 391 - protein, 107 aa, Artificial Sequence [sequence omitted]

SEQ ID NO: 392 - protein, 112 aa, Artificial Sequence
  Synthetic sequence nCoV-10 mAb VL-v7 (Vk)
DVVMTQSPLSLPVTLGQPASISCRSSQSLVYSDGNIYLNWFQQRPGQSPMRLIYRVSNRDSGVPDRFSGSGSGTDFTLKISRVEAEDVGIYYCMQGTHYPPTFGGGTKVEIK

SEQ ID NO: 393 - protein, 112 aa, Artificial Sequence
  Synthetic sequence nCoV-10 mAb VL-v8 (Vk)
DVVMTQSPLSLPVTLGQPASISCRSSQSLVYSEGNIYLNWFQQRPGQSPMRLIYRVSNRDSGVPDRFSGSGSGTDFTLKISRVEAEDVGIYYCMQGTHYPPTFGGGTKVEIK

SEQ ID NO: 394 - protein, 9 aa, Artificial Sequence
  Synthetic sequence nCoV-10 mAb VL-v7/8 (Vk) CDRL3
MQGTHYPPT

SEQ ID NO: 395 - protein, 330 aa, Artificial Sequence
  Synthetic sequence nCov10 CH1-CH3 G1m17; IgHG1-01_MLNS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 396 - protein, 125 aa, Artificial Sequence
  Synthetic sequence nCoV-10 mAb VH-v5
QVQLVESGGGVVQPGRSLRLSCAASGFTFSNYGMHWVRQAPGKGLEWLAVISSDGRIKFYADSVKGRFTMSRDSSKNTLYLQMNSLRAEDTAVYYCAKDRFQFARSYYGDYFDYWGQGTQVTVSS

SEQ ID NO: 397 - protein, 18 aa, Artificial Sequence
  Synthetic sequence nCoV-10 mAb VH-v5 CDRH3
AKDRFQFARSYYGDYFDY

SEQ ID NO: 398 - protein, 11 aa, Artificial Sequence
  Synthetic sequence nCoV-10 mAb VL-v8 (Vk) CDRL1
QSLVYSEGNIY

SEQ ID NO: 399 - protein, 11 aa, Artificial Sequence
  Synthetic sequence nCoV-10 mAb VL-v9
QALVYSDGNIY

SEQ ID NO: 400 - protein, 112 aa, Artificial Sequence
  Synthetic sequencenCoV-10 mAb VL-v9 (Vk)
AVAMTQSPLSLPVTLGQPASISCRSNQALVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 401 - protein, 11 aa, Artificial Sequence
  Synthetic sequence  nCoV-10 mAb VL-v10 (Vk) CDRL1
QGLVYSDGNIY

SEQ ID NO: 402 - protein, 112 aa, Artificial Sequence
  Synthetic sequence  nCoV-10 mAb VL-v10 (Vk)
AVAMTQSPLSLPVTLGQPASISCRSNQGLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 403 - protein, 11 aa, Artificial Sequence
  Synthetic sequence  nCoV-10 mAb VL-v11 (Vk) CDRL1
QVLVYSDGNIY

SEQ ID NO: 404 - protein, 112 aa, Artificial Sequence
  Synthetic sequence  nCoV-10 mAb VL-v11
AVAMTQSPLSLPVTLGQPASISCRSNQVLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 405 - protein, 11 aa, Artificial Sequence
  Synthetic sequence  nCoV-10 mAb VL-v12 (Vk) CDRL1
PSLVYSDGNIY

SEQ ID NO: 406 - protein, 112 aa, Artificial Sequence
  Synthetic sequence nCoV-10 mAb VL-v12 (Vk)
AVAMTQSPLSLPVTLGQPASISCRSNPSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 407 - protein, 112 aa, Artificial Sequence
  Synthetic sequence nCoV-10 mAb VL-v13 (Vk)
AVAMTQSPLSLPVTLGQPASISCRSDQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 408 - protein, 112 aa, Artificial Sequence
  Synthetic sequence nCoV-10 mAb VL-v14 (Vk)
AVAMTQSPLSLPVTLGQPASISCRSEQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 409 - protein, 112 aa, Artificial Sequence
  Synthetic sequence nCoV-10 mAb VL-v15 (Vk)
DVVMTQSPLSLPVTLGQPASISCRSNQALVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 410 - protein, 112 aa, Artificial Sequence
  Synthetic sequence nCoV-10 mAb VL-v16 (Vk)
DVVMTQSPLSLPVTLGQPASISCRSNQGLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 411 - protein, 112 aa, Artificial Sequence
  Synthetic sequence nCoV-10 mAb VL-v17 (Vk)
DVVMTQSPLSLPVTLGQPASISCRSNQVLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 412 - protein, 112 aa, Artificial Sequence
  Synthetic sequence nCoV-10 mAb VL-v18 (Vk)
DVVMTQSPLSLPVTLGQPASISCRSNPSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 413 - protein, 112 aa, Artificial Sequence
  Synthetic sequence nCoV-10 mAb VL-v19 (Vk)
DVVMTQSPLSLPVTLGQPASISCRSDQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 414 - protein, 112 aa, Artificial Sequence
  Synthetic sequence nCoV-10 mAb VL-v20 (Vk)
DVVMTQSPLSLPVTLGQPASISCRSEQSLVYSDGNIYLNWFQQRPGQSPMRLIYRVSNRDSGVPDRFSGSGSGTDFTLKISRVEAEDVGIYYCMQGTHWPPTFGGGTKVEIK

SEQ ID NO: 415 - protein, 125 aa, Artificial Sequence
  Synthetic sequence nCoV-10 mAb VH-v21
QVQLVESGGGVVQPGRSLRLSCAASGFTFSNYGMHWVRQAPGKGLEWLAVISFDGRIKFYADSVKGRFTMSRDSSKNTLYLQMNSLRAEDTAVYYCAKDRFQFARSWYGDYFDYWGQGTQVTVSS

SEQ ID NO: 416 - protein, 8 aa, Artificial Sequence
  Synthetic sequence nCoV-10 mAb VH-v21 CDRH2
ISFDGRIK

SEQ ID NO: 417 - DNA, 421 bp, Artificial Sequence
  Synthetic sequence nCoV-10 mAb VH-v21
gccagtgtcaggtgcagctggtggagtctgggggaggcgtggtccagcctgggaggtccctgagactctcctgtgcagcctctggattcaccttcagtaactatggcatgcactgggtccgccaggctccaggcaaggggctggagtggctggcagttatatcatttgatggaagaattaagttctatgcagactccgtgaagggccgattcaccatgtccagagacagttccaagaacacgctgtatctgcaaatgaacagcctgagagctgaggacacggctgtgtattactgtgcgaaagatcggttccagtttgccagaagctggtacggtgactactttgactactggggccagggaacccaggtcaccgtctcctcagcctccaccaagggcccatcggtcttccccctggcacc

(19) United States
(12) Patent Application Publication
(10) Pub. No.: US 2025/0263466 A1
(43) Pub. Date: Aug. 21, 2025

(54) ANTIBODIES AGAINST SARS-COV-2 AND METHODS OF USING THE SAME

(71) Applicant: VIR BIOTECHNOLOGY, INC., San Francisco, CA (US)

(72) Inventors: Davide CORTI, Bellinzona (CH); Katja FINK, Bellinzona (CH); Martina BELTRAMELLO, Bellinzona (CH); Elisabetta CAMERONI, Bellinzona (CH); Dora PINTO, Bellinzona (CH); Siro BIANCHI, Bellinzona (CH); Gyorgy SNELL, San Francisco, CA (US)

(21) Appl. No.: 17/760,032

(22) PCT Filed: Feb. 2, 2021
(86) PCT No.: PCT/US2021/016172

§ 371 Date: Aug. 3, 2022

Related U.S. Application Data
(60) Provisional application No. 62/969,592, filed on Feb. 3, 2020.
(60) Provisional application No. 62/970,062, filed on Feb. 4, 2020.
(60) Provisional application No. 62/971,552, filed on Feb. 7, 2020.
(60) Provisional application No. 62/977,941, filed on Feb. 18, 2020.
(60) Provisional application No. 63/016,228, filed on Apr. 27, 2020.
(60) Provisional application No. 63/023,858, filed on May 12, 2020.

(51) Int. Cl. C07K 16/10; A61P 31/14; G01N 33/569

(52) U.S. Cl. CPC C07K 16/1003; A61P 31/14; G01N 33/56983; C07K 2317/21; C07K 2317/24; C07K 2317/31; G01N 2333/165; G01N 2469/10

(57) ABSTRACT

The instant disclosure provides antibodies and antigen-binding fragments thereof that can bind to a SARS-CoV-2 antigen and, in certain embodiments, are capable of neutralizing a SARS-CoV-2 infection in a subject. Also provided are polynucleotides that encode an antibody or antigen-binding fragment, vectors and host cells that comprise a polynucleotide, pharmaceutical compositions, and methods of using the presently disclosed antibodies, antigen-binding fragments, polynucleotides, vectors, host cells, and compositions to treat or diagnose a sarbecovirus and/or SARS-CoV-2 infection.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930585_401WO_SEQUENCE_LISTING.txt. The text file is 269 KB, was created on Jan. 31, 2021, and is being submitted electronically via EFS-Web.

### BACKGROUND

A novel betacoronavirus emerged in Wuhan, China, in late 2019. As of Jan. 23, 2021, approximately 98,573,000 cases of infection by this virus (termed, among other names, SARS-CoV-2), were confirmed worldwide, and had resulted in approximately 2,116,000 deaths. Therapies for preventing or treating SARS-CoV-2 infection, and diagnostic tools for detecting and diagnosis a SARS-CoV-2 infection, are needed.

### BRIEF DESCRIPTION OF THE DRAWINGS

FIGS. 1A-ID show binding of exemplary antibodies of the present disclosure to recombinant SARS-CoV S1 protein, as measured by ELISA. Antibody nCoV-1 comprises the VH sequence of SEQ ID NO: 325 and the VL sequence of SEQ ID NO:333. Antibody nCoV-2 comprises the VH sequence of SEQ ID NO:293 and the VL sequence of SEQ ID NO:301. Antibody nCoV-20 comprises the VH sequence of SEQ ID NO:101 and the VL sequence of SEQ ID NO:109. Antibody nCoV-18 comprises the VH sequence of SEQ ID NO:245 and the VL sequence of SEQ ID NO:253. Antibody nCoV-19 comprises the VH sequence of SEQ ID NO:309 and the VL sequence of SEQ ID NO:317. (FIG. 1A.) Antibody nCoV-16 comprises the VH sequence of SEQ ID NO:85 and the VL sequence of SEQ ID NO:93. Antibody nCoV-10 comprises the VH sequence of SEQ ID NO:21 and the VL sequence of SEQ ID NO:29. Antibody nCoV-6 comprises the VH sequence of SEQ ID NO:69 and the VL sequence of SEQ ID NO:77. Antibody nCoV-3 comprises the VH sequence of SEQ ID NO:5 and the VL sequence of SEQ ID NO:13. Antibody nCoV-14 comprises the VH sequence of SEQ ID NO:213 and the VL sequence of SEQ ID NO:221. (FIG. 1B.) Antibody nCoV-4 comprises the VH sequence of SEQ ID NO:117 and the VL sequence of SEQ ID NO:125. Antibody nCoV-5 comprises the VH sequence of SEQ ID NO:197 and the VL sequence of SEQ ID NO:205. Antibody nCoV-12 comprises the VH sequence of SEQ ID NO:181 and the VL sequence of SEQ ID NO:189. Antibody nCoV-9 comprises the VH sequence of SEQ ID NO:229 and the VL sequence of SEQ ID NO:237. (FIG. 1C.) Antibody nCoV-8 comprises the VH sequence of SEQ ID NO:261 and the VL sequence of SEQ ID NO:269. Antibody nCoV-7 comprises the VH sequence of SEQ ID NO:277 and the VL sequence of SEQ ID NO:285. Antibody nCoV-11 comprises the VH sequence of SEQ ID NO:341 and the VL sequence of SEQ IDNO:349. (FIG. 1D.)

FIGS. 2A and 2B show staining of SARS-CoV-2-S, SARS-CoV-S, MERS-CoV-S and Mock transfected cells using a panel of exemplary human monoclonal antibodies (mAbs), expressed as recombinant IgG1. All mAbs were tested at 10 μg/ml with the following exceptions: nCoV-5 (1 μg/ml); nCoV-7 (2 μg/ml); nCoV-8 (1.5 μg/ml); nCoV-9 (8.5 μg/ml); nCoV-12 (0.8 μg/ml); nCoV-13 (7 μg/ml); and nCoV-15 (0.8 μg/ml). Antibody nCoV-13 comprises the VH sequence of SEQ ID NO:357 and the VL sequence of SEQ ID NO:365. Antibody nCoV-15 comprises the VH sequence of SEQ ID NO:165 and the VL sequence of SEQ ID NO:173. (A) Percentage of staining-positive cells. (B) Percentage of staining-positive cells normalized to mode.

FIGS. 3A and 3B show binding by exemplary antibody nCoV-10 at different concentrations to surface-expressed S protein from viruses SARS-CoV-2, SARS-CoV, and MERS-CoV. (A) Histograms depict the number of cells vs. the fluorescence intensity of antibody binding to S-transfected cells. (B) Percentage of positive binding at 12 concentrations of nCov-10 antibody (10 μg/ml down to 0.004 μg/ml), as defined by differential staining of CoV-S-transfectants versus mock-transfectants.

FIGS. 4A and 4B show binding by exemplary antibody nCoV-6 at different concentrations to surface-expressed S protein from viruses SARS-CoV-2, SARS-CoV, and MERS-CoV. (A) Histograms depict the number of cells vs. the fluorescence intensity of antibody binding to S-transfected cells. (B) Graphs depict the percentage of positive binding at 12 concentrations of nCoV-6 antibody (10 μg/ml down to 0.004 μg/ml), as defined by differential staining of CoV-S-transfectants versus mock-transfectants.

FIG. 4C shows binding by nCoV-10, nCoV-6, and nCoV-1 to SARS-CoV S protein or SARS-CoV-2 S protein expressed on cells 24 hours after transfection.

FIGS. 5A-5F show results of cross-competition assays using Octet (BLI) to investigate the epitopes recognized by SARS-CoV-2 cross-reactive antibodies nCoV-6 and nCoV-10 and the SARS-CoV-specific antibodies nCoV-4 and nCoV-1. SARS-CoV Receptor Binding Domain (RBD) was first immobilized on anti-His sensors (step 1), then sensors were moved into wells containing antibody 1 (step 2) and finally into wells containing antibody 2 (step 3). If a binding event is detected at step 3, antibody 2 has a non-overlapping epitope than the epitope recognized by antibody 1; if no binding is detected at step 3, antibody 1 and 2 share overlapping epitopes. The order of the antibodies used in steps 2 and 3 was as shown above the graph in each of (A)-(F). FIGS. 5A-5D show cross-competition assays using nCoV-6 and nCoV-10. In FIG. 5E, nCoV-4 Fab was used as antibody 1, and nCoV-6 or nCoV-10 was used as antibody 2. FIG. 5F shows data from cross-competition assays among nCoV-10, nCoV-6, and nCoV-1. The antibody used as antibody 1 is indicated in each panel of FIG. 5F.

FIG. 6A shows an alignment of the S protein RBD from multiple SARS-CoV isolates (Urbani, CHUK-1, GZ02, HC_SZ_61_03, A031G), the SARS-like bat CoV WIV1, and SARS-CoV-2. Indicated in dark grey are residues L443 (F455 in SARS-CoV-2), F460 (Y473 in SARS-CoV-2) and P462 (A475 in SARS-CoV-2). Indicated in in light grey is the Receptor Binding Motif (RBM). Residue numbering shown for each row of the figure is with respect to the RBD, not the full S protein.

FIG. 6B shows the positioning of SARS-CoV (RBD) residues P462, F460, and L443 when RBD is in complex with human ACE2 (pdb, 2AJF).

FIG. 7 provides an illustration of SARS-CoV RBD bound to human ACE2 (pdb, 2AJF), with residues P462 (corresponding to residue 475 in SARS-CoV-2) and V354 (corresponding to residue 376 in SARS-CoV-2) indicated.

FIG. 8 shows SARS-CoV RBD with: (top, left) the ACE2 footprint on RBD indicated, with nCoV-10 SARS CoV escape mutant residues identified; (top, center) the nCoV-1 footprint on RBD indicated; and (top, right) amino acid residue differences between SARS-CoV-2 RBD and SARS-CoV RBD. At bottom is an alignment showing SARS CoV and SARS-CoV-2 RBM amino acid sequences.

FIG. 9 shows that exemplary antibody nCoV-10 of the present disclosure can inhibit binding of a coronavirus RBD to human ACE2.

FIG. 10 shows illustrations of the structures of SARS CoV RBD (3-d space-filling models in center) and human ACE2 (ribbon diagrams, outside relative to center).

FIGS. 11A and 11B show the ability of monoclonal antibodies nCoV-1 and nCoV-10 to inhibit association of SARS-S1 protein with human ACE2, as assayed by Octet (BLI). FIG. 11A shows % inhibition on the y-axis. FIG. 11B shows response on the y-axis.

### DETAILED DESCRIPTION

Provided herein are antibodies and antigen-binding fragments that bind to SARS-CoV-2 (e.g., a SARS-CoV-2 surface glycoprotein and/or RBD, as described herein, in a SARS-CoV-2 virion and/or expressed on the surface of a cell infected by SARS-CoV-2). In certain embodiments, presently disclosed antibodies and antigen-binding fragments can neutralize a SARS-CoV-2 infection in an in vitro model of infection and/or in a human subject. Also provided are polynucleotides that encode the antibodies and antigen-binding fragments, vectors, host cells, and related compositions, as well as methods of using the antibodies, nucleic acids, vectors, host cells, and related compositions to treat (e.g., reduce, delay, eliminate, or prevent) a SARS-CoV-2 infection in a subject and/or in the manufacture of a medicament for treating or preventing a SARS-CoV-2 infection in a subject.

Prior to setting forth this disclosure in more detail, it may be helpful to an understanding thereof to provide definitions of certain terms to be used herein. Additional definitions are set forth throughout this disclosure.

As used herein, “SARS-CoV-2”, also referred to herein as “Wuhan seafood market pneumonia virus”, or “Wuhan coronavirus” or “Wuhan CoV”, or “novel CoV”, or “nCoV”, or “2019 nCoV”, or “Wuhan nCoV” is a betacoronavirus believed to be of lineage B (sarbecovirus). SARS-CoV-2 was first identified in Wuhan, Hubei province, China, in late 2019 and spread within China and to other parts of the world by early 2020. Symptoms of SARS-CoV-2 infection include fever, dry cough, dyspnea, fatigue, body aches, headache, new loss of taste or smell, sore throat, congestions or runny nose, nausea or vomiting, diarrhea, persistent pressure or pain in the chest, new confusion, inability to wake or stay awake, and bluish lips or face.

The genomic sequence of SARS-CoV-2 isolate Wuhan-Hu-1 is provided in SEQ ID NO.:369 (see also GenBank MN908947.3, Jan. 23, 2020), and the amino acid translation of the genome is provided in SEQ ID NO.:370 (see also GenBank QHD43416.1, Jan. 23, 2020). Like other coronaviruses (e.g., SARS CoV), SARS-CoV-2 comprises a “spike” or surface (“S”) type I transmembrane glycoprotein containing a receptor binding domain (RBD). RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus RBD is believed to interact with ACE2.

The amino acid sequence of the SARS-CoV-2 Wuhan-Hu-1 surface glycoprotein is provided in SEQ ID NO.:371. The amino acid sequence of SARS-CoV-2 RBD is provided in SEQ ID NO.:382. SARS-CoV-2 S protein has approximately 73% amino acid sequence identity with SARS-CoV. The amino acid sequence of SARS-CoV-2 RBM is provided in SEQ ID NO.:390. SARS-CoV-2 RBD has approximately 75% to 77% amino acid sequence similarity to SARS coronavirus RBD, and SARS-CoV-2 RBM has approximately 50% amino acid sequence similarity to SARS coronavirus RBM.

Unless otherwise indicated herein, SARS-CoV-2 refers to a virus comprising the amino acid sequence set forth in any one or more of SEQ ID NOs.:370, 371, and 382, optionally with the genomic sequence set forth in SEQ ID NO.:369.

There have been a number of emerging SARS-CoV-2 variants. Some SARS-CoV-2 variants contain an N439K mutation, which has enhanced binding affinity to the human ACE2 receptor (Thomson, E. C., et al., The circulating SARS-CoV-2 spike variant N439K maintains fitness while evading antibody-mediated immunity. bioRxiv, 2020). Some SARS-CoV-2 variants contain an N501Y mutation, which is associated with increased transmissibility, including the lineages B.1.1.7 (also known as 20I/501Y.V1 and VOC 202012/01) and B.1.351 (also known as 20H/501Y.V2), which were discovered in the United Kingdom and South Africa, respectively (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640; Leung, K., et al., Early empirical assessment of the N501Y mutant strains of SARS-CoV-2 in the United Kingdom, October to November 2020. medRxiv, 2020: p. 2020.12.20.20248581). B.1.351 also includes two other mutations in the RBD domain of SARS-CoV2 spike protein, K417N and E484K (Tegally, H., et al., Emergence and rapid spread of a new severe acute respiratory syndrome-related coronavirus 2 (SARS-CoV-2) lineage with multiple spike mutations in South Africa. medRxiv, 2020: p. 2020.12.21.20248640). Other SARS-CoV-2 variants include the Lineage B.1.1.28, which was first reported in Brazil; the Variant P.1, lineage B.1.1.28 (also known as 20J/501Y.V3), which was first reported in Japan; Variant L452R, which was first reported in California in the United States (Pan American Health Organization, Epidemiological update: Occurrence ofvariants of SARS-CoV-2 in the Americas, Jan. 20, 2021, available at https://reliefweb.int/sites/reliefweb.int/files/resources/2021-jan-20-phe-epi-update-SARS-CoV-2.pdf). Other SARS-CoV-2 variants include a SARS CoV-2 of clade 19A; SARS CoV-2 of clade 19B; a SARS CoV-2 of clade 20A; a SARS CoV-2 of clade 20B; a SARS CoV-2 of clade 20C; a SARS CoV-2 of clade 20D; a SARS CoV-2 of clade 20E (EU1); a SARS CoV-2 of clade 20F; a SARS CoV-2 of clade 20G; and SARS CoV-2 B1.1.207; and other SARS CoV-2 lineages described in Rambaut, A., et al., A dynamic nomenclature proposal for SARS-CoV-2 lineages to assist genomic epidemiology. Nat Microbiol 5, 1403-1407 (2020).

Other coronaviruses are believed to enter cells by binding to other receptors (e.g., 9-O-Ac-Sia receptor analog; DPP4; APN).

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. Any of the presently disclosed compositions (e.g., antibody, antigen-binding fragment, pharmaceutical composition, polynucleotide, vector, host cell) can be provided in isolated form.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (K_{D}) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding an antibody or antigen-binding fragment or other polypeptide, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mátés et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked to a gene of interest if they are contiguous with the gene of interest or if they act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based antibody or antigen-binding fragment-encoding plasmid vectors for direct administration to a subject, are described further herein.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In the context of a SARS-CoV-2 infection, a “host” refers to a cell or a subject infected with SARS-CoV-2.

“Antigen” or “Ag”, as used herein, refers to an immunogenic molecule that provokes an immune response. This immune response may involve antibody production, activation of specific immunologically-competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in SARS-CoV-2 (e.g., a surface glycoprotein or portion thereof), such as present in a virion, or expressed or presented on the surface of a cell infected by SARS-CoV-2.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

### Antibodies and Antigen-Binding Fragments

In one aspect, the present disclosure provides an isolated antibody, or an antigen-binding fragment thereof, that comprises a heavy chain variable domain (VH) comprising a CDRH1, a CDRH2, and a CDRH3, and a light chain variable domain (VL) comprising a CDRL1, a CDRL2, and a CDRL3, and is capable of binding to a surface glycoprotein of SARS-CoV-2. In certain embodiments, the antibody or antigen-binding fragment is capable of binding to a surface glycoprotein of SARS-CoV-2 expressed on a cell surface of a host cell and/or on a SARS-CoV-2 virion.

In certain embodiments, an antibody or antigen-binding fragment of the present disclosure associates with or unites with a SARS-CoV-2 surface glycoprotein epitope or antigen comprising the epitope, while not significantly associating or uniting with any other molecules or components in a sample.

In certain embodiments, an antibody or antigen-binding fragment of the present disclosure associates with or unites (e.g., binds) to a SARS-CoV-2 surface glycoprotein epitope, and can also associate with or unite with an epitope from another coronavirus (e.g., SARS CoV) present in the sample, but does not significantly associate or unite with any other molecules or components in the sample. In other words, in certain embodiments, an antibody or antigen binding fragment of the present disclosure is cross-reactive for SARS-CoV-2 and one or more additional coronavirus, and is specific for the SARS-CoV-2 and one or more additional coronavirus.

In certain embodiments, an antibody or antigen-binding fragment of the present disclosure specifically binds to a SARS-CoV-2 surface glycoprotein. As used herein, “specifically binds” refers to an association or union of an antibody or antigen-binding fragment to an antigen with an affinity or Kₐ (i.e., an equilibrium association constant of a particular binding interaction with units of 1/M) equal to or greater than 10⁻⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (Kₐ) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10⁻¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (Kₐ) of a particular binding interaction with units of M (e.g., 10⁻⁵ M to 10⁻¹³ M).

A variety of assays are known for identifying antibodies of the present disclosure that bind a particular target, as well as determining binding domain or binding protein affinities, such as Western blot, ELISA, analytical ultracentrifugation, spectroscopy, and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known.

In certain examples, binding can be determined by recombinantly expressing a SARS-CoV-2 antigen in a host cell (e.g., by transfection) and immunostaining the (e.g., fixed, or fixed and permeabilized) host cell with antibody and analyzing binding by flow cytometry (e.g., using a ZE5 Cell Analyzer (BioRad®) and FlowJo software (TreeStar). In some embodiments, positive binding can be defined by differential staining by antibody of SARS-CoV-2-expressing cells versus control (e.g., mock) cells.

In certain embodiments, an antibody of the present disclosure is capable of neutralizing infection by SARS-CoV-2. As used herein, a “neutralizing antibody” is one that can neutralize, i.e., prevent, inhibit, reduce, impede, or interfere with, the ability of a pathogen to initiate and/or perpetuate an infection in a host. The terms “neutralizing antibody” and “an antibody that neutralizes” or “antibodies that neutralize” are used interchangeably herein. In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

In certain embodiments, the antibody or antigen-binding fragment (i) recognizes an epitope in the ACE2 receptor binding motif (RBM, SEQ ID NO.:390) of SARS-CoV-2; (ii) is capable of blocking an interaction between SARS-CoV-2 and ACE2; (ii) is capable of binding to SARS-CoV-2 S protein with greater avidity than to SARS coronavirus S protein; (iv) is capable of staining about 30%, about 35%, about 40%, about 50%, about 55%, about 56%, about 57%, about 58%, about 59%, about 60%, or more of target cells expressing SARS-CoV-2 surface glycoprotein in a sample comprising about 50,000 of the target cells (e.g., ExpiCHO cells) in approximately 100 μL when the antibody or antigen-binding fragment is present at 10 μg/ml (e.g., staining as determined by a flow cytometry ELISA); (v) recognizes an epitope that is conserved in the ACE2 RBM of SARS-CoV-2 and in an ACE2 RBM of SARS coronavirus; (vi) is cross-reactive against SARS-CoV-2 and SARS coronavirus; (vii) recognizes an epitope in the SARS-CoV-2 \surface glycoprotein that is not in the ACE2 RBM; or (viii) any combination of (i)-(vii).

Terms understood by those in the art of antibody technology are each given the meaning acquired in the art, unless expressly defined differently herein. For example, the term “antibody” refers to an intact antibody comprising at least two heavy (H) chains and two light (L) chains inter-connected by disulfide bonds, as well as any antigen-binding portion or fragment of an intact antibody that has or retains the ability to bind to the antigen target molecule recognized by the intact antibody, such as an scFv, Fab, or Fab′2 fragment. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

The terms “VL” or “VL” and “VH” or “VH” refer to the variable binding region from an antibody light chain and an antibody heavy chain, respectively. In certain embodiments, a VL is a kappa (κ) class (also “VK” herein). In certain embodiments, a VL is a lambda (k) class. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary structure by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

As used herein, a “variant” of a CDR refers to a functional variant of a CDR sequence having up to 1-3 amino acid substitutions (e.g., conservative or non-conservative substitutions), deletions, or combinations thereof.

Numbering of CDR and framework regions may be according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Pluckthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. In certain embodiments, an antibody or antigen-binding fragment is provided that comprises the three CDRs of a VH sequence according to any one of SEQ ID NOs.: 5, 21, 37, 53, 69, 85, 101, 117, 133, 149, 165, 181, 197, 213, 229, 245, 261, 277, 293, 309, 325, 341, 357, 374, 377, 395, or 415, and/or the three CDRs of a VL sequence according to any one of SEQ ID NOs.: 13, 29, 45, 61, 77, 93, 109, 125, 141, 157, 173, 189, 205, 221, 237, 253, 269, 285, 301, 317, 333, 349, 365, 380, 383, 385, 388, 392, 393, 400, 402, 404, and 406-414, as determined using any known CDR numbering method, including the Kabat, Chothia, EU, IMGT, Martin (Enhanced Chothia), Contact, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software (www.chemcomp.com).

In certain embodiments, an antibody or an antigen-binding fragment is provided that comprises a heavy chain variable domain (VH) comprising a CDRH1, a CDRH2, and a CDRH3, and a light chain variable domain (VL) comprising a CDRL1, a CDRL2, and a CDRL3, wherein: (i) the CDRH1 comprises or consists of the amino acid sequence according to any one of SEQ ID NOs.: 6, 22, 38, 54, 70, 86, 102, 118, 134, 150, 166, 182, 198, 214, 230, 246, 262, 278, 294, 310, 326, 342, or 358, ora sequence variant thereof comprising one, two, or three acid substitutions, one or more of which substitutions is optionally a conservative substitution and/or is a substitution to a germline-encoded amino acid; (ii) the CDRH2 comprises or consists of the amino acid sequence according to any one of SEQ ID NOs.: 7, 23, 39, 55, 71, 87, 103, 119, 135, 151, 167, 183, 199, 215, 231, 247, 263, 279, 295, 311, 327, 343, 359, or 416, or a sequence variant thereof comprising one, two, or three amino acid substitutions, one or more of which substitutions is optionally a conservative substitution and/or is a substitution to a germline-encoded amino acid; (iii) the CDRH3 comprises or consists of the amino acid sequence according to any one of SEQ ID NOs.: 8, 24, 40, 56, 72, 88, 104, 120, 136, 152, 168, 184, 200, 216, 232, 248, 264, 280, 296, 312, 328, 344, 360, 375, 378, or 397, or a sequence variant thereof comprising one, two, or three amino acid substitutions, one or more of which substitutions is optionally a conservative substitution and/or is a substitution to a germline-encoded amino acid; (iv) the CDRL1 comprises or consists of the amino acid sequence according to any one of SEQ ID NOs.: 14, 30, 46, 62, 78, 94, 110, 126, 142, 158, 174, 190, 206, 222, 238, 254, 270, 286, 302, 318, 334, 350, 366, 398, 399, 401, 403, or 405, or a sequence variant thereof comprising one, two, or three amino acid substitutions, one or more of which substitutions is optionally a conservative substitution and/or is a substitution to a germline-encoded amino acid; (v) the CDRL2 comprises or consists of the amino acid sequence according to any one of SEQ ID NOs.: 15, 31, 47, 63, 79, 95, 111, 127, 143, 159, 175, 191, 207, 223, 239, 255, 271, 287, 303, 319, 335, 351, or 367, orasequence variant thereof comprising one, two, or three amino acid substitutions, one or more of which substitutions is optionally a conservative substitution and/or is a substitution to a germline-encoded amino acid; and/or (vi) the CDRL3 comprises or consists of the amino acid sequence according to any one of SEQ ID NOs.: 16, 32, 48, 64, 80, 96, 112, 128, 144, 160, 176, 192, 208, 224, 240, 256, 272, 288, 304, 320, 336, 352, 358, 386, or 394, or a sequence variant thereof comprising having one, two, or three amino acid substitutions, one or more of which substitutions is optionally a conservative substitution and/or is a substitution to a germline-encoded amino acid, wherein the antibody or antigen binding fragment is capable of binding to a surface glycoprotein of SARS-CoV-2 expressed on a cell surface of a host cell.

In any of the presently disclosed embodiments, the antibody or antigen-binding fragment is capable of preventing and/or neutralizing a SARS-CoV-2 infection in an in vitro model of infection and/or in an in vivo animal model of infection and/or in a human.

In any of the presently disclosed embodiments, the antibody or antigen-binding fragment comprises CDRH1, CDRH2, CDRH3, CDRL1, CDRL2, and CDRL3 amino acid sequences according to SEQ ID NOs.: (i) 6-8 and 14-16, respectively; (ii) 22-24 and 30-32, respectively; (iii) 22-24, 398, 31, and 32, respectively; (iv) 22-24, 399, 31, and 32, respectively; (v) 22-24, 401, 31, and 32, respectively; (vi) 22-24, 403, 31, and 32, respectively; (v) 22-24, 405, 31, and 32, respectively; (vi) 38-40 and 46-48, respectively; (vii) 38-40, 398, 47, and 48, respectively; (viii) 38-40, 399, 47, and 48, respectively; (viv) 38-40, 401, 47, and 48, respectively; (x) 38-40, 403, 47, and 48, respectively; (xi) 38-40, 405, 47, and 48, respectively; (xii) 54-56 and 62-64, respectively; (xiii) 70-72 and 78-80, respectively; (xiv) 86-88 and 94-96, respectively; (xv) 102-104 and 110-112, respectively; (xvi) 118-120 and 126-128, respectively; (xvii) 134-136 and 142-144, respectively; (xviii) 150-152 and 158-160, respectively; (xix) 166-168 and 174-176, respectively; (xx) 182-184 and 190-192, respectively; (xxi) 198-200 and 206-208, respectively; (xxii) 214-216 and 222-224, respectively; (xxiii) 230-232 and 238-240, respectively; (xxiv) 246-248 and 254-256, respectively; (xxv) 262-264 and 270-272, respectively; (xxxvi) 278-280 and 286-288, respectively; (xxvii) 294-296 and 302-304, respectively; (xxviii) 310-312 and 318-320, respectively; (xxix) 326-328 and 334-336, respectively; (xxx) 342-344 and 350-352, respectively; (xxxi) 358-360 and 366-368, respectively; (xxxii) 22, 23, 375, and 30-32, respectively; (xxxiii) 22, 23, 375, 398, 31, and 32, respectively; (xxxiv) 22, 23, 375, 399, 31, and 32, respectively; (xxxv) 22, 23, 375, 401, 31, and 32, respectively; (xxxvi) 22, 23, 375, 403, 31, and 32, respectively; (xxxvii) 22, 23, 375, 405, 31, and 32, respectively; (xxxviii) 22, 23, 378, and 30-32, respectively; (xxxix) 22, 23, 378, 398, 31, and 32, respectively; (xl) 22, 23, 378, 399, 31, and 32, respectively; (xli) 22, 23, 378, 401, 31, and 32, respectively; (xlii) 22, 23, 378, 403, 31, and 32, respectively; (xliii) 22, 23, 378, 405, 31, and 32, respectively; (xliv) 22-24, 30, 31, and 386, respectively; (xlv) 22-24, 398, 31, and 386, respectively; (xlvi) 22-24, 399, 31, and 386, respectively; (xlvii) 22-24, 401, 31, and 386, respectively; (xlviii) 22-24, 403, 31, and 386, respectively; (xlix) 22-24, 405, 31, and 386, respectively; (1) 38-40, 46, 47, and 386, respectively; (li) 38-40, 398, 47, and 386, respectively; (lii) 38-40, 399, 47, and 386, respectively; (liii) 38-40, 401, 47, and 386, respectively; (liv) 38-40, 403, 47, and 386, respectively; (lv) 38-40, 405, 47, and 386, respectively; (lvi) 22-24, 30, 31, and 394, respectively; (lvii) 22-24, 398, 31, and 394, respectively; (lviii) 22-24, 399, 31, and 394, respectively; (lix) 22-24, 401, 31, and 394, respectively; (lx) 22-24, 403, 31, and 394, respectively; (lxi) 22-24, 405, 31, and 394, respectively; (lxii) 22, 23, 375, 30, 31, and 394, respectively; (lxiii) 22, 23, 375, 30, 31, and 386, respectively; (lxiv) 22, 23, 375, 398, 31, and 386, respectively; (lxv) 22, 23, 375, 399, 31, and 386, respectively; (lxvi) 22, 23, 375, 401, 31, and 386, respectively; (lxvii) 22, 23, 375, 403, 31, and 386, respectively; (lxviii) 22, 23, 375, 405, 31, and 386, respectively; (lxix) 22, 23, 378, 30, 31, and 394, respectively; (lxx) 22, 23, 378, 398, 31, and 394, respectively; (lxxi) 22, 23, 378, 399, 31, and 394, respectively; (lxxii) 22, 23, 378, 401, 31, and 394, respectively; (lxxiii) 22, 23, 378, 403, 31, and 394, respectively; (lxxiv) 22, 23, 378, 405, 31, and 394, respectively; (lxxv) 22, 23, 378, 30, 31, and 386, respectively; (lxxvi) 22, 23, 378, 398, 31, and 386, respectively; (lxxvii) 22, 23, 378, 399, 31, and 386, respectively; (lxxviii) 22, 23, 378, 401, 31, and 386, respectively; (lxxix) 22, 23, 378, 403, 31, and 386, respectively; (lxxx) 22, 23, 378, 405, 31, and 386, respectively; (lxxxi) 22, 23, 397, and 30-32, respectively; (lxxxii) 22, 23, 397, 398, 31, and 32, respectively; (lxxxiii) 22, 23, 397, 399, 31, and 32, respectively; (lxxxiv) 22, 23, 397, 401, 31, and 32, respectively; (lxxxv) 22, 23, 397, 403, 31, and 32, respectively; (lxxxvi) 22, 23, 397, 405, 31, and 32, respectively; (lxxxvii) 22, 23, 397, 30, 31, and 386, respectively; (lxxxviii) 22, 23, 397, 398, 31, and 386, respectively; (lxxxix) 22, 23, 397, 399, 31, and 386, respectively; (xc) 22, 23, 397, 401, 31, and 386, respectively; (xci) 22, 23, 397, 403, 31, and 386, respectively; (xcii) 22, 23, 397, 405, 31, and 386, respectively; (xciii) 22, 23, 397, 30, 31, and 394, respectively; (xciv) 22, 23, 397, 398, 31, and 394, respectively; (xcv) 22, 23, 397, 399, 31, and 394, respectively; (xcvi) 22, 23, 397, 401, 31, and 394, respectively; (xcvii) 22, 23, 397, 403, 31, and 394, respectively; (xcviii) 22, 23, 397, 405, 31, and 394, respectively; (xcix) 22, 416, 24, 30, 31, and 32, respectively; (c) 22, 416, 24, 30, 31, and 386, respectively; (ci) 22, 416, 24, 30, 31, and 394, respectively; (cii) 22, 416, 24, 398, 31, and 32, respectively; (ciii) 22, 416, 24, 398, 31, and 386, respectively; (civ) 22, 416, 24, 398, 31, and 394, respectively; (cv) 22, 416, 24, 399, 31, and 32, respectively; (cvi) 22, 416, 24, 399, 31, and 386, respectively; (cvii) 22, 416, 24, 399, 31, and 394, respectively; (cviii) 22, 416, 24, 401, 31, and 32, respectively; (cix) 22, 416, 24, 401, 31, and 386, respectively; (cx) 22, 416, 24, 401, 31, and 394, respectively; (cxi) 22, 416, 24, 403, 31, and 32, respectively; (cxii) 22, 416, 24, 403, 31, and 386, respectively; (cxiii) 22, 416, 24, 403, 31, and 394, respectively; (cxiv) 22, 416, 24, 405, 31, and 32, respectively; (cxv) 22, 416, 24, 405, 31, and 386, respectively; (cxvi) 22, 416, 24, 405, 31, and 394, respectively; (cxvii) 22, 416, 375, 30, 31, and 32, respectively; (cxviii) 22, 416, 375, 398, 31, and 386, respectively; (cxix) 22, 416, 375, 398, 31, and 394, respectively; (cxx) 22, 416, 375, 399, 31, and 386, respectively; (cxxi) 22, 416, 375, 399, 31, and 394, respectively; (cxxii) 22, 416, 375, 401, 31, and 386, respectively; (cxxiii) 22, 416, 375, 401, 31, and 394, respectively; (cxxiv) 22, 416, 375, 403, 31, and 386, respectively; (cxxv) 22, 416, 375, 403, 31, and 394, respectively; (cxxvi) 22, 416, 375, 405, 31, and 386, respectively; (cxxvii) 22, 416, 375, 405, 31, and 394, respectively; (cxxviii) 22, 416, 378, 30, 31, and 32, respectively; (cxxix) 22, 416, 378, 398, 31, and 386, respectively; (cxxx) 22, 416, 378, 398, 31, and 394, respectively; (cxxxi) 22, 416, 378, 399, 31, and 386, respectively; (cxxxii) 22, 416, 378, 399, 31, and 394, respectively; (cxxxiii) 22, 416, 378, 401, 31, and 386, respectively; (cxxxiv) 22, 416, 378, 401, 31, and 394, respectively; (cxxxv) 22, 416, 375, 403, 31, and 386, respectively; (cxxxvi) 22, 416, 378, 403, 31, and 394, respectively; (cxxxvii) 22, 416, 378, 405, 31, and 386, respectively; (cxxxviii) 22, 416, 378, 405, 31, and 394, respectively; (cxxxix) 22, 416, 397, 30, 31, and 32, respectively; (cxl) 22, 416, 397, 398, 31, and 386, respectively; (cxli) 22, 416, 397, 398, 31, and 394, respectively; (cxlii) 22, 416, 397, 399, 31, and 386, respectively; (ccxliii) 22, 416, 397, 399, 31, and 394, respectively; (cxliv) 22, 416, 397, 401, 31, and 386, respectively; (cxlv) 22, 416, 397, 401, 31, and 394, respectively; (cxlvi) 22, 416, 375, 403, 31, and 386, respectively; (cxlvii) 22, 416, 397, 403, 31, and 394, respectively; (cxlviii) 22, 416, 397, 405, 31, and 386, respectively; or (cxlix) 22, 416, 397, 405, 31, and 394, respectively.

In certain embodiments, an antibody or an antigen-binding fragment of the present disclosure comprises a CDRH1, a CDRH2, a CDRH3, a CDRL1, a CDRL2, and a CDRL3, wherein each CDR is independently selected from a corresponding CDR of nCoV-3 mAb, nCoV-17 mAb, nCoV-6 mAb, nCoV-16 mAb, nCoV-20 mAb, nCoV-4 mAb, nCoV-4-v2 mAb, nCoV-4-v3 mAb, nCoV-15 mAb, nCoV-12 mAb, nCoV-5 mAb, nCoV-14 mAb, nCoV-9 mAb, nCoV-18 mAb, nCoV-8 mAb, nCoV-7 mAb, nCoV-2 mAb, nCoV-19 mAb, nCoV-1 mAb, nCoV-11 mAb, nCoV-13 mAb, nCoV-10 mAb, nCoV-10-v2 mAb, nCoV-10 mAb VL-v3, nCoV-10 mAb VL-v4, nCoV-10 mAb VL-v5, nCoV-10 mAb VL-v6, nCoV-10 mAb VL-v7, nCoV-10 mAb VL-v8, nCoV-10 mAb VL-v9, nCoV-10 mAb VL-v10, nCoV-10 mAb VL-v11, nCoV-10 mAb VL-v12, nCoV-10 mAb VL-v13, nCoV-10 mAb VL-v14, nCoV-10 mAb VL-v15, nCoV-10 mAb VL-v16, nCoV-10 mAb VL-v17, nCoV-10 mAb VL-v18, nCoV-10 mAb VL-v19, nCoV-10 mAb VL-v20, nCoV-10 mAb VH-v3, nCoV-10 mAb VH-v4, nCoV-10 mAb VH-v5, or nCoV-10 mAb VH-v21, as provided in Table 1. That is, all combinations of CDRs from the SARS-CoV-2 mAbs and the variant sequences thereof provided in Table 1 are contemplated. Several different naming conventions for antibodies may be used herein. For example, antibody nCoV-x mAb can also be referred to as nCoV-x, nCoVx, or nCoVx mAb. Antibody nCoV-x-v2 mAb can also be referred to as nCoV-x-v2, nCoVx-v2, nCoVx-v2 mAb, nCoV-x mAb v2, or nCoVx mAb v2. Antibody nCoV-x mAb VH-v2 can also be referred to as nCoV-x VH-v2 or nCoV-x VH-v2 mAb.

The term “CL” refers to an “immunoglobulin light chain constant region” or a “light chain constant region,” i.e., a constant region from an antibody light chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). The Fc region of an antibody heavy chain is described further herein. In any of the presently disclosed embodiments, an antibody or antigen-binding fragment of the present disclosure comprises any one or more of CL, a CH1, a CH2, and a CH3. In certain embodiments, a CL comprises an amino acid sequence having 90%, 91%, 92%, 93%, 94%, 95%, 96%, 97%, 98%, 99%, or 100% identity to the amino acid sequence of SEQ ID NO.:391. In certain embodiments, a CH1-CH2-CH3 comprises an amino acid sequence having 90%, 91%, 92%, 93%, 94%, 95%, 96%, 97%, 98%, 99%, or 100% identity to the amino acid sequence of SEQ ID NO.:395.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of a full-length antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments can be produced as pairs of Fab′ fragments that have hinge cysteines between them. Other chemical couplings of antibody fragments are also known.

“Fv” is a small antibody fragment that contains a complete antigen-recognition and antigen-binding site. This fragment consists of a dimer of one heavy-chain variable domain and one light-chain variable domain in tight, non-covalent association. However, even a single variable domain (or half of an Fv comprising only three CDRs specific for an antigen) has the ability to recognize and bind antigen, although typically at a lower affinity than the entire binding site. “Fd” refers to VH+CH1.

“Single-chain Fv” also abbreviated as “sFv” or “scFv”, are antibody fragments that comprise the VH and VL antibody domains preferably connected into a single polypeptide chain. Preferably, the sFv polypeptide further comprises a polypeptide linker between the VH and VL domains that enables the sFv to form the desired structure for antigen binding. Additionally or alternatively, Fv can have a disulfide bond formed between the VH and the VL. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

During antibody development, DNA in the germline variable (V), joining (J), and diversity (D) gene loci may be rearranged and insertions and/or deletions of nucleotides in the coding sequence may occur. Somatic mutations may be encoded by the resultant sequence, and can be identified by reference to a corresponding known germline sequence. In some contexts, somatic mutations that are not critical to a desired property of the antibody (e.g., binding to a SARS-CoV-2 antigen), or that confer an undesirable property upon the antibody (e.g., an increased risk of immunogenicity in a subject administered the antibody), or both, may be replaced by the corresponding germline-encoded amino acid, or by a different amino acid, so that a desirable property of the antibody is improved or maintained and the undesirable property of the antibody is reduced or abrogated. Thus, in some embodiments, the antibody or antigen-binding fragment of the present disclosure comprises at least one more germline-encoded amino acid in a variable region as compared to a parent antibody or antigen-binding fragment, provided that the parent antibody or antigen binding fragment comprises one or more somatic mutations. Variable region and CDR amino acid sequences of exemplary anti-SARS-CoV-2 antibodies of the present disclosure are provided in Table 1 herein.

In certain embodiments, an antibody or antigen-binding fragment comprises an amino acid modification (e.g., a substitution mutation) to remove an undesired risk of oxidation, deamidation, and/or isomerization.

Also provided herein are variant antibodies that comprise one or more amino acid alterations (e.g. substitutions) in a variable region (e.g., VH, VL, framework or CDR) as compared to a presently disclosed (“parent” or “reference”) antibody, wherein the variant antibody is capable of binding to a SARS-CoV-2 antigen.

In certain embodiments, the VH comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 5, 21, 37, 53, 69, 85, 101, 117, 133, 149, 165, 181, 197, 213, 229, 245, 261, 277, 293, 309, 325, 341, 357, 374, 377, 396, or 415, wherein the variation as compared to the reference VH SEQ ID NO., if present, is optionally limited to one or more framework regions and/or the variation, if present, comprises one or more substitution to a germline-encoded amino acid; and/or (ii) the VL comprises or consists of an amino acid sequence having at least 85% (i.e., 85%, 86, 87, 88, 89, 90, 91, 92, 93, 94, 95, 96, 97, 98, 99, or 100%) identity to the amino acid sequence according to any one of SEQ ID NOs.: 13, 29, 45, 61, 77, 93, 109, 125, 141, 157, 173, 189, 205, 221, 237, 253, 269, 285, 301, 317, 333, 349, 365, 380, 383, 385, 388, 392, 393, 400, 402, 404, and 406-414, wherein the variation as compared to the reference VL SEQ ID NO., if present, is optionally limited to one or more framework regions and/or the variation, if present, comprises one or more substitution to a germline-encoded amino acid.

In some embodiments, the VH and the VL comprise or consist of an amino acid sequence having at least 85%, 86%, 87%, 88%, 89%, 90%, 91%, 92%, 93%, 94%, 95%, 96%, 97%, 98%, or 99% to the amino acid sequence set forth in SEQ ID NOs.: (i) 5 and 13, respectively; (ii) 21 and any one of 29, 45, 380, 383, 385, 388, 392, 393, 400, 402, 404, 406, 407, 408, 409, 410, 411, 412, 413, and 414, respectively; (iii) 415 and any one of 29, 45, 380, 383, 385, 388, 392, 400, 402, 404, 406, 407, 408, 409, 410, 411, 412, 413, and 414, respectively; (iv) 37 and any one of 29, 45, 380, 383, 385, 388, 392, 400, 402, 404, 406, 407, 408, 409, 410, 411, 412, 413, and 414, respectively; (v) 53 and 61, respectively; (vi) 69 and 77, respectively; (vii) 85 and 93, respectively; (viii) 101 and 109, respectively; (ix) 117 and any one of 125, 141, and 157, respectively; (x) 133 and any one of 125, 141, and 157, respectively; (xi) 149 and any one of 125, 141, and 157, respectively; (xii) 165 and 173, respectively; (xiii) 181 and 189, respectively; (xiv) 197 and 205, respectively; (xv) 213 and 221, respectively; (xvi) 229 and 237, respectively; (xvii) 245 and 253, respectively; (xviii) 261 and 269, respectively; (xix) 277 and 285, respectively; (xx) 293 and 301, respectively; (xxi) 309 and 317, respectively; (xxii) 325 and 333, respectively; (xxiii) 341 and 349, respectively; (xxiv) 357 and 365, respectively; (xxv) 374 and any one of 29, 45, 380, 383, 385, 392, 393, 400, 402, 404, 406, 407, 408, 409, 410, 411, 412, 413, and 414, respectively; (xxvi) 377 and any one of 29, 45, 380, 383, 385, 392, 393, 400, 402, 404, 406, 407, 408, 409, 410, 411, 412, 413, and 414, respectively; (xxvii) 415 and any one of 29, 45, 380, 383, 385, 392, 393, 400, 402, 404, 406, 407, 408, 409, 410, 411, 412, 413, and 414, respectively; or (xxviii) 396 and any one of 29, 37, 45, 380, 383, 385, 388, 392, 393, 400, 402, 404, 406, 407, 408, 409, 410, 411, 412, 413, and 414, respectively.

In certain embodiments, the VH comprises or consists of any VH amino acid sequence set forth in Table 1, and the VL comprises or consists of any VL amino acid sequence set forth in Table 1. In particular embodiments, the VH and the VL comprise or consist of the amino acid sequences according to SEQ ID NOs.: (i) 5 and 13, respectively; (ii) 21 and any one of 29, 45, 380, 383, 385, 388, 392, 393, 400, 402, 404, 406, 407, 408, 409, 410, 411, 412, 413, and 414, respectively; (iii) 415 and any one of 29, 45, 380, 383, 385, 388, 392, 400, 402, 404, 406, 407, 408, 409, 410, 411, 412, 413, and 414, respectively; (iv) 37 and any one of 29, 45, 380, 383, 385, 388, 392, 400, 402, 404, 406, 407, 408, 409, 410, 411, 412, 413, and 414, respectively; (v) 53 and 61, respectively; (vi) 69 and 77, respectively; (vii) 85 and 93, respectively; (viii) 101 and 109, respectively; (ix) 117 and any one of 125, 141, and 157, respectively; (x) 133 and any one of 125, 141, and 157, respectively; (xi) 149 and any one of 125, 141, and 157, respectively; (xii) 165 and 173, respectively; (xiii) 181 and 189, respectively; (xiv) 197 and 205, respectively; (xv) 213 and 221, respectively; (xvi) 229 and 237, respectively; (xvii) 245 and 253, respectively; (xviii) 261 and 269, respectively; (xix) 277 and 285, respectively; (xx) 293 and 301, respectively; (xxi) 309 and 317, respectively; (xxii) 325 and 333, respectively; (xxiii) 341 and 349, respectively; (xxiv) 357 and 365, respectively; (xxv) 374 and any one of 29, 45, 380, 383, 385, 392, 393, 400, 402, 404, 406, 407, 408, 409, 410, 411, 412, 413, and 414, respectively; (xxvi) 377 and any one of 29, 45, 380, 383, 385, 392, 393, 400, 402, 404, 406, 407, 408, 409, 410, 411, 412, 413, and 414, respectively; (xxvii) 415 and any one of 29, 45, 380, 383, 385, 392, 393, 400, 402, 404, 406, 407, 408, 409, 410, 411, 412, 413, and 414, respectively; or (xxviii) 396 and any one of 29, 37, 45, 380, 383, 385, 388, 392, 393, 400, 402, 404, 406, 407, 408, 409, 410, 411, 412, 413, and 414, respectively.

In certain embodiments, an antibody or antigen-binding fragment of the present disclosure is a multispecific antibody, such as a bispecific or trispecific antibody. Formats for bispecific antibodies are disclosed in, for example, Spiess et al., Mol. Immunol. 67(2):95 (2015), and in Brinkmann and Kontermann, mAbs 9(2):182-212 (2017), which bispecific formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, k-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

A bispecific or multispecific antibody may comprise one, two, or more SARS-CoV-2 antigen-binding domains or sequences (e.g., CDRS, and/or a VH and a VL) of the instant disclosure, optionally in combination with another SARS-CoV-2 binding domain of the instant disclosure, or in combination with a different binding domain that specifically binds to SARS-CoV-2 (e.g., at a same or a different epitope), or with a binding domain that binds to a different antigen.

In any of the presently disclosed embodiments, the antibody or antigen-binding fragment can be multispecific; e.g., bispecific, trispecific, or the like.

In certain embodiments, the antibody or antigen-binding fragment comprises: (i) a first VH and a first VL; and (ii) a second VH and a second VL, wherein the first VH and the second VH are different and each independently comprise an amino acid sequence having at least 85% (i.e., 85%, 86%, 87%, 88%, 89%, 90%, 91%, 92%, 93%, 94%, 95%, 96%, 97%, 98%, 99%, or 100%) identity to the amino acid sequence set forth in any one of SEQ ID NOs.: 5, 21, 37, 53, 69, 85, 101, 117, 133, 149, 165, 181, 197, 213, 229, 245, 261, 277, 293, 309, 325, 341, 357, 374, 377, 396, or 415, and wherein the first VL and the second VL are different and each independently comprise an amino acid sequence having at least 85% (i.e., 85%, 86%, 87%, 88%, 89%, 90%, 91%, 92%, 93%, 94%, 95%, 96%, 97%, 98%, 99%, or 100%) identity to the amino acid sequence set forth in any one of SEQ ID NOs.: 13, 29, 45, 61, 77, 93, 109, 125, 141, 157, 173, 189, 205, 221, 237, 253, 269, 285, 301, 317, 333, 349, 365, 380, 383, 385, 388, 392, 393, 400, 402, 404, and 406-414, and wherein the first VH and the first VL together form a first antigen-binding site, and wherein the second VH and the second VL together form a second antigen-binding site.

In particular embodiments, (i) the first VH comprises or consists of the amino acid sequence set forth in any one of SEQ ID NOs.:21, 37, 374, 377, 396, and 415, and the first VL comprises or consists of the amino acid sequence set forth in any one of SEQ ID NOs.:29, 45, 380, 383, 385, 388, 392, 393, 400, 402, 404, and 406-414, and (ii) the second VH comprises or consists of the amino acid sequence set forth in SEQ ID NO.:69 and the second VL comprises or consists of the amino acid sequence set forth in SEQ ID NO.:77.

In certain embodiments, the antibody or antigen-binding fragment comprises an Fc polypeptide, or a fragment thereof. The “Fc” fragment or Fc polypeptide comprises the carboxy-terminal portions (i.e., the CH2 and CH3 domains of IgG) of both antibody H chains held together by disulfides. Antibody “effector functions” refer to those biological activities attributable to the Fc region (a native sequence Fc region or amino acid sequence variant Fc region) of an antibody, and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptor); and B cell activation. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure). Such functions include, for example, Fc receptor (FcR) binding, antibody half-life modulation (e.g., by binding to FcRn), ADCC function, protein A binding, protein G binding, and complement binding. Amino acid modifications that modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/I332E, P257I/Q311, K326W/E333S, S239D/I332E/G236A, N297Q, K322A, S228P, L235E+E318A/K320A/K322A, L234A/L235A (also referred to herein as “LALA”), and L234A/L235A/P329G mutations, which mutations are summarized and annotated in “Engineered Fe Regions”, published by InvivoGen (2011) and available online at invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

For example, to activate the complement cascade, the Clq protein complex can bind to at least two molecules of IgG1 or one molecule of IgM when the immunoglobulin molecule(s) is attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys 322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on cells including hematopoietic cells. Fc receptors belong to the immunoglobulin superfamily, and shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., JLab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Fc moieties providing cross-linking of receptors (e.g., FcγR) are contemplated herein. In humans, three classes of FcγR have been characterized to-date, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is believed to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologous; and (iii) FcγRIII (CD16), which binds IgG with medium to low affinity and has been found in two forms: FcγRIIIA, which has been found on NK cells, macrophages, eosinophils, and some monocytes and T cells, and is believed to mediate ADCC; and FcγRIIIB, which is highly expressed on neutrophils.

FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, it has been shown that 75% of all FcγRIIB is found in the liver (Ganesan, L. P. et al., 2012: “FcγRIIb on liver sinusoidal endothelium clears small immune complexes,” Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

In some embodiments, the antibodies disclosed herein and the antigen-binding fragments thereof comprise an Fc polypeptide or fragment thereof for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). In some embodiments, the antibodies of the present disclosure, or the antigen binding fragments thereof, comprise an engineered Fc moiety with the mutations S267E and L328F, in particular as described by Chu, S. Y. et al., 2008: Inhibition of B cell receptor-mediated activation of primary human B cells by coengagement of CD19 and FcgammaRIIb with Fc-engineered antibodies. Molecular Immunology 45, 3926-3933.

On B cells, FcγRIIB may function to suppress further immunoglobulin production and isotype switching to, for example, the IgE class. On macrophages, FcγRIIB is thought to inhibit phagocytosis as mediated through FcγRIIA. On eosinophils and mast cells, the B form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into corresponding positions IgG1 and IgG4, reduces binding of IgG1 and IgG4 to FcγRI by 10³-fold and eliminated the human monocyte response to antibody-sensitized red blood cells (Armour, K. L., et al. Eur. J. Immunol. 29 (1999) 2613-2624).

Regarding FcγRII binding, reduced binding for FcγRIIA is found, e.g., for IgG mutation of at least one of E233-G236, P238, D265, N297, A327, P329, D270, Q295, A327, R292 and K414.

Two allelic forms of human FcγRIIA are the “H131” variant, which binds to IgG1 Fc with high affinity, and the “R131” variant, which binds to IgG1 Fc with low affinity. See, e.g., Bruhns et al., Blood 113:3716-3725 (2009).

Regarding FcγRIII binding, reduced binding to FcγRIIIA is found, e.g., for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above-mentioned mutation sites, and methods for measuring binding to FcγRI and FcγRIIA, are described in Shields, R. L., et al., J. Biol. Chem. 276 (2001) 6591-6604.

Two allelic forms of human FcγRIIIA are the “F158” variant, which binds to IgG1 Fc with low affinity, and the “V158” variant, which binds to IgG1 Fc with high affinity. See, e.g., Bruhns et al., Blood 113:3716-3725 (2009).

Regarding binding to FcγRII, two regions of native IgG Fc appear to be involved in interactions between FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

Also contemplated are mutations that increase binding affinity of an Fc polypeptide or fragment thereof of the present disclosure to a (i.e., one or more) Fc7 receptor (e.g., as compared to a reference Fc polypeptide or fragment thereof or containing the same that does not comprise the mutation(s)). See, e.g., Delillo and Ravetch, Cell 161(5):1035-1045 (2015) and Ahmed et al., J. Struc. Biol. 194(1):78 (2016), the Fc mutations and techniques of which are incorporated herein by reference.

In any of the herein disclosed embodiments, an antibody or antigen-binding fragment can comprise a Fc polypeptide or fragment thereof comprising a mutation selected from G236A; S239D; A330L; and I332E; or a combination comprising any two or more of the same; e.g., S239D/I332E; S239D/A330L/I332E; G236A/S239D/I332E; G236A/A330L/I332E (also referred to herein as “GAALIE”); or G236A/S239D/A330L/I332E. In some embodiments, the Fc polypeptide or fragment thereof does not comprise S239D. In some embodiments, the Fc polypeptide or fragment comprises a Ser at position 239 (EU numbering).

In certain embodiments, the Fc polypeptide or fragment thereof may comprise or consist of at least a portion of an Fc polypeptide or fragment thereof that is involved in binding to FcRn binding. In certain embodiments, the Fc polypeptide or fragment thereof comprises one or more amino acid modifications that improve binding affinity for (e.g., enhance binding to) FcRn (e.g., at a pH of about 6.0) and, in some embodiments, thereby extend in vivo half-life of a molecule comprising the Fc polypeptide or fragment thereof (e.g., as compared to a reference Fc polypeptide or fragment thereof or antibody that is otherwise the same but does not comprise the modification(s)). In certain embodiments, the Fc polypeptide or fragment thereof comprises or is derived from a IgG Fe and a half-life-extending mutation comprises any one or more of: M428L; N434S; N434H; N434A; N434S; M252Y; S254T; T256E; T250Q; P257I Q311I; D376V; T307A; E380A (EU numbering). In certain embodiments, a half-life-extending mutation comprises M428L/N434S (also referred to herein as “MLNS”). In certain embodiments, a half-life-extending mutation comprises M252Y/S254T/T256E. In certain embodiments, a half-life-extending mutation comprises T250Q/M428L. In certain embodiments, a half-life-extending mutation comprises P257I/Q311I. In certain embodiments, a half-life-extending mutation comprises P257I/N434H. In certain embodiments, a half-life-extending mutation comprises D376V/N434H. In certain embodiments, a half-life-extending mutation comprises T307A/E380A/N434A.

In some embodiments, an antibody or antigen-binding fragment includes an Fc moiety that comprises the substitution mutations M428L/N434S. In some embodiments, an antibody or antigen-binding fragment includes an Fc polypeptide or fragment thereof that comprises the substitution mutations G236A/A330L/I332E. In certain embodiments, an antibody or antigen-binding fragment includes a (e.g., IgG) Fc moiety that comprises a G236A mutation, an A330L mutation, and an I332E mutation (GAALIE), and does not comprise a S239D mutation (e.g., comprises a native S at position 239). In particular embodiments, an antibody or antigen-binding fragment includes an Fc polypeptide or fragment thereof that comprises the substitution mutations: M428L/N434S and G236A/A330L/I332E, and optionally does not comprise S239D (e.g., comprises a Ser at position 239). In certain embodiments, an antibody or antigen-binding fragment includes a Fc polypeptide or fragment thereof that comprises the substitution mutations: M428L/N434S and G236A/S239D/A330L/I332E, and, optionally, does comprise any further other substitution mutations.

In certain embodiments, the antibody or antigen-binding fragment comprises a mutation that alters glycosylation, wherein the mutation that alters glycosylation comprises N297A, N297Q, or N297G, and/or the antibody or antigen-binding fragment is partially or fully aglycosylated and/or is partially or fully afucosylated. Host cell lines and methods of making partially or fully aglycosylated or partially or fully afucosylated antibodies and antigen-binding fragments are known (see, e.g., PCT Publication No. WO 2016/181357; Suzuki et al. Clin. Cancer Res. 13(6):1875-82 (2007); Huang et al. MAbs 6:1-12 (2018)).

In certain embodiments, the antibody or antigen-binding fragment is capable of eliciting continued protection in vivo in a subject even once no detectable levels of the antibody or antigen-binding fragment can be found in the subject (i.e., when the antibody or antigen-binding fragment has been cleared from the subject following administration). Such protection is referred to herein as a vaccinal effect. Without wishing to be bound by theory, it is believed that dendritic cells can internalize complexes of antibody and antigen and thereafter induce or contribute to an endogenous immune response against antigen. In certain embodiments, an antibody or antigen-binding fragment comprises one or more modifications, such as, for example, mutations in the Fc comprising G236A, A330L, and I332E, that are capable of activating dendritic cells that may induce, e.g., T cell immunity to the antigen.

In any of the presently disclosed embodiments, the antibody or antigen-binding fragment comprises a Fc polypeptide or a fragment thereof, including a CH2 (or a fragment thereof, a CH3 (or a fragment thereof), or a CH2 and a CH3, wherein the CH2, the CH3, or both can be of any isotype and may contain amino acid substitutions or other modifications as compared to a corresponding wild-type CH2 or CH3, respectively. In certain embodiments, an Fc polypeptide of the present disclosure comprises two CH2-CH3 polypeptides that associate to form a dimer.

In any of the presently disclosed embodiments, the antibody or antigen-binding fragment can be monoclonal. The term “monoclonal antibody” (mAb) as used herein refers to an antibody obtained from a population of substantially homogeneous antibodies, i.e., individual antibodies comprising the population are identical except for possible naturally occurring mutations that may be present, in some cases in minor amounts. Monoclonal antibodies are highly specific, being directed against a single antigenic site. Furthermore, in contrast to polyclonal antibody preparations that include different antibodies directed against different epitopes, each monoclonal antibody is directed against a single epitope of the antigen. In addition to their specificity, the monoclonal antibodies are advantageous in that they may be synthesized uncontaminated by other antibodies. The term “monoclonal” is not to be construed as requiring production of the antibody by any particular method. For example, monoclonal antibodies useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example. Monoclonal antibodies may also be obtained using methods disclosed in PCT Publication No. WO 2004/076677A2.

Antibodies and antigen-binding fragments of the present disclosure include “chimeric antibodies” in which a portion of the heavy chain and/or the light chain is identical with or homologous to a corresponding sequence or sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, U.S. Pat. Nos. 4,816,567; 5,530,101 and 7,498,415; and Morrison et al., Proc. Natl. Acad. Sci. USA, 81:6851-6855 (1984)). For example, chimeric antibodies may comprise human and non-human residues. Furthermore, chimeric antibodies may comprise residues that are not found in the recipient antibody or in the donor antibody. These modifications are made to further refine antibody performance. For further details, see Jones et al., Nature 321:522-525 (1986); Riechmann et al., Nature 332:323-329 (1988); and Presta, Curr. Op. Struct. Biol. 2:593-596 (1992). Chimeric antibodies also include primatized and humanized antibodies.

A “humanized antibody” is generally considered to be a human antibody that has one or more amino acid residues introduced into it from a source that is non-human. These non-human amino acid residues are typically taken from a variable domain. Humanization may be performed, for example, following the method of Winter and co-workers (Jones et al., Nature, 321:522-525 (1986); Reichmann et al., Nature, 332:323-327 (1988); Verhoeyen et al., Science, 239:1534-1536 (1988)), by substituting non-human variable sequences for the corresponding sequences of a human antibody. Accordingly, such “humanized” antibodies are chimeric antibodies (e.g., U.S. Pat. Nos. 4,816,567; 5,530,101 and 7,498,415) wherein substantially less than an intact human variable domain has been substituted by the corresponding sequence from a non-human species. In some instances, a “humanized” antibody is one which is produced by a non-human cell or animal and comprises human sequences, e.g., Hc domains.

A “human antibody” is an antibody containing only sequences that are present in an antibody that is produced by a human. However, as used herein, human antibodies may comprise residues or modifications not found in a naturally occurring human antibody (e.g., an antibody that is isolated from a human), including those modifications and variant sequences described herein. These may be made to further refine or enhance antibody performance. In some instances, human antibodies are produced by transgenic animals. For example, see U.S. Pat. Nos. 5,770,429; 6,596,541 and 7,049,426.

In certain embodiments, an antibody or antigen-binding fragment of the present disclosure is chimeric, humanized, or human.

### Polynucleotides, Vectors, and Host Cells

In another aspect, the present disclosure provides isolated polynucleotides that encode any of the presently disclosed antibodies or an antigen-binding fragment thereof, or a portion thereof (e.g., a CDR, a VH, a VL, a heavy chain, or a light chain). In certain embodiments, the polynucleotide is codon-optimized for expression in a host cell. Once a coding sequence is known or identified, codon optimization can be performed using known techniques and tools, e.g., using the GenScript® OptimiumGene™ tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

It will also be appreciated that polynucleotides encoding antibodies and antigen-binding fragments of the present disclosure may possess different nucleotide sequences while still encoding a same antibody or antigen-binding fragment due to, for example, the degeneracy of the genetic code, splicing, and the like.

In certain embodiments, the polynucleotide comprises a polynucleotide having at least 50% (i.e., 50%, 55%, 60%, 65%, 70%, 75%, 80%, 85%, 90%, 91%, 92%, 93%, 94%, 95%, 96%, 97%, 98%, 99%, or 100%) identity to the polynucleotide sequence according to any one or more of SEQ ID NOs.:1-4, 9-12, 17-20, 25-28, 33-36, 41-44, 49-52, 57-60, 65-68, 73-76, 81-84, 89-92, 97-100, 105-108, 113-116, 121-124, 129-132, 137-140, 145-148, 153-156, 161-164, 169-172, 177-180, 185-188, 193-196, 201-204, 209-212, 217-220, 225-228, 233-236, 241-244, 249-252, 257-260, 265-268, 273-276, 281-284, 289-292, 297-300, 305-308, 313-316, 321-324, 329-332, 337-340, 345-348, 353-356, 361-364, 372, 373, 376, 379, 381, 384, 387, 389, and 417.

In any of the presently disclosed embodiments, the polynucleotide can comprise deoxyribonucleic acid (DNA) or ribonucleic acid (RNA). In some embodiments, the RNA comprises messenger RNA (mRNA).

Vectors are also provided, wherein the vectors comprise or contain a polynucleotide as disclosed herein (e.g., a polynucleotide that encodes an antibody or antigen-binding fragment that binds to SARS-CoV-2). A vector can comprise any one or more of the vectors disclosed herein. In particular embodiments, a vector is provided that comprises a DNA plasmid construct encoding the antibody or antigen-binding fragment, or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody or antigen-binding fragment, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody or antigen-binding fragment are encoded by a polynucleotide comprised in a single plasmid. In other embodiments, the substituent components of the antibody or antigen-binding fragment are encoded by a polynucleotide comprised in two or more plasmids (e.g., a first plasmid comprises a polynucleotide encoding a heavy chain, VH, or VH+CH, and a second plasmid comprises a polynucleotide encoding the cognate light chain, VL, or VL+CL). In certain embodiments, a single plasmid comprises a polynucleotide encoding a heavy chain and/or a light chain from two or more antibodies or antigen-binding fragments of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

In a further aspect, the present disclosure also provides a host cell expressing an antibody or antigen-binding fragment according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR- CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing an antibody, or an antigen-binding fragment thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express an antibody or antigen-binding fragment of the present disclosure. For example, the cell may be of a species that is different to the species from which the antibody was fully or partially obtained (e.g., CHO cells expressing a human antibody or an engineered human antibody). In some embodiments, the cell type of the host cell does not express the antibody or antigen-binding fragment in nature. Moreover, the host cell may impart a post-translational modification (PTM; e.g., glycosylation or fucosylation, or reduced glycosylation or fucosylation) on the antibody or antigen-binding fragment that is not present in a native state of the antibody or antigen-binding fragment (or in a native state of a parent antibody from which the antibody or antigen binding fragment was engineered or derived). Such a PTM may result in a functional difference (e.g., reduced immunogenicity). Accordingly, an antibody or antigen-binding fragment of the present disclosure that is produced by a host cell as disclosed herein may include one or more post-translational modification that is distinct from the antibody (or parent antibody) in its native state (e.g., a human antibody produced by a CHO cell can comprise one or more post-translational modification that is distinct from the antibody when isolated from the human and/or produced by the native human B cell or plasma cell).

Insect cells useful expressing an antibody or antigen-binding fragment of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodopterafrugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210⁻²¹⁵ (2006).

Plant cells can also be utilized as hosts for expressing an antibody or antigen-binding fragment of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

In a related aspect, the present disclosure provides methods for producing an antibody, or antigen-binding fragment, wherein the methods comprise culturing a host cell of the present disclosure under conditions and for a time sufficient to produce the antibody, or the antigen-binding fragment. Methods useful for isolating and purifying recombinantly produced antibodies, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody or antigen-binding fragment described herein include batch cell culture, which is monitored and controlled to maintain appropriate culture conditions. Purification of soluble antibodies may be performed according to methods described herein and known in the art and that comport with laws and guidelines of domestic and foreign regulatory agencies.

### Compositions

Also provided herein are compositions that comprise any one or more of the presently disclosed antibodies, antigen-binding fragments, polynucleotides, vectors, or host cells, singly or in any combination, and can further comprise a pharmaceutically acceptable carrier, excipient, or diluent. Carriers, excipients, and diluents are discussed in further detail herein.

In certain embodiments, a composition comprises two or more different antibodies or antigen-binding fragments according to the present disclosure. In some embodiments, comprising a first antibody or antigen-binding fragment and a second antibody or antigen-binding fragment, wherein: (i) the first antibody or antigen-binding fragment comprises a VH comprising or consisting of the amino acid sequence set forth in any one of SEQ ID NOs.:21, 37, 374, 377, 396, and 415, and a VL comprising or consisting of the amino acid sequence set forth in any one of SEQ ID NOs.:29, 45, 380, 383, 385, 388, 392, 393, 400, 402, 404, and 406-414; and (ii) the second antibody or antigen-binding fragment comprises a VH comprising or consisting of the amino acid sequence set forth in SEQ ID NO.:69, and a VL comprising or consisting of the amino acid sequence set forth in SEQ ID NO.:77.

In certain embodiments, a composition comprises a first vector comprising a first plasmid, and a second vector comprising a second plasmid, wherein the first plasmid comprises a polynucleotide encoding a heavy chain, VH, or VH+CH, and a second plasmid comprises a polynucleotide encoding the cognate light chain, VL, or VL+CL of the antibody or antigen-binding fragment thereof.

In certain embodiments, a composition comprises a polynucleotide (e.g., mRNA) coupled to a suitable delivery vehicle or carrier. Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol: polyethylene glycol lipid), and subcutaneous, intramuscular, intradermal, intravenous, intraperitoneal, and intratracheal administration of the same, are incorporated herein by reference.

### Methods and Uses

Also provided herein are methods for use of an antibody or antigen-binding fragment, nucleic acid, vector, cell, or composition of the present disclosure in the diagnosis of sarbecovirus and/or SARS-CoV-2 infection (e.g., in a human subject, or in a sample obtained from a human subject).

Methods of diagnosis (e.g., in vitro, ex vivo) may include contacting an antibody, antibody fragment (e.g., antigen binding fragment) with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood. The methods of diagnosis may also include the detection of an antigen/antibody complex, in particular following the contacting of an antibody or antibody fragment with a sample. Such a detection step can be performed at the bench, i.e. without any contact to the human or animal body. Examples of detection methods are well-known to the person skilled in the art and include, e.g., ELISA (enzyme-linked immunosorbent assay), including direct, indirect, and sandwich ELISA.

Also provided herein are methods of treating a subject using an antibody or antigen-binding fragment of the present disclosure, or a composition comprising the same, wherein the subject has, is believed to have, or is at risk for having an infection by SARS-CoV-2. “Treat,” “treatment,” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. In certain embodiments, therapeutic or prophylactic/preventive benefit includes reduction or prevention of hospitalization for treatment of a SARS-CoV-2 infection (i.e., in a statistically significant manner). In certain embodiments, therapeutic or prophylactic/preventive benefit includes a reduced duration of hospitalization for treatment of a SARS-CoV-2 infection (i.e., in a statistically significant manner). In certain embodiments, therapeutic or prophylactic/preventive benefit includes a reduced or abrogated need for respiratory intervention, such as intubation and/or the use of a respirator device. In certain embodiments, therapeutic or prophylactic/preventive benefit includes reversing a late-stage disease pathology and/or reducing mortality.

A “therapeutically effective amount” or “effective amount” of an antibody, antigen-binding fragment, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously. A combination may comprise, for example, two different antibodies that specifically bind a SARS-CoV-2 antigen, which in certain embodiments, may be the same or different SARS-CoV-2 antigen, and/or can comprise the same or different epitopes.

Accordingly, in certain embodiments, methods are provided for treating a SARS-CoV-2 infection in a subject, wherein the methods comprise administering to the subject an effective amount of an antibody, antigen-binding fragment, polynucleotide, vector, host cell, or composition as disclosed herein, or any combination thereof.

Subjects that can be treated by the present disclosure are, in general, human and other primate subjects, such as monkeys and apes for veterinary medicine purposes. Other model organisms, such as mice and rats, may also be treated according to the present disclosure. In any of the aforementioned embodiments, the subject may be a human subject. The subjects can be male or female and can be any suitable age, including infant, juvenile, adolescent, adult, and geriatric subjects.

A number of criteria are believed to contribute to high risk for severe symptoms or death associated with a SARS CoV-2 infection. These include, but are not limited to, age, occupation, general health, pre-existing health conditions, and lifestyle habits. In some embodiments, a subject treated according to the present disclosure comprises one or more risk factors.

In certain embodiments, a human subject treated according to the present disclosure is an infant, a child, a young adult, an adult of middle age, or an elderly person. In certain embodiments, a human subject treated according to the present disclosure is less than 1 year old, or is 1 to 5 years old, or is between 5 and 125 years old (e.g., 5, 10, 15, 20, 25, 30, 35, 40, 45, 50, 55, 60, 65, 70, 75, 80, 85, 90, 95, 100, 105, 110, 115, or 125 years old, including any and all ages therein or therebetween). In certain embodiments, a human subject treated according to the present disclosure is 0-19 years old, 20-44 years old, 45-54 years old, 55-64 years old, 65-74 years old, 75-84 years old, or 85 years old, or older. Persons of middle, and especially of elderly age are believed to be at particular risk. In particular embodiments, the human subject is 45-54 years old, 55-64 years old, 65-74 years old, 75-84 years old, or 85 years old, or older. In some embodiments, the human subject is male. In some embodiments, the human subject is female.

In certain embodiments, a human subject treated according to the present disclosure is a resident of a nursing home or a long-term care facility, is a hospice care worker, is a healthcare provider or healthcare worker, is a first responder, is a family member or other close contact of a subject diagnosed with or suspected of having a SARS-CoV-2 infection, is overweight or clinically obese, is or has been a smoker, has or had chronic obstructive pulmonary disease (COPD), is asthmatic (e.g., having moderate to severe asthma), has an autoimmune disease or condition (e.g., diabetes), and/or has a compromised or depleted immune system (e.g., due to AIDS/HIV infection, a cancer such as a blood cancer, a lymphodepleting therapy such as a chemotherapy, a bone marrow or organ transplantation, or a genetic immune condition), has chronic liver disease, has cardiovascular disease, has a pulmonary or heart defect, works or otherwise spends time in close proximity with others, such as in a factory, shipping center, hospital setting, or the like.

In certain embodiments, a subject treated according to the present disclosure has received a vaccine for SARS-CoV-2 and the vaccine is determined to be ineffective, e.g., by post-vaccine infection or symptoms in the subject, by clinical diagnosis or scientific or regulatory criteria.

In certain embodiments, treatment is administered as peri-exposure prophylaxis. In certain embodiments, treatment is administered to a subject with mild-to-moderate disease, which may be in an outpatient setting. For example, human subjects with mild COVID-19 can include individuals who have any of various signs and symptoms, e.g., fever, cough, sore throat, malaise, headache, muscle pain, without shortness of breath, dyspnea, or abnormal imaging. Human subjects with moderate COVID-19 can include individuals who have evidence of lower respiratory disease by clinical assessment or imaging and a saturation of oxygen (SaO2) greater than (>) 93 percent (%) on room air at sea level. In some embodiments, the subject is at risk for contracting COVID-19. In some embodiments, the subject has COVID-19, e.g., a subject who has a positive SARS-CoV-2 viral testing result. In some embodiments, the human subject is at high risk for progressing to severe COVID-19 and/or hospitalization, e.g., the human subject (i) is 65 years of age or older (≥65); (ii) has a body mass index (BMI) of 35 or greater (≥35); (iii) has chronic kidney disease; (iv) has diabetes; (v) has immunosuppressive disease, (vi) is receiving immunosuppressive treatment; (vii) is 55 years of age or older (≥55) and has cardiovascular disease, hypertension, chronic obstructive pulmonary disease, or other chronic respiratory disease; or (viii) is 12-17 years of age and have a BMI≥85% for their age and gender, or sickle cell disease, congenital or acquired heart disease, neurodevelopmental disorders (e.g., cerebral palsy), a medical-related technological dependence (e.g., tracheostomy, gastrostomy, or positive pressure ventilation not related to COVID-19), or asthma, reactive airway or other chronic respiratory disease that requires daily medication for control.

In certain embodiments, treatment is administered to a subject with moderate-to-severe disease, such as requiring hospitalization.

Typical routes of administering the presently disclosed compositions thus include, without limitation, oral, topical, transdermal, inhalation, parenteral, sublingual, buccal, rectal, vaginal, and intranasal. The term “parenteral”, as used herein, includes subcutaneous injections, intravenous, intramuscular, intrasternal injection or infusion techniques. In certain embodiments, administering comprises administering by a route that is selected from oral, intravenous, parenteral, intragastric, intrapleural, intrapulmonary, intrarectal, intradermal, intraperitoneal, intratumoral, subcutaneous, topical, transdermal, intracisternal, intrathecal, intranasal, and intramuscular. In particular embodiments, a method comprises orally administering the antibody, antigen-binding fragment, polynucleotide, vector, host cell, or composition to the subject.

Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units, where for example, a tablet may be a single dosage unit, and a container of a herein described an antibody or antigen-binding in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of an antibody or antigen-binding fragment, polynucleotide, vector, host cell, or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi solid, semi liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion or suspension. The liquid may be for oral administration or for delivery by injection, as two examples. When intended for oral administration, preferred compositions contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

Liquid pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of an antibody or antigen-binding fragment as herein disclosed such that a suitable dosage will be obtained. Typically, this amount is at least 0.01% of the antibody or antigen-binding fragment in the composition. When intended for oral administration, this amount may be varied to be between 0.1 and about 70% of the weight of the composition. Certain oral pharmaceutical compositions contain between about 4% and about 75% of the antibody or antigen-binding fragment. In certain embodiments, pharmaceutical compositions and preparations according to the present invention are prepared so that a parenteral dosage unit contains between 0.01 to 10% by weight of antibody or antigen-binding fragment prior to dilution.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the antibody or antigen-binding fragment of the disclosure and thereby assists in the delivery of the compound. Suitable agents that may act in this capacity include monoclonal or polyclonal antibodies, one or more proteins or a liposome. The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi phasic, or tri phasic systems in order to deliver the active ingredient(s). Delivery of the aerosol includes the necessary container, activators, valves, subcontainers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises an antibody, antigen-binding fragment thereof, or antibody conjugate as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the peptide composition so as to facilitate dissolution or homogeneous suspension of the antibody or antigen-binding fragment thereof in the aqueous delivery system.

In general, an appropriate dose and treatment regimen provide the composition(s) in an amount sufficient to provide therapeutic and/or prophylactic benefit (such as described herein, including an improved clinical outcome (e.g., a decrease in frequency, duration, or severity of diarrhea or associated dehydration, or inflammation, or longer disease-free and/or overall survival, or a lessening of symptom severity). For prophylactic use, a dose should be sufficient to prevent, delay the onset of, or diminish the severity of a disease associated with disease or disorder. Prophylactic benefit of the compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Compositions are administered in an effective amount (e.g., to treat a SARS-CoV-2 infection), which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Generally, a therapeutically effective daily dose of an antibody or antigen binding fragment is (for a 70 kg mammal) from about 0.001 mg/kg (i.e., 0.07 mg) to about 100 mg/kg (i.e., 7.0 g); preferably a therapeutically effective dose is (for a 70 kg mammal) from about 0.01 mg/kg (i.e., 0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., 1.75 g). For polynucleotides, vectors, host cells, and related compositions of the present disclosure, a therapeutically effective dose may be different than for an antibody or antigen-binding fragment.

In certain embodiments, a method comprises administering the antibody, antigen-binding fragment, polynucleotide, vector, host cell, or composition to the subject at 2, 3, 4, 5, 6, 7, 8, 9, 10 times, or more.

In certain embodiments, a method comprises administering the antibody, antigen-binding fragment, or composition to the subject a plurality of times, wherein a second or successive administration is performed at about 6, about 7, about 8, about 9, about 10, about 11, about 12, about 24, about 48, about 74, about 96 hours, or more, following a first or prior administration, respectively.

In certain embodiments, a method comprises administering the antibody, antigen-binding fragment, polynucleotide, vector, host cell, or composition at least one time prior to the subject being infected by SARS-CoV-2.

Compositions comprising an antibody, antigen-binding fragment, polynucleotide, vector, host cell, or composition of the present disclosure may also be administered simultaneously with, prior to, or after administration of one or more other therapeutic agents. Such combination therapy may include administration of a single pharmaceutical dosage formulation which contains a compound of the invention and one or more additional active agents, as well as administration of compositions comprising an antibody or antigen-binding fragment of the disclosure and each active agent in its own separate dosage formulation. For example, an antibody or antigen-binding fragment thereof as described herein and the other active agent can be administered to the patient together in a single oral dosage composition such as a tablet or capsule, or each agent administered in separate oral dosage formulations. Similarly, an antibody or antigen-binding fragment as described herein and the other active agent can be administered to the subject together in a single parenteral dosage composition such as in a saline solution or other physiologically acceptable solution, or each agent administered in separate parenteral dosage formulations. Where separate dosage formulations are used, the compositions comprising an antibody or antigen-binding fragment and one or more additional active agents can be administered at essentially the same time, i.e., concurrently, or at separately staggered times, i.e., sequentially and in any order; combination therapy is understood to include all these regimens.

In certain embodiments, a combination therapy is provided that comprises one or more anti-SARS-CoV-2 antibody or antigen-binding fragment (or one or more nucleic acid, host cell, vector, or composition) of the present disclosure and one or more anti-inflammatory agent and/or one or more anti-viral agent. In particular embodiments, the one or more anti-inflammatory agent comprises a corticosteroid such as, for example, dexamethasone, prednisone, or the like. In some embodiments, the one or more anti-inflammatory agents comprise a cytokine antagonist such as, for example, an antibody or antigen-binding fragment that binds to IL6 (such as siltuximab), or to IL-6R (such as tocilizumab), or to IL-1β, IL-7, IL-8, IL-9, IL-10, FGF, G-CSF, GM-CSF, IFN-7, IP-10, MCP-1, MIP-1A, MIP1-B, PDGR, TNF-α, or VEGF. In some embodiments, anti-inflammatory agents such as ruxolitinib and/or anakinra are used. In some embodiments, the one or more anti-viral agents comprise nucleotide analogs or nucleotide analog prodrugs such as, for example, remdesivir, sofosbuvir, acyclovir, and zidovudine. In particular embodiments, an anti-viral agent comprises lopinavir, ritonavir, favipiravir, leronlimab or any combination thereof. Other anti-inflammatory agents for use in a combination therapy of the present disclosure include non-steroidal anti-inflammatory drugs (NSAIDS). It will be appreciated that in such a combination therapy, the one or more antibody or antigen-binding fragment (or one or more nucleic acid, host cell, vector, or composition) and the one or more anti-inflammatory agent and/or one or the more antiviral agent can be administered in any order and any sequence, or together.

In some embodiments, an antibody or antigen-binding fragment (or one or more nucleic acid, host cell, vector, or composition) is administered to a subject who has previously received one or more anti-inflammatory agent and/or one or more antiviral agent. In some embodiments, one or more anti-inflammatory agent and/or one or more antiviral agent is administered to a subject who has previously received an antibody (or one or more nucleic acid, host cell, vector, or composition).

In certain embodiments, a combination therapy is provided that comprises one or more anti-SARS-CoV-2 antibody or antigen-binding fragment thereof (or one or more nucleic acid, host cell, vector, or composition) of the present disclosure and an anti-CCR5 antibody or antigen binding fragments, such as leronlimab.

In certain embodiments, a combination therapy is provided that comprises two or more anti-SARS-CoV-2 antibodies of the present disclosure. A method can comprise administering a first antibody to a subject who has received a second antibody, or can comprise administering two or more antibodies together. For example, in particular embodiments, a method is provided that comprises administering to the subject (a) a first antibody or antigen-binding fragment, when the subject has received a second antibody or antigen-binding fragment; (b) the second antibody or antigen-binding fragment, when the subject has received the first antibody or antigen-binding fragment; or (c) the first antibody or antigen-binding fragment, and the second antibody or antigen-binding fragment. In certain further embodiments, (i) the first antibody or antigen-binding fragment comprises a VH comprising or consisting of the amino acid sequence set forth in any one of SEQ ID NOs.:21, 37, 374, 377, 396, and 415, and a VL comprising or consisting of the amino acid sequence set forth in any one of SEQ ID NOs.:29, 45, 380, 383, 385, 388, 392, 393, 400, 402, 404, and 406-414; and (ii) the second antibody or antigen-binding fragment comprises a VH comprising or consisting of the amino acid sequence set forth in SEQ ID NO.:69, and a VL comprising or consisting of the amino acid sequence set forth in SEQ ID NO.:77. In certain embodiments, the first antibody or antigen-binding fragment comprises three heavy chain CDRs and three light chain CDRS of a first antibody disclosed herein, and the second antibody or antigen-binding fragment independently comprises three heavy chain CDRs and three light chain CDRs of a second, different antibody disclosed herein.

In a related aspect, uses of the presently disclosed antibodies, antigen-binding fragments, vectors, host cells, and compositions are provided.

In certain embodiments, an antibody, antigen-binding fragment, polynucleotide, vector, host cell, or composition is provided for use in a method of treating a SARS-CoV-2 infection in a subject.

In certain embodiments, an antibody, antigen-binding fragment, or composition is provided for use in a method of manufacturing or preparing a medicament for treating a SARS-CoV-2 infection in a subject.

TABLE 1
Sequences

| Sequence | SEQ ID | |
|---|---|---|
| Description | NO. | Sequence |
| nCoV-3 mAb | 1 | gaagtgcagttgttggagtctgggggaggcttggttcagccgggggg |
| VH (nt) | | gtccctgagactctcctgtacagcctctggattcacctttgccagctat |
| | | gccatgacctgggtccgccaggctccaggcaaggggctggagtgggt |
| | | ctcaactattagtggtggtggtggtgacacatactccgcagactccgt |
| | | gaagggccggttcaccatctccagagacaattccaagagcacgctgta |
| | | tttgcaaatgaacagcctgagagccgcggacacggccctatattactgt |
| | | gcgagattggaaagcgcgacgcagcccctcggctactatttctacg |
| | | gtatggacgtctggggccaagggactacggtcaccgtctcctcag |
| nCoV-3 mAb | 2 | ggattcacctttgccagctatgcc |
| CDRH1 (nt) | | |
| nCoV-3 mAb | 3 | attagtggtggtggtggtgacaca |
| CDRH2 (nt) | | |
| nCoV-3 mAb | 4 | gcgagattggaaagcgcgacgcagcccctcggctactatttctacggt |
| CDRH3 (nt) | | atggacgtc |
| nCoV-3 mAb | 5 | EVQLLESGGGLVQPGGSLRLSCTASGFTFASYA |
| VH (aa) | | MTWVRQAPGKGLEWVSTISGGGGDTYSADSV |
| | | KGRFTISRDNSKSTLYLQMNSLRAADTALYYC |
| | | ARLESATQPLGYYFYGMDVWGQGTTVTVSS |
| nCoV-3 mAb | 6 | GFTFASYA |
| CDRH1 (aa) | | |
| nCoV-3 mAb | 7 | ISGGGGDT |
| CDRH2 (aa) | | |
| nCoV-3 mAb | 8 | ARLESATQPLGYYFYGMDV |
| CDRH3 (aa) | | |
| nCoV-3 mAb | 9 | tcctatgagctgacacagccaccctcggtgtcagtgtccccaggacagac |
| VL (nt) | | ggccaggatcacctgctctggagatgcattgccaaagcaatatgctttttg |
| | | gtaccagcagaggccaggccaggcccctgtgttggtgatatctaaagac |
| | | agtgagaggccctcagggatccctgagcgattctctggctccagctcagg |
| | | gacaacagtcacgttgaccatcagtggagtccaggcagaagacgaggct |
| | | gactattactgtcattcagcagacatcagtgctacttcttgggttttcggc |
| | | ggagggaccaagctgaccgtcgtt |
| nCoV-3 mAb | 10 | gcattgccaaagcaatat |
| CDRL1 (nt) | | |
| nCoV-3 mAb | 11 | aaagacagt |
| CDRL2 (nt) | | |
| nCoV-3 mAb | 12 | cattcagcagacatcagtgctacttcttgggtt |
| CDRL3 (nt) | | |
| nCoV-3 mAb | 13 | SYELTQPPSVSVSPGQTARITCSGDALPKQYAF |
| VL (aa) | | WYQQRPGQAPVLVISKDSERPSGIPERFSGSSSG |
| | | TTVTLTISGVQAEDEADYYCHSADISATSWVFG |
| | | GGTKLTVV |
| nCoV-3 mAb | 14 | ALPKQY |
| CDRL1 (aa) | | |
| nCoV-3 mAb | 15 | KDS |
| CDRL2 (aa) | | |
| nCoV-3 mAb | 16 | HSADISATSWV |
| CDRL3 (aa) | | |
| nCoV-10 | 17 | caggtgcagctggtggagtctgggggaggcgtggtccagcctgggaggt |
| mAb VH (nt- | | ccctgagactctcctgtgcagcctctggattcaccttcagtaactatggca |
| WT) | | tgcactgggtccgccaggctccaggcaaggggctggagtggctggcagt |
| | | tatatcatctgatggaagaattaagttctatgcagactccgtgaagggcc |
| | | gattcaccatgtccagagacagttccaagaacacgctgtatctgcaaatga |
| | | acagcctgagagctgaggacacggctgtgtattactgtgcgaaagatcg |
| | | gttccagtttgccagaagctggtacggtgactactttgactactggggc |
| | | cagggaacccaggtcaccgtctcctcag |
| nCoV-10 | 18 | ggattcaccttcagtaactatggc |
| mAb CDRH1 | | |
| (nt) | | |
| nCoV-10 | 19 | atatcatctgatggaagaattaag |
| mAb CDRH2 | | |
| (nt) | | |
| nCoV-10 | 20 | gcgaaagatcggttccagtttgccagaagctggtacggtgactacttt |
| mAb CDRH3 | | gactac |
| (nt) | | |
| nCoV-10 | 21 | QVQLVESGGGVVQPGRSLRLSCAASGFTFSNY |
| mAb VH (aa) | | GMHWVRQAPGKGLEWLAVISSDGRIKFYADS |
| | | VKGRFTMSRDSSKNTLYLQMNSLRAEDTAVYY |
| | | CAKDRFQFARSWYGDYFDYWGQGTQVTVSS |
| nCoV-10 | 22 | GFTFSNYG |
| mAb CDRH1 | | |
| (aa) | | |
| nCoV-10 | 23 | ISSDGRIK |
| mAb CDRH2 | | |
| (aa) | | |
| nCoV-10 | 24 | AKDRFQFARSWYGDYFDY |
| mAb CDRH3 | | |
| (aa) | | |
| nCoV-10 | 25 | gctgttgcgatgactcagtctccactctccctgcccgtcacccttggacagc |
| mAb VL (Vk) | | cggcctccatctcctgcaggtctaatcaaagcctcgtatacagtgatgga |
| (nt) | | aacatctacttgaattggtttcaacagaggccaggccaatctccaatgcgc |
| | | ctaatttatagggtttctaaccgggactctggggtcccagacagattcagc |
| | | ggcagtgggtcaggcactgatttcacactgaaaatcagcagggtggaggc |
| | | tgaagatgttgggatttattactgcatgcaagggacacactggcctccga |
| | | ctttcggcggagggaccaaggtggagatcaaac |
| nCoV-10 | 26 | caaagcctcgtatacagtgatggaaacatctac |
| mAb CDRL1 | | |
| (nt) | | |
| nCoV-10 | 27 | agggtttct |
| mAb CDRL2 | | |
| (nt) | | |
| nCoV-10 | 28 | atgcaagggacacactggcctccgact |
| mAb CDRL3 | | |
| (nt) | | |
| nCoV-10 | 29 | AVAMTQSPLSLPVTLGQPASISCRSNQSLVYSD |
| mAb VL (Vk) | | GNIYLNWFQQRPGQSPMRLIYRVSNRDSGVPD |
| (aa) | | RFSGSGSGTDFTLKISRVEAEDVGIYYCMQGTH |
| | | WPPTFGGGTKVEIK |
| nCoV-10 | 30 | QSLVYSDGNIY |
| mAb CDRL1 | | |
| (aa) | | |
| nCoV-10 | 31 | RVS |
| mAb CDRL2 | | |
| (aa) | | |
| nCoV-10 | 32 | MQGTHWPPT |
| mAb CDRL3 | | |
| (aa) | | |
| nCoV-10-v2 | 33 | caggtgcagctggtggagtctgggggaggcgtggtccagcctgggaggt |
| mAb VH (nt) | | ccctgagactctcctgtgcagcctctggattcaccttcagtaactatggca |
| | | tgcactgggtccgccaggctccaggcaaggggctggagtggctggcagt |
| | | tatatcatctgatggaagaattaagttctatgcagactccgtgaagggcc |
| | | gattcaccatgtccagagacagttccaagaacacgctgtatctgcaaatga |
| | | acagcctgagagctgaggacacggctgtgtattactgtgcgaaagatcg |
| | | gttccagtttgccagaagctggtacggtgactactttgactactggggc |
| | | cagggaacccaggtcaccgtctcctcag |
| nCoV-10-v2 | 34 | ggattcaccttcagtaactatggc |
| mAb CDRH1 | | |
| (nt) | | |
| nCoV-10-v2 | 35 | atatcatctgatggaagaattaag |
| mAb CDRH2 | | |
| (nt | | |
| nCoV-10-v2 | 36 | gcgaaagatcggttccagtttgccagaagctggtacggtgactacttt |
| mAb CDRH3 | | gactac |
| (nt) | | |
| nCoV-10-v2 | 37 | QVQLVESGGGVVQPGRSLRLSCAASGFTFSNY |
| mAb VH (aa) | | GMHWVRQAPGKGLEWLAVISSDGRIKFYADS |
| | | VKGRFTMSRDSSKNTLYLQMNSLRAEDTAVYY |
| | | CAKDRFQFARSWYGDYFDYWGQGTQVTVSS |
| nCoV-10-v2 | 38 | GFTFSNYG |
| mAb CDRH1 | | |
| (aa) | | |
| nCoV-10-v2 | 39 | ISSDGRIK |
| mAb CDRH2 | | |
| (aa) | | |
| nCoV-10-v2 | 40 | AKDRFQFARSWYGDYFDY |
| mAb CDRH3 | | |
| (aa) | | |
| nCoV-10-v2 | 41 | gctgttgcgatgactcagtctccactctccctgcccgtcacccttggacagc |
| (N→S) | | cggcctccatctcctgcaggtctagtcaaagcctcgtatacagtgatgga |
| mAb VL (Vk) | | aacatctacttgaattggtttcaacagaggccaggccaatctccaatgcgc |
| (nt) | | ctaatttatagggtttctaaccgggactctggggtcccagacagattcagc |
| | | ggcagtgggtcaggcactgatttcacactgaaaatcagcagggtggaggc |
| | | tgaagatgttgggatttattactgcatgcaagggacacactggcctccga |
| | | ctttcggcggagggaccaaggtggagatcaaac |
| nCoV-10-v2 | 42 | caaagcctcgtatacagtgatggaaacatctac |
| mAb CDRL1 | | |
| (nt) | | |
| nCoV-10-v2 | 43 | agggtttct |
| mAb CDRL2 | | |
| (nt) | | |
| nCoV-10-v2 | 44 | atgcaagggacacactggcctccgact |
| mAb CDRL3 | | |
| (nt) | | |
| nCoV-10-v2 | 45 | AVAMTQSPLSLPVTLGQPASISCRSSQSLVYSD |
| (N→S) | | GNIYLNWFQQRPGQSPMRLIYRVSNRDSGVPD |
| mAb VL (Vk) | | RFSGSGSGTDFTLKISRVEAEDVGIYYCMQGTH |
| (aa) | | WPPTFGGGTKVEIK |
| nCoV-10-v2 | 46 | QSLVYSDGNIY |
| mAb CDRL1 | | |
| (aa) | | |
| nCoV-10-v2 | 47 | RVS |
| mAb CDRL2 | | |
| (aa) | | |
| nCoV-10-v2 | 48 | MQGTHWPPT |
| mAb CDRL3 | | |
| (aa) | | |
| nCoV-17 | 49 | caggtgcagctggtggagtcggggggaggcgtggtccagcctgggagg |
| mAb VH (nt) | | tccctgagactctcctgtgcagcctctggattcactttcagttcctatgctat |
| | | acactgggtccgccaggctccaggcaagggtctggagtggctggcagtt |
| | | atatcatttgatggaagacataaatactacgcagactccgtgaagggcc |
| | | gattcaccatctccagagacaattccaagaacacggtgtatctgcaaatga |
| | | acagcctgagagttgaggacacggctgtctattcctgtgcgagagatattc |
| | | gtcatttgatagtagtggtttccgatatggacgtctggggccaagggac |
| | | cacggtcaccgtctcctcag |
| nCoV-17 | 50 | ggattcactttcagttcctatgct |
| mAb CDRH1 | | |
| (nt) | | |
| nCoV-17 | 51 | atatcatttgatggaagacataaa |
| mAb CDRH2 | | |
| (nt) | | |
| nCoV-17 | 52 | gcgagagatattcgtcatttgatagtagtggtttccgatatggacgtc |
| mAb CDRH3 | | |
| (nt) | | |
| nCoV-17 | 53 | QVQLVESGGGVVQPGRSLRLSCAASGFTFSSY |
| mAb VH (aa) | | AIHWVRQAPGKGLEWLAVISFDGRHKYYADS |
| | | VKGRFTISRDNSKNTVYLQMNSLRVEDTAVYS |
| | | CARDIRHLIVVVSDMDVWGQGTTVTVSS |
| nCoV-17 | 54 | GFTFSSYA |
| mAb CDRH1 | | |
| (aa) | | |
| nCoV-17 | 55 | ISFDGRHK |
| mAb CDRH2 | | |
| (aa) | | |
| nCoV-17 | 56 | ARDIRHLIVVVSDMDV |
| mAb CDRH3 | | |
| (aa) | | |
| nCoV-17 | 57 | gatgttgtgatgactcagtctccactctccctgcccgtcacccttggacagc |
| mAb VL (Vk) | | cggcctccatctcctgcaggtctagtcaaagcctcgtaaacagtgatgg |
| (nt) | | agacacctccttgaattggtttcagcagaggccaggccaatctccaaggc |
| | | gcctgatttataaggtttctaaccgggactctggggtcccagacagaatca |
| | | gcggcagtgggtcaggcactgatttcacactgaaaatcagcagggtggag |
| | | gctgaggatgttggggtttattactgcatgcaaggtacacactggcctcc |
| | | gacttttggccaggggaccaagctggagatcaaac |
| nCoV-17 | 58 | caaagcctcgtaaacagtgatggagacacctcc |
| mAb CDRL1 | | |
| (nt) | | |
| nCoV-17 | 59 | caaagcctcgtaaacagtgatggagacacctcc |
| mAb CDRL2 | | |
| (nt) | | |
| nCoV-17 | 60 | atgcaaggtacacactggcctccgact |
| mAb CDRL3 | | |
| (nt) | | |
| nCoV-17 | 61 | DVVMTQSPLSLPVTLGQPASISCRSSQSLVNSD |
| mAb VL (Vk) | | GDTSLNWFQQRPGQSPRRLIYKVSNRDSGVPD |
| (aa) | | RISGSGSGTDFTLKISRVEAEDVGVYYCMQGT |
| | | HWPPTFGQGTKLEIK |
| nCoV-17 | 62 | QSLVNSDGDTS |
| mAb CDRL1 | | |
| (aa) | | |
| nCoV-17 | 63 | KVS |
| mAb CDRL2 | | |
| (aa) | | |
| nCoV-17 | 64 | MQGTHWPPT |
| mAb CDRL3 | | |
| (aa) | | |
| nCoV-6 mAb | 65 | caggtcaccttgaaggagtctggtcctgtgctggtgaaacccacagagac |
| VH (nt) | | cctcacgctgacctgcaccgtctctgtgttctcactcagcaatgctagaat |
| | | gggtgtgagctggatccgtcagcccccagggaaggccctggagtggctt |
| | | gcacacattttttcgagtgaccaaaaatcctacagcacatctctgaagag |
| | | caggctcaccatctccaaggacacctccaaaagccaggtggtccttaccat |
| | | gaccaacatggaccctgaggacacaggcacatattactgtgcacgaata |
| | | aacacggcggcgtatgattatgatagtaccacctttgatatctggggcc |
| | | aagggacaatggtcaccgtctcttcag |
| nCoV-6 mAb | 66 | gtgttctcactcagcaatgctagaatgggt |
| CDRH1 (nt) | | |
| nCoV-6 mAb | 67 | attttttcgagtgaccaaaaa |
| CDRH2 (nt) | | |
| nCoV-6 mAb | 68 | gcacgaataaacacggcggcgtatgattatgatagtaccacctttgat |
| CDRH3 (nt) | | atc |
| nCoV-6 mAb | 69 | QVTLKESGPVLVKPTETLTLTCTVSVFSLSNAR |
| VH (aa) | | MGVSWIRQPPGKALEWLAHIFSSDQKSYSTSL |
| | | KSRLTISKDTSKSQVVLTMTNMDPEDTGTYYC |
| | | ARINTAAYDYDSTTFDIWGQGTMVTVSS |
| nCoV-6 mAb | 70 | VFSLSNARMG |
| CDRH1 (aa) | | |
| nCoV-6 mAb | 71 | IFSSDQK |
| CDRH2 (aa) | | |
| nCoV-6 mAb | 72 | ARINTAAYDYDSTTFDI |
| CDRH3 (aa) | | |
| nCoV-6 mAb | 73 | gacatccagatgacccagtctccatcctccctgtctgcatctgtaggagaca |
| VL (Vk) (nt) | | gagtcaccatcacttgccgggcaagtcagaccattagcaactatttaaatt |
| | | ggtatcagcagaaaccagggaaagcccctaagctcctgctctatgctgca |
| | | tccagtttgcaaagtggggtcccatcaaggttcagtggcagtggatctggg |
| | | acagatttcactctcaccatcagcagtctgcaacttgaagattttgcaacttac |
| | | tactgtcaacagagttacagtacccctcccactttcggcggagggacca |
| | | aggtggagatcaaac |
| nCoV-6 mAb | 74 | cagaccattagcaactat |
| CDRL1 (nt) | | |
| nCoV-6 mAb | 75 | gctgcatcc |
| CDRL2 (nt) | | |
| nCoV-6 mAb | 76 | caacagagttacagtacccctcccact |
| CDRL3 (nt) | | |
| nCoV-6 mAb | 77 | DIQMTQSPSSLSASVGDRVTITCRASQTISNYLN |
| VL (Vk) (aa) | | WYQQKPGKAPKLLLYAASSLQSGVPSRFSGSGS |
| | | GTDFTLTISSLQLEDFATYYCQQSYSTPPTFGG |
| | | GTKVEIK |
| nCoV-6 mAb | 78 | QTISNY |
| CDRL1 (aa) | | |
| nCoV-6 mAb | 79 | AAS |
| CDRL2 (aa) | | |
| nCoV-6 mAb | 80 | QQSYSTPPT |
| CDRL3 (aa) | | |
| nCoV-16 | 81 | caggtgcagctggtggagtctgggggaggcgtggtccagcctgggaggt |
| mAb VH (nt) | | ccctgagactctcctgtgcagcctctggattcaccttcagtagctatggca |
| | | tgcactgggtccgccaggctccaggcaaggggctggagtgggtggcattt |
| | | atatcatatgatggaagtaatatacagtatgcagactccgtgcagggcc |
| | | gattcaccatctccagagacaattccaagaccacgttgtatctgcaaatgaa |
| | | cagcctgagagttgaggacacggctatgtactactgtgcgaaagatctttt |
| | | cggatattgtaggagtaccagctgcgagtctcttgacgactggggcca |
| | | gggagccctggtcaccgtctcctcag |
| nCoV-16 | 82 | ggattcaccttcagtagctatggc |
| mAb CDRH1 | | |
| (nt) | | |
| nCoV-16 | 83 | atatcatatgatggaagtaatata |
| mAb CDRH2 | | |
| (nt) | | |
| nCoV-16 | 84 | gcgaaagatcttttcggatattgtaggagtaccagctgcgagtctcttg |
| mAb CDRH3 | | acgac |
| (nt) | | |
| nCoV-16 | 85 | QVQLVESGGGVVQPGRSLRLSCAASGFTFSSY |
| mAb VH (aa) | | GMHWVRQAPGKGLEWVAFISYDGSNIQYADS |
| | | VQGRFTISRDNSKTTLYLQMNSLRVEDTAMYY |
| | | CAKDLFGYCRSTSCESLDDWGQGALVTVSS |
| nCoV-16 | 86 | GFTFSSYG |
| mAb CDRH1 | | |
| (aa) | | |
| nCoV-16 | 87 | ISYDGSNI |
| mAb CDRH2 | | |
| (aa) | | |
| nCoV-16 | 88 | AKDLFGYCRSTSCESLDD |
| mAb CDRH3 | | |
| (aa) | | |
| nCoV-16 | 89 | gacatccagttgacccagtctccatccttcctgtctgcatctgtaggagaca |
| mAb VL (Vk) | | gagtcaccatcacttgccgggccagtcagggcattagcagttatttagcc |
| (nt) | | tggtatcagcaaaaaccagggaaagcccctaagctcctgatctatgctgca |
| | | tccactttgcaaagtggggtcccatcaaggttcagcggcactggatatggg |
| | | acagaattcactctcacaatcagcagcctgcagcctgaagattttgcaactt |
| | | attactgtcaacagcttaataattaccctctcactttcggcggagggacc |
| | | aaggtggagatcaaac |
| nCoV-16 | 90 | cagggcattagcagttat |
| mAb CDRL1 | | |
| (nt) | | |
| nCoV-16 | 91 | gctgcatcc |
| mAb CDRL2 | | |
| (nt) | | |
| nCoV-16 | 92 | caacagcttaataattaccctctcact |
| mAb CDRL3 | | |
| (nt) | | |
| nCoV-16 | 93 | DIQLTQSPSFLSASVGDRVTITCRASQGISSYLA |
| mAb VL (Vk) | | WYQQKPGKAPKLLIYAASTLQSGVPSRFSGTGY |
| (aa) | | GTEFTLTISSLQPEDFATYYCQQLNNYPLTFGG |
| | | GTKVEIK |
| nCoV-16 | 94 | QGISSY |
| mAb CDRL1 | | |
| (aa) | | |
| nCoV-16 | 95 | AAS |
| mAb CDRL2 | | |
| (aa) | | |
| nCoV-16 | 96 | QQLNNYPLT |
| mAb CDRL3 | | |
| (aa) | | |
| nCoV-20 | 97 | caggtcaccttgcgggagtctggtcctgcgctggtgaaacccacacagac |
| mAb VH (nt) | | cctcacactgacctgcaccttctctgggttctcactcaccactactgaaat |
| | | gtgtgtgggctggatccgtcagcccccagggaaggccctggagtggctt |
| | | gcactcattgattgggatgatgataaatactacagcacatctctgaggac |
| | | caggctcaccatctccaaggacacctccaaaaaccaggtggtccttaccat |
| | | gaccaacatggaccctgtggacacagccacatattactgtgcacggatgt |
| | | tcgggcgaaagcgtctggcaccaactgggttcgacccctggggccag |
| | | ggaaccctggtcaccgtctcctcag |
| nCoV-20mAb | 98 | gggttctcactcaccactactgaaatgtgt |
| CDRH1 (nt) | | |
| nCoV-20 | 99 | attgattgggatgatgataaa |
| mAb CDRH2 | | |
| (nt) | | |
| nCoV-20 | 100 | gcacggatgttcgggcgaaagcgtctggcaccaactgggttcgaccc |
| mAb CDRH3 | | c |
| (nt) | | |
| nCoV-20 | 101 | QVTLRESGPALVKPTQTLTLTCTFSGFSLTTTE |
| mAb VH (aa) | | MCVGWIRQPPGKALEWLALIDWDDDKYYSTS |
| | | LRTRLTISKDTSKNQVVLTMTNMDPVDTATYY |
| | | CARMFGRKRLAPTGFDPWGQGTLVTVSS |
| nCoV-20 | 102 | GFSLTTTEMC |
| mAb CDRH1 | | |
| (aa) | | |
| nCoV-20mAb | 103 | IDWDDDK |
| CDRH2 (aa) | | |
| nCoV-20 | 104 | ARMFGRKRLAPTGFDP |
| mAb CDRH3 | | |
| (aa) | | |
| nCoV-20 | 105 | gatattgtgatgactcagtctccactctccctgcccgtcacccctggagagc |
| mAb VL (Vk) | | cggcctccatctcctgcaggtctagtcagagcctcctgcatagtaatgga |
| (nt) | | aagaactatttggattggtacctgcagaagccagggcagtctccacagct |
| | | cctgatctgtttgggttcttatcgggcctccggggtccctgacaggttcagt |
| | | ggcagtggatcaggcacagattttacactgaaaatcagcagagtggaggc |
| | | tgaagatgttggggtttattactgcatgcaagctctacaaactccctggac |
| | | gttcggccaagggaccaaggtggaaatcaaac |
| nCoV-20 | 106 | cagagcctcctgcatagtaatggaaagaactat |
| mAb CDRL1 | | |
| (nt) | | |
| nCoV-20 | 107 | ttgggttct |
| mAb CDRL2 | | |
| (nt) | | |
| nCoV-20 | 108 | atgcaagctctacaaactccctggacg |
| mAb CDRL3 | | |
| (nt) | | |
| nCoV-20 | 109 | DIVMTQSPLSLPVTPGEPASISCRSSQSLLHSNG |
| mAb VL (Vk) | | KNYLDWYLQKPGQSPQLLICLGSYRASGVPDR |
| (aa) | | FSGSGSGTDFTLKISRVEAEDVGVYYCMQALQ |
| | | TPWTFGQGTKVEIK |
| nCoV-20 | 110 | QSLLHSNGKNY |
| mAb CDRL1 | | |
| (aa) | | |
| nCoV-20 | 111 | LGS |
| mAb CDRL2 | | |
| (aa) | | |
| nCoV-20 | 112 | MQALQTPWT |
| mAb CDRL3 | | |
| (aa) | | |
| nCoV-4 mAb | 113 | caggtgcaactggtggagtctgggggaggcgtggtccagcctgggaggt |
| VH (nt) | | ccctgagactctcctgtgaagcctctggattcaccctcagaaccagtagt |
| | | ctccactgggtccgccaggctccaggcaaggggctggagtgggtggca |
| | | gttatatcaaatgatggagccactaaattctacgcagacgccgtgaagg |
| | | gccgattcaccatctccagagacaactccaacaacaaaatatatctgcaact |
| | | gaacggcctgaaacctgaggacacggctgtctattactgtgcgagagaa |
| | | acacgtcattacagccatggtttgaactggttcgacccctggggccag |
| | | ggaaccctggtcaacgtctcctcag |
| nCoV-4 mAb | 114 | ggattcaccctcagaaccagtagt |
| CDRH1 (nt) | | |
| nCoV-4 mAb | 115 | atatcaaatgatggagccactaaa |
| CDRH2 (nt) | | |
| nCoV-4 mAb | 116 | gcgagagaaacacgtcattacagccatggtttgaactggttcgacccc |
| CDRH3 (nt) | | |
| nCoV-4 mAb | 117 | GFTLRTSSLHWVRQAPGKGLEWVAVISNDGA |
| VH (aa) | | TKFYADAVKGRFTISRDNSNNKIYLQLNGLKPE |
| | | DTAVYYCARETRHYSHGLNWFDPWGQGTLV |
| | | NVSS |
| nCoV-4 mAb | 118 | GFTLRTSS |
| CDRHI (aa) | | |
| nCoV-4 mAb | 119 | ISNDGATK |
| CDRH2 (aa) | | |
| nCoV-4 mAb | 120 | ARETRHYSHGLNWFDP |
| CDRH3 (aa) | | |
| nCoV-4 mAb | 12 | gaaagagtgatgacgcagtctccagtcaccctgtctgtgtctccaggggaa |
| | | agagccaccctctcctgcagggccagtcagagtgttagcagcgacttag |
| VL (Vk) (nt) | | cctggtaccagcagaaacctggccaggctcccaggctcctcatctatggt |
| | | gcatccaccagggccactggtatcccagccaggttcagtggcagtgggtc |
| | | tgggacagagttcactctcaccatcagcagcctgcagtctgaagattttgca |
| | | gtttattactgtcagcagtataataactggccgaccaccttcggccaagg |
| | | gacacgactggacattaaac |
| nCoV-4 mAb | 122 | cagagtgttagcagcgac |
| CDRL1 (nt) | | |
| nCoV-4 mAb | 123 | ggtgcatcc |
| CDRL2 (nt) | | |
| nCoV-4 mAb | 124 | cagcagtataataactggccgaccacc |
| CDRL3 (nt) | | |
| nCoV-4 mAb | 125 | ERVMTQSPVTLSVSPGERATLSCRASQSVSSDL |
| VL (Vk) (aa) | | AWYQQKPGQAPRLLIYGASTRATGIPARFSGSG |
| | | SGTEFTLTISSLQSEDFAVYYCQQYNNWPTTFG |
| | | QGTRLDIK |
| nCoV-4 mAb | 126 | QSVSSD |
| CDRL1 (aa) | | |
| nCoV-4 mAb | 127 | GAS |
| CDRL2 (aa) | | |
| nCoV-4 mAb | 128 | QQYNNWPTT |
| CDRL3 (aa) | | |
| nCoV-4-v2 | 129 | caggtgcaactggtggagtctgggggaggcgtggtccagcctgggaggt |
| (N→T) mAb | | ccctgagactctcctgtgaagcctctggattcaccctcagaaccagtagt |
| VH(nt) | | ctccactgggtccgccaggctccaggcaaggggctggagtgggtggca |
| | | gttatatcaaatgatggagccactaaattctacgcagacgccgtgaaggg |
| | | ccgattcaccatctccagagacaactccaacaacaaaatatatctgcaac |
| | | tgaacggcctgaaacctgaggacacggctgtctattactgtgcgagagaa |
| | | acacgtcattacagccatggtttgaactggttcgacccctggggccag |
| | | ggaaccctggtcaccgtctcctcag |
| nCoV-4-v2 | 130 | ggattcaccctcagaaccagtagt |
| mAb CDRH1 | | |
| (nt) | | |
| nCoV-4-v2 | 131 | atatcaaatgatggagccactaaa |
| mAb CDRH2 | | |
| (nt) | | |
| nCoV-4-v2 | 132 | gcgagagaaacacgtcattacagccatggtttgaactggttcgacccc |
| mAb CDRH3 | | |
| (nt) | | |
| nCoV-4-v2 | 133 | QVQLVESGGGVVQPGRSLRLSCEASGFTLRTSS |
| (NVS→ TVS) | | LHWVRQAPGKGLEWVAVISNDGATKFYADAV |
| mAb VH (aa) | | KGRFTISRDNSNNKIYLQLNGLKPEDTAVYYCA |
| | | RETRHYSHGLNWFDPWGQGTLVTVSS |
| nCoV-4-v2 | 134 | GFTLRTSS |
| mAb CDRH1 | | |
| (aa) | | |
| nCoV-4-v2 | 135 | ISNDGATK |
| mAb CDRH2 | | |
| (aa) | | |
| nCoV-4-v2 | 136 | ARETRHYSHGLNWFDP |
| mAb CDRH3 | | |
| (aa) | | |
| nCoV-4-v2 | 137 | gaaagagtgatgacgcagtctccagtcaccctgtctgtgtctccaggggaa |
| mAb VL (Vk) | | agagccaccctctcctgcagggccagtcagagtgttagcagcgacttag |
| (nt) | | cctggtaccagcagaaacctggccaggctcccaggctcctcatctatggt |
| | | gcatccaccagggccactggtatcccagccaggttcagtggcagtgggtc |
| | | tgggacagagttcactctcaccatcagcagcctgcagtctgaagattttgca |
| | | gtttattactgtcagcagtataataactggccgaccaccttcggccaagg |
| | | gacacgactggacattaaac |
| nCoV-4-v2 | 138 | cagagtgttagcagcgac |
| mAb CDRL1 | | |
| (nt) | | |
| nCoV-4-v2 | 139 | ggtgcatcc |
| mAb CDRL2 | | |
| (nt) | | |
| nCoV-4-v2 | 140 | cagcagtataataactggccgaccacc |
| mAb CDRL3 | | |
| (nt) | | |
| nCoV-4-v2 | 141 | ERVMTQSPVTLSVSPGERATLSCRASQSVSSDL |
| mAb VL (Vk) | | AWYQQKPGQAPRLLIYGASTRATGIPARFSGSG |
| (aa) | | SGTEFTLTISSLQSEDFAVYYCQQYNNWPTTFG |
| | | QGTRLDIK |
| nCoV-4-v2 | 142 | QSVSSD |
| mAb CDRL1 | | |
| (aa) | | |
| nCoV-4-v2 | 143 | GAS |
| mAb CDRL2 | | |
| (aa) | | |
| nCoV-4-v2 | 144 | QQYNNWPTT |
| mAb CDRL3 | | |
| (aa) | | |
| nCoV-4-v3 | 145 | caggtgcaactggtggagtctgggggaggcgtggtccagcctgggaggt |
| (N→T) | | ccctgagactctcctgtgaagcctctggattcaccctcagaaccagtagt |
| mAb VH (nt) | | ctccactgggtccgccaggctccaggcaaggggctggagtgggtggca |
| | | gttatatcaaatgatggagccactaaattctacgcagacgccgtgaagg |
| | | gccgattcaccatctccagagacaactccaacaacaaaatatatctgcaact |
| | | gaacggcctgaaacctgaggacacggctgtctattactgtgcgagagaa |
| | | acacgtcattacagccatggtttgaactggttcgacccctggggccag |
| | | ggaaccctggtcaccgtctcctcag |
| nCoV-4-v3 | 146 | ggattcaccctcagaaccagtagt |
| mAb CDRH1 | | |
| (nt) | | |
| nCoV-4-v3 | 147 | atatcaaatgatggagccactaaa |
| mAb CDRH2 | | |
| (nt) | | |
| nCoV-4-v3 | 148 | gcgagagaaacacgtcattacagccatggtttgaactggttegacccc |
| mAb CDRH3 | | |
| (nt) | | |
| nCoV-4-v3 | 149 | QVQLVESGGGVVQPGRSLRLSCEASGFTLRTSS |
| (N=>T) | | LHWVRQAPGKGLEWVAVISNDGATKFYADAV |
| mAb VH (aa) | | KGRFTISRDNSNNKIYLQLNGLKPEDTAVYYCA |
| | | RETRHYSHGLNWFDPWGQGTLVTVSS |
| nCoV-4-v3 | 150 | GFTLRTSS |
| mAb CDRH1 | | |
| (aa) | | |
| nCoV-4-v3 | 151 | ISNDGATK |
| mAb CDRH2 | | |
| (aa) | | |
| nCoV-4-v3 | 152 | ARETRHYSHGLNWFDP |
| mAb CDRH3 | | |
| (aa) | | |
| nCoV-4-v3 | 153 | gaaagagtgatgacgcagtctccagtcaccctgtctgtgtctccaggggaa |
| (W→F) | | agagccaccctctcctgcagggccagtcagagtgttagcagcgacttag |
| mAb VL | | cctggtaccagcagaaacctggccaggctcccaggctcctcatctatggt |
| (Vk) (nt) | | gcatccaccagggccactggtatcccagccaggttcagtggcagtgggtc |
| | | tgggacagagttcactctcaccatcagcagcctgcagtctgaagattttgca |
| | | gtttattactgtcagcagtataataacttcccgaccaccttcggccaaggg |
| | | acacgactggacattaaac |
| nCoV-4-v3 | 154 | cagagtgttagcagcgac |
| mAb CDRL1 | | |
| (nt) | | |
| nCoV-4-v3 | 155 | ggtgcatcc |
| mAb CDRL2 | | |
| (nt) | | |
| nCoV-4-v3 | 156 | cagcagtataataacttcccgaccacc |
| mAb CDRL3 | | |
| (nt) | | |
| nCoV-4-v3 | 157 | ERVMTQSPVTLSVSPGERATLSCRASQSVSSDL |
| (W→F) | | AWYQQKPGQAPRLLIYGASTRATGIPARFSGSG |
| mAb VL (Vk) | | SGTEFTLTISSLQSEDFAVYYCQQYNNFPTTFG |
| (aa) | | QGTRLDIK |
| nCoV-4-v3 | 158 | QSVSSD |
| mAb CDRL1 | | |
| (aa) | | |
| nCoV-4-v3 | 159 | GAS |
| mAb CDRL2 | | |
| (aa) | | |
| nCoV-4-v3 | 160 | QQYNNFPTT |
| mAb CDRL3 | | |
| (aa) | | |
| nCoV-15 | 161 | gaggtgcagctggtggagtctgggggaggcttggtaacgccagggcggt |
| mAb VH (nt) | | ccctgagactctcctgtacagcttctggattcacgtttggtgattatgctct |
| | | gagctggttccgccaggctccagggaaggggctggagtgggtagctttc |
| | | attagaagcaaagcttatggtgggacaacagaatacgccgcgtctgtg |
| | | aaaggcagattcaccatctcaagagatgattcgcaaatcattgtctatctgca |
| | | aatggacagcctgaaaaccgaagacacagccgtatattattgtagttggat |
| | | acaccggatagtctcctggggccagggaaccctggtcaccgtctcctca |
| | | g |
| nCoV-15 | 162 | ggattcacgtttggtgattatgct |
| mAb CDRH1 | | |
| (nt) | | |
| nCoV-15 | 163 | attagaagcaaagcttatggtgggacaaca |
| mAb CDRH2 | | |
| (nt) | | |
| nCoV-15 | 164 | agttggatacaccggatagtctcc |
| mAb CDRH3 | | |
| (nt) | | |
| nCoV-15 | 165 | EVQLVESGGGLVTPGRSLRLSCTASGFTFGDY |
| | | ALSWFRQAPGKGLEWVAFIRSKAYGGTTEYA |
| mAb VH (aa) | | ASVKGRFTISRDDSQIIVYLQMDSLKTEDTAVY |
| | | YCSWIHRIVSWGQGTLVTVSS |
| nCoV-15 | 166 | GFTFGDYA |
| mAb CDRH1 | | |
| (aa) | | |
| nCoV-15 | 167 | IRSKAYGGTT |
| mAb CDRH2 | | |
| (aa) | | |
| nCoV-15 | 168 | SWIHRIVS |
| mAb CDRH3 | | |
| (aa) | | |
| nCoV-15 | 169 | gacatccagatgacccagtctccatcctccctgtctgcatctgaaggagac |
| mAb VL (Vk) | | agagtcaccatcacttgccaggcgagtcaggacattagcatctatttaaat |
| (nt) | | tggtttcagcagaaaccagggaaagcccctaagctcctgatctacgaggc |
| | | atccaatttgaaaacaggggtcccatcaaggttcagtggaagtggatctgg |
| | | gacagattttactttcaccatcagcagcctgcagcctgaagatattgcaacat |
| | | attactgtcaacagtatgataatctcccctacacttttggccaggggacca |
| | | agctggagatcaaac |
| nCoV-15 | 170 | caggacattagcatctat |
| mAb CDRL1 | | |
| (nt) | | |
| nCoV-15 | 171 | gaggcatcc |
| mAb CDRL2 | | |
| (nt) | | |
| nCoV-15 | 172 | caacagtatgataatctcccctacact |
| mAb CDRL3 | | |
| (nt) | | |
| nCoV-15 | 173 | DIQMTQSPSSLSASEGDRVTITCQASQDISIYLN |
| mAb VL (Vk) | | WFQQKPGKAPKLLIYEASNLKTGVPSRFSGSGS |
| (aa) | | GTDFTFTISSLQPEDIATYYCQQYDNLPYTFGQ |
| | | GTKLEIK |
| nCoV-15 | 174 | QDISIY |
| mAb CDRL1 | | |
| (aa) | | |
| nCoV-15 | 175 | EAS |
| mAb CDRL2 | | |
| (aa) | | |
| nCoV-15 | 176 | QQYDNLPYT |
| mAb CDRL3 | | |
| (aa) | | |
| nCoV-12 | 177 | caggtgcagctggtggagtctgggggaggcgtggtccagcctgggaggt |
| mAb VH (nt) | | ccctgagactctcctgtgcaggctctggattcaccttcagtagctttggttt |
| | | gcactgggtccgccaggcgccaggcaagggactggagtggttggcactt |
| | | atttcagatgagggacgcattaaatactacgcaaactccgtgaagggcc |
| | | gattctttatctccagagacaattccaagaacacgctgtatctgcaaatgaac |
| | | agcctgagaggtgaggacacggctgtatattactgtgcgagagatgtcaa |
| | | aggacatattgtggtgatgacttctcttgactactggggccagggagcc |
| | | ctggtcaccgtctcctcag |
| nCoV-12 | 178 | ggattcaccttcagtagctttggt |
| mAb CDRH1 | | |
| (nt) | | |
| nCoV-12 | 179 | atttcagatgagggacgcattaaa |
| mAb CDRH2 | | |
| (nt) | | |
| nCoV-12 | 180 | gcgagagatgtcaaaggacatattgtggtgatgacttctcttgactac |
| mAb CDRH3 | | |
| (nt) | | |
| nCoV-12 | 181 | QVQLVESGGGVVQPGRSLRLSCAGSGFTFSSF |
| mAb VH (aa) | | GLHWVRQAPGKGLEWLALISDEGRIKYYANS |
| | | VKGRFFISRDNSKNTLYLQMNSLRGEDTAVYY |
| | | CARDVKGHIVVMTSLDYWGQGALVTVSS |
| nCoV-12 | 182 | GFTFSSFG |
| mAb CDRH1 | | |
| (aa) | | |
| nCoV-12 | 183 | ISDEGRIK |
| mAb CDRH2 | | |
| (aa) | | |
| nCoV-12 | 184 | ARDVKGHIVVMTSLDY |
| mAb CDRH3 | | |
| (aa) | | |
| nCoV-12 | 185 | gatgttgtgatgactcagtctccactctccctgcccgtcacccttggacagc |
| mAb VL (Vk) | | cggcctccatctcctgtaggtctagtcaaagcctcgtctccagtgatgga |
| (nt) | | gacacctccttgagttggtttcagcagaggccaggccaatctccaaggcg |
| | | cctaatttatgaggtttctaaccgggactctggggtcccagacagattcag |
| | | cggcagtgggtcaggcactgatttcacactgaaaatcagcagggtggagg |
| | | ctgaggatgttggggtttattactgcatgcaaggtacacactggcctccg |
| | | acgttcggccaagggaccaaggtggaaatcaaac |
| nCoV-12 | 186 | caaagcctcgtctccagtgatggagacacctcc |
| mAb CDRL1 | | |
| (nt) | | |
| nCoV-12 | 187 | gaggtttct |
| mAb CDRL2 | | |
| (nt) | | |
| nCoV-12 | 188 | atgcaaggtacacactggcctccgacg |
| mAb CDRL3 | | |
| (nt) | | |
| nCoV-12 | 189 | DVVMTQSPLSLPVTLGQPASISCRSSQSLVSSDG |
| mAb VL (Vk) | | DTSLSWFQQRPGQSPRRLIYEVSNRDSGVPDRF |
| (aa) | | SGSGSGTDFTLKISRVEAEDVGVYYCMQGTH |
| | | WPPTFGQGTKVEIK |
| nCoV-12 | 190 | QSLVSSDGDTS |
| mAb CDRL1 | | |
| (aa) | | |
| nCoV-12 | 191 | EVS |
| mAb CDRL2 | | |
| (aa) | | |
| nCoV-12 | 192 | MQGTHWPPT |
| mAb CDRL3 | | |
| (aa) | | |
| nCoV-5 mAb | 193 | caaatgcagctggtgcagtctgggcctgagatgaagaagcctgggacctc |
| VH (nt) | | agtgaaggtctcctgcaaggcctctggattcacttttagtaaaactacctt |
| | | gcagtgggtacgtcaggctcgtggacaacgccctgagtggataggatgg |
| | | atcgtccttggcagcggtaacacaaactacgcacattacttccaggcaag |
| | | agtcaccattaccagggacatgtccacgagcacagcctacatggaactga |
| | | gcagcctgagttccgaggacacggccgtctattactgtgcggcagagatg |
| | | gctacaattcagaattactattactactatggtatggacgtctggggcc |
| | | aagggaccacggtcaccgtctcctcag |
| nCoV-5 mAb | 194 | ggattcacttttagtaaaactacc |
| CDRH1 (nt) | | |
| nCoV-5 mAb | 195 | atcgtccttggcagcggtaacaca |
| CDRH2 (nt) | | |
| nCoV-5 mAb | 196 | gcggcagagatggctacaattcagaattactattactactatggtatgg |
| CDRH3 (nt) | | acgtc |
| nCoV-5 mAb | 197 | QMQLVQSGPEMKKPGTSVKVSCKASGFTFSKT |
| VH (aa) | | TLQWVRQARGQRPEWIGWIVLGSGNTNYAHY |
| | | FQARVTITRDMSTSTAYMELSSLSSEDTAVYYC |
| | | AAEMATIQNYYYYYGMDVWGQGTTVTVSS |
| nCoV-5 mAb | 198 | GFTFSKTT |
| CDRH1 (aa) | | |
| nCoV-5 mAb | 199 | IVLGSGNT |
| CDRH2 (aa) | | |
| nCoV-5 mAb | 200 | AAEMATIQNYYYYYGMDV |
| CDRH3 (aa) | | |
| nCoV-5 mAb | 201 | gacatccagatgacccagtctccatcctccctgtctgcatctgtcggggaca |
| VL (Vk) (nt) | | gagtcaccatcacttgccgggcaagtcagagcattaactactatttaaatt |
| | | ggtatcagcagaaacctgggaaagcccctaacctcctgatctatgctgcat |
| | | ccagtttgcctggtggggtcccatcaaggttcagtggcagtggatctggga |
| | | cagatttcactctcaccatcaccagtctgcaacctgaggattttgcaacttact |
| | | tctgtcaacagagttacagtacccctccgacttttggccaggggaccaa |
| | | gctggagatcaaac |
| nCoV-5 mAb | 202 | cagagcattaactactat |
| CDRL1 (nt) | | |
| nCoV-5 mAb | 203 | gctgcatcc |
| CDRL2 (nt) | | |
| nCoV-5 mAb | 204 | caacagagttacagtacccctccgact |
| CDRL3 (nt) | | |
| nCoV-5 mAb | 205 | DIQMTQSPSSLSASVGDRVTITCRASQSINYYLN |
| VL (Vk) (aa) | | WYQQKPGKAPNLLIYAASSLPGGVPSRFSGSGS |
| | | GTDFTLTITSLQPEDFATYFCQQSYSTPPTFGQG |
| | | TKLEIK |
| nCoV-5 mAb | 206 | QSINYY |
| CDRL1 (aa) | | |
| nCoV-5 mAb | 207 | AAS |
| CDRL2 (aa) | | |
| nCoV-5 mAb | 208 | QQSYSTPPT |
| CDRL3 (aa) | | |
| nCoV-14 | 209 | caggtgcagctggtgcactctggggctgaggtgaagaagcctggggcct |
| mAb VH (nt) | | cagtgagggtctcctgcaaggcctctggatacacctacaccgcctactat |
| | | atacactggctgcggcaggcccctggacaagggcttgagtggatgggat |
| | | ggatcaacgctgacaatggtgacacacactctgtgcagaagtttcaggg |
| | | cagggtcaccatgaccagggacacgtccatcaatacagcctacctggag |
| | | gtgagcaggctgaagtctgacgacacggccgtctattattgtgcgagagg |
| | | agacgttccagttggtactggctgggtctttgacttctggggccaggga |
| | | accctggtcaccgtctcctcag |
| nCoV-14 | 210 | ggatacacctacaccgcctactat |
| mAb CDRH1 | | |
| (nt) | | |
| nCoV-14 | 211 | atcaacgctgacaatggtgacaca |
| mAb CDRH2 | | |
| (nt) | | |
| nCoV-14 | 212 | gcgagaggagacgttccagttggtactggctgggtctttgacttc |
| mAb CDRH3 | | |
| (nt) | | |
| nCoV-14 | 213 | QVQLVHSGAEVKKPGASVRVSCKASGYTYTA |
| mAb VH (aa) | | YYIHWLRQAPGQGLEWMGWINADNGDTHSV |
| | | QKFQGRVTMTRDTSINTAYLEVSRLKSDDTAV |
| | | YYCARGDVPVGTGWVFDFWGQGTLVTVSS |
| nCoV-14 | 214 | GYTYTAYY |
| mAb CDRH1 | | |
| (aa) | | |
| nCoV-14 | 215 | INADNGDT |
| mAb CDRH2 | | |
| (aa) | | |
| nCoV-14 | 216 | ARGDVPVGTGWVFDF |
| mAb CDRH3 | | |
| (aa) | | |
| nCoV-14 | 217 | gacatccagatgacccagtctccatcctccctgtctgcatctgtgggagaca |
| mAb VL (Vk) | | gcgtcaccatcagttgccgggcaagtcagaccattaacaactttttaaatt |
| (nt) | | ggtaccagcagaagccagggaaagcccctaacctcctgatctatggtgca |
| | | tacaatttgcaaagtggggtcccatcaaggttcagtggcagtggatccggg |
| | | acagatttcactctcaccatcagcagcctgcaacctgaggatgttgcaattta |
| | | ctattgtcaacagagtctcagtatggtcaccttcggccaagggacacgac |
| | | tggacattaaac |
| nCoV-14 | 218 | cagaccattaacaacttt |
| mAb CDRL1 | | |
| (nt) | | |
| nCoV-14 | 219 | ggtgcatac |
| mAb CDRL2 | | |
| (nt) | | |
| nCoV-14 | 220 | caacagagtctcagtatggtcacc |
| mAb CDRL3 | | |
| (nt) | | |
| nCoV-14 | 221 | DIQMTQSPSSLSASVGDSVTISCRASQTINNFLN |
| mAb VL (Vk) | | WYQQKPGKAPNLLIYGAYNLQSGVPSRFSGSG |
| (aa) | | SGTDFTLTISSLQPEDVAIYYCQQSLSMVTFGQ |
| | | GTRLDIK |
| nCoV-14 | 222 | QTINNF |
| mAb CDRL1 | | |
| (aa) | | |
| nCoV-14 | 223 | GAY |
| mAb CDRL2 | | |
| (aa) | | |
| nCoV-14 | 224 | QQSLSMVT |
| mAb CDRL3 | | |
| (aa) | | |
| nCoV-9 mAb | 225 | caggtgcttctggtggagtctgggggaggcgtggtccagcctgggatgtc |
| VH (nt) | | cctgagactctcctgtgcagcctctggattcaccttcagttcctatgctatg |
| | | cactgggtccgccaggctccaggcaaggggctggagtgggtggcagtta |
| | | tctcatatgatggaagcactaaattctacgcagactccgtgaggggccg |
| | | attccccatctccagagacaattccaagaacacggtgtatctgcaaatgaac |
| | | agcctgagacctgaggacacggcagtctattactgtgcgacagttagtgt |
| | | cgaggggtataccagtggctggtatttgggaacccttgacttctggggc |
| | | cagggaaccccggtcaccgtctcctcag |
| nCoV-9 mAb | 226 | ggattcaccttcagttcctatgct |
| CDRH1 (nt) | | |
| nCoV-9 mAb | 227 | atctcatatgatggaagcactaaa |
| CDRH2 (nt) | | |
| nCoV-9 mAb | 228 | gcgacagttagtgtcgaggggtataccagtggctggtatttgggaacc |
| CDRH3 (nt) | | cttgacttc |
| nCoV-9 mAb | 229 | QVLLVESGGGVVQPGMSLRLSCAASGFTFSSY |
| VH (aa) | | AMHWVRQAPGKGLEWVAVISYDGSTKFYADS |
| | | VRGRFPISRDNSKNTVYLQMNSLRPEDTAVYY |
| | | CATVSVEGYTSGWYLGTLDFWGQGTPVTVSS |
| nCoV-9 mAb | 230 | GFTFSSYA |
| CDRH1 (aa) | | |
| nCoV-9 mAb | 231 | ISYDGSTK |
| CDRH2 (aa) | | |
| nCoV-9 mAb | 232 | CATVSVEGYTSGWYLGTLDF |
| CDRH3 (aa) | | |
| nCoV-9 mAb | 233 | gaaagagtgatgacgcagtttccagccaccctgtctgtgtctccaggggaa |
| VL (Vk) (nt) | | agagccaccctctcctgcagggccagtcagagtgttagtagcaacttag |
| | | cctggtaccagcagaaacctggccaggctcccaggctcctcatctatggt |
| | | gcatccaccagggccattggtgtcccagccaggttcagtggcagtgggtc |
| | | tgggacagagttcactctcaccatcagcagcctgcagtctgaagattttgca |
| | | gtttattactgtcagcagtataataactggccgggaacttttggccaggg |
| | | gaccaagctggagatcaaac |
| nCoV-9 mAb | 234 | cagagtgttagtagcaac |
| CDRL1 (nt) | | |
| nCoV-9 mAb | 235 | ggtgcatcc |
| CDRL2 (nt) | | |
| nCoV-9 mAb | 236 | cagcagtataataactggccgggaact |
| CDRL3 (nt) | | |
| nCoV-9 mAb | 237 | ERVMTQFPATLSVSPGERATLSCRASQSVSSNL |
| VL (Vk) (aa) | | AWYQQKPGQAPRLLIYGASTRAIGVPARFSGSG |
| | | SGTEFTLTISSLQSEDFAVYYCQQYNNWPGTFG |
| | | QGTKLEIK |
| nCoV-9 mAb | 238 | QSVSSN |
| CDRL1 (aa) | | |
| nCoV-9 mAb | 239 | GAS |
| CDRL2 (aa) | | |
| nCoV-9 mAb | 240 | QQYNNWPGT |
| CDRL3 (aa) | | |
| nCoV-18 | 241 | caggttcagctggtgcagtctggagctgaggtgaggcagcctgggacctc |
| mAb VH (nt) | | agtgaaggtctcctgcaaggcttctggttacacctttatgaataatgatat |
| | | cacctgggtgcgacaggcccctggacaggtgcttcagtggatggggtgg |
| | | atcaggccttccaatggaaacacaaactacgcacagaagttccagggc |
| | | agagtcaccctgaccacagacacatccacgaacacagcccacatggaac |
| | | tgaggagcctgaaatctgacgacacggccgtgtattattgtgcgagacaa |
| | | tctcattcgacgcggggtggctggcacttcagcccctggggccaggga |
| | | accctggtcaccgtctcctcag |
| nCoV-18 | 242 | ggttacacctttatgaataatgat |
| mAb CDRH1 | | |
| (nt) | | |
| nCoV-18 | 243 | atcaggccttccaatggaaacaca |
| mAb CDRH2 | | |
| (nt) | | |
| nCoV-18 | 244 | gcgagacaatctcattcgacgcggggggctggcacttcagcccc |
| mAb CDRH3 | | |
| (nt) | | |
| nCoV-18 | 245 | QVQLVQSGAEVRQPGTSVKVSCKASGYTFMN |
| mAb VH (aa) | | NDITWVRQAPGQVLQWMGWIRPSNGNTNYA |
| | | QKFQGRVTLTTDTSTNTAHMELRSLKSDDTAV |
| | | YYCARQSHSTRGGWHFSPWGQGTLVTVSS |
| nCoV-18 | 246 | GYTFMNND |
| mAb CDRH1 | | |
| (aa) | | |
| nCoV-18 | 247 | IRPSNGNT |
| mAb CDRH2 | | |
| (aa) | | |
| nCoV-18 | 248 | ARQSHSTRGGWHFSP |
| mAb CDRH3 | | |
| (aa) | | |
| nCoV-18 | 249 | gacatccagatgacccagtctccatcctccctgtctgcatctgtaggagaca |
| mAb VL (Vk) | | gagtcaccatcacttgccggccaagtcaacccattagctactttttaaattg |
| (nt) | | gtatcagcagacaccagggaaagcccctaagctcctcatctatgctgcatc |
| | | cagtttgcaaagtggggtcccatcaaggttcagtggcagtggatctgggac |
| | | agatttcactctcaccatcagcagtctgcaacctgaagattctgcaacttact |
| | | actgtcaacagagttatagtgtcccgtacacttttggccaggggaccaa |
| | | gctggagatcaaac |
| nCoV-18 | 250 | caacccattagctacttt |
| mAb CDRL1 | | |
| (nt) | | |
| nCoV-18 | 251 | gctgcatcc |
| mAb CDRL2 | | |
| (nt) | | |
| nCoV-18 | 252 | caacagagttatagtgtcccgtacact |
| mAb CDRL3 | | |
| (nt) | | |
| nCoV-18 | 253 | DIQMTQSPSSLSASVGDRVTITCRPSQPISYFLN |
| mAb VL (Vk) | | WYQQTPGKAPKLLIYAASSLQSGVPSRFSGSGS |
| (aa) | | GTDFTLTISSLQPEDSATYYCQQSYSVPYTFGQ |
| | | GTKLEIK |
| nCoV-18 | 254 | QPISYF |
| mAb CDRL1 | | |
| (aa) | | |
| nCoV-18 | 255 | AAS |
| mAb CDRL2 | | |
| (aa) | | |
| nCoV-18 | 256 | QQSYSVPYT |
| mAb CDRL3 | | |
| (aa) | | |
| nCoV-8 mAb | 257 | gaagtgctcctggtggagtctgggggaggcttggtacagcctggcaggtc |
| VH (nt) | | cctgagactctcctgtgtagtctctggattcacctttgattattatgccatgc |
| | | actgggtccggcaagctccagggaagggcctggagtgggtctcaggtat |
| | | tagttggaatagtgataacacagactatgcggactctgtgaagggccga |
| | | ttcaccatctccagagacaacgccaagaactccctgtatctgcaaatgaac |
| | | agtctgaaaactgaggacacggccttgtattactgtgcaaaagatattagt |
| | | ctagttttttggagtgttaaccctccccgtaacggaatggacgtctggg |
| | | gccaagggaccacggtcaccgtctcctcag |
| nCoV-8 mAb | 258 | ggattcacctttgattattatgcc |
| CDRH1 (nt) | | |
| nCoV-8 mAb | 259 | attagttggaatagtgataacaca |
| CDRH2 (nt) | | |
| nCoV-8 mAb | 260 | gcaaaagatattagtctagttttttggagtgttaaccctccccgtaacg |
| CDRH3 (nt) | | gaatggacgtc |
| nCoV-8 mAb | 261 | EVLLVESGGGLVQPGRSLRLSCVVSGFTFDYY |
| VH (aa) | | AMHWVRQAPGKGLEWVSGISWNSDNTDYAD |
| | | SVKGRFTISRDNAKNSLYLQMNSLKTEDTALY |
| | | YCAKDISLVFWSVNPPRNGMDVWGQGTTVT |
| | | VSS |
| nCoV-8 mAb | 262 | GFTFDYYAM |
| CDRH1 (aa) | | |
| nCoV-8 mAb | 263 | ISWNSDNT |
| CDRH2 (aa) | | |
| nCoV-8 mAb | 264 | AKDISLVFWSVNPPRNGMDV |
| CDRH3 (aa) | | |
| nCoV-8 mAb | 265 | gacatccagatgacccagtctccatcctccctgtctgcatctgtaggagaca |
| VL (Vk) (nt) | | gagtcaccatcacttgccgggcaagtcagagcattcgcagctatttaaatt |
| | | ggtatcagcagaaaccagggaaagcccctaacctcctgatctatactgca |
| | | tccagtttgcaaagtggggtcccatcaaggttcagtggcagtggatctggg |
| | | acagatttcactctcaccatcagcagtctgcaacctgaagattttgcgactta |
| | | ctactgtcaacagagttacagttcccctctcactttcggcggagggacca |
| | | aggtggagatcaaac |
| nCoV-8 mAb | 266 | cagagcattcgcagctat |
| CDRL1 (nt) | | |
| nCoV-8 mAb | 267 | actgcatcc |
| CDRL2 (nt) | | |
| nCoV-8 mAb | 268 | caacagagttacagttcccctctcact |
| CDRL3 (nt) | | |
| nCoV-8 mAb | 269 | DIQMTQSPSSLSASVGDRVTITCRASQSIRSYLN |
| VL (Vk) (aa) | | WYQQKPGKAPNLLIYTASSLQSGVPSRFSGSGS |
| | | GTDFTLTISSLQPEDFATYYCQQSYSSPLTFGGG |
| | | TKVEIK |
| nCoV-8 mAb | 270 | QSIRSY |
| CDRL1 (aa) | | |
| nCoV-8 mAb | 271 | TAS |
| CDRL2 (aa) | | |
| nCoV-8 mAb | 272 | QQSYSSPLT |
| CDRL3 (aa) | | |
| nCoV-7 mAb | 273 | caggagcaggtggtggagtctgggggaggcgtggtccagcctgggaag |
| VH (nt) | | tccctgagactctcctgtgcagcctctggattcagcttgaatgactttgcta |
| | | tccactgggtccgccaggctccaggcaagaggctggagtgggtggcaat |
| | | catatcatacgatggaagggttaaattttacgcagactcagtgaagggc |
| | | cgattcaccatctcccgagactcttccgtgcatctgcaaatggacagcctga |
| | | gacctgaggacacgggtctgtattactgtgcgagagactcttcgtggcag |
| | | agcactgggtggcctataaactggttcgaccgctggggccggggaac |
| | | cctggtcaccgtctcctcag |
| nCoV-7 mAb | 274 | ggattcagcttgaatgactttgct |
| CDRH1 (nt) | | |
| nCoV-7 mAb | 275 | atatcatacgatggaagggttaaa |
| CDRH2 (nt) | | |
| nCoV-7 mAb | 276 | gcgagagactcttcgtggcagagcactgggggcctataaactggttc |
| CDRH3 (nt) | | gaccgc |
| nCoV-7 mAb | 277 | QEQVVESGGGVVQPGKSLRLSCAASGFSLNDF |
| VH (aa) | | AIHWVRQAPGKRLEWVAIISYDGRVKFYADSV |
| | | KGRFTISRDSSVHLQMDSLRPEDTGLYYCARDS |
| | | SWQSTGWPINWFDRWGRGTLVTVSS |
| nCoV-7 mAb | 278 | GFSLNDFA |
| CDRH1 (aa) | | |
| nCoV-7 mAb | 279 | ISYDGRVK |
| CDRH2 (aa) | | |
| nCoV-7 mAb | 280 | ARDSSWQSTGWPINWFDR |
| CDRH3 (aa) | | |
| nCoV-7 mAb | 281 | gaaattgtgttgacacagtctccagccaccctgtctttgtctccagggcaaa |
| VL (Vk) (nt) | | gagccaccctctcctgcagggccagtcagagtgttctcagctccttagcc |
| | | tggtaccagcacaaacctggccaggctcccaggctcctcatctatgatgc |
| | | atccagcagggccactggcgtcccagccaggttcagtggcagtgggtct |
| | | gagacagacttcactctcaccatcagcagcctagagcctgaagattttgca |
| | | gtttattactgtcagcagcgtagcaactggcctccgacgttcggccaagg |
| | | gaccaaggtggaaatcaaac |
| nCoV-7 mAb | 282 | cagagtgttctcagctcc |
| CDRL1 (nt) | | |
| nCoV-7 mAb | 283 | gatgcatcc |
| CDRL2 (nt) | | |
| nCoV-7 mAb | 284 | cagcagcgtagcaactggcctccgacg |
| CDRL3 (nt) | | |
| nCoV-7 mAb | 285 | EIVLTQSPATLSLSPGQRATLSCRASQSVLSSLA |
| VL (Vk) (aa) | | WYQHKPGQAPRLLIYDASSRATGVPARFSGSGS |
| | | ETDFTLTISSLEPEDFAVYYCQQRSNWPPTFGQ |
| | | GTKVEIK |
| nCoV-7 mAb | 286 | QSVLSS |
| CDRL1 (aa) | | |
| nCoV-7 mAb | 287 | DAS |
| CDRL2 (aa) | | |
| nCoV-7 mAb | 288 | QQRSNWPPT |
| CDRL3 (aa) | | |
| nCoV-2 mAb | 289 | gacgtgcagctgttggagtctgggggaggcttggtacagcctggggggtc |
| VH (nt) | | cctgagactctcctgtgcagcctctggattcagctttagcagctatgccat |
| | | gacctgggtccgccaggctccagggaaggggctggagtgggtcgcaac |
| | | tatgagtgctagtggggatagcacaaacgacgcagactccgtgaaggg |
| | | ccggttcaccatctccagagacaattccaagaacacgctgtttctgcaaatg |
| | | aacagcctcagacccgaggacacggccgtatattactgtgcgtcccccct |
| | | tcggaattatggtgacttgctctactggggccagggaaccctggtcacc |
| | | gtctcctccg |
| nCoV-2 mAb | 290 | ggattcagctttagcagctatgcc |
| CDRH1 (nt) | | |
| nCoV-2 mAb | 291 | atgagtgctagtggggatagcaca |
| CDRH2 (nt) | | |
| nCoV-2 mAb | 292 | gcgtccccccttcggaattatggtgacttgctctac |
| CDRH3 (nt) | | |
| nCoV-2 mAb | 293 | DVQLLESGGGLVQPGGSLRLSCAASGFSFSSYA |
| VH (aa) | | MTWVRQAPGKGLEWVATMSASGDSTNDADS |
| | | VKGRFTISRDNSKNTLFLQMNSLRPEDTAVYYC |
| | | ASPLRNYGDLLYWGQGTLVTVSS |
| nCoV-2 mAb | 294 | GFSFSSYA |
| CDRH1 (aa) | | |
| nCoV-2 mAb | 295 | MSASGDST |
| CDRH2 (aa) | | |
| nCoV-2 mAb | 296 | ASPLRNYGDLLY |
| CDRH3 (aa) | | |
| nCoV-2 mAb | 297 | gacatccagatgacccagtctccttccaccctgtctgcatctgtaggagaca |
| VL (Vk) (nt) | | gagtcaccatcacttgccgggccagtcagaatattcatcgttttttggcct |
| | | ggtatcagcagaaaccagggaaagcccctaaactcctgatctatacggcg |
| | | tctagtttagaaagtggggtcccatcaaggttcagcggcagtggatttggg |
| | | acagaattcactctcaccatcagcagcctgcagcctgatgattttgcaactta |
| | | ttactgccaacaatataatagttactcgtggacgttcggccaagggacc |
| | | aaggtggaaatcaaac |
| nCoV-2 mAb | 298 | cagaatattcatcgtttt |
| CDRL1 (nt) | | |
| nCoV-2 mAb | 299 | acggcgtct |
| CDRL2 (nt) | | |
| nCoV-2 mAb | 300 | caacaatataatagttactcgtggacg |
| CDRL3 (nt) | | |
| nCoV-2 mAb | 301 | DIQMTQSPSTLSASVGDRVTITCRASQNIHRFL |
| VL (Vk) (aa) | | AWYQQKPGKAPKLLIYTASSLESGVPSRFSGSG |
| | | FGTEFTLTISSLQPDDFATYYCQQYNSYSWTFG |
| | | QGTKVEIK |
| nCoV-2 mAb | 302 | QNIHRF |
| CDRL1 (aa) | | |
| nCoV-2 mAb | 303 | TAS |
| CDRL2 (aa) | | |
| nCoV-2 mAb | 304 | QQYNSYSWT |
| CDRL3 (aa) | | |
| nCoV-19 | 305 | caggtgaagctggtggagtctgggggaggcgtggtccagcctgggaggt |
| mAb VH (nt) | | ccctgagactctcctgtgcagcctctggattcaccttcagtagcaatgcta |
| | | tgcactgggtccgccagactccaggcaaggggctggagtgggtggcact |
| | | tatatcatatgatgaaaggaataaatactacgcagagtccgtgaagggc |
| | | cgattcaccatctccagagacaattccaagaacacgctgtatctgcaaatga |
| | | acagcctgagacatgaggacacggctgtgtattactgtgcgagagatctg |
| | | caaatgagagtagtggttgtttcaaactttgactactggggccagggaa |
| | | ccctggtcaccgtctcctcag |
| nCoV-19 | 306 | ggattcaccttcagtagcaatgct |
| mAb CDRH1 | | |
| (nt) | | |
| nCoV-19 | 307 | atatcatatgatgaaaggaataaa |
| mAb CDRH2 | | |
| (nt) | | |
| nCoV-19 | 308 | gcgagagatctgcaaatgagagtagtggttgtttcaaactttgactact |
| mAb CDRH3 | | |
| (nt) | | |
| nCoV-19 | 309 | QVKLVESGGGVVQPGRSLRLSCAASGFTFSSN |
| mAb VH (aa) | | AMHWVRQTPGKGLEWVALISYDERNKYYAES |
| | | VKGRFTISRDNSKNTLYLQMNSLRHEDTAVYY |
| | | CARDLQMRVVVVSNFDYWGQGTLVTVSS |
| nCoV-19 | 310 | GFTFSSNA |
| mAb CDRH1 | | |
| (aa) | | |
| nCoV-19 | 311 | ISYDERNK |
| mAb CDRH2 | | |
| (aa) | | |
| nCoV-19 | 312 | ARDLQMRVVVVSNFDY |
| mAb CDRH3 | | |
| (aa) | | |
| nCoV-19 | 313 | gatgttgtgatgactcagtctccactctccctgcccgtcacccttggacagc |
| mAb VL (Vk) | | cggcctccatctcctgcaggtctagtcaaagcctcgtatacagtgatgga |
| (nt) | | aacacctacttgaattggtttcagcagaggccaggccaatctccaaggcg |
| | | cctaatttatgaggtttctaactgggactctggggtcccagacagattcagc |
| | | ggcagtgggtcaggcactgatttcacactgaaaatcagcagggtggaggc |
| | | tgaggatgttggggtttattactgcatgcaagctacacactggcctcccac |
| | | ttttggccaggggaccaagctggagatcaaac |
| nCoV-19 | 314 | caaagcctcgtatacagtgatggaaacacctac |
| mAb CDRL1 | | |
| (nt) | | |
| nCoV-19 | 315 | gaggtttct |
| mAb CDRL2 | | |
| (nt) | | |
| nCoV-19 | 316 | atgcaagctacacactggcctcccact |
| mAb CDRL3 | | |
| (nt) | | |
| nCoV-19 | 317 | DVVMTQSPLSLPVTLGQPASISCRSSQSLVYSD |
| mAb VL (Vk) | | GNTYLNWFQQRPGQSPRRLIYEVSNWDSGVPD |
| (aa) | | RFSGSGSGTDFTLKISRVEAEDVGVYYCMQAT |
| | | HWPPTFGQGTKLEIK |
| nCoV-19 | 318 | QSLVYSDGNTY |
| mAb CDRL1 | | |
| (aa) | | |
| nCoV-19 | 319 | EVS |
| mAb CDRL2 | | |
| (aa) | | |
| nCoV-19 | 320 | MQATHWPPT |
| mAb CDRL3 | | |
| (aa) | | |
| nCoV-1 mAb | 321 | caggcgcaactggtggagtctgggggagccttggtccagcctgggaggt |
| VH (nt) | | ccctgagactctcctgtgcagcctctggattcaccttcaggaattatgcta |
| | | tgcactgggtccgccaggctccagccacggggctgcagtggctggcagt |
| | | cataacatctgatggaaggaataaattctatgcagactccgtgaagggc |
| | | cgattcaccatctccagagaggattccaagaacacgctgtatctgcaaatg |
| | | gatagcctgagaggagaggacacggctgtctactactgcgtgacacagc |
| | | gtgataatagtcgcgattacttcccccactacttccacgacatggacgt |
| | | ctggggccaagggaccacggtcgccgtctcctcag |
| nCoV-1 mAb | 322 | ggattcaccttcaggaattatgct |
| CDRH1 (nt) | | |
| nCoV-1 mAb | 323 | ataacatctgatggaaggaataaa |
| CDRH2 (nt) | | |
| nCoV-1 mAb | 324 | gtgacacagcgtgataatagtcgcgattacttcccccactacttccacg |
| CDRH3 (nt) | | acatggacgtc |
| nCoV-1 mAb | 325 | QAQLVESGGALVQPGRSLRLSCAASGFTFRNY |
| VH (aa) | | AMHWVRQAPATGLQWLAVITSDGRNKFYADS |
| | | VKGRFTISREDSKNTLYLQMDSLRGEDTAVYY |
| | | CVTQRDNSRDYFPHYFHDMDVWGQGTTVAV |
| | | SS |
| nCoV-1 mAb | 326 | GFTFRNYA |
| CDRH1 (aa) | | |
| nCoV-1 mAb | 327 | ITSDGRNK |
| CDRH2 (aa) | | |
| nCoV-1 mAb | 328 | VTQRDNSRDYFPHYFHDMDV |
| CDRH3 (aa) | | |
| nCoV-1 mAb | 329 | gatgttgtgctgactcagtctccactctccctgcccgtcacccttggacagc |
| VL (Vk) (nt) | | cggcctccatctcctgcaggtctagtcaaagcctcgtttacagtgatgga |
| | | gacacctacttgaattggtttcagcagaggccaggccaatctccaaggcg |
| | | cctaatttatcaggtttctaatcgggactctggggtcccagacagatttagc |
| | | ggcagtgggtcaggcactgatttcacactgaaaatcagcagggtggaggc |
| | | tgaggatgttggggtttattactgcatgcaaggttcacactggcctccgac |
| | | gttcggccaagggaccaaggtggaaatcaaac |
| nCoV-1 mAb | 330 | caaagcctcgtttacagtgatggagacacctac |
| CDRL1 (nt) | | |
| nCoV-1 mAb | 331 | caggtttct |
| CDRL2 (nt) | | |
| nCoV-1 mAb | 332 | atgcaaggttcacactggcctccgacg |
| CDRL3 (nt) | | |
| nCoV-1 mAb | 333 | DVVLTQSPLSLPVTLGQPASISCRSSQSLVYSDG |
| VL (Vk) (aa) | | DTYLNWFQQRPGQSPRRLIYQVSNRDSGVPDR |
| | | FSGSGSGTDFTLKISRVEAEDVGVYYCMQGSH |
| | | WPPTFGQGTKVEIK |
| nCoV-1 mAb | 334 | QSLVYSDGDTY |
| CDRL1 (aa) | | |
| nCoV-1 mAb | 335 | QVS |
| CDRL2 (aa) | | |
| nCoV-1 mAb | 336 | MQGSHWPPT |
| CDRL3 (aa) | | |
| nCoV-11 | 337 | ctggtgcaactggtagagtctgggggaggcgtggtccagcctgggaggt |
| mAb VH (nt) | | ccctgagactctcctgtgcaggctctggattcacctttagcagctatggca |
| | | tgcactgggtccgccagactccaggcaaggggctggagtgggtggcagt |
| | | tatatcgtttgatggaaggaacaaattctacgcagaccccgtgaagggt |
| | | cgattcaccatctccagagacaattccaagaacacggtgttcttggaattgg |
| | | atagcctgacaactgaggacacggctttttattactgtgcgagagacgaca |
| | | acttggacagacactggccccttcgactcgggggttactggggccagg |
| | | gaaccctggtcaccgtctcctcag |
| nCoV-11 | 338 | ggattcacctttagcagctatggc |
| mAb CDRH1 | | |
| (nt) | | |
| nCoV-11 | 339 | atatcgtttgatggaaggaacaaa |
| mAb CDRH2 | | |
| (nt) | | |
| nCoV-11 | 340 | gcgagagacgacaacttggacagacactggccccttcgactcggggg |
| mAb CDRH3 | | ttac |
| (nt) | | |
| nCoV-11 | 341 | LVQLVESGGGVVQPGRSLRLSCAGSGFTFSSY |
| mAb VH (aa) | | GMHWVRQTPGKGLEWVAVISFDGRNKFYADP |
| | | VKGRFTISRDNSKNTVFLELDSLTTEDTAFYYC |
| | | ARDDNLDRHWPLRLGGYWGQGTLVTVSS |
| nCoV-11 | 342 | GFTFSSYG |
| mAb CDRH1 | | |
| (aa) | | |
| nCoV-11 | 343 | ISFDGRNK |
| mAb CDRH2 | | |
| (aa) | | |
| nCoV-11 | 344 | ARDDNLDRHWPLRLGGY |
| mAb CDRH3 | | |
| (aa) | | |
| nCoV-11 | 345 | gaaattgtgatgactcagtctccactctccctgcccgtcacccttggacagc |
| mAb VL (Vk) | | cggcctccatctcctgcaggtctagt caagacctcctatacaatgatgga |
| (nt) | | ggcaccgacttgaactggtttcagcagaggccaggccaatctccaaggc |
| | | gcctaatttacagggtttctaaccgggactctggggtcccagacagattca |
| | | gcggcagtgggtcaggcagtgatttcacactgaaaatcagcagggtgga |
| | | ggctgaggatgttggaatttattactgcatgcaaggtgcacactggcctcc |
| | | gactttcggccctgggaccaaagtggagatcaaac |
| nCoV-11 | 346 | caagacctcctatacaatgatggaggcaccgac |
| mAb CDRL1 | | |
| (nt) | | |
| nCoV-11 | 347 | agggtttct |
| mAb CDRL2 | | |
| (nt) | | |
| nCoV-11 | 348 | atgcaaggtgcacactggcctccgact |
| mAb CDRL3 | | |
| (nt) | | |
| nCoV-11 | 349 | EIVMTQSPLSLPVTLGQPASISCRSSQDLLYNDG |
| mAb VL (Vk) | | GTDLNWFQQRPGQSPRRLIYRVSNRDSGVPDR |
| (aa) | | FSGSGSGSDFTLKISRVEAEDVGIYYCMQGAH |
| | | WPPTFGPGTKVEIK |
| nCoV-11 | 350 | QDLLYNDGGTD |
| mAb CDRL 1 | | |
| (aa) | | |
| nCoV-11 | 351 | RVS |
| mAb CDRL2 | | |
| (aa) | | |
| nCoV-11 | 352 | MQGAHWPPT |
| mAb CDRL3 | | |
| (aa) | | |
| nCoV-13 | 353 | gaggtgcagctggtggagtctgggggaggcctggtcaagcctggggggt |
| mAb VH (nt) | | ccctgagactctcctgtgcagcctctggattcaccttcagtaactataaca |
| | | tgaactgggtccgccaggctccagggaaggggctggagtgggtctcatc |
| | | cattggtagtagtagcacttacatatactccgcagactcagtgaagggc |
| | | cgattcaccatctccagagacaacgccatgaactcgctgtttctgcaaatga |
| | | agagcctgagagccgaggacacggctgtgtattactgtgcgagaggtttc |
| | | gagaggtactactttgactcctggggccagggaaccctggtcaccgtct |
| | | cctcag |
| nCoV-13 | 354 | ggattcaccttcagtaactataac |
| mAb CDRH1 | | |
| (nt) | | |
| nCoV-13 | 355 | attggtagtagtagcacttacata |
| mAb CDRH2 | | |
| (nt) | | |
| nCoV-13 | 356 | gcgagaggtttcgagaggtactactttgactcc |
| mAb CDRH3 | | |
| (nt) | | |
| nCoV-13 | 357 | EVQLVESGGGLVKPGGSLRLSCAASGFTFSNY |
| mAb VH (aa) | | NMNWVRQAPGKGLEWVSSIGSSSTYIYSADSV |
| | | KGRFTISRDNAMNSLFLQMKSLRAEDTAVYYC |
| | | ARGFERYYFDSWGQGTLVTVSS |
| nCoV-13 | 358 | GFTFSNYN |
| mAb CDRH1 | | |
| (aa) | | |
| nCoV-13 | 359 | IGSSSTYI |
| mAb CDRH2 | | |
| (aa) | | |
| nCoV-13 | 360 | ARGFERYYFDS |
| mAb CDRH3 | | |
| (aa) | | |
| nCoV-13 | 361 | cagtctgtgctgactcagccaccctcagcgtctgggacccccgggcagag |
| mAb VL (nt) | | ggtcaccatctcttgttctggaagcacctccaacatcggaactaatgttgt |
| | | aaactggtaccagcagctcccaggaacggcccccaaactcctcatctata |
| | | gtaataatcagcggccctcttgggtccctgaccgattctctggctccaagt |
| | | ctggcacctcagcctccctggccatcagtgggctccagtctgaggatgag |
| | | gctgattattactgtgtggcatgggatgacatcctgaatgctgtggtcttc |
| | | gccggagggaccaagctgaccgtcctag |
| nCoV-13 | 362 | acctccaacatcggaactaatgtt |
| mAb CDRL1 | | |
| (nt) | | |
| nCoV-13 | 363 | agtaataat |
| mAb CDRL2 | | |
| (nt) | | |
| nCoV-13 | 364 | gtggcatgggatgacatcctgaatgctgtggtc |
| mAb CDRL3 | | |
| (nt) | | |
| nCoV-13 | 365 | QSVLTQPPSASGTPGQRVTISCSGSTSNIGTNVV |
| mAb VL (aa) | | NWYQQLPGTAPKLLIYSNNQRPSWVPDRFSGS |
| | | KSGTSASLAISGLQSEDEADYYCVAWDDILNA |
| | | VVFAGGTKLTVL |
| nCoV-13 | 366 | TSNIGTNV |
| mAb CDRL1 | | |
| (aa) | | |
| nCoV-13 | 367 | SNN |
| mAb CDRL2 | | |
| (aa) | | |
| nCoV-13 | 368 | VAWDDILNAVV |
| mAb CDRL3 | | |
| (aa) | | |
| Wuhan | 369 | 1 attaaaggtt tataccttcc caggtaacaa accaaccaac tttcgatctc ttgtagatct |
| seafood | | 61 gttctctaaa cgaactttaa aatctgtgtg gctgtcactc ggctgcatgc ttagtgcact |
| market | | 121 cacgcagtat aattaataac taattactgt cgttgacagg acacgagtaa ctcgtctatc |
| pneumonia | | 181 ttctgcaggc tgcttacggt ttcgtccgtg ttgcagccga tcatcagcac atctaggttt |
| virus isolate | | 241 cgtccgggtg tgaccgaaag gtaagatgga gagccttgtc cctggtttca acgagaaaac |
| Wuhan-Hu-1 | | 301 acacgtccaa ctcagtttgc ctgttttaca ggttcgcgac gtgctcgtac gtggctttgg |
| genomic | | 361 agactccgtg gaggaggtct tatcagaggc cgtcaacat cttaaagatg gcacttgtgg |
| sequence | | 421 cttagtagaa gttgaaaaag gcgttttgcc tcaacttgaa cagccctatg tgttcatcaa |
| (GenBank: | | 481 acgttcggat gctcgaactg cacctcatgg tcatgttatg gttgagctgg tagcagaact |
| MN908947.3; | | 541 cgaaggcatt cagtacggtc gtagtggtga gacacttggt gtccttgtcc ctcatgtggg |
| Jan. 23, | | 601 cgaaatacca gtggcttacc gcaaggttct tcttcgtaag aacggtaata aaggagctgg |
| 2020) | | 661 tggccatagt tacggcgccg atctaaagtc atttgactta ggcgacgagc ttggcactga |
| | | 721 tccttatgaa gattttcaag aaaactggaa cactaaacat agcagtggtg ttacccgtga |
| | | 781 actcatgcgt gagcttaacg gaggggcata cactcgctat gtcgataaca acttctgtgg |
| | | 841 ccctgatggc taccctcttg agtgcattaa agaccttcta gcacgtgctg gtaaagcttc |
| | | 901 atgcactttg tccgaacaac tggactttat tgacactaag aggggtgtat actgctgccg |
| | | 961 tgaacatgag catgaaattg cttggtacac ggaacgttct gaaaagagct atgaattgca |
| | | 1021 gacacctttt gaaattaaat tggcaaagaa atttgacacc ttcaatgggg aatgtccaaa |
| | | 1081 ttttgtattt cccttaaatt ccataatcaa gactattcaa ccaagggttg aaaagaaaaa |
| | | 1141 gcttgatggc tttatgggta gaattcgatc tgtctatcca gttgcgtcac caaatgaatg |
| | | 1201 caaccaaatg tgcctttcaa ctctcatgaa gtgtgatcat tgtggtgaaa cttcatggca |
| | | 1261 gacgggcgat tttgttaaag ccacttgcga attttgtggc actgagaatt tgactaaaga |
| | | 1321 aggtgccact acttgtggtt acttacccca aaatgctgtt gttaaaattt attgtccagc |
| | | 1381 atgtcacaat tcagaagtag gacctgagca tagtcttgcc gaataccata atgaatctgg |
| | | 1441 cttgaaaacc attcttcgta agggtggtcg cactattgcc tttggaggct gtgtgttctc |
| | | 1501 ttatgttggt tgccataaca agtgtgccta ttgggttcca cgtgctagcg ctaacatagg |
| | | 1561 ttgtaaccat acaggtgttg ttggagaagg ttccgaaggt cttaatgaca accttcttga |
| | | 1621 aatactccaa aaagagaaag tcaacatcaa tattgttggt gactttaaac ttaatgaaga |
| | | 1681 gatcgccatt attttggcat ctttttctgc ttccacaagt gcttttgtgg aaactgtgaa |
| | | 1741 aggtttggat tataaagcat tcaaacaaat tgttgaatcc tgtggtaatt ttaaagttac |
| | | 1801 aaaaggaaaa gctaaaaaag gtgcctggaa tattggtgaa cagaaatcaa tactgagtcc |
| | | 1861 tctttatgca tttgcatcag aggctgctcg tgttgtacga tcaattttct cccgcactct |
| | | 1921 tgaaactgct caaaattctg tgcgtgtttt acagaaggcc gctataacaa tactagatgg |
| | | 1981 aatttcacag tattcactga gactcattga tgctatgatg ttcacatctg atttggctac |
| | | 2041 taacaatcta gttgtaatgg cctacattac aggtggtgtt gttcagttga cttcgcagtg |
| | | 2101 gctaactaac atctttggca ctgtttatga aaaactcaaa cccgtccttg attggcttga |
| | | 2161 agagaagttt aaggaaggtg tagagtttct tagagacggt tgggaaattg ttaaatttat |
| | | 2221 ctcaacctgt gcttgtgaaa ttgtcggtgg acaaattgtc acctgtgcaa aggaaattaa |
| | | 2281 ggagagtgtt cagacattct ttaagcttgt aaataaattt ttggctttgt gtgctgactc |
| | | 2341 tatcattatt ggtggagcta aacttaaagc cttgaattta ggtgaaacat ttgtcacgca |
| | | 2401 ctcaaaggga ttgtacagaa agtgtgttaa atccagagaa gaaactggcc tactcatgcc |
| | | 2461 tctaaaagcc ccaaaagaaa ttatcttctt agagggagaa acacttccca cagaagtgtt |
| | | 2521 aacagaggaa gttgtcttga aaactggtga tttacaacca ttagaacaac ctactagtga |
| | | 2581 agctgttgaa gctccattgg ttggtacacc agtttgtatt aacgggctta tgttgctcga |
| | | 2641 aatcaaagac acagaaaagt actgtgccct tgcacctaat atgatggtaa caaacaatac |
| | | 2701 cttcacactc aaaggcggtg caccaacaaa ggttactttt ggtgatgaca ctgtgataga |
| | | 2761 agtgcaaggt tacaagagtg tgaatatcac ttttgaactt gatgaaagga ttgataaagt |
| | | 2821 acttaatgag aagtgctctg cctatacagt tgaactcggt acagaagtaa atgagttcgc |
| | | 2881 ctgtgttgtg gcagatgctg tcataaaaac tttgcaacca gtatctgaat tacttacacc |
| | | 2941 actgggcatt gatttagatg agtggagtat ggctacatac tacttatttg atgagtctgg |
| | | 3001 tgagtttaaa ttggcttcac atatgtattg ttctttctac cctccagatg aggatgaaga |
| | | 3061 agaaggtgat tgtgaagaag aagagtttga gccatcaact caatatgagt atggtactga |
| | | 3121 agatgattac caaggtaaac ctttggaatt tggtgccact tctgctgctc ttcaacctga |
| | | 3181 agaagagcaa gaagaagatt ggttagatga tgatagtcaa caaactgttg gtcaacaaga |
| | | 3241 cggcagtgag gacaatcaga caactactat tcaaacaatt gttgaggttc aacctcaatt |
| | | 3301 agagatggaa cttacaccag ttgttcagac tattgaagtg aatagtttta gtggttattt |
| | | 3361 aaaacttact gacaatgtat acattaaaaa tgcagacatt gtggaagaag ctaaaaaggt |
| | | 3421 aaaaccaaca gtggttgtta atgcagccaa tgtttacctt aaacatggag gaggtgttgc |
| | | 3481 aggagcctta aataaggcta ctaacaatgc catgcaagtt gaatctgatg attacatagc |
| | | 3541 tactaatgga ccacttaaag tgggtggtag ttgtgtttta agcggacaca atcttgctaa |
| | | 3601 acactgtctt catgttgtcg gcccaaatgt taacaaaggt gaagacattc aacttcttaa |
| | | 3661 gagtgcttat gaaaatttta atcagcacga agttctactt gcaccattat tatcagctgg |
| | | 3721 tatttttggt gctgacccta tacattcttt aagagtttgt gtagatactg ttcgcacaaa |
| | | 3781 tgtctactta gctgtctttg ataaaaatct ctatgacaaa cttgtttcaa gctttttgga |
| | | 3841 aatgaagagt gaaaagcaag ttgaacaaaa gatcgctgag attcctaaag aggaagttaa |
| | | 3901 gccatttata actgaaagta aaccttcagt tgaacagaga aaacaagatg ataagaaaat |
| | | 3961 caaagcttgt gttgaagaag ttacaacaac tctggaagaa actaagttcc tcacagaaaa |
| | | 4021 cttgttactt tatattgaca ttaatggcaa tcttcatcca gattctgcca ctcttgttag |
| | | 4081 tgacattgac atcactttct taaagaaaga tgctccatat atagtgggtg atgttgttca |
| | | 4141 agagggtgtt ttaactgctg tggttatacc tactaaaaag gctggtggca ctactgaaat |
| | | 4201 gctagcgaaa gctttgagaa aagtgccaac agacaattat ataaccactt acccgggtca |
| | | 4261 gggtttaaat ggttacactg tagaggaggc aaagacagtg cttaaaaagt gtaaaagtgc |
| | | 4321 cttttacatt ctaccatcta ttatctctaa tgagaagcaa gaaattcttg gaactgtttc |
| | | 4381 ttggaatttg cgagaaatgc ttgcacatgc agaagaaaca cgcaaattaa tgcctgtctg |
| | | 4441 tgtggaaact aaagccatag tttcaactat acagcgtaaa tataagggta ttaaaataca |
| | | 4501 agagggtgtg gttgattatg gtgctagatt ttacttttac accagtaaaa caactgtagc |
| | | 4561 gtcacttatc aacacactta acgatctaaa tgaaactctt gttacaatgc cacttggcta |
| | | 4621 tgtaacacat ggcttaaatt tggaagaagc tgctcggtat atgagatctc tcaaagtgcc |
| | | 4681 agctacagtt tctgtttctt cacctgatgc tgttacagcg tataatggtt atcttacttc |
| | | 4741 ttcttctaaa acacctgaag aacattttat tgaaaccatc tcacttgctg gttcctataa |
| | | 4801 agattggtcc tattctggac aatctacaca actaggtata gaatttctta agagaggtga |
| | | 4861 taaaagtgta tattacacta gtaatcctac cacattccac ctagatggtg aagttatcac |
| | | 4921 ctttgacaat cttaagacac ttctttcttt gagagaagtg aggactatta aggtgtttac |
| | | 4981 aacagtagac aacattaacc tccacacgca agttgtggac atgtcaatga catatggaca |
| | | 5041 acagtttggt ccaacttatt tggatggagc tgatgttact aaaataaaac ctcataattc |
| | | 5101 acatgaaggt aaaacatttt atgttttacc taatgatgac actctacgtg ttgaggcttt |
| | | 5161 tgagtactac cacacaactg atcctagttt tctgggtagg tacatgtcag cattaaatca |
| | | 5221 cactaaaaag tggaaatacc cacaagttaa tggtttaact tctattaaat gggcagataa |
| | | 5281 caactgttat cttgccactg cattgttaac actccaacaa atagagttga agtttaatcc |
| | | 5341 acctgctcta caagatgctt attacagagc aagggctggt gaagctgcta acttttgtgc |
| | | 5401 acttatctta gcctactgta ataagacagt aggtgagtta ggtgatgtta gagaaacaat |
| | | 5461 gagttacttg tttcaacatg ccaatttaga ttcttgcaaa agagtcttga acgtggtgtg |
| | | 5521 taaaacttgt ggacaacagc agacaaccct taagggtgta gaagctgtta tgtacatggg |
| | | 5581 cacactttct tatgaacaat ttaagaaagg tgttcagata ccttgtacgt gtggtaaaca |
| | | 5641 agctacaaaa tatctagtac aacaggagtc accttttgtt atgatgtcag caccacctgc |
| | | 5701 tcagtatgaa cttaagcatg gtacatttac ttgtgctagt gagtacactg gtaattacca |
| | | 5761 gtgtggtcac tataaacata taacttctaa agaaactttg tattgcatag acggtgcttt |
| | | 5821 acttacaaag tcctcagaat acaaaggtcc tattacggat gttttctaca aagaaaacag |
| | | 5881 ttacacaaca accataaaac cagttactta taaattggat ggtgttgttt gtacagaaat |
| | | 5941 tgaccctaag ttggacaatt attataagaa agacaattct tatttcacag agcaaccaat |
| | | 6001 tgatcttgta ccaaaccaac catatccaaa cgcaagcttc gataatttta agtttgtatg |
| | | 6061 tgataatatc aaatttgctg atgatttaaa ccagttaact ggttataaga aacctgcttc |
| | | 6121 aagagagctt aaagttacat ttttccctga cttaaatggt gatgtggtgg ctattgatta |
| | | 6181 taaacactac acaccctctt ttaagaaagg agctaaattg ttacataaac ctattgtttg |
| | | 6241 gcatgttaac aatgcaacta ataaagccac gtataaacca aatacctggt gtatacgttg |
| | | 6301 tctttggagc acaaaaccag ttgaaacatc aaattcgttt gatgtactga agtcagagga |
| | | 6361 cgcgcaggga atggataatc ttgcctgcga agatctaaaa ccagtctctg aagaagtagt |
| | | 6421 ggaaaatcct accatacaga aagacgttct tgagtgtaat gtgaaaacta ccgaagttgt |
| | | 6481 aggagacatt atacttaaac cagcaaataa tagtttaaaa attacagaag aggttggcca |
| | | 6541 cacagatcta atggctgctt atgtagacaa ttctagtctt actattaaga aacctaatga |
| | | 6601 attatctaga gtattaggtt tgaaaaccct tgctactcat ggtttagctg ctgttaatag |
| | | 6661 tgtcccttgg gatactatag ctaattatgc taagcctttt cttaacaaag ttgttagtac |
| | | 6721 aactactaac atagttacac ggtgtttaaa ccgtgtttgt actaattata tgccttattt |
| | | 6781 ctttacttta ttgctacaat tgtgtacttt tactagaagt acaaattcta gaattaaagc |
| | | 6841 atctatgccg actactatag caaagaatac tgttaagagt gtcggtaaat tttgtctaga |
| | | 6901 ggcttcattt aattatttga agtcacctaa tttttctaaa ctgataaata ttataatttg |
| | | 6961 gtttttacta ttaagtgttt gcctaggttc tttaatctac tcaaccgctg ctttaggtgt |
| | | 7021 tttaatgtct aatttaggca tgccttctta ctgtactggt tacagagaag gctatttgaa |
| | | 7081 ctctactaat gtcactattg caacctactg tactggttct ataccttgta gtgtttgtct |
| | | 7141 tagtggttta gattctttag acacctatcc ttctttagaa actatacaaa ttaccatttc |
| | | 7201 atcttttaaa tgggatttaa ctgcttttgg cttagttgca gagtggtttt tggcatatat |
| | | 7261 tcttttcact aggtttttct atgtacttgg attggctgca atcatgcaat tgtttttcag |
| | | 7321 ctattttgca gtacatttta ttagtaattc ttggcttatg tggttaataa ttaatcttgt |
| | | 7381 acaaatggcc ccgatttcag ctatggttag aatgtacatc ttctttgcat cattttatta |
| | | 7441 tgtatggaaa agttatgtgc atgttgtaga cggttgtaat tcatcaactt gtatgatgtg |
| | | 7501 ttacaaacgt aatagagcaa caagagtcga atgtacaact attgttaatg gtgttagaag |
| | | 7561 gtccttttat gtctatgcta atggaggtaa aggcttttgc aaactacaca attggaattg |
| | | 7621 tgttaattgt gatacattct gtgctggtag tacatttatt agtgatgaag ttgcgagaga |
| | | 7681 cttgtcacta cagtttaaaa gaccaataaa tcctactgac cagtcttctt acatcgttga |
| | | 7741 tagtgttaca gtgaagaatg gttccatcca tctttacttt gataaagctg gtcaaaagac |
| | | 7801 ttatgaaaga cattctctct ctcattttgt taacttagac aacctgagag ctaataacac |
| | | 7861 taaaggttca ttgcctatta atgttatagt ttttgatggt aaatcaaaat gtgaagaatc |
| | | 7921 atctgcaaaa tcagcgtctg tttactacag tcagcttatg tgtcaaccta tactgttact |
| | | 7981 agatcaggca ttagtgtctg atgttggtga tagtgcggaa gttgcagtta aaatgtttga |
| | | 8041 tgcttacgtt aatacgtttt catcaacttt taacgtacca atggaaaaac tcaaaacact |
| | | 8101 agttgcaact gcagaagctg aacttgcaaa gaatgtgtcc ttagacaatg tcttatctac |
| | | 8161 ttttatttca gcagctcggc aagggtttgt tgattcagat gtagaaacta aagatgttgt |
| | | 8221 tgaatgtctt aaattgtcac atcaatctga catagaagtt actggcgata gttgtaataa |
| | | 8281 ctatatgctc acctataaca aagttgaaaa catgacaccc cgtgaccttg gtgcttgtat |
| | | 8341 tgactgtagt gcgcgtcata ttaatgcgca ggtagcaaaa agtcacaaca ttgctttgat |
| | | 8401 atggaacgtt aaagatttca tgtcattgtc tgaacaacta cgaaaacaaa tacgtagtgc |
| | | 8461 tgctaaaaag aataacttac cttttaagtt gacatgtgca actactagac aagttgttaa |
| | | 8521 tgttgtaaca acaaagatag cacttaaggg tggtaaaatt gttaataatt ggttgaagca |
| | | 8581 gttaattaaa gttacacttg tgttcctttt tgttgctgct attttctatt taataacacc |
| | | 8641 tgttcatgtc atgtctaaac atactgactt ttcaagtgaa atcataggat acaaggctat |
| | | 8701 tgatggtggt gtcactcgtg acatagcatc tacagatact tgttttgcta acaaacatgc |
| | | 8761 tgattttgac acatggttta gccagcgtgg tggtagttat actaatgaca aagcttgccc |
| | | 8821 attgattgct gcagtcataa caagagaagt gggttttgtc gtgcctggtt tgcctggca |
| Wuhan | 370 | MESLVPGFNEKTHVQLSLPVLQVRDVLVRGFGDSV |
| seafood | | EEVLSEARQHLKDGTCGLVEVEKGVLPQLEQPYVF |
| market | | IKRSDARTAPHGHVMVELVAELEGIQYGRSGETLG |
| pneumonia | | VLVPHVGEIPVAYRKVLLRKNGNKGAGGHSYGADL |
| virus isolate | | KSFDLGDELGTDPYEDFQENWNTKHSSGVTRELMR |
| Wuhan-Hu-1 | | ELNGGAYTRYVDNNFCGPDGYPLECIKDLLARAGK |
| genomic | | ASCTLSEQLDFIDTKRGVYCCREHEHEIAWYTERS |
| sequence | | EKSYELQTPFEIKLAKKFDTFNGECPNFVFPLNSI |
| (GenBank: | | IKTIQPRVEKKKLDGFMGRIRSVYPVASPNECNQM |
| MN908947.3; | | CLSTLMKCDHCGETSWQTGDFVKATCEFCGTENLT |
| Jan. 23, | | KEGATTCGYLPQNAVVKIYCPACHNSEVGPEHSLA |
| 2020)-amino | | EYHNESGLKTILRKGGRTIAFGGCVFSYVGCHNKC |
| acid | | AYWVPRASANIGCNHTGVVGEGSEGLNDNLLEILQ |
| translation | | KEKVNINIVGDFKLNEEIAIILASFSASTSAFVET |
| | | VKGLDYKAFKQIVESCGNFKVTKGKAKKGAWNIGE |
| | | QKSILSPLYAFASEAARVVRSIFSRTLETAQNSVR |
| | | VLQKAAITILDGISQYSLRLIDAMMFTSDLATNNL |
| | | VVMAYITGGVVQLTSQWLTNIFGTVYEKLKPVLDW |
| | | LEEKFKEGVEFLRDGWEIVKFISTCACEIVGGQIV |
| | | TCAKEIKESVQTFFKLVNKFLALCADSIIIGGAKL |
| | | KALNLGETFVTHSKGLYRKCVKSREETGLLMPLKA |
| | | PKEIIFLEGETLPTEVLTEEVVLKTGDLQPLEQPT |
| | | SEAVEAPLVGTPVCINGLMLLEIKDTEKYCALAPN |
| | | MMVTNNTFTLKGGAPTKVTFGDDTVIEVQGYKSVN |
| | | ITFELDERIDKVLNEKCSAYTVELGTEVNEFACVV |
| | | ADAVIKTLQPVSELLTPLGIDLDEWSMATYYLFDE |
| | | SGEFKLASHMYCSFYPPDEDEEEGDCEEEEFEPST |
| | | QYEYGTEDDYQGKPLEFGATSAALQPEEEQEEDWL |
| | | DDDSQQTVGQQDGSEDNQTTTIQTIVEVQPQLEME |
| | | LTPVVQTIEVNSFSGYLKLTDNVYIKNADIVEEAK |
| | | KVKPTVVVNAANVYLKHGGGVAGALNKATNNAMQV |
| | | ESDDYIATNGPLKVGGSCVLSGHNLAKHCLHVVGP |
| | | NVNKGEDIQLLKSAYENFNQHEVLLAPLLSAGIFG |
| | | ADPIHSLRVCVDTVRTNVYLAVFDKNLYDKLVSSF |
| | | LEMKSEKQVEQKIAEIPKEEVKPFITESKPSVEQR |
| | | KODDKKIKACVEEVTTTLEETKFLTENLLLYIDIN |
| | | GNLHPDSATLVSDIDITFLKKDAPYIVGDVVQEGV |
| | | LTAVVIPTKKAGGTTEMLAKALRKVPTDNYITTYP |
| | | GQGLNGYTVEEAKTVLKKCKSAFYILPSIISNEKQ |
| | | EILGTVSWNLREMLAHAEETRKLMPVCVETKAIVS |
| | | TIQRKYKGIKIQEGVVDYGARFYFYTSKTTVASLI |
| | | NTLNDLNETLVTMPLGYVTHGLNLEEAARYMRSLK |
| | | VPATVSVSSPDAVTAYNGYLTSSSKTPEEHFIETI |
| | | SLAGSYKDWSYSGQSTQLGIEFLKRGDKSVYYTSN |
| | | PTTFHLDGEVITFDNLKTLLSLREVRTIKVFTTVD |
| | | NINLHTQVVDMSMTYGQQFGPTYLDGADVTKIKPH |
| | | NSHEGKTFYVLPNDDTLRVEAFEYYHTTDPSFLGR |
| | | YMSALNHTKKWKYPQVNGLTSIKWADNNCYLATAL |
| | | LTLQQIELKFNPPALQDAYYRARAGEAANFCALIL |
| | | AYCNKTVGELGDVRETMSYLFQHANLDSCKRVLNV |
| | | VCKTCGQQQTTLKGVEAVMYMGTLSYEQFKKGVQI |
| | | PCTCGKQATKYLVQQESPFVMMSAPPAQYELKHGT |
| | | FTCASEYTGNYQCGHYKHITSKETLYCIDGALLTK |
| | | SSEYKGPITDVFYKENSYTTTIKPVTYKLDGVVCT |
| | | EIDPKLDNYYKKDNSYFTEQPIDLVPNQPYPNASF |
| | | DNFKFVCDNIKFADDLNQLTGYKKPASRELKVTFF |
| | | PDLNGDVVAIDYKHYTPSFKKGAKLLHKPIVWHVN |
| | | NATNKATYKPNTWCIRCLWSTKPVETSNSFDVLKS |
| | | EDAQGMDNLACEDLKPVSEEVVENPTIQKDVLECN |
| | | VKTTEVVGDIILKPANNSLKITEEVGHTDLMAAYV |
| | | DNSSLTIKKPNELSRVLGLKTLATHGLAAVNSVPW |
| | | DTIANYAKPFLNKVVSTTTNIVTRCLNRVCTNYMP |
| | | YFFTLLLQLCTFTRSTNSRIKASMPTTIAKNTVKS |
| | | VGKFCLEASFNYLKSPNFSKLINIIIWFLLLSVCL |
| | | GSLIYSTAALGVLMSNLGMPSYCTGYREGYLNSTN |
| | | VTIATYCTGSIPCSVCLSGLDSLDTYPSLETIQIT |
| | | ISSFKWDLTAFGLVAEWFLAYILFTRFFYVLGLAA |
| | | IMQLFFSYFAVHFISNSWLMWLIINLVQMAPISAM |
| | | VRMYIFFASFYYVWKSYVHVVDGCNSSTCMMCYKR |
| | | NRATRVECTTIVNGVRRSFYVYANGGKGFCKLHNW |
| | | NCVNCDTFCAGSTFISDEVARDLSLQFKRPINPTD |
| | | QSSYIVDSVTVKNGSIHLYFDKAGQKTYERHSLSH |
| | | FVNLDNLRANNTKGSLPINVIVFDGKSKCEESSAK |
| | | SASVYYSQLMCQPILLLDQALVSDVGDSAEVAVKM |
| | | FDAYVNTFSSTFNVPMEKLKTLVATAEAELAKNVS |
| | | LDNVLSTFISAARQGFVDSDVETKDVVECLKLSHQ |
| | | SDIEVTGDSCNNYMLTYNKVENMTPRDLGACIDCS |
| | | ARHINAQVAKSHNIALIWNVKDFMSLSEQLRKQIR |
| | | SAAKKNNLPFKLTCATTRQVVNVVTTKIALKGGKI |
| | | VNNWLKQLIKVTLVFLFVAAIFYLITPVHVMSKHT |
| | | DFSSEIIGYKAIDGGVTRDIASTDTCFANKHADFD |
| | | TWFSQRGGSYTNDKACPLIAAVITREVGFVVPGLP |
| | | GTILRTTNGDFLHFLPRVFSAVGNICYTPSKLIEY |
| | | TDFATSACVLAAECTIFKDASGKPVPYCYDTNVLE |
| | | GSVAYESLRPDTRYVLMDGSIIQFPNTYLEGSVRV |
| | | VTTFDSEYCRHGTCERSEAGVCVSTSGRWVLNNDY |
| | | YRSLPGVFCGVDAVNLLTNMFTPLIQPIGALDISA |
| | | SIVAGGIVAIVVTCLAYYFMRFRRAFGEYSHVVAF |
| | | NTLLFLMSFTVLCLTPVYSFLPGVYSVIYLYLTFY |
| | | LTNDVSFLAHIQWMVMFTPLVPFWITIAYIICIST |
| | | KHFYWFFSNYLKRRVVFNGVSFSTFEEAALCTFLL |
| | | NKEMYLKLRSDVLLPLTQYNRYLALYNKYKYFSGA |
| | | MDTTSYREAACCHLAKALNDFSNSGSDVLYQPPQT |
| | | SITSAVLQSGFRKMAFPSGKVEGCMVQVTCGTTTL |
| | | NGLWLDDVVYCPRHVICTSEDMLNPNYEDLLIRKS |
| | | NHNFLVQAGNVQLRVIGHSMQNCVLKLKVDTANPK |
| | | TPKYKFVRIQPGQTFSVLACYNGSPSGVYQCAMRP |
| | | NFTIKGSFLNGSCGSVGFNIDYDCVSFCYMHHMEL |
| | | PTGVHAGTDLEGNFYGPFVDRQTAQAAGTDTTITV |
| | | NVLAWLYAAVINGDRWFLNRFTTTLNDFNLVAMKY |
| | | NYEPLTQDHVDILGPLSAQTGIAVLDMCASLKELL |
| | | QNGMNGRTILGSALLEDEFTPFDVVRQCSGVTFQS |
| | | AVKRTIKGTHHWLLLTILTSLLVLVQSTQWSLFFF |
| | | LYENAFLPFAMGIIAMSAFAMMFVKHKHAFLCLFL |
| | | LPSLATVAYFNMVYMPASWVMRIMTWLDMVDTSLS |
| | | GFKLKDCVMYASAVVLLILMTARTVYDDGARRVWT |
| | | LMNVLTLVYKVYYGNALDQAISMWALIISVTSNYS |
| | | GVVTTVMFLARGIVFMCVEYCPIFFITGNTLQCIM |
| | | LVYCFLGYFCTCYFGLFCLLNRYFRLTLGVYDYLV |
| | | STQEFRYMNSQGLLPPKNSIDAFKLNIKLLGVGGK |
| | | PCIKVATVQSKMSDVKCTSVVLLSVLQQLRVESSS |
| | | KLWAQCVQLHNDILLAKDTTEAFEKMVSLLSVLLS |
| | | MQGAVDINKLCEEMLDNRATLQAIASEFSSLPSYA |
| | | AFATAQEAYEQAVANGDSEVVLKKLKKSLNVAKSE |
| | | FDRDAAMQRKLEKMADQAMTQMYKQARSEDKRAKV |
| | | TSAMQTMLFTMLRKLDNDALNNIINNARDGCVPLN |
| | | IIPLTTAAKLMVVIPDYNTYKNTCDGTTFTYASAL |
| | | WEIQQVVDADSKIVQLSEISMDNSPNLAWPLIVTA |
| | | LRANSAVKLQNNELSPVALRQMSCAAGTTQTACTD |
| | | DNALAYYNTTKGGRFVLALLSDLQDLKWARFPKSD |
| | | GTGTIYTELEPPCRFVTDTPKGPKVKYLYFIKGLN |
| | | NLNRGMVLGSLAATVRLQAGNATEVPANSTVLSFC |
| | | AFAVDAAKAYKDYLASGGQPITNCVKMLCTHTGTG |
| | | QAITVTPEANMDQESFGGASCCLYCRCHIDHPNPK |
| | | GFCDLKGKYVQIPTTCANDPVGFTLKNTVCTVCGM |
| | | WKGYGCSCDQLREPMLQSADAQSFLNRVCGVSAAR |
| | | LTPCGTGTSTDVVYRAFDIYNDKVAGFAKFLKTNC |
| | | CRFQEKDEDDNLIDSYFVVKRHTFSNYQHEETIYN |
| | | LLKDCPAVAKHDFFKFRIDGDMVPHISRQRLTKYT |
| | | MADLVYALRHFDEGNCDTLKEILVTYNCCDDDYFN |
| | | KKDWYDFVENPDILRVYANLGERVRQALLKTVQFC |
| | | DAMRNAGIVGVLTLDNQDLNGNWYDFGDFIQTTPG |
| | | SGVPVVDSYYSLLMPILTLTRALTAESHVDTDLTK |
| | | PYIKWDLLKYDFTEERLKLFDRYFKYWDQTYHPNC |
| | | VNCLDDRCILHCANFNVLFSTVFPPTSFGPLVRKI |
| | | FVDGVPFVVSTGYHFRELGVVHNQDVNLHSSRLSF |
| | | KELLVYAADPAMHAASGNLLLDKRTTCFSVAALTN |
| | | NVAFQTVKPGNFNKDFYDFAVSKGFFKEGSSVELK |
| | | HFFFAQDGNAAISDYDYYRYNLPTMCDIRQLLFVV |
| | | EVVDKYFDCYDGGCINANQVIVNNLDKSAGFPFNK |
| | | WGKARLYYDSMSYEDQDALFAYTKRNVIPTITQMN |
| | | LKYAISAKNRARTVAGVSICSTMTNRQFHQKLLKS |
| | | IAATRGATVVIGTSKFYGGWHNMLKTVYSDVENPH |
| | | LMGWDYPKCDRAMPNMLRIMASLVLARKHTTCCSL |
| | | SHRFYRLANECAQVLSEMVMCGGSLYVKPGGTSSG |
| | | DATTAYANSVFNICQAVTANVNALLSTDGNKIADK |
| | | YVRNLQHRLYECLYRNRDVDTDFVNEFYAYLRKHF |
| | | SMMILSDDAVVCFNSTYASQGLVASIKNFKSVLYY |
| | | QNNVFMSEAKCWTETDLTKGPHEFCSQHTMLVKQG |
| | | DDYVYLPYPDPSRILGAGCFVDDIVKTDGTLMIER |
| | | FVSLAIDAYPLTKHPNQEYADVFHLYLQYIRKLHD |
| | | ELTGHMLDMYSVMLTNDNTSRYWEPEFYEAMYTPH |
| | | TVLQAVGACVLCNSQTSLRCGACIRRPFLCCKCCY |
| | | DHVISTSHKLVLSVNPYVCNAPGCDVTDVTQLYLG |
| | | GMSYYCKSHKPPISFPLCANGQVFGLYKNTCVGSD |
| | | NVTDFNAIATCDWTNAGDYILANTCTERLKLFAAE |
| | | TLKATEETFKLSYGIATVREVLSDRELHLSWEVGK |
| | | PRPPLNRNYVFTGYRVTKNSKVQIGEYTFEKGDYG |
| | | DAVVYRGTTTYKLNVGDYFVLTSHTVMPLSAPTLV |
| | | PQEHYVRITGLYPTLNISDEFSSNVANYQKVGMQK |
| | | YSTLQGPPGTGKSHFAIGLALYYPSARIVYTACSH |
| | | AAVDALCEKALKYLPIDKCSRIIPARARVECFDKF |
| | | KVNSTLEQYVFCTVNALPETTADIVVFDEISMATN |
| | | YDLSVVNARLRAKHYVYIGDPAQLPAPRTLLTKGT |
| | | LEPEYFNSVCRLMKTIGPDMFLGTCRRCPAEIVDT |
| | | VSALVYDNKLKAHKDKSAQCFKMFYKGVITHDVSS |
| | | AINRPQIGVVREFLTRNPAWRKAVFISPYNSQNAV |
| | | ASKILGLPTQTVDSSQGSEYDYVIFTQTTETAHSC |
| | | NVNRFNVAITRAKVGILCIMSDRDLYDKLQFTSLE |
| | | IPRRNVATLQAENVTGLFKDCSKVITGLHPTQAPT |
| | | HLSVDTKFKTEGLCVDIPGIPKDMTYRRLISMMGF |
| | | KMNYQVNGYPNMFITREEAIRHVRAWIGFDVEGCH |
| | | ATREAVGTNLPLQLGFSTGVNLVAVPTGYVDTPNN |
| | | TDFSRVSAKPPPGDQFKHLIPLMYKGLPWNVVRIK |
| | | IVQMLSDTLKNLSDRVVFVLWAHGFELTSMKYFVK |
| | | IGPERTCCLCDRRATCFSTASDTYACWHHSIGFDY |
| | | VYNPFMIDVQQWGFTGNLQSNHDLYCQVHGNAHVA |
| | | SCDAIMTRCLAVHECFVKRVDWTIEYPIIGDELKI |
| | | NAACRKVQHMVVKAALLADKFPVLHDIGNPKAIKC |
| | | VPQADVEWKFYDAQPCSDKAYKIEELFYSYATHSD |
| | | KFTDGVCLFWNCNVDRYPANSIVCRFDTRVLSNLN |
| | | LPGCDGGSLYVNKHAFHTPAFDKSAFVNLKQLPFF |
| | | YYSDSPCESHGKQVVSDIDYVPLKSATCITRONLG |
| | | GAVCRHHANEYRLYLDAYNMMISAGFSLWVYKQFD |
| | | TYNLWNTFTRLQSLENVAFNVVNKGHFDGQQGEVP |
| | | VSIINNTVYTKVDGVDVELFENKTTLPVNVAFELW |
| | | AKRNIKPVPEVKILNNLGVDIAANTVIWDYKRDAP |
| | | AHISTIGVCSMTDIAKKPTETICAPLTVFFDGRVD |
| | | GQVDLFRNARNGVLITEGSVKGLQPSVGPKQASLN |
| | | GVTLIGEAVKTQFNYYKKVDGVVQQLPETYFTQSR |
| | | NLQEFKPRSQMEIDFLELAMDEFIERYKLEGYAFE |
| | | HIVYGDFSHSQLGGLHLLIGLAKRFKESPFELEDF |
| | | IPMDSTVKNYFITDAQTGSSKCVCSVIDLLLDDFV |
| | | EIIKSQDLSVVSKVVKVTIDYTEISFMLWCKDGHV |
| | | ETFYPKLQSSQAWQPGVAMPNLYKMQRMLLEKCDL |
| | | QNYGDSATLPKGIMMNVAKYTQLCQYLNTLTLAVP |
| | | YNMRVIHFGAGSDKGVAPGTAVLRQWLPTGTLLVD |
| | | SDLNDFVSDADSTLIGDCATVHTANKWDLIISDMY |
| | | DPKTKNVTKENDSKEGFFTYICGFIQQKLALGGSV |
| | | AIKITEHSWNADLYKLMGHFAWWTAFVTNVNASSS |
| | | EAFLIGCNYLGKPREQIDGYVMHANYIFWRNTNPI |
| | | QLSSYSLFDMSKFPLKLRGTAVMSLKEGQINDMIL |
| | | SLLSKGRLIIRENNRVVISSDVLVNN |
| surface | 371 | mfvflvllpl vssqcvnltt rtqlppaytn sftrgvyypd kvfrssvlhs tqdlflpffs 61 |
| glycoprotein | | nvtwfhaihv sgtngtkrfd npvlpfndgv yfasteksni irgwifgttl dsktqslliv 121 |
| [Wuhan | | nnatnvvikv cefqfcndpf lgvyyhknnk swmesefrvy ssannctfey vsqpflmdle 181 |
| seafood | | gkqgnfknlr efvfknidgy fkiyskhtpi nlvrdlpqgf saleplvdlp iginitrfqt 241 |
| market | | llalhrsylt pgdsssgwta gaaayyvgyl qprtfllkyn engtitdavd caldplsetk 301 |
| pneumonia | | ctlksftvek giyqtsnfrv qptesivrfp nitnlcpfge vfnatrfasv yawnrkrisn 361 |
| virus]; | | cvadysvlyn sasfstfkcy gvsptklndl cftnvyadsf virgdevrqi apgqtgkiad 421 |
| GenBank: | | ynyklpddft gcviawnsnn ldskvggnyn ylyrlfrksn lkpferdist eiyqagstpc 481 |
| QHD43416.1; | | ngvegfncyf plqsygfqpt ngvgyqpyrv vvlsfellha patvcgpkks tnlvknkcvn 541 |
| Jan. 23, | | fnfngltgtg vltesnkkfl pfqqfgrdia dttdavrdpq tleilditpc sfggvsvitp 601 |
| 2020 | | gtntsnqvav lyqdvnctev pvaihadqlt ptwrvystgs nvfqtragcl igaehvnnsy 661 |
| | | ecdipigagi casyqtqtns prrarsvasq siiaytmslg aensvaysnn siaiptnfti 721 |
| | | svtteilpvs mtktsvdctm yicgdstecs nlllqygsfc tqlnraltgi aveqdkntqe 781 |
| | | vfaqvkqiyk tppikdfggf nfsqilpdps kpskrsfied llfnkvtlad agfikqygdc 841 |
| | | lgdiaardli caqkfngltv lpplltdemi aqytsallag titsgwtfga gaalqipfam 901 |
| | | qmayrfngig vtqnvlyenq klianqfnsa igkiqdslss tasalgklqd vvnqnaqaln 961 |
| | | tlvkqlssnf gaissvlndi lsrldkveae vqidrlitgr lqslqtyvtq qliraaeira 1021 |
| | | sanlaatkms ecvlgqskrv dfcgkgyhlm sfpqsaphgv vflhvtyvpa qeknfttapa 1081 |
| | | ichdgkahfp regvfvsngt hwfvtqrnfy epqiittdnt fvsgncdvvi givnntvydp 1141 |
| | | lqpeldsfke eldkyfknht spdvdlgdis ginasvvniq keidrlneva knlneslidl 1201 |
| | | qelgkyeqyi kwpwyiwlgf iagliaivmv timlccmtsc csclkgccsc gscckfdedd 1261 |
| | | sepvlkgvkl hyt |
| nCoV-10 | 372 | CAAGTGCAACTCGTGGAGTCCGGCGGCGGCG |
| mAb VH (nt-CO) | | TCGTCCAGCCAGGTCGGTCTCTAAGGCTGTCG |
| | | TGCGCTGCAAGTGGGTTTACATTCTCTAATTA |
| | | TGGTATGCATTGGGTGAGGCAGGCTCCCGGC |
| | | AAGGGGCTGGAATGGCTCGCTGTGATTTCAA |
| | | GCGATGGTCGAATTAAATTTTATGCTGACTCT |
| | | GTTAAAGGCAGATTTACTATGAGTAGAGACT |
| | | CATCAAAAAACACTCTGTACCTGCAAATGAA |
| | | TAGCCTGCGCGCCGAGGATACGGCTGTCTAC |
| | | TACTGCGCCAAAGACCGGTTCCAATTCGCAA |
| | | GATCATGGTATGGAGACTACTTCGATTATTGG |
| | | GGGCAAGGTACTCAGGTGACTGTCTCCTCA |
| nCoV-10 | 373 | CAGGTGCAACTTGTGGAAAGTGGCGGAGGCG |
| mAb VH-v3 | | TGGTGCAGCCGGGGCGATCCTTGCGCCTCTCC |
| (nt-CO) | | TGTGCCGCCAGCGGGTTTACATTTTCCAATTA |
| | | TGGCATGCACTGGGTGCGTCAGGCCCCCGGG |
| | | AAGGGCCTGGAATGGCTTGCCGTGATCAGCA |
| | | GTGACGGACGCATTAAATTCTACGCCGATTCT |
| | | GTCAAGGGTCGGTTCACCATGAGTAGAGACT |
| | | CCTCAAAGAACACTCTGTACCTGCAGATGAA |
| | | TAGCCTGCGGGCCGAAGACACCGCTGTGTAC |
| | | TACTGTGCTAAGGACCGGTTTCAATTTGCTAG |
| | | ATCATTCTACGGGGACTACTTCGACTATTGGG |
| | | GCCAAGGCACGCAGGTGACAGTCAGCTCA |
| nCoV-10 | 374 | QVQLVESGGGVVQPGRSLRLSCAASGFTFSNY |
| mAb VH-v3 | | GMHWVRQAPGKGLEWLAVISSDGRIKFYADS |
| (aa) | | VKGRFTMSRDSSKNTLYLQMNSLRAEDTAVYY |
| | | CAKDRFQFARSFYGDYFDYWGQGTQVTVSS |
| nCoV-10 | 375 | AKDRFQFARSFYGDYFDY |
| mAb VH-v3 | | |
| CDRH3 (aa) | | |
| nCoV-10 | 376 | CAAGTCCAGCTTGTCGAGTCTGGAGGGGGCG |
| mAb VH-v4 | | TAGTGCAGCCCGGGCGCAGTTTGAGATTAAG |
| (nt-CO) | | CTGCGCCGCCTCTGGTTTCACGTTCTCCAATT |
| | | ACGGTATGCACTGGGTGCGACAGGCACCCGG |
| | | CAAGGGGCTGGAATGGCTGGCCGTCATTAGT |
| | | TCAGACGGGCGGATCAAATTTTACGCTGACA |
| | | GTGTGAAGGGTAGGTTTACCATGTCAAGAGA |
| | | CTCCAGCAAAAACACATTATACCTGCAGATG |
| | | AACAGCCTGCGCGCCGAGGACACCGCCGTAT |
| | | ATTATTGCGCTAAGGACCGCTTTCAGTTCGCT |
| | | AGGTCTGCCTACGGTGACTATTTTGATTATTG |
| | | GGGACAGGGAACTCAGGTAACGGTCAGCTCA |
| nCoV-10 | 377 | QVQLVESGGGVVQPGRSLRLSCAASGFTFSNY |
| mAb VH-v4 | | GMHWVRQAPGKGLEWLAVISSDGRIKFYADS |
| (aa) | | VKGRFTMSRDSSKNTLYLQMNSLRAEDTAVYY |
| | | CAKDRFQFARSAYGDYFDYWGQGTQVTVSS |
| nCoV-10 | 378 | AKDRFQFARSAYGDYFDY |
| mAb VH-v4 | | |
| CDRH3 (aa) | | |
| nCoV-10 | 379 | GCGGTTGCAATGACTCAGTCCCCACTCTCCTT |
| mAb VL (Vk) | | GCCCGTGACACTCGGGCAACCGGCCTCCATC |
| (nt-CO) | | TCTTGCCGGAGCAATCAGTCCCTTGTTTATAG |
| | | CGACGGGAATATCTATCTGAATTGGTTCCAGC |
| | | AGAGGCCCGGGCAGTCACCAATGCGCCTCAT |
| | | CTATCGGGTGTCCAACCGGGATTCAGGTGTG |
| | | CCCGACCGGTTTTCTGGCAGCGGATCAGGAA |
| | | CGGACTTTACACTCAAAATTTCCCGCGTGGAG |
| | | GCTGAGGATGTCGGAATCTATTATTGCATGCA |
| | | GGGCACGCACTGGCCTCCAACCTTCGGCGGA |
| | | GGAACCAAGGTGGAGATCAAG |
| nCoV-10 | 380 | DVVMTQSPLSLPVTLGQPASISCRSQQSLVYSD |
| mAb VL-v3 | | GNIYLNWFQQRPGQSPMRLIYRVSNRDSGVPD |
| (Vk) (aa) | | RFSGSGSGTDFTLKISRVEAEDVGIYYCMQGTH |
| | | WPPTFGGGTKVEIK |
| nCoV-10 | 381 | GATGTGGTAATGACCCAAAGCCCCCTGTCATT |
| mAb VL-v3 | | GCCAGTGACACTGGGCCAGCCGGCCTCAATT |
| (Vk) (nt-CO) | | AGTTGCAGGTCTCAGCAGTCACTGGTGTACTC |
| | | CGATGGCAATATCTACCTAAACTGGTTCCAGC |
| | | AGCGGCCTGGGCAATCTCCTATGCGACTTATA |
| | | TATAGAGTGAGTAACCGGGACAGTGGGGTCC |
| | | CTGATAGATTTTCAGGTAGCGGCAGTGGCAC |
| | | AGATTTCACCCTGAAGATATCTCGGGTAGAA |
| | | GCAGAGGACGTGGGGATATATTATTGCATGC |
| | | AGGGAACGCACTGGCCCCCTACCTTCGGCGG |
| | | CGGAACAAAAGTGGAAATAAAA |
| surface | 382 | nitnlcpfgevfnatrfasvyawnrkrisncvadysvlynsasfstfkcyg |
| glycoprotein | | vsptklndlcftnvyadsfvirgdevrqiapgqtgkiadynyklpddftg |
| RBD [Wuhan | | cviawnsnnldskvggnynylyrlfrksnlkpferdisteiyqagstpcn |
| seafood | | gvegfncyfplqsygfqptngvgyqpyrvvvlsfellhapatvcgpkks |
| market | | tnlvknkcvnfnfngltgtg |
| pneumonia | | |
| virus]; | | |
| GenBank: | | |
| QHD43416.1; | | |
| Jan. 23, | | |
| 2020 | | |
| nCoV-10 | 383 | DVVMTQSPLSLPVTLGQPASISCRSSQSLVYSD |
| mAb VL-v4 | | GNIYLNWFQQRPGQSPMRLIYRVSNRDSGVPD |
| (Vk) (aa) | | RFSGSGSGTDFTLKISRVEAEDVGIYYCMQGTH |
| | | WPPTFGGGTKVEIK |
| nCoV-10 | 384 | GACGTGGTGATGACACAGAGCCCACTATCCC |
| mAb VL-v4 | | TTCCGGTCACCTTGGGCCAGCCAGCAAGCATT |
| (Vk) (nt-CO) | | TCCTGTCGGTCGAGCCAGTCTCTGGTCTATAG |
| | | TGACGGAAACATTTATTTGAATTGGTTTCAAC |
| | | AGAGGCCCGGACAGTCACCAATGCGCCTCAT |
| | | CTACCGCGTCTCTAACAGAGACTCTGGAGTCC |
| | | CCGATCGCTTTTCCGGTAGCGGGTCTGGGACC |
| | | GATTTTACATTGAAGATCTCCAGGGTGGAAG |
| | | CCGAGGACGTTGGTATCTACTACTGCATGCA |
| | | GGGTACCCATTGGCCACCGACCTTCGGCGGG |
| | | GGCACCAAAGTGGAGATCAAA |
| nCoV-10 | 385 | DVVMTQSPLSLPVTLGQPASISCRSSQSLVYSD |
| mAb VL-v5 | | GNIYLNWFQQRPGQSPMRLIYRVSNRDSGVPD |
| (Vk) (aa) | | RFSGSGSGTDFTLKISRVEAEDVGIYYCMQGTH |
| | | FPPTFGGGTKVEIK |
| nCoV-10 | 386 | MQGTHFPPT |
| mAb VL-v5 | | |
| (Vk) CDRL3 | | |
| (aa) | | |
| nCoV-10 | 387 | GATGTCGTGATGACACAGTCGCCACTGAGTC |
| mAb VL-v5 | | TACCGGTGACACTGGGGCAGCCGGCCTCCAT |
| (Vk) (nt-CO) | | TTCATGTAGGAGCAGTCAGTCGTTAGTGTACT |
| | | CTGACGGTAACATCTATCTTAATTGGTTCCAG |
| | | CAAAGACCTGGCCAGTCACCTATGCGGCTGA |
| | | TATATAGAGTTTCCAACAGAGACTCCGGGGT |
| | | GCCAGACAGGTTCTCTGGAAGTGGCTCTGGA |
| | | ACTGACTTTACACTGAAGATCAGCAGGGTGG |
| | | AGGCAGAGGACGTGGGTATCTATTACTGTAT |
| | | GCAAGGGACTCATTTCCCTCCAACATTCGGA |
| | | GGAGGGACAAAAGTGGAGATAAAA |
| nCoV-10 | 388 | DVVMTQSPLSLPVTLGQPASISCRSQQSLVYSD |
| mAb VL-v6 | | GNIYLNWFQQRPGQSPMRLIYRVSNRDSGVPD |
| (Vk) (aa) | | RFSGSGSGTDFTLKISRVEAEDVGIYYCMQGTH |
| | | FPPTFGGGTKVEIK |
| nCoV-10 | 389 | GACGTGGTGATGACTCAATCACCCCTCTCCCT |
| mAb VL-v6 | | ACCGGTCACACTGGGCCAGCCAGCAAGCATT |
| (Vk) (nt-CO) | | TCATGCCGTTCGCAGCAGTCCCTCGTCTATTC |
| | | GGACGGCAATATTTACCTCAACTGGTTTCAGC |
| | | AGCGACCCGGGCAGTCACCCATGCGACTTAT |
| | | CTACAGGGTGAGCAACAGAGATTCCGGGGTC |
| | | CCCGATCGCTTCTCAGGCAGTGGCAGCGGCA |
| | | CCGATTTCACCCTAAAAATTAGCCGCGTGGA |
| | | AGCGGAAGATGTGGGTATCTACTACTGCATG |
| | | CAGGGAACTCACTTTCCACCCACTTTTGGAGG |
| | | GGGGACCAAAGTTGAAATCAAG |
| Receptor | 390 | nsnnldskvggnynylyrlfrksnlkpferdistei |
| Binding Motif | | yqagstpcngvegfncyfplqsygfqptngvgyqpy |
| (RBM) in | | |
| surface | | |
| glycoprotein | | |
| RBD [Wuhan | | |
| seafood | | |
| market | | |
| pneumonia | | |
| virus]; | | |
| GenBank: | | |
| QHD43416.1; | | |
| Jan. 23, | | |
| 2020 | | |
| nCoV10 mAb | 391 | RTVAAPSVFIFPPSDEQLKSGTASVVCLLNNFYP |
| CL (Ck), | | REAKVQWKVDNALQSGNSQESVTEQDSKDST |
| k1m3, | | YSLSSTLTLSKADYEKHKVYACEVTHQGLSSPV |
| IGKC*01 | | TKSFNRGEC |
| nCoV-10 | 392 | DVVMTQSPLSLPVTLGQPASISCRSSQSLVYSD |
| mAb VL-v7 | | GNIYLNWFQQRPGQSPMRLIYRVSNRDSGVPD |
| (Vk) (aa) | | RFSGSGSGTDFTLKISRVEAEDVGIYYCMQGTH |
| | | YPPTFGGGTKVEIK |
| nCoV-10 | 393 | DVVMTQSPLSLPVTLGQPASISCRSSQSLVYSE |
| mAb VL-v8 | | GNIYLNWFQQRPGQSPMRLIYRVSNRDSGVPD |
| (Vk) (aa) | | RFSGSGSGTDFTLKISRVEAEDVGIYYCMQGTH |
| | | YPPTFGGGTKVEIK |
| nCoV-10 | 394 | MQGTHYPPT |
| mAb VL-v7/8 | | |
| (Vk) CDRL3 | | |
| (aa) | | |
| nCov10 CH1- | 395 | ASTKGPSVFPLAPSSKSTSGGTAALGCLVKDYF |
| CH3 G1m17; | | PEPVTVSWNSGALTSGVHTFPAVLQSSGLYSLS |
| IgHG1*01_M | | SVVTVPSSSLGTQTYICNVNHKPSNTKVDKKVE |
| LNS | | PKSCDKTHTCPPCPAPELLGGPSVFLFPPKPKDT |
| | | LMISRTPEVTCVVVDVSHEDPEVKFNWYVDGV |
| | | EVHNAKTKPREEQYNSTYRVVSVLTVLHQDWL |
| | | NGKEYKCKVSNKALPAPIEKTISKAKGQPREPQ |
| | | VYTLPPSRDELTKNQVSLTCLVKGFYPSDIAVE |
| | | WESNGQPENNYKTTPPVLDSDGSFFLYSKLTVD |
| | | KSRWQQGNVFSCSVLHEALHSHYTQKSLSLSP |
| | | GK |
| nCoV-10 | 396 | QVQLVESGGGVVQPGRSLRLSCAASGFTFSNY |
| mAb VH-v5 | | GMHWVRQAPGKGLEWLAVISSDGRIKFYADS |
| (aa) | | VKGRFTMSRDSSKNTLYLQMNSLRAEDTAVYY |
| | | CAKDRFQFARSYYGDYFDYWGQGTQVTVSS |
| nCoV-10 | 397 | AKDRFQFARSYYGDYFDY |
| mAb VH-v5 | | |
| CDRH3 (aa) | | |
| nCoV-10 | 398 | QSLVYSEGNIY |
| mAb VL-v8 | | |
| (Vk) CDRL1 | | |
| (aa) | | |
| nCoV-10 | 399 | QALVYSDGNIY |
| mAb VL-v9 | | |
| (Vk) CDRL1 | | |
| (aa) | | |
| nCoV-10 | 400 | AVAMTQSPLSLPVTLGQPASISCRSNQALVY |
| mAb VL-v9 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10 | 401 | QGLVYSDGNIY |
| mAb VL-v10 | | |
| (Vk) CDRL1 | | |
| (aa) | | |
| nCoV-10 | 402 | AVAMTQSPLSLPVTLGQPASISCRSNQGLVY |
| mAb VL-v10 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10 | 403 | QVLVYSDGNIY |
| mAb VL-v11 | | |
| (Vk) CDRL1 | | |
| (aa) | | |
| nCoV-10 | 404 | AVAMTQSPLSLPVTLGQPASISCRSNQVLVY |
| mAb VL-v11 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10 | 405 | PSLVYSDGNIY |
| mAb VL-v12 | | |
| (Vk) CDRL1 | | |
| (aa) | | |
| nCoV-10 | 406 | AVAMTQSPLSLPVTLGQPASISCRSNPSLVYS |
| mAb VL-v12 | | DGNIYLNWFQQRPGQSPMRLIYRVSNRDSG |
| (Vk) (aa) | | VPDRFSGSGSGTDFTLKISRVEAEDVGIYYC |
| | | MQGTHWPPTFGGGTKVEIK |
| nCoV-10 | 407 | AVAMTQSPLSLPVTLGQPASISCRSDQSLVY |
| mAb VL-v13 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10 | 408 | AVAMTQSPLSLPVTLGQPASISCRSEQSLVYS |
| mAb VL-v14 | | DGNIYLNWFQQRPGQSPMRLIYRVSNRDSG |
| (Vk) (aa) | | VPDRFSGSGSGTDFTLKISRVEAEDVGIYYC |
| | | MQGTHWPPTFGGGTKVEIK |
| nCoV-10 | 409 | DVVMTQSPLSLPVTLGQPASISCRSNQALVY |
| mAb VL-v15 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10 | 410 | DVVMTQSPLSLPVTLGQPASISCRSNQGLVY |
| mAb VL-v16 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10mAb | 411 | DVVMTQSPLSLPVTLGQPASISCRSNQVLVY |
| VL-v17 (Vk) | | SDGNIYLNWFQQRPGQSPMRLIYRVSNRDS |
| (aa) | | GVPDRFSGSGSGTDFTLKISRVEAEDVGIYYC |
| | | MQGTHWPPTFGGGTKVEIK |
| nCoV-10 | 412 | DVVMTQSPLSLPVTLGQPASISCRSNPSLVYS |
| mAb VL-v18 | | DGNIYLNWFQQRPGQSPMRLIYRVSNRDSG |
| (Vk) (aa) | | VPDRFSGSGSGTDFTLKISRVEAEDVGIYYC |
| | | MQGTHWPPTFGGGTKVEIK |
| nCoV-10 | 413 | DVVMTQSPLSLPVTLGQPASISCRSDQSLVY |
| mAb VL-v19 | | SDGNIYLNWFQQRPGQSPMRLIYRVSNRDS |
| (Vk) (aa) | | GVPDRFSGSGSGTDFTLKISRVEAEDVGIYYC |
| | | MQGTHWPPTFGGGTKVEIK |
| nCoV-10 | 414 | DVVMTQSPLSLPVTLGQPASISCRSEQSLVYS |
| mAb VL-v20 | | DGNIYLNWFQQRPGQSPMRLIYRVSNRDSG |
| (Vk) (aa) | | VPDRFSGSGSGTDFTLKISRVEAEDVGIYYC |
| | | MQGTHWPPTFGGGTKVEIK |
| nCoV-10 | 415 | QVQLVESGGGVVQPGRSLRLSCAASGFTFS |
| mAb VH-v21 | | NYGMHWVRQAPGKGLEWLAVISFDGRIKF |
| (aa) | | YADSVKGRFTMSRDSSKNTLYLQMNSLRAE |
| | | DTAVYYCAKDRFQFARSWYGDYFDYWGQ |
| | | GTQVTVSS |
| nCoV-10 | 416 | ISFDGRIK |
| mAb VH-v21 | | |
| CDRH2 (aa) | | |
| nCoV-10 | 417 | GCCAGTGTCAGGTGCAGCTGGTGGAGTCT |
| mAb VH-v21 | | GGGGGAGGCGTGGTCCAGCCTGGGAGGTC |
| (nt) | | CCTGAGACTCTCCTGTGCAGCCTCTGGATT |
| | | CACCTTCAGTAACTATGGCATGCACTGGGT |
| | | CCGCCAGGCTCCAGGCAAGGGGCTGGAGT |
| | | GGCTGGCAGTTATATCATTTGATGGAAGAA |
| | | TTAAGTTCTATGCAGACTCCGTGAAGGGCC |
| | | GATTCACCATGTCCAGAGACAGTTCCAAG |
| | | AACACGCTGTATCTGCAAATGAACAGCCT |
| | | GAGAGCTGAGGACACGGCTGTGTATTACT |
| | | GTGCGAAAGATCGGTTCCAGTTTGCCAGA |
| | | AGCTGGTACGGTGACTACTTTGACTACTGG |
| | | GGCCAGGGAACCCAGGTCACCGTCTCCTC |
| | | AGCCTCCACCAAGGGCCCATCGGTCTTCCC |
| | | CCTGGCACC |

### EXAMPLES

### Example 1

### Cross-Reactivity of Human Monoclonal Antibodies Against the Spike Protein of SARS-CoV-2

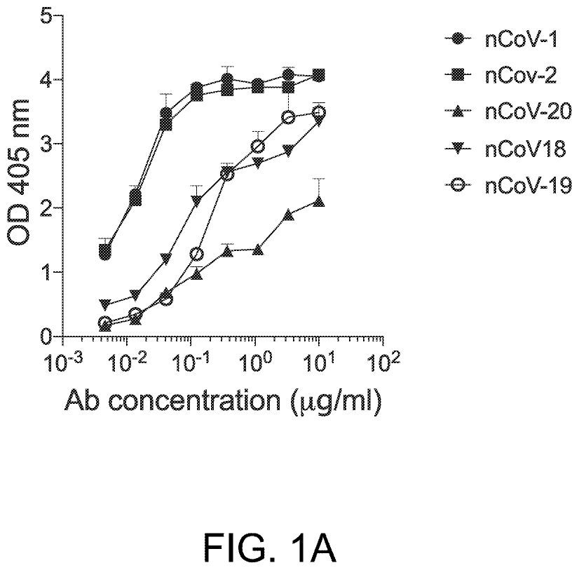

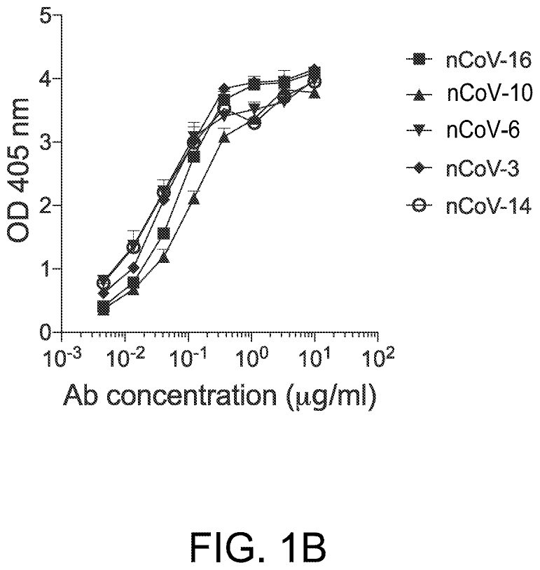

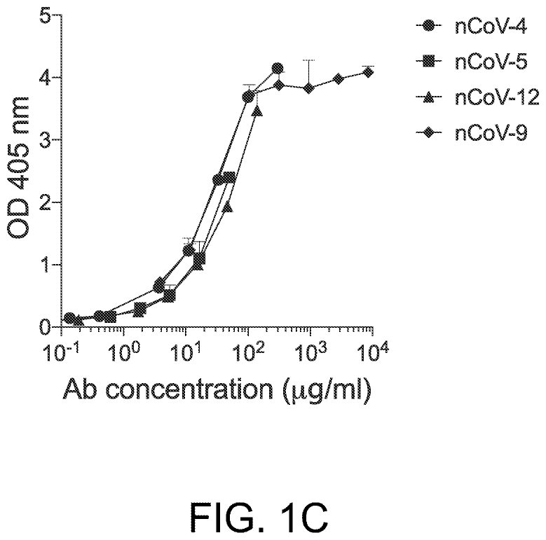

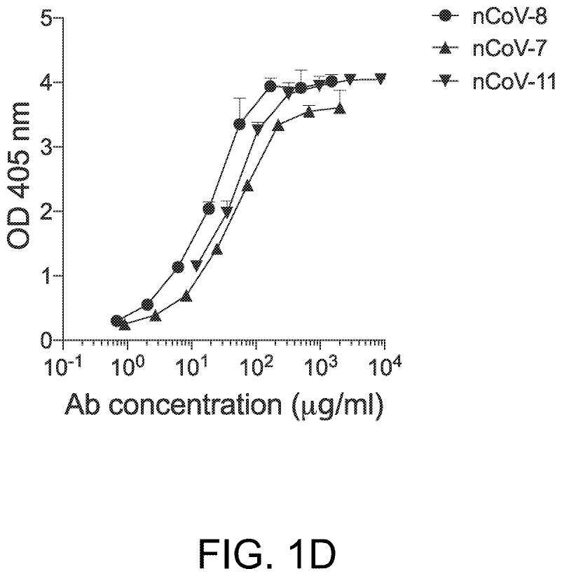

A panel of twenty human SARS-CoV-neutralizing monoclonal antibodies recognizing SARS-CoV RBD were initially tested for binding to SARS-CoV Spike protein by ELISA (FIGS. 1A-1D). All antibodies were confirmed to bind to SARS-CoV Spike protein. The antibodies comprise the VH and VL sequences as shown in Table 2.

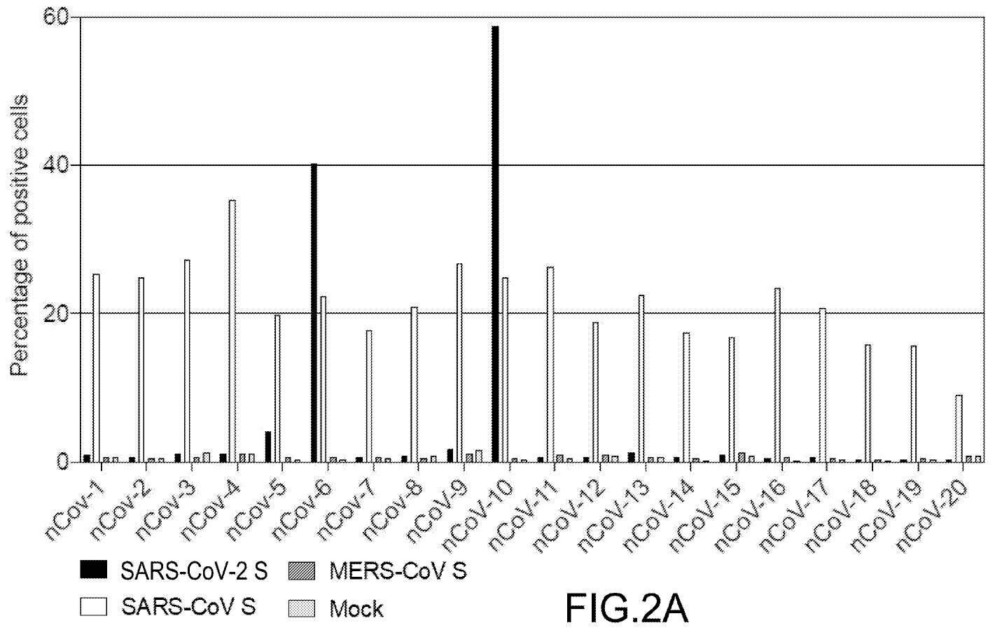

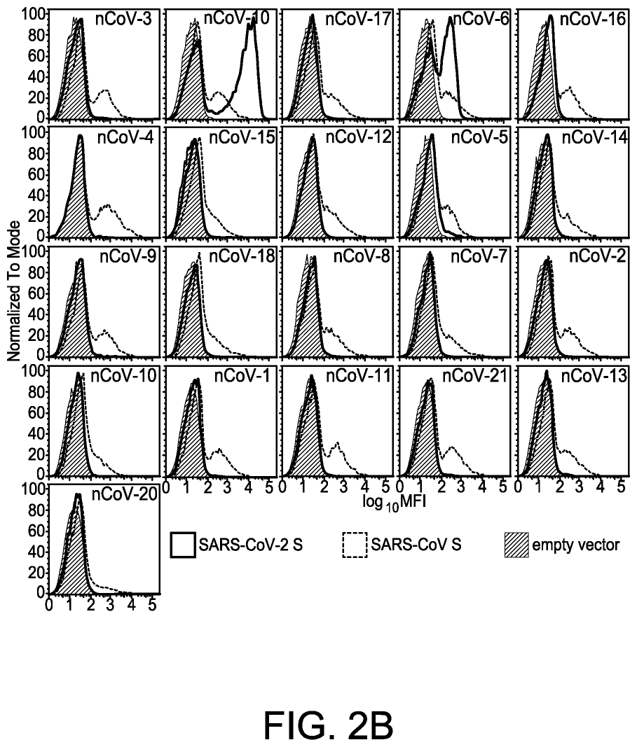

To test the ability of the twenty anti-SARS-CoV antibodies to cross-react with the spike (S) protein of SARS-CoV-2, a flow cytometry-based assay was utilized. ExpiCHO cells were transfected with S protein of SARS-CoV-2, SARS-CoV and MERS-CoV, or with an empty plasmid as a negative control. The twenty monoclonal antibodies were then tested by flow cytometry at 10 μg/ml for their ability to stain ExpiCHO cells expressing the S protein of SARS-CoV-2, SARS-CoV, MERS-CoV or Mock cell transfectants. As expected, all twenty antibodies recognized SARS-CoV Spike protein on cells, and none recognized MERS-CoV or Mock transfectants (FIGS. 2A and 2B). Two antibodies, nCoV-6 and nCoV-10, recognized SARS-CoV-2 Spike protein on well over 30% (nCoV-6, approximately 40%; nCov-10, approximately 60%) of the cells using this assay.

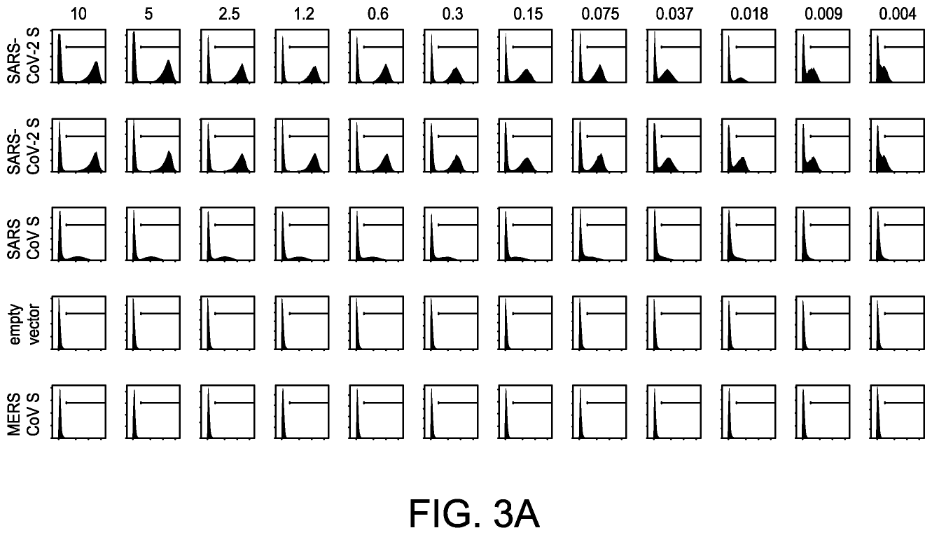

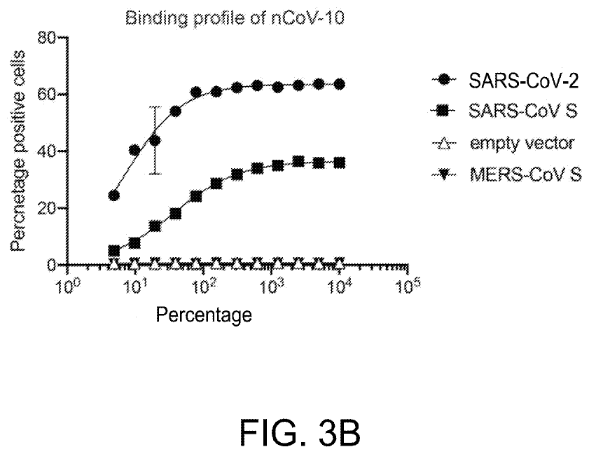

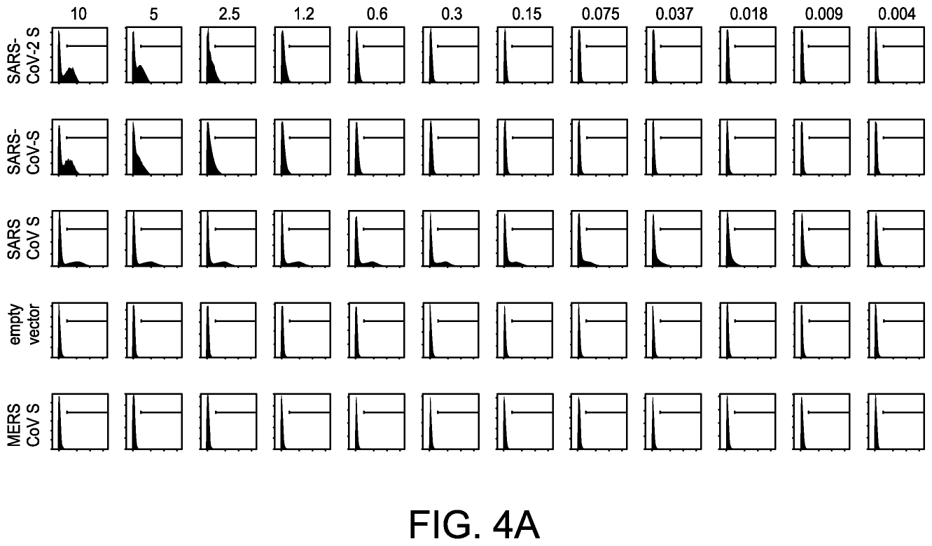

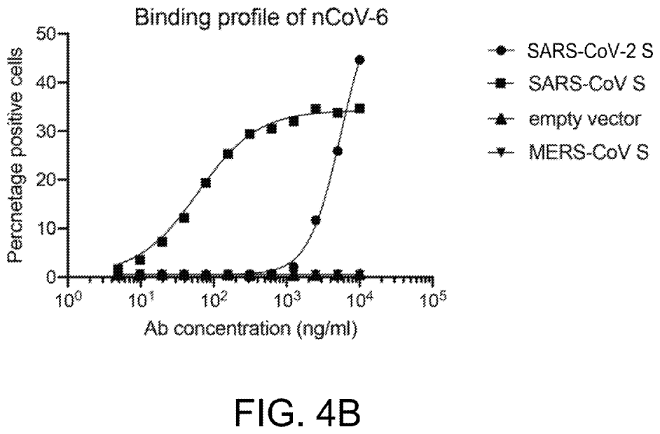

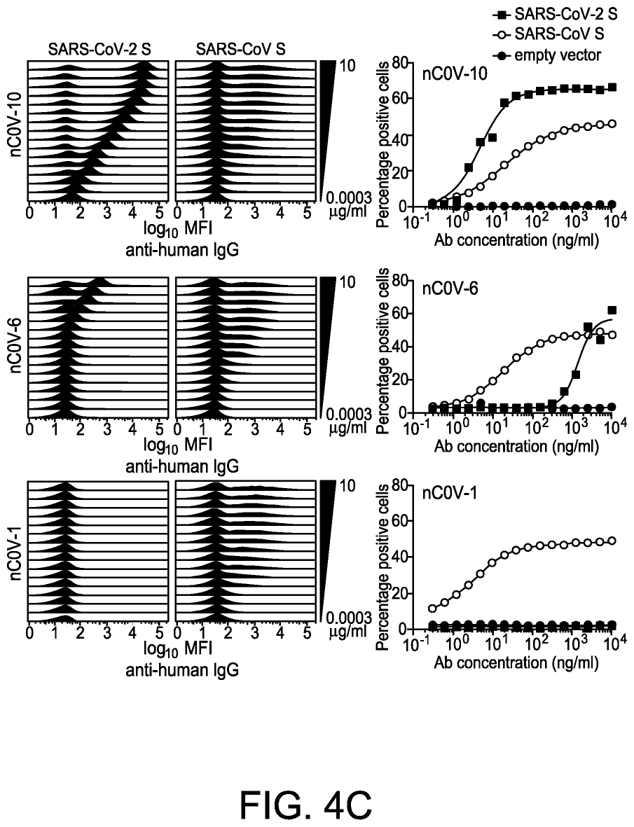

The binding of nCoV-6 and nCoV-10 to SARS-CoV-2, SARS-CoV and MERS-CoV S proteins on cells was further investigated by testing multiple concentrations of each antibody. As shown in FIGS. 3A and 3B, antibody nCoV-10 recognizes SARS-CoV-2 S protein displayed 24 hours after transfection with higher avidity and similarly to its binding to SARS-CoV. As shown in FIGS. 4A and 4B, antibody nCoV-6 binds to SARS-CoV-2 S with lower avidity than it binds to SARS-CoV S.

The same experiment was repeated on cells 24 hours after transfection, and similar results were obtained (FIG. 4C).

Variants of antibody nCoV-10 and antibody nCoV-2 were constructed. The variant antibodies comprise the VH and VL sequences shown in Table 3. Certain of the nCoV-10 variants comprise a variant VH which may be combined with nCoV-10 wild type VL or any nCoV-10 variant VL, and others comprise a variant VL which may be combined with nCoV-10 wild type VH or any nCoV-10 variant VH.

TABLE 2

SEQ ID NO.

| Antibody | VH | VL |
|---|---|---|
| nCoV-3 | 5 | 13 |
| nCoV-10 | 21 | 29 |
| nCoV-17 | 53 | 61 |
| nCoV-6 | 69 | 77 |
| nCoV-16 | 85 | 93 |
| nCoV-20 | 101 | 109 |
| nCoV-4 | 117 | 125 |
| nCoV-15 | 165 | 173 |
| nCoV-12 | 181 | 189 |
| nCoV-5 | 197 | 205 |
| nCoV-14 | 213 | 221 |
| nCoV-9 | 229 | 237 |
| nCoV-18 | 245 | 253 |
| nCoV-8 | 261 | 269 |
| nCoV-7 | 277 | 285 |
| nCoV-2 | 293 | 301 |
| nCoV-19 | 309 | 317 |
| nCoV-1 | 325 | 333 |
| nCoV-11 | 341 | 349 |
| nCoV-13 | 357 | 365 |

TABLE 3

SEQ ID NO.

| Antibody | VH | VL |
|---|---|---|
| nCoV-4-v2 | 133 | 141 |
| nCoV-4-v3 | 149 | 157 |
| nCoV-10-v2 | 37 | 45 |
| nCoV-10 VH-v3 | 374 | |
| nCoV-10 VH-v4 | 377 | |
| nCoV-10 VL-v3 | | 380 |
| nCoV-10 VL-v4 | | 383 |
| nCoV-10 VL-v5 | | 385 |
| nCoV-10 VL-v6 | | 388 |
| nCoV-10 VL-v7 | | 392 |
| nCoV-10 VL-v8 | | 393 |
| nCoV-10 VL-v9 | | 400 |
| nCoV-10 VL-v10 | | 402 |
| nCoV-10 VL-v11 | | 404 |
| nCoV-10 VL-v12 | | 406 |
| nCoV-10 VL-v13 | | 407 |
| nCoV-10 VL-v14 | | 408 |
| nCoV-10 VL-v15 | | 409 |
| nCoV-10 VL-v16 | | 410 |
| nCoV-10 VL-v17 | | 411 |
| nCoV-10 VL-v18 | | 412 |
| nCoV-10 VL-v19 | | 413 |
| nCoV-10 VL-v20 | | 414 |
| nCoV-10 VH-v21 | 415 | |

### Example 2

### Epitope Study of NcoV-1 Monoclonal Antibody

Epitope analysis of monoclonal antibody nCoV-1 for SARS-CoV RBD was performed using low resolution cryo-EM and SARS CoV S glycoprotein (crystal structure 6NB7). Based on these studies, the SARS CoV S glycoprotein epitope residues of nCoV1 comprise Thr402, Gly403, Val404, Asp407, Tyr408, Tyr442, Arg444, His445, Gly446, Lys447, Ser461, Pro462, Asp463, Gly464, Cys467, Leu472, Asn473, Cys474, Tyr475, Leu478, and Asn479.

### Example 3

### Cross-Competition Analysis of Binding of Antibodies NCoV-6 and NCoV-10 to SARS-CoV RBD

To test whether two antibodies nCoV-6 and nCoV-10 bind distinct epitopes, competition experiments were conducted using the receptor binding domain (RBD) of SARS-CoV. The Octet binding profile demonstrated that nCoV-10 does not compete with nCoV-6, which indicates that the two antibodies bind to different sites on the RBD. As a control, homologous competition of the antibodies was conducted. Based on the observation that nCoV-10, but not nCoV-4, bound to 2019-nCoV, competition of the Fab fragment of nCoV-4 with nCoV-10 or nCoV-6 was also conducted. nCoV-10 competes with nCoV-4, but not with nCoV-6. This experiment was repeated using monoclonal antibody nCoV-1 for which the epitope was defined using low resolution cryo-EM and SARS CoV S glycoprotein, and found to map in the receptor binding motif (corresponding to the footprint of human ACE2 receptor on SARS-CoV S protein). In this experiment, SARS-CoV S1 domain (that comprises RBD) was used.

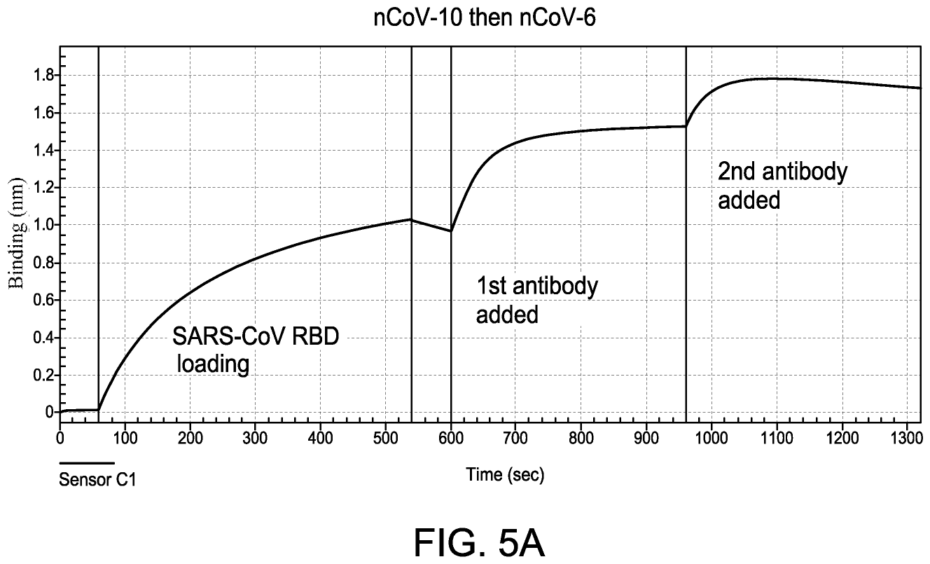

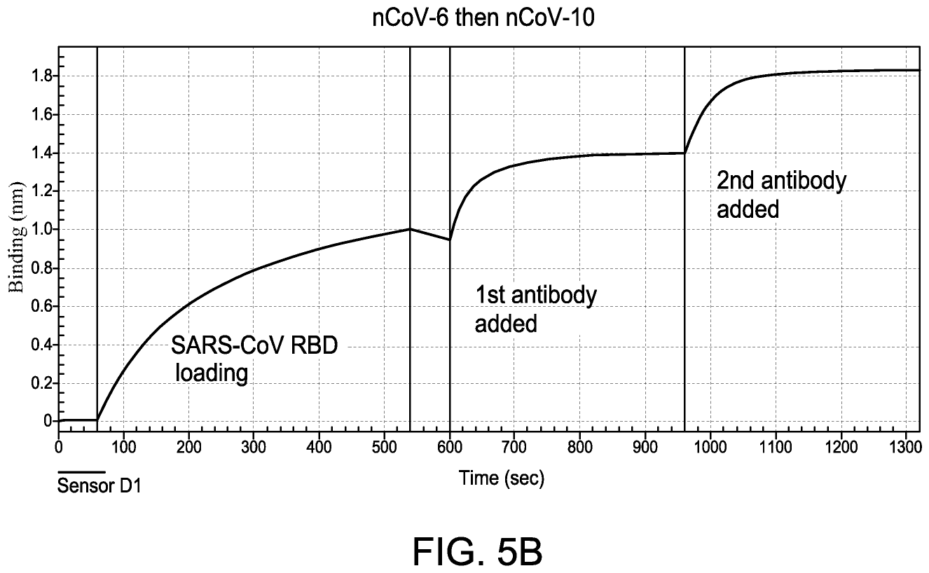

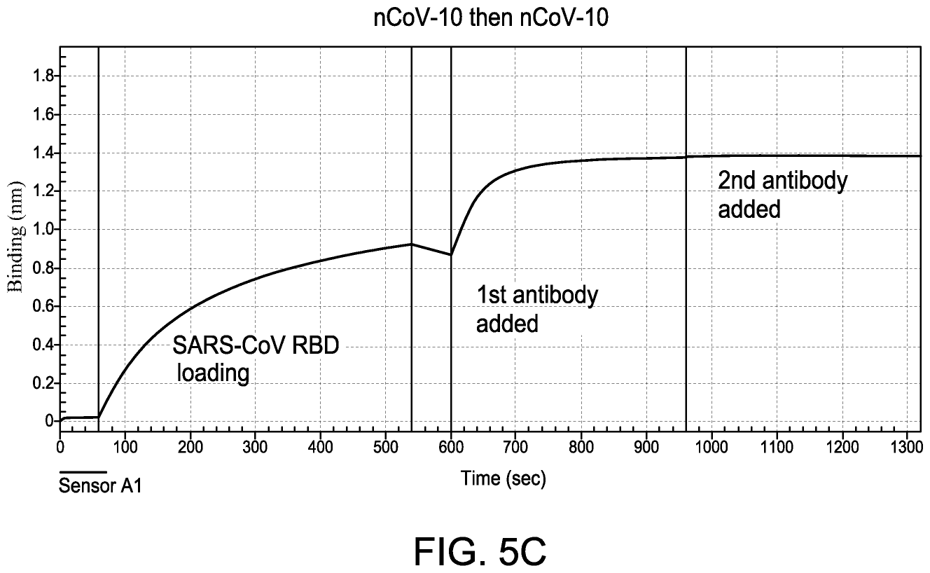

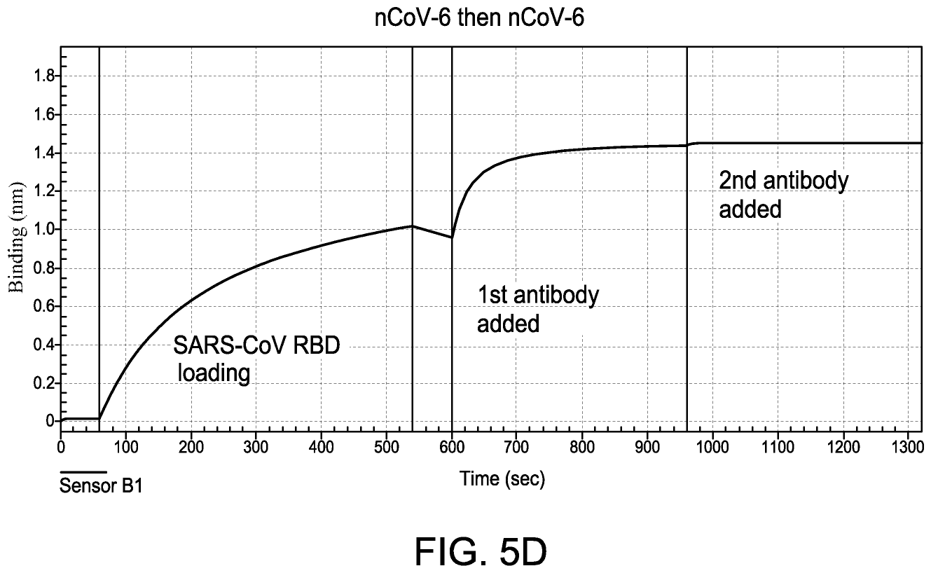

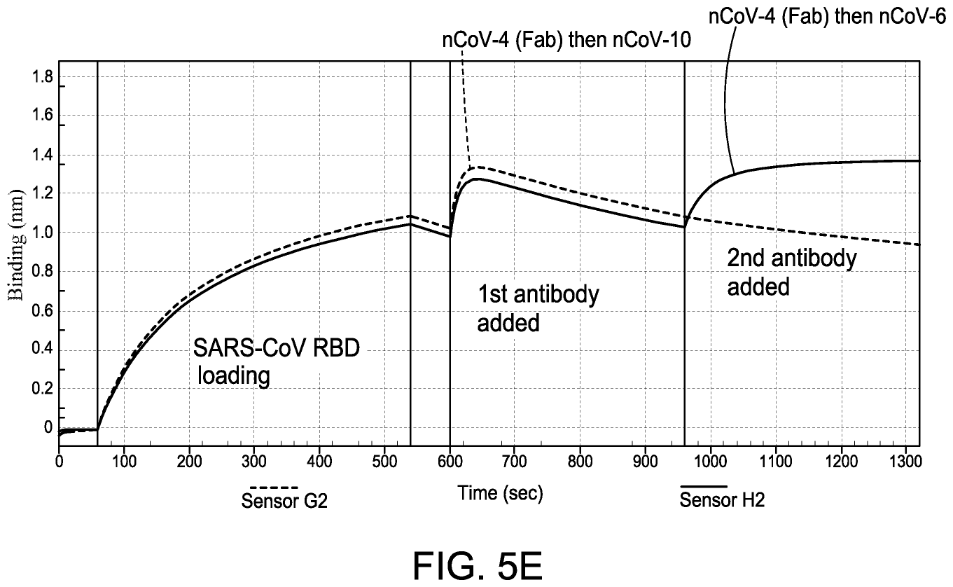

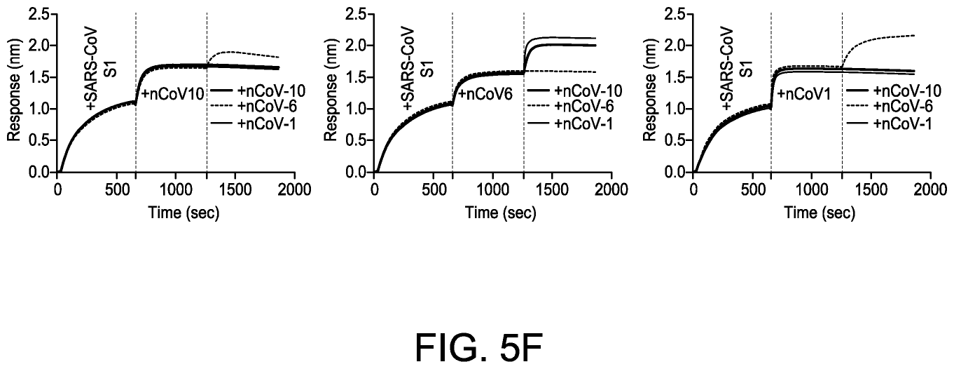

The results confirmed that nCoV-10 does not compete with nCoV-6, and also showed that nCoV-10 competes with nCoV-1 (FIGS. 5A-5F). This finding further suggests that nCoV-10 recognizes an epitope on the S glycoprotein of SARS-CoV and SARS-CoV-2 that is located in the receptor binding motif.

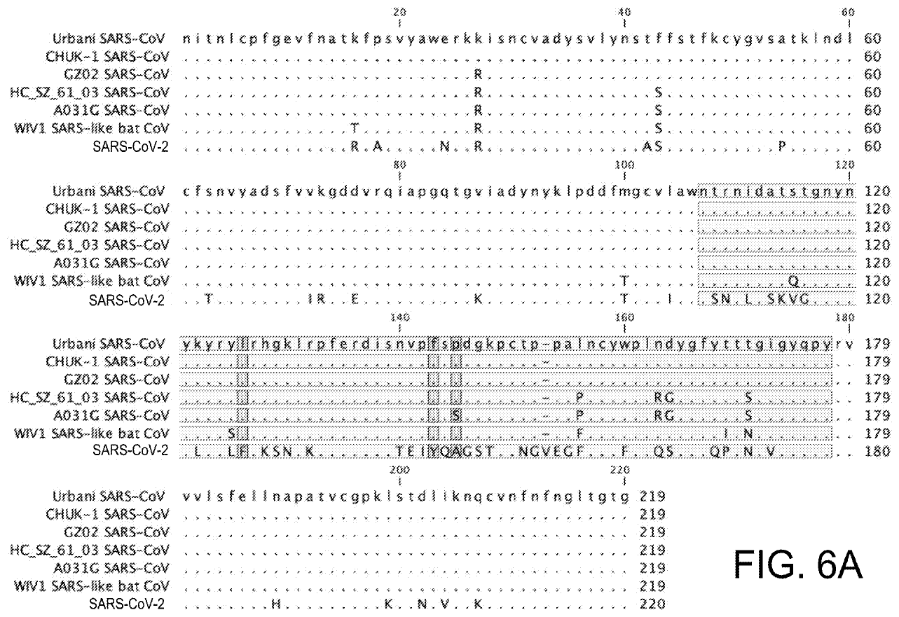

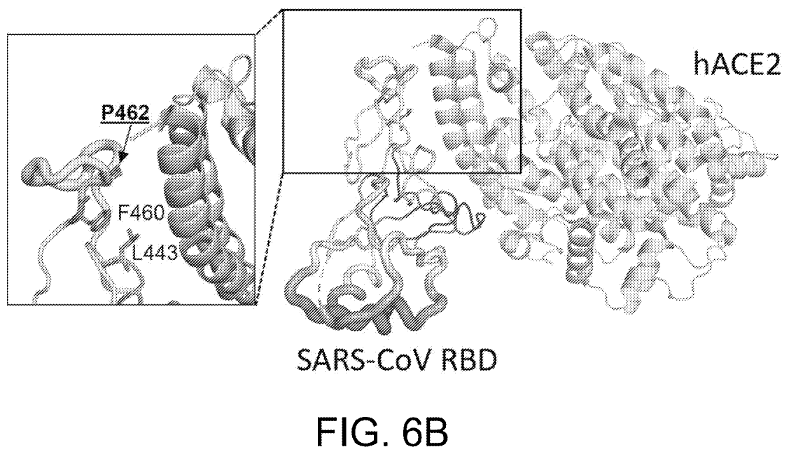

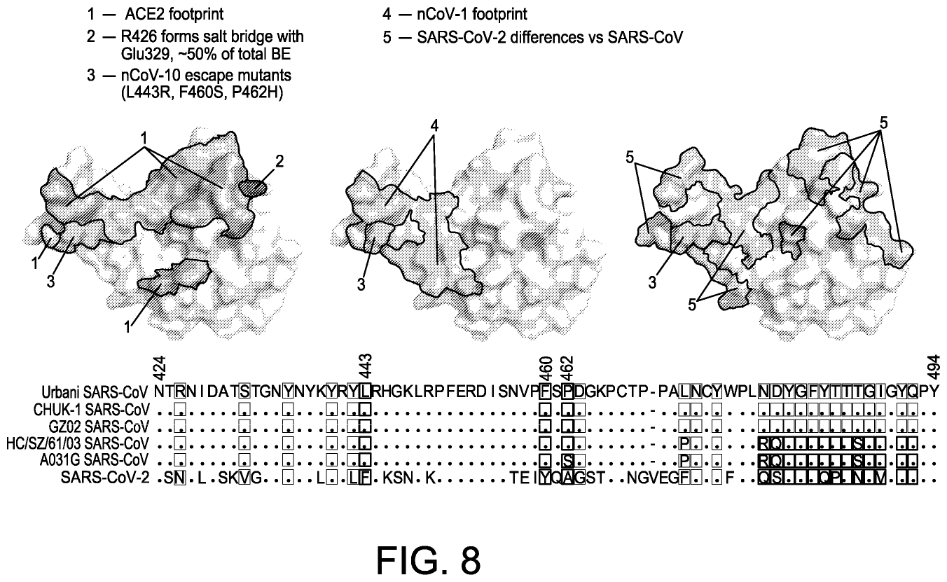

Of note, the SARS-CoV RBD and the SARS-CoV-2 RBD have differences at certain amino acid sequence positions. Consistent with the induction of the same SARS-CoV escape mutant (i.e. P462H), nCoV-10 and nCoV4 recognize overlapping epitopes in SARS-CoV RBD. Of note, the SARS-CoV RBD L443, F460 and P462 residues found to be critical for nCoV-10 neutralization (based on the lack of neutralization of SARS-CoV escape mutants L443R, F460C and P462H) are not conserved in SARS-CoV-2. The SARS-CoV escape mutant L443R was selected using nCoV-1. In particular, residue 443 (455 according to SARS-CoV-2 numbering) is phenylalanine instead of leucine, residue 460 (473 according to SARS-CoV-2 numbering) is tyrosine instead of phenylalanine, and the residue 462 (475 according to SARS-CoV-2 numbering) is alanine instead of proline (FIGS. 6A and 6B). These three critical residues (443, 460 and 462) are all in the interface with ACE2 and therefore are part of the receptor binding motif (FIG. 6A, 6B, and 7). These data, combined with the competition data between nCoV-10 and nCoV-1 (for which antibody the epitope is defined at the structural level), provide further evidence that nCoV-10 recognizes an epitope in the receptor binding motif of SARS-CoV-2 (FIG. 8).

The overall sequence similarity between SARS-CoV-2 S and SARS-CoV S is around 75%-77% for the RBD, and only approximately 50% for the RBM (FIG. 6A). Taken together, these findings indicate that nCoV-10 is uniquely able to cross-react to epitopes in the receptor binding motif (i.e. at the interface between RBD and ACE2) of both SARS-CoV and SARS-CoV-2. The SARS-CoV neutralizing antibody nCoV-6 might recognize a distinct epitope outside the RBM and due to the lack of competition with nCoV-10 could be combined (as a mix or in multi-specific formats) with it to increase barrier to resistance as well as to provide potential additive or synergistic effects on SARS-CoV-2 neutralization.

### Example 4

### Blocking Binding of RBD to the Human ACE2 Receptor

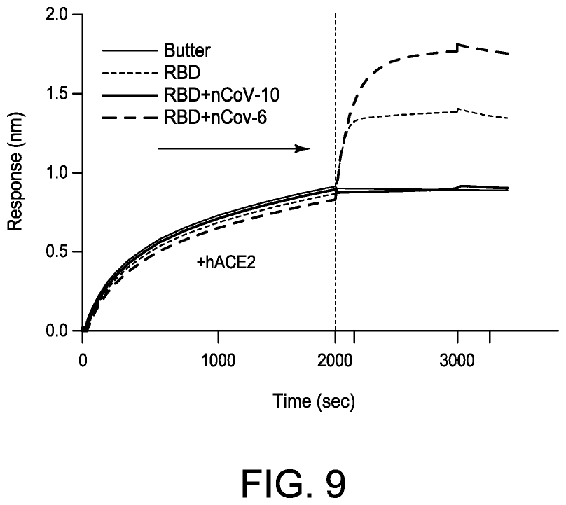

To test whether nCoV-10 and nCoV-6 can inhibit binding of the RBD to the human ACE2 receptor, Octet experiments were conducted in which SARS-CoV RBD was incubated with nCoV-6 or nCoV-10 antibodies and the formed complexes were then evaluated for binding to solid-phase hACE2 (i.e. on Octet pins). By this analysis, nCOV-10, but not nCoV-6, could clearly block the binding of SARS-CoV RBD to hACE2 (FIG. 9). This inhibitory activity of nCoV-10 is expected to correlate with the neutralizing activity of the antibody against SARS-CoV (for which hACE2 is the principal receptor for viral entry) and possibly also against SARS-CoV-2 (for which hACE2 is currently also described to be the principal receptor for viral entry).

### Example 5

### Conservation of RBD in SARS-CoV-2 Sequences

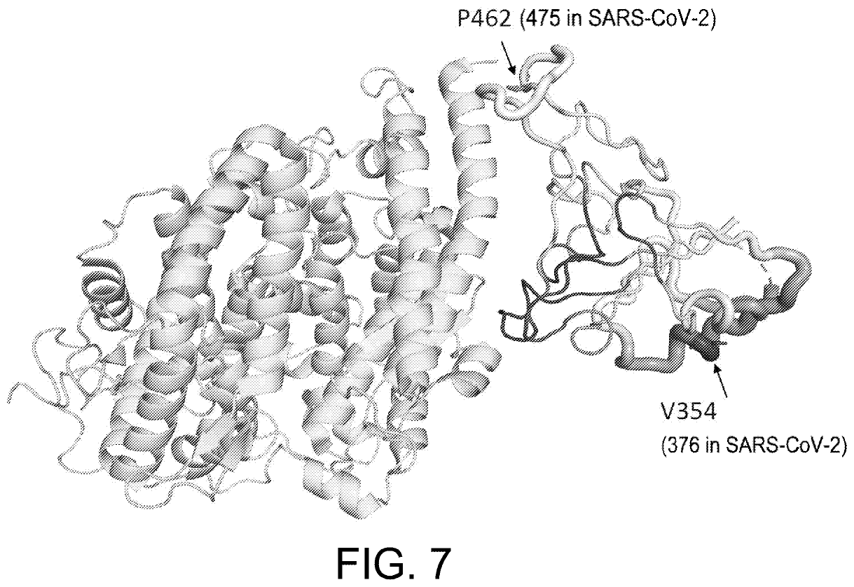

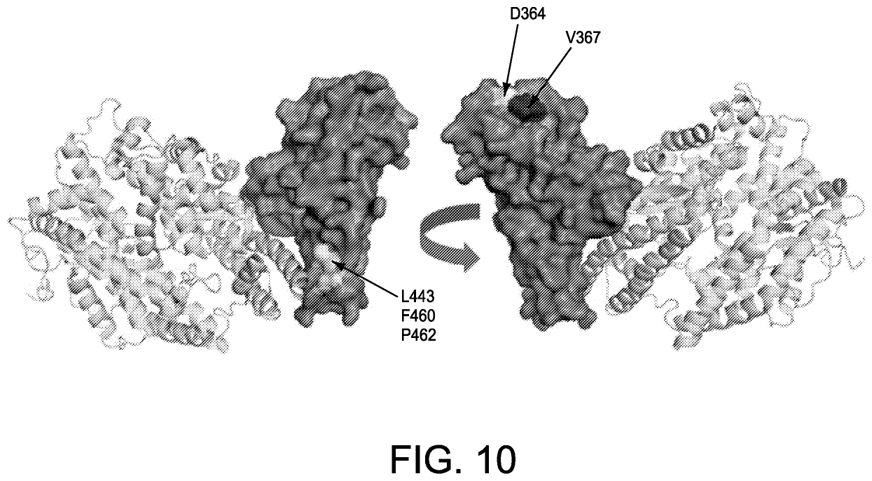

Analysis of all publicly available full genome sequences of SARS-CoV-2 as to Feb. 7, 2020 (n=71) revealed that SARS-CoV-2 RBD is highly conserved. The only exception is the appearance of mutations V367F (V354 numbering in SARS-CoV RBD) in two isolates from France (BetaCoV/France/IDF0372/2020 and BetaCoV/France/IDF0373/2020) and D364Y (V351 numbering in SARS-CoV RBD) (FIG. 7) in other isolates recently published. The residue V367 (V354 in SARS-CoV) was analysed on the structure of SARS-CoV RBD (pdb, 2AJF). Both residues D364 and V367 were found to be positioned outside of the receptor binding motif and opposite in the RBD to the residues L443, F460 and P462 (475 in SARS-CoV-2) (FIG. 10).

### Example 6

### SARS-CoV-2 Neutralization Assay

Replication-incompetent viruses pseudotyped with the SARS-CoV-2 S gene (isolate BetaCoV/Wuhan-Hu-1/2019; accession number MN908947) were produced using methods as previously described (Temperton NJ, et al. (2005) Longitudinally profiling neutralizing antibody response to SARS coronavirus with pseudotypes. Emerg Infect Dis 11(3):411-416.). Briefly, HEK293T/17 was cotransfected with a SARS-CoV-2 S-expressing plasmid (phCMV1, Genlantis) and with a complementing viral-genome reporter gene vector, pNL4-3. Luc+.E-R+. A single-cycle infectivity assay was used to measure the neutralization of luciferase-encoding virions pseudotyped with the SARS-CoV-2 S protein, as previously described (Temperton NJ, et al. (2007). A sensitive retroviral pseudotype assay for influenza H5N1-neutralizing antibodies. Influenza Other Respi Viruses 1(3):105-112.). Briefly, appropriate dilutions of the virion-containing culture supernatants were preincubated at 37° C. for 1 h with antibodies at various concentrations and the virus-mAb mixtures was then added to Vero E6 cells that were seeded the day before infection. The cells were then lysed with Steady-Glo reagent (Promega, E2520), and the relative luminescence units (RLU) in the cell lysates was determined on a luminometer microplate reader (Synergy H1 Hybrid Multi-Mode Reader; Biotek). The reduction of infectivity was determined by comparing the RLU in the presence and absence of antibody and expressed as percentage of neutralization.

### Example 7

### Inhibition of SARS-S1 Association with ACE2

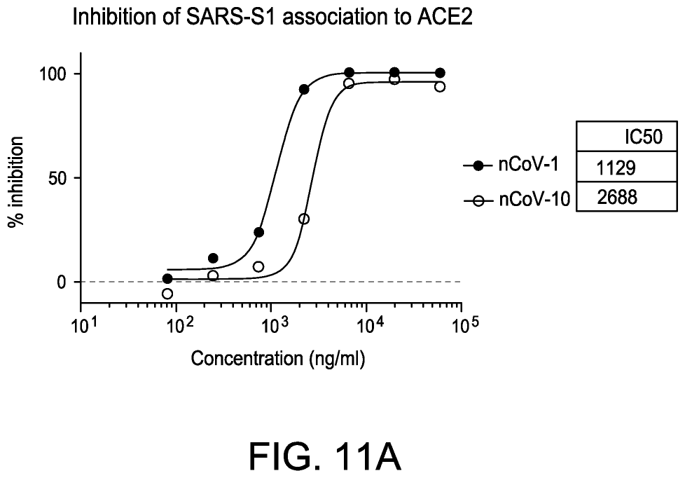

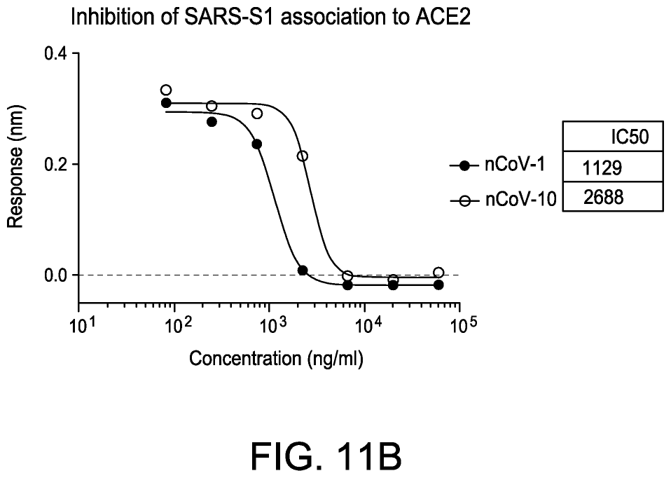

The ability of monoclonal antibodies nCoV-1 and nCoV-10 to inhibit association of SARS-S1 protein with ACE2 was assayed by Octet (BLI). SARS-CoV S1 was incubated with nCoV-1 or nCoV-10 antibodies and the formed complexes were then evaluated for binding to solid-phase hACE2 (i.e. on Octet pins). Results are shown in FIGS. 11A and 11B and in Table 4. FIG. 11A shows % inhibition on the y-axis. FIG. 11B shows response on the y-axis. In these experiments, the IC50 for nCoV-1 was calculated to be 1129 ng/ml. The IC50 for nCoV-10 was calculated to be 2688 ng/ml.

TABLE 4

| | Concentration | Response | % Inhibition |
|---|---|---|---|
| Antibody | (ng/ml) | (60 sec) | (60 sec) |
| nCoV-1 | 60000 | −0.0174 | 100 |
| | 20000 | −0.0181 | 100.2115443 |
| | 6666.7 | −0.0181 | 100.2115443 |
| | 2222.2 | 0.0086 | 92.14264128 |
| | 740.74 | 0.2353 | 23.63251738 |
| | 246.91 | 0.2758 | 11.39317014 |
| | 82.30 | 0.3091 | 1.32970686 |
| | No mAb | 0.3165 | −0.906618314 |
| nCoV-10 | 60000 | 0.0037 | 93.62345119 |
| | 20000 | −0.0081 | 97.18948323 |
| | 6666.7 | −0.0014 | 95.16470233 |
| | 2222.2 | 0.2141 | 30.03928679 |
| | 740.74 | 0.2898 | 7.162284678 |
| | 246.91 | 0.3037 | 2.961619825 |
| | 82.30 | 0.3328 | −5.832577818 |
| | No mAb | 0.3105 | 0.906618314 |

### Example 8

### Materials and Methods

### Transient Expression of Recombinant SARS-CoV-2 Protein

The full-length S gene of SARS-CoV-2 strain (SARS-CoV-2-S) isolate BetaCoV/Wuhan-Hu-1/2019 (accession number MN908947) was codon optimized for human cell expression and cloned into the phCMV1 expression vector (Genlantis). Expi-CHO cells were transiently transfected with phCMV1-2019-nCoV-S, phCMV1-MERS-CoV-S (Londonl/2012), pl.18-SARS-CoV-S (Urbani strain) or the empty phCMV1 (Mock) using Expifectamine CHO Enhancer. One and two days after transfection, cells were collected, fixed, or fixed and permeabilized with saponin for immunostaining with a panel of 21 monoclonal antibodies reactive to SARS-CoV Receptor Binding Domain (RBD). An Alexa647-labelled secondary antibody anti-human IgG Fc was used for detection. Binding of antibodies to transfected cells was analyzed by flow-cytometry using a ZE5 Cell Analyzer (Biorad) and FlowJo software (TreeStar). Positive binding was defined by differential staining of CoV-S-transfectants versus mock-transfectants.

### Competition Experiments Using Octet (BLI, Biolayer Interferometry)

Anti-His sensors (BIOSENSOR ANTI-PENTA-HIS (HIS1K)) were used to immobilize the S1 subunit protein of SARS-CoV (Sino Biological Europe GmbH). Sensors were hydrated for 10 min with Kinetics Buffer (KB; 0.01% endotoxin-free BSA, 0.002\hat{ } Tween-20, 0.005% NaN3 in PBS). SARS-CoV S1 subunit protein was then loaded for 8 min at a concentration of 10 μg/ml in KB. Antibodies were associated for 6 min at 15 μg/ml for full length mAbs nCoV-10 and nCov-6 mAbs or 5 μg/ml for Fab nCoV-4, and in a subsequent experiment comprising nCoV-1 all at 10 μg/ml. Competing antibodies were then associated at the same concentration for additional 6 minutes.

### Competition Experiments Using Octet (BLI, Biolayer Interferometry)

Anti-His sensors (BIOSENSOR ANTI-PENTA-HIS (HIS1K)) were used to immobilize human ACE2 (at 5 μg/ml; R&D). Sensors were hydrated for 10 min with Kinetics Buffer (KB; 0.01% endotoxin-free BSA, 0.002\hat{ } Tween-20, 0.005% NaN3 in PBS). Human ACE2 was then loaded for 30 min at a concentration of 5 μg/ml in KB. Antibodies (2 μg/ml) were mixed with SARS-CoV RBD-Fc (Sino Biological Europe GmbH, 2 μg/ml) and exposed to human ACE2 loaded sensors for an association time of 15 min, followed by a dissociation step of 6 minutes.

### ELISA Binding

The reactivities of mAbs with SARS-CoV Spike S1 Subunit Protein (strain WH20) protein were determined by enzyme-linked immunosorbent assays (ELISA). Briefly, 96-well plates were coated with 3 μg/ml of recombinant SARS-CoV Spike S1 Subunit Protein (Sino. Biological). Wells were washed and blocked with PBS+1% BSA for 1 h at room temperature and were then incubated with serially diluted mAbs for 1 h at room temperature. Bound mAbs were detected by incubating alkaline phosphatase-conjugated goat anti-human IgG (Southern Biotechnology: 2040-04) for 1 h at room temperature and were developed by 1 mg/ml p-nitrophenylphosphate substrate in 0.1 M glycine buffer (pH 10.4) for 30 min at room temperature. The optical density (OD) values were measured at a wavelength of 405 nm in an ELISA reader (Powerwave 340/96 spectrophotometer, BioTek).

### Example 9

### Additional Experiments

SARS-CoV-2 RBDs (two variants) are synthesized with C-terminal peptide tags (Strep-Tag II; His-Tag) for purification and labelling. A full-length spike protein from SARS-CoV-2 is synthesized to produce SARS-CoV-2 pseudoviruses, and the pseudoviruses are used in neutralization assays and membrane expression studies.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 62/969,592, filed Feb. 3, 2020, U.S. Patent Application No. 62/970,062, filed Feb. 4, 2020, U.S. Patent Application No. 62/971,552, filed Feb. 7, 2020, U.S. Patent Application No. 62/977,941, filed Feb. 18, 2020, U.S. Patent Application No. 63/016,228, filed Apr. 27, 2020, and U.S. Patent Application No. 63/023,858, filed May 12, 2020, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antibody, or an antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein the antibody or antigen binding fragment is capable of binding to a surface glycoprotein of SARS-CoV-2 expressed on a cell surface of a host cell and/or on a virion.

2. The antibody, or an antigen-binding fragment of claim 1, wherein the antibody or antigen binding fragment is capable of binding to the Receptor Binding Domain (RBD) of a surface glycoprotein of SARS-CoV-2 and/or SARS-CoV expressed on a cell surface of a host cell and/or on a virion.

3. The antibody or antigen-binding fragment of claim 1, which is capable of neutralizing a SARS-CoV-2 infection in an in vitro model of infection and/or in an in vivo animal model of infection and/or in a human.

4. An antibody, or antigen-binding fragment thereof, comprising a heavy chain variable domain (VH) comprising a CDRH1, a CDRH2, and a CDRH3, and a light chain variable domain (VL) comprising a CDRL1, a CDRL2, and a CDRL3, wherein:
(i) the CDRH1 comprises or consists of the amino acid sequence according to any one of SEQ ID NOs.: 6, 22, 38, 54, 70, 86, 102, 118, 134, 150, 166, 182, 198, 214, 230, 246, 262, 278, 294, 310, 326, 342, or 358, or a sequence variant thereof comprising one, two, or three acid substitutions, one or more of which substitutions is optionally a conservative substitution and/or is a substitution to a germline-encoded amino acid;
(ii) the CDRH2 comprises or consists of the amino acid sequence according to any one of SEQ ID NOs.: 7, 23, 39, 55, 71, 87, 103, 119, 135, 151, 167, 183, 199, 215, 231, 247, 263, 279, 295, 311, 327, 343, 359, or 416, or a sequence variant thereof comprising one, two, or three amino acid substitutions, one or more of which substitutions is optionally a conservative substitution and/or is a substitution to a germline-encoded amino acid;
(iii) the CDRH3 comprises or consists of the amino acid sequence according to any one of SEQ ID NOs.: 8, 24, 40, 56, 72, 88, 104, 120, 136, 152, 168, 184, 200, 216, 232, 248, 264, 280, 296, 312, 328, 344, 360, 375, 378, or 397, or a sequence variant thereof comprising one, two, or three amino acid substitutions, one or more of which substitutions is optionally a conservative substitution and/or is a substitution to a germline-encoded amino acid;
(iv) the CDRL1 comprises or consists of the amino acid sequence according to any one of SEQ ID NOs.: 14, 30, 46, 62, 78, 94, 110, 126, 142, 158, 174, 190, 206, 222, 238, 254, 270, 286, 302, 318, 334, 350, 366, 398, 399, 401, 403, or 405, or a sequence variant thereof comprising one, two, or three amino acid substitutions, one or more of which substitutions is optionally a conservative substitution and/or is a substitution to a germline-encoded amino acid;
(v) the CDRL2 comprises or consists of the amino acid sequence according to any one of SEQ ID NOs.: 15, 31, 47, 63, 79, 95, 111, 127, 143, 159, 175, 191, 207, 223, 239, 255, 271, 287, 303, 319, 335, 351, or 367, or a sequence variant thereof comprising one, two, or three amino acid substitutions, one or more of which substitutions is optionally a conservative substitution and/or is a substitution to a germline-encoded amino acid; and/or
(vi) the CDRL3 comprises or consists of the amino acid sequence according to any one of SEQ ID NOs.: 16, 32, 48, 64, 80, 96, 112, 128, 144, 160, 176, 192, 208, 224, 240, 256, 272, 288, 304, 320, 336, 352, 358, 386, or 394, or a sequence variant thereof comprising having one, two, or three amino acid substitutions, one or more of which substitutions is optionally a conservative substitution and/or is a substitution to a germline-encoded amino acid,
wherein the antibody or antigen binding fragment is capable of binding to a surface glycoprotein of SARS-CoV-2 expressed on a cell surface of a host cell and/or on a virion.

5. The antibody or antigen-binding fragment of claim 4, which is capable of neutralizing a SARS-CoV-2 infection in an in vitro model of infection and/or in an in vivo animal model of infection and/or in a human.

6. The antibody or antigen-binding fragment of claim 4, comprising CDRH1, CDRH2, CDRH3, CDRL1, CDRL2, and CDRL3 amino acid sequences according to SEQ ID NOs.:
(i) 6-8 and 14-16, respectively;
(ii) 22-24 and 30-32, respectively;
(iii) 22-24, 398, 31, and 32, respectively;
(iv) 22-24, 399, 31, and 32, respectively;
(v) 22-24, 401, 31, and 32, respectively;
(vi) 22-24, 403, 31, and 32, respectively;
(v) 22-24, 405, 31, and 32, respectively;
(vi) 38-40 and 46-48, respectively;
(vii) 38-40, 398, 47, and 48, respectively;
(viii) 38-40, 399, 47, and 48, respectively;
(viv) 38-40, 401, 47, and 48, respectively;
(x) 38-40, 403, 47, and 48, respectively;
(xi) 38-40, 405, 47, and 48, respectively;
(xii) 54-56 and 62-64, respectively;
(xiii) 70-72 and 78-80, respectively;
(xiv) 86-88 and 94-96, respectively;
(xv) 102-104 and 110-112, respectively;
(xvi) 118-120 and 126-128, respectively;
(xvii) 134-136 and 142-144, respectively;
(xviii) 150-152 and 158-160, respectively;
(xix) 166-168 and 174-176, respectively;
(xx) 182-184 and 190-192, respectively;
(xxi) 198-200 and 206-208, respectively;
(xxii) 214-216 and 222-224, respectively;
(xxiii) 230-232 and 238-240, respectively;
(xxiv) 246-248 and 254-256, respectively;
(xxv) 262-264 and 270-272, respectively;
(xxxvi) 278-280 and 286-288, respectively;
(xxvii) 294-296 and 302-304, respectively;
(xxviii) 310-312 and 318-320, respectively;
(xxix) 326-328 and 334-336, respectively;
(xxx) 342-344 and 350-352, respectively;
(xxxi) 358-360 and 366-368, respectively;
(xxxii) 22, 23, 375, and 30-32, respectively;
(xxxiii) 22, 23, 375, 398, 31, and 32, respectively;
(xxxiv) 22, 23, 375, 399, 31, and 32, respectively;
(xxxv) 22, 23, 375, 401, 31, and 32, respectively;
(xxxvi) 22, 23, 375, 403, 31, and 32, respectively;
(xxxvii) 22, 23, 375, 405, 31, and 32, respectively;
(xxxviii) 22, 23, 378, and 30-32, respectively;
(xxxix) 22, 23, 378, 398, 31, and 32, respectively;
(xl) 22, 23, 378, 399, 31, and 32, respectively;
(xli) 22, 23, 378, 401, 31, and 32, respectively;
(xlii) 22, 23, 378, 403, 31, and 32, respectively;
(xliii) 22, 23, 378, 405, 31, and 32, respectively;
(xliv) 22-24, 30, 31, and 386, respectively;
(xlv) 22-24, 398, 31, and 386, respectively;
(xlvi) 22-24, 399, 31, and 386, respectively;
(xlvii) 22-24, 401, 31, and 386, respectively;
(xlviii) 22-24, 403, 31, and 386, respectively;
(xlix) 22-24, 405, 31, and 386, respectively;
(l) 38-40, 46, 47, and 386, respectively;
(li) 38-40, 398, 47, and 386, respectively;
(lii) 38-40, 399, 47, and 386, respectively;
(liii) 38-40, 401, 47, and 386, respectively;
(liv) 38-40, 403, 47, and 386, respectively;
(lv) 38-40, 405, 47, and 386, respectively;
(lvi) 22-24, 30, 31, and 394, respectively;
(lvii) 22-24, 398, 31, and 394, respectively;
(lviii) 22-24, 399, 31, and 394, respectively;
(lix) 22-24, 401, 31, and 394, respectively;
(lx) 22-24, 403, 31, and 394, respectively;
(lxi) 22-24, 405, 31, and 394, respectively;
(lxii) 22, 23, 375, 30, 31, and 394, respectively;
(lxiii) 22, 23, 375, 30, 31, and 386, respectively;
(lxiv) 22, 23, 375, 398, 31, and 386, respectively;
(lxv) 22, 23, 375, 399, 31, and 386, respectively;
(lxvi) 22, 23, 375, 401, 31, and 386, respectively;
(lxvii) 22, 23, 375, 403, 31, and 386, respectively;
(lxviii) 22, 23, 375, 405, 31, and 386, respectively;
(lxix) 22, 23, 378, 30, 31, and 394, respectively;
(lxx) 22, 23, 378, 398, 31, and 394, respectively;
(lxxi) 22, 23, 378, 399, 31, and 394, respectively;
(lxxii) 22, 23, 378, 401, 31, and 394, respectively;
(lxxiii) 22, 23, 378, 403, 31, and 394, respectively;
(lxxiv) 22, 23, 378, 405, 31, and 394, respectively;
(lxxv) 22, 23, 378, 30, 31, and 386, respectively;
(lxxvi) 22, 23, 378, 398, 31, and 386, respectively;
(lxxvii) 22, 23, 378, 399, 31, and 386, respectively;
(lxxviii) 22, 23, 378, 401, 31, and 386, respectively;
(lxxix) 22, 23, 378, 403, 31, and 386, respectively;
(lxxx) 22, 23, 378, 405, 31, and 386, respectively;
(lxxxi) 22, 23, 397, and 30-32, respectively;
(lxxxii) 22, 23, 397, 398, 31, and 32, respectively;
(lxxxiii) 22, 23, 397, 399, 31, and 32, respectively;
(lxxxiv) 22, 23, 397, 401, 31, and 32, respectively;
(lxxxv) 22, 23, 397, 403, 31, and 32, respectively;
(lxxxvi) 22, 23, 397, 405, 31, and 32, respectively;
(lxxxvii) 22, 23, 397, 30, 31, and 386, respectively;
(lxxxviii) 22, 23, 397, 398, 31, and 386, respectively;
(lxxxix) 22, 23, 397, 399, 31, and 386, respectively;
(xc) 22, 23, 397, 401, 31, and 386, respectively;
(xci) 22, 23, 397, 403, 31, and 386, respectively;
(xcii) 22, 23, 397, 405, 31, and 386, respectively;
(xciii) 22, 23, 397, 30, 31, and 394, respectively;
(xciv) 22, 23, 397, 398, 31, and 394, respectively;
(xcv) 22, 23, 397, 399, 31, and 394, respectively;
(xcvi) 22, 23, 397, 401, 31, and 394, respectively;
(xcvii) 22, 23, 397, 403, 31, and 394, respectively;
(xcviii) 22, 23, 397, 405, 31, and 394, respectively;
(xcix) 22, 416, 24, 30, 31, and 32, respectively;
(c) 22, 416, 24, 30, 31, and 386, respectively;
(ci) 22, 416, 24, 30, 31, and 394, respectively;
(cii) 22, 416, 24, 398, 31, and 32, respectively;
(ciii) 22, 416, 24, 398, 31, and 386, respectively;
(civ) 22, 416, 24, 398, 31, and 394, respectively;
(cv) 22, 416, 24, 399, 31, and 32, respectively;
(cvi) 22, 416, 24, 399, 31, and 386, respectively;
(cvii) 22, 416, 24, 399, 31, and 394, respectively;
(cviii) 22, 416, 24, 401, 31, and 32, respectively;
(cix) 22, 416, 24, 401, 31, and 386, respectively;
(cx) 22, 416, 24, 401, 31, and 394, respectively;
(cxi) 22, 416, 24, 403, 31, and 32, respectively;
(cxii) 22, 416, 24, 403, 31, and 386, respectively;
(cxiii) 22, 416, 24, 403, 31, and 394, respectively;
(cxiv) 22, 416, 24, 405, 31, and 32, respectively;
(cxv) 22, 416, 24, 405, 31, and 386, respectively;
(cxvi) 22, 416, 24, 405, 31, and 394, respectively;
(cxvii) 22, 416, 375, 30, 31, and 32, respectively;
(cxviii) 22, 416, 375, 398, 31, and 386, respectively;
(cxix) 22, 416, 375, 398, 31, and 394, respectively;
(cxx) 22, 416, 375, 399, 31, and 386, respectively;
(cxxi) 22, 416, 375, 399, 31, and 394, respectively;
(cxxii) 22, 416, 375, 401, 31, and 386, respectively;
(cxxiii) 22, 416, 375, 401, 31, and 394, respectively;
(cxxiv) 22, 416, 375, 403, 31, and 386, respectively;
(cxxv) 22, 416, 375, 403, 31, and 394, respectively;
(cxxvi) 22, 416, 375, 405, 31, and 386, respectively;
(cxxvii) 22, 416, 375, 405, 31, and 394, respectively;
(cxxviii) 22, 416, 378, 30, 31, and 32, respectively;
(cxxix) 22, 416, 378, 398, 31, and 386, respectively;
(cxxx) 22, 416, 378, 398, 31, and 394, respectively;
(cxxxi) 22, 416, 378, 399, 31, and 386, respectively;
(cxxxii) 22, 416, 378, 399, 31, and 394, respectively;
(cxxxiii) 22, 416, 378, 401, 31, and 386, respectively;
(cxxxiv) 22, 416, 378, 401, 31, and 394, respectively;
(cxxxv) 22, 416, 375, 403, 31, and 386, respectively;
(cxxxvi) 22, 416, 378, 403, 31, and 394, respectively;
(cxxxvii) 22, 416, 378, 405, 31, and 386, respectively;
(cxxxviii) 22, 416, 378, 405, 31, and 394, respectively;
(cxxxix) 22, 416, 397, 30, 31, and 32, respectively;
(cxl) 22, 416, 397, 398, 31, and 386, respectively;
(cxli) 22, 416, 397, 398, 31, and 394, respectively;
(cxlii) 22, 416, 397, 399, 31, and 386, respectively;
(ccxliii) 22, 416, 397, 399, 31, and 394, respectively;
(cxliv) 22, 416, 397, 401, 31, and 386, respectively;
(cxlv) 22, 416, 397, 401, 31, and 394, respectively;
(cxlvi) 22, 416, 375, 403, 31, and 386, respectively;
(cxlvii) 22, 416, 397, 403, 31, and 394, respectively;
(cxlviii) 22, 416, 397, 405, 31, and 386, respectively; or
(cxlix) 22, 416, 397, 405, 31, and 394, respectively.

7. The antibody or antigen-binding fragment of claim 1, wherein:
(i) the VH comprises or consists of an amino acid sequence having at least 85% identity to the amino acid sequence according to any one of SEQ ID NOs.: 5, 21, 37, 53, 69, 85, 101, 117, 133, 149, 165, 181, 197, 213, 229, 245, 261, 277, 293, 309, 325, 341, 357, 374, 377, 396, and 415, wherein the variation, if present, is optionally limited to one or more framework regions and/or the variation, if present, comprises one or more substitution to a germline-encoded amino acid; and/or
(ii) the VL comprises or consists of an amino acid sequence having at least 85% identity to the amino acid sequence according to any one of SEQ ID NOs.: 13, 29, 45, 61, 77, 93, 109, 125, 141, 157, 173, 189, 205, 221, 237, 253, 269, 285, 301, 317, 333, 349, 365, 380, 383, 385, 388, 392, 393, 400, 402, 404, and 406-414, wherein the variation, if present, is optionally limited to one or more framework regions and/or the variation, if present, comprises one or more substitution to a germline-encoded amino acid.

8. The antibody or antigen-binding fragment of claim 1, wherein the VH comprises or consists of any VH amino acid sequence set forth in Table 1, and wherein the VL comprises or consists of any VL amino acid sequence set forth in Table 1, wherein, optionally, the VH and the VL comprise or consist of the amino acid sequences according to SEQ ID NOs.:
(i) 5 and 13, respectively;
(ii) 21 and any one of 29, 45, 380, 383, 385, 388, 392, 393, 400, 402, 404, 406, 407, 408, 409, 410, 411, 412, 413, and 414, respectively;
(iii) 415 and any one of 29, 45, 380, 383, 385, 388, 392, 400, 402, 404, 406, 407, 408, 409, 410, 411, 412, 413, and 414, respectively;
(iv) 37 and any one of 29, 45, 380, 383, 385, 388, 392, 400, 402, 404, 406, 407, 408, 409, 410, 411, 412, 413, and 414, respectively;
(v) 53 and 61, respectively;
(vi) 69 and 77, respectively;
(vii) 85 and 93, respectively;
(viii) 101 and 109, respectively;
(ix) 117 and any one of 125, 141, and 157, respectively;
(x) 133 and any one of 125, 141, and 157, respectively;
(xi) 149 and any one of 125, 141, and 157, respectively;
(xii) 165 and 173, respectively;
(xiii) 181 and 189, respectively;
(xiv) 197 and 205, respectively;
(xv) 213 and 221, respectively;
(xvi) 229 and 237, respectively;
(xvii) 245 and 253, respectively;
(xviii) 261 and 269, respectively;
(xix) 277 and 285, respectively;
(xx) 293 and 301, respectively;
(xxi) 309 and 317, respectively;
(xxii) 325 and 333, respectively;
(xxiii) 341 and 349, respectively;
(xxiv) 357 and 365, respectively;
(xxv) 374 and any one of 29, 45, 380, 383, 385, 392, 393, 400, 402, 404, 406, 407, 408, 409, 410, 411, 412, 413, and 414, respectively;
(xxvi) 377 and any one of 29, 45, 380, 383, 385, 392, 393, 400, 402, 404, 406, 407, 408, 409, 410, 411, 412, 413, and 414, respectively;
(xxvii) 415 and any one of 29, 45, 380, 383, 385, 392, 393, 400, 402, 404, 406, 407, 408, 409, 410, 411, 412, 413, and 414, respectively; or
(xxviii) 396 and any one of 29, 37, 45, 380, 383, 385, 388, 392, 393, 400, 402, 404, 406, 407, 408, 409, 410, 411, 412, 413, and 414, respectively.

9. The antibody or antigen-binding fragment of claim 1, which:
(i) recognizes an epitope in the ACE2 receptor binding motif (RBM, SEQ ID NO.:390) of SARS-CoV-2;
(ii) is capable of blocking an interaction between SARS-CoV-2 (e.g., SARS-CoV-2 RBM) and ACE2;
(ii) is capable of binding to SARS-CoV-2 S protein with greater avidity than to SARS coronavirus S protein;
(iv) is capable of staining about 30%, about 35%, about 40%, about 50%, about 55%, about 56%, about 57%, about 58%, about 59%, about 60%, or more of target cells expressing SARS-CoV-2 surface glycoprotein in a sample comprising about 50,000 of the target cells in approximately 100 μL when the antibody or antigen-binding fragment is present at 10 μg/ml;
(v) recognizes an epitope that is conserved in the ACE2 RBM of SARS-CoV-2 and in an ACE2 RBM of SARS coronavirus;
(vi) is cross-reactive against SARS-CoV-2 and SARS coronavirus;
(vii) recognizes an epitope in the SARS-CoV-2 surface glycoprotein that is not in the ACE2 RBM; or
(viii) any combination of (i)-(vii).

10. The antibody or antigen-binding fragment of claim 4, which is a IgG, IgA, IgM, IgE, or IgD isotype.

11. The antibody or antigen-binding fragment of claim 4, which is an IgG isotype selected from IgG1, IgG2, IgG3, and IgG4.

12. The antibody or antigen-binding fragment of claim 4, which is human, humanized, or chimeric.

13. The antibody or antigen-binding fragment of claim 4, wherein the antibody, or the antigen-binding fragment, comprises a human antibody, a monoclonal antibody, a purified antibody, a single chain antibody, a Fab, a Fab′, a F(ab′)2, a Fv, or a scFv.

14. The antibody or antigen-binding fragment of claim 4, wherein the antibody or antigen-binding fragment is a multi-specific antibody or antigen binding fragment.

15. The antibody or antigen-binding fragment of claim 14, wherein the antibody or antigen binding fragment is a bispecific antibody or antigen-binding fragment.

16. The antibody or antigen-binding fragment of claim 14, comprising:
(i) a first VH and a first VL; and
(ii) a second VH and a second VL,
wherein the first VH and the second VH are different and each independently comprise an amino acid sequence having at least 85% identity to the amino acid sequence set forth in any one of SEQ ID NOs.: 5, 21, 37, 53, 69, 85, 101, 117, 133, 149, 165, 181, 197, 213, 229, 245, 261, 277, 293, 309, 325, 341, 357, 374, 377, 396, and 415, and
wherein the first VL and the second VL are different and each independently comprise an amino acid sequence having at least 85% identity to the amino acid sequence set forth in any one of SEQ ID NOs.: 13, 29, 45, 61, 77, 93, 109, 125, 141, 157, 173, 189, 205, 221, 237, 253, 269, 285, 301, 317, 333, 349, 365, 380, 383, 385, 388, 392, 393, 400, 402, 404, and 406-414,
and wherein the first VH and the first VL together form a first antigen-binding site, and wherein the second VH and the second VL together form a second antigen-binding site.

17. The antibody or antigen-binding fragment of claim 16, wherein:
(i) the first VH comprises or consists of the amino acid sequence set forth in any one of SEQ ID NOs.:21, 37, 374, 377, 396, and 415, and the first VL comprises or consists of the amino acid sequence set forth in any one of SEQ ID NOs.:29, 45, 380, 383, 385, 388, 392, 393, 400, 402, 404, and 406-414; and
(ii) the second VH comprises or consists of the amino acid sequence set forth in SEQ ID NO.:69 and the second VL comprises or consists of the amino acid sequence set forth in SEQ ID NO.: 77.

18. The antibody or antigen-binding fragment of claim 4, wherein the antibody or antigen-binding fragment further comprises a Fc polypeptide or a fragment thereof.

19. The antibody or antigen-binding fragment of claim 18, wherein the Fc polypeptide or fragment thereof comprises:
(i) a mutation that enhances binding to a FcRn as compared to a reference Fc polypeptide that does not comprise the mutation; and/or
(ii) a mutation that enhances binding to a FcγR as compared to a reference Fc polypeptide that does not comprise the mutation.

20. The antibody or antigen-binding fragment of claim 19, wherein the mutation that enhances binding to a FcRn comprises: M428L; N434S; N434H; N434A; N434S; M252Y; S254T; T256E; T250Q; P257I; Q311I; D376V; T307A; E380A; or any combination thereof.

21. The antibody or antigen-binding fragment of claim 19, wherein the mutation that enhances binding to FcRn comprises:
(i) M428L/N434S;
(ii) M252Y/S254T/T256E;
(iii) T250Q/M428L;
(iv) P257I/Q311I;
(v) P257I/N434H;
(vi) D376V/N434H;
(vii) T307A/E380A/N434A; or
(viii) any combination of (i)-(vii).

22. The antibody or antigen-binding fragment of claim 19, wherein the mutation that enhances binding to FcRn comprises M428L/N434S.

23. The antibody or antigen-binding fragment of claim 19, wherein the mutation that enhances binding to a FcγR comprises: S239D; I332E; A330L; G236A; or any combination thereof.

24. The antibody or antigen-binding fragment of claim 19, wherein the mutation that enhances binding to a FcγR comprises:
(i) S239D/I332E;
(ii) S239D/A330L/I332E;
(iii) G236A/S239D/I332E; or
(iv) G236A/A330L/I332E.

25. The antibody or antigen-binding fragment claim 4, which comprises a mutation that alters glycosylation, wherein the mutation that alters glycosylation comprises N297A, N297Q, or N297G, and/or which is aglycosylated and/or afucosylated.

26. An isolated polynucleotide encoding the antibody or antigen-binding fragment of claim 1, or encoding a VH, a heavy chain, a VL, and/or a light chain of the antibody or the antigen-binding fragment.

27. The polynucleotide of claim 28, wherein the polynucleotide comprises deoxyribonucleic acid (DNA) or ribonucleic acid (RNA), wherein the RNA optionally comprises messenger RNA (mRNA).

28. The polynucleotide of claim 26, which is codon-optimized for expression in a host cell.

29. The polynucleotide of claim 26, comprising a polynucleotide having at least 50% identity to the polynucleotide sequence according to any one or more of SEQ ID NOs.:1-4, 9-12, 17-20, 25-28, 33-36, 41-44, 49-52, 57-60, 65-68, 73-76, 81-84, 89-92, 97-100, 105-108, 113-116, 121-124, 129-132, 137-140, 145-148, 153-156, 161-164, 169-172, 177-180, 185-188, 193-196, 201-204, 209-212, 217-220, 225-228, 233-236, 241-244, 249-252, 257-260, 265-268, 273-276, 281-284, 289-292, 297-300, 305-308, 313-316, 321-324, 329-332, 337-340, 345-348, 353-356, 361-364, 372, 373, 376, 379, 381, 384, 387, 389 and 417.

30. A recombinant vector comprising the polynucleotide of claim 29.

31. A host cell comprising the polynucleotide of claim 26, wherein the polynucleotide is heterologous to the host cell.

32. A human B cell comprising the polynucleotide of claim 26, wherein the polynucleotide is heterologous to the human B cell and/or wherein the human B cell is immortalized.

33. A composition comprising:
the antibody or antigen-binding fragment of claim 4,
and a pharmaceutically acceptable excipient, carrier, or diluent.

34. The composition of claim 33, comprising two or more different antibodies or antigen-binding fragments of claim 4.

35. The composition of claim 34, comprising a first antibody or antigen-binding fragment and a second antibody or antigen-binding fragment, wherein:
(i) the first antibody or antigen-binding fragment comprises a VH comprising or consisting of the amino acid sequence set forth in any one of SEQ ID NOs.:21, 37, 374, 377, 396 and 415, and a VL comprising or consisting of the amino acid sequence set forth in any one of SEQ ID NOs.:29, 45, 380, 383, 385, 388, 392, 393, 400, 402, 404, and 406-414; and
(ii) the second antibody or antigen-binding fragment comprises a VH comprising or consisting of the amino acid sequence set forth in SEQ ID NO.:69, and a VL comprising or consisting of the amino acid sequence set forth in SEQ ID NO.:77.

36. A composition comprising the polynucleotide of claim 26 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

37. A method of treating a SARS-CoV-2 infection in a subject, the method comprising administering to the subject an effective amount of
the antibody or antigen-binding fragment of claim 4.

38. The method of claim 37, comprising administering to the subject
(a) a first antibody or antigen-binding fragment of claim 4, when the subject has received a second antibody or antigen-binding fragment of claim 4;
(b) a second antibody or antigen-binding fragment of claim 4, when the subject has received a first antibody or antigen-binding fragment of claim 4; or
(c) a first antibody or antigen-binding fragment of claim 4, and a second antibody or antigen-binding fragment of claim 4,
wherein
(i) the first antibody or antigen-binding fragment comprises a VH comprising or consisting of the amino acid sequence set forth in any one of SEQ ID NOs.:21, 37, 374, 377, 396 and 415, and a VL comprising or consisting of the amino acid sequence set forth in any one of SEQ ID NOs.:29, 45, 380, 383, 385, 388, 392, 393, 400, 402, 404, and 406-414; and
(ii) the second antibody or antigen-binding fragment comprises a VH comprising or consisting of the amino acid sequence set forth in SEQ ID NO.:69, and a VL comprising or consisting of the amino acid sequence set forth in SEQ ID NO.:77.

39.-40. (canceled)

41. A method for in vitro diagnosis of a sarbecovirus infection, the method comprising:
(i) contacting a sample from a subject with an antibody or antigen-binding fragment of claim 4; and
(ii) detecting a complex comprising an antigen and the antibody, or comprising an antigen and the antigen-binding fragment.

42. The method of claim 41, wherein the sarbecovirus is SARS-CoV or SARS-CoV-2.

43. The method of claim 41, wherein the sample comprises blood isolated from the subject.

44. A method of treating a SARS-CoV-2 infection in a subject, the method comprising administering to the subject an effective amount of the antibody or antigen-binding fragment of claim 1.
